SEQUENCE LISTING (47 sequences)

SEQ ID NO: 1 - protein, 483 aa, Artificial Sequence
  Description of Artificial Sequence/note = synthetic construct
MATIEEIAHQIIEQQMGEIVTEQQTGQKIQIVTALDHNTQGKQFILTNHDGSTPSKVILARQDSTPGKVFLTTPDAAGVNQLFFTTPDLSAQHLQLLTDNSPDQGPNKVFDLCVVCGDKASGRHYGAVTCEGCKGFFKRSIRKNLVYSCRGSKDCIINKHHRNRCQYCRLQRCIAFGMKQDSVQCERKPIEVSREKSSNCAASTEKIYIRKDLRSPLTATPTFVTDSESTRSTGLLDSGMFMNIHPSGVKTESAVLMTSDKAESCQGDLSTLANVVTSLANLGKTKDLSQNSNEMSMIESLSNDDTSLCEFQEMQTNGDVSRAFDTLAKALNPGESTACQSSVAGMEGSVHLITGDSSINYTEKEGPLLSDSHVAFRLTMPSPMPEYLNVHYIGESASRLLFLSMHWALSIPSFQALGQENSISLVKAYWNELFTLGLAQCWQVMNVATILATFVNCLHNSLQQDAKVIAALIHFTRRAITDL

SEQ ID NO: 2 - protein, 467 aa, Artificial Sequence
  Description of Artificial Sequence/note = synthetic construct
MATIEEIAHQIIEQQMGEIVTEQQTGQKIQIVTALDHNTQGKQFILTNHDGSTPSKVILARQDSTPGKVFLTTPDAAGVNQLFFTTPDLSAQHLQLLTDNSPDQGPNKVFDLCVVCGDKASGRHYGAVTCEGCKGFFKRSIRKNLVYSCRGSKDCIINKHHRNRCQYCRLQRCIAFGMKQDSVQCERKPIEVSREKSSNCAASTEKIYIRKDLRSPLTATPTFVTDSESTRSTGLLDSGMFMNIHPSGVKTESAVLMTSDKAESCQGDLSTLANVVTSLANLGKTKDLSQNSNEMSMIESLSNDDTSLCEFQEMQTNGDVSRAFDTLAKALNPGESTACQSSVAGMEGSVHLITGDSSINYTEKEGPLLSDSHVAFRLTMPSPMPEYLNVHYIGESASRLLFLSMHWALSIPSFQALGQENSISLVKAYWNELFTLGLAQCWQVMNVATILATFVNCLHNSLQQAEG

SEQ ID NO: 3 - protein, 603 aa, Artificial Sequence
  Description of Artificial Sequence/note = synthetic construct
MATIEEIAHQIIEQQMGEIVTEQQTGQKIQIVTALDHNTQGKQFILTNHDGSTPSKVILARQDSTPGKVFLTTPDAAGVNQLFFTTPDLSAQHLQLLTDNSPDQGPNKVFDLCVVCGDKASGRHYGAVTCEGCKGFFKRSIRKNLVYSCRGSKDCIINKHHRNRCQYCRLQRCIAFGMKQDSVQCERKPIEVSREKSSNCAASTEKIYIRKDLRSPLTATPTFVTDSESTRSTGLLDSGMFMNIHPSGVKTESAVLMTSDKAESCQGDLSTLANVVTSLANLGKTKDLSQNSNEMSMIESLSNDDTSLCEFQEMQTNGDVSRAFDTLAKALNPGESTACQSSVAGMEGSVHLITGDSSINYTEKEGPLLSDSHVAFRLTMPSPMPEYLNVHYIGESASRLLFLSMHWALSIPSFQALGQENSISLVKAYWNELFTLGLAQCWQVMNVATILATFVNCLHNSLQQDKMSTERRKLLMEHIFKLQEFCNSMVKLCIDGYEYAYLKAIVLFSPDHPSLENMELIEKFQEKAYVEFQDYITKTYPDDTYRLSRLLLRLPALRLMNATITEELFFKGLIGNIRIDSVIPHILKMEPADYNSQIIGHIS

SEQ ID NO: 4 - protein, 483 aa, Artificial Sequence; identical to SEQ ID NO: 1
  Description of Artificial Sequence/note = synthetic construct

SEQ ID NO: 5 - protein, 629 aa, Artificial Sequence
  Description of Artificial Sequence/note = synthetic construct
MATNMEGLVQHRVGTQQVAEVPRTQTSWPESPGMTSPSPRIQIISTDSAVASPQRIQIVTDQQTGQKIQIVTAVDASGSSKQQFILTSPDGAGTGKVILASPETSSAKQLIFTTSDNLVPGRIQIVTDSASVERLLGKADVQRPQVVEYCVVCGDKASGRHYGAVSCEGCKGFFKRSVRKNLTYSCRSSQDCIINKHHRNRCQFCRLKKCLEMGMKMESVQSERKPFDVQREKPSNCAASTEKIYIRKDLRSPLIATPTFVADKDGARQTGLLDPGMLVNIQQPLIREDGTVLLAADSKAETSQGALGTLANVVTSLANLSESLNNGDASEMQPEDQSASEITRAFDTLAKALNTTDSASPPSLADGIDASGGGSIHVISRDQSTPIIEVEGPLLSDTHVTFKLTMPSPMPEYLNVHYICESASRLLFLSMHWARSIPAFQALGQDCNTSLVRACWNELFTLGLAQCAQVMSLSTILAAIVNHLQNSIQEDKLSGDRIKQVMEHIWKLQEFCNSMAKLDIDGYEYAYLKAIVLFSPDHPGLTGTSQIEKFQEKAQMELQDYVQKTYSEDTYRLARILVRLPALRLMSSNITEELFFTGLIGNVSIDSIIPYILKMETAEYNGQITGASL

SEQ ID NO: 6 - protein, 467 aa, Artificial Sequence; identical to SEQ ID NO: 2
  Description of Artificial Sequence/note = synthetic construct

SEQ ID NO: 7 - DNA, 1785 bp, Artificial Sequence
  Description of Artificial Sequence/note = synthetic construct
gaattcgggcccgtcggctttcttcaaccctctcttcccggagcgcccccaatccacgagtggcagccgcgggactgtcgcgtcggcgcccgacgcggagtcagcaggggcgaaaagcggtagatcatggcaaccatagaagaaattgcacatcaaattattgaacaacagatgggagagattgttacagagcagcaaactgggcagaaaatccagattgtgacagcacttgatcataatacccaaggcaagcagttcattctgacaaatcacgacggctctactccaagcaaagtcattctggccaggcaagattccactccgggaaaagttttccttacaactccagatgcagcaggtgtcaaccagttattttttaccactcctgatctgtctgcacaacacctgcagctcctaacagataattctccagaccaaggaccaaataaggtttttgatctttgcgtagtatgtggagacaaagcatcaggacgtcattatggagcagtaacttgtgaaggctgcaaaggattttttaaaagaagcatccgaaaaaatttagtatattcatgtcgaggatcaaaggattgtattattaataagcaccaccgaaaccgctgtcaatactgcaggttacagagatgtattgcgtttggaatgaagcaagactctgtccaatgtgaaagaaaacccattgaagtatcacgagaaaaatcttccaactgtgccgcttcaacagaaaaaatctatatccgaaaggaccttcgtagcccattaactgcaactccaacttttgtaacagatagtgaaagtacaaggtcaacaggactgttagattcaggaatgttcatgaatattcatccatctggagtaaaaactgagtcagctgtgctgatgacatcagataaggctgaatcatgtcagggagatttaagtacattggccaatgtggttacatcattagcgaatcttggaaaaactaaagatctttctcaaaatagtaatgaaatgtctatgattgaaagcttaagcaatgatgatacctctttgtgtgaatttcaagaaatgcagaccaacggtgatgtttcaagggcatttgacactcttgcaaaagcattgaatcctggagagagcacagcctgccagagctcagtagcgggcatggaaggaagtgtacacctaatcactggagattcaagcataaattacaccgaaaaagaggggccacttctcagcgattcacatgtagctttcaggctcaccatgccttctcctatgcctgagtacctgaatgtgcactacattggggagtctgcctccagactgctgttcttatcaatgcactgggcactttcgattccttctttccaggctctagggcaagaaaacagcatatcactggtgaaagcttactggaatgaactttttactcttggtcttgcccagtgctggcaagtgatgaatgtagcaactatattagcaacatttgtcaattgtcttcacaatagtcttcaacaagcagaggggtaatcaccttaaaatgtcatcaaaaatagatctactagaaggcagcatcacattcccatcttacttatggactcctacccctggttcatgtcttatatgcctgtaatggttataaagcctaccttcaggaaagctatggttgactaattactaatggatgggttttaaacatgtccctctacaataaattaaaatctttcaatgtttgaatataatgtggaggtgtttacctgagggcctctctatctccccgaattc

SEQ ID NO: 8 - protein, 603 aa, Artificial Sequence
  Description of Artificial Sequence/note = synthetic construct
MATIEEIAHQIIEQQMGEIVTEQQTGQKIQIVTALDHNTQGKQFILTNHDGSTPSKVILARQDSTPGKVFLTTPDAAGVNQLFFTTPDLSAQHLQLLTDNSPDQGPNKVFDLCVVCGDKASGRHYGAVTCEGCKGFFKRSIRKNLVYSCRGSKDCIINKHHRNRCQYCRLQRCIAFGMKQDSVQCERKPIEVSREKSSNCAASTEKIYIRKDLRSPLTATPTFVTDSESTRSTGLLDSGMFMNIHPSGVKTESAVLMTSDKAESCQGDLSTLANVVTSLANLGKTKDLSQNSNEMSMIESLSNDDTSLCEFQEMQTNGDVSRAFDTLAKALNPGESTACQSSVAGMEGSVHLITGDSSINYTEKEGPLLSDSHVAFRLTMPSPMPEYLNVHYIGESASRLLFLSMHWALSIPSFQALGQENSISLVKAYWNELFTLGLAQCWQVMNVATILATFVNCLHNSLQQDKMSTERRKLLMEHIFKLQEFCNSMVKLCIDGYEYAYLKAIVLFSPDHPSLENMELIEKFQEKAYVEFQDYITKTYPDDTYRLSRLLLRLPALRLMNATITEELFFKGLIGNIRIDSVIPHILKMEPADYNSQIIGHSI

SEQ ID NO: 9 - DNA, 2202 bp, Artificial Sequence
  Description of Artificial Sequence/note = synthetic construct
gggactgtcgcgtcggcgcccgacgcggagtcagcaggggcgaaaagcggtagatcatggcaaccatagaagaaattgcacatcaaattattgaacaacagatgggagagattgttacagagcagcaaactgggcagaaaatccagattgtgacagcacttgatcataatacccaaggcaagcagttcattctgacaaatcacgacggctctactccaagcaaagtcattctggccaggcaagattccactccgggaaaagttttccttacaactccagatgcagcaggtgtcaaccagttattttttaccactcctgatctgtctgcacaacacctgcagctcctaacagataattctccagaccaaggaccaaataaggtttttgatctttgcgtagtatgtggagacaaagcatcaggacgtcattatggagcagtaacttgtgaaggctgcaaaggattttttaaaagaagcatccgaaaaaatttagtatattcatgtcgaggatcaaaggattgtattattaataagcaccaccgaaaccgctgtcaatactgcaggttacagagatgtattgcgtttggaatgaagcaagactctgtccaatgtgaaagaaaacccattgaagtatcacgagaaaaatcttccaactgtgccgcttcaacagaaaaaatctatatccgaaaggaccttcgtagcccattaactgcaactccaacttttgtaacagatagtgaaagtacaaggtcaacaggactgttagattcaggaatgttcatgaatattcatccatctggagtaaaaactgagtcagctgtgctgatgacatcagataaggctgaatcatgtcagggagatttaagtacattggccaatgtggttacatcattagcgaatcttggaaaaactaaagatctttctcaaaatagtaatgaaatgtctatgattgaaagcttaagcaatgatgatacctctttgtgtgaatttcaagaaatgcagaccaacggtgatgtttcaagggcatttgacactcttgcaaaagcattgaatcctggagagagcacagcctgccagagctcagtagcgggcatggaaggaagtgtacacctaatcactggagattcaagcataaattacaccgaaaaagaggggccacttctcagcgattcacatgtagctttcaggctcaccatgccttctcctatgcctgagtacctgaatgtgcactacattggggagtctgcctccagactgctgttcttatcaatgcactgggcactttcgattccttctttccaggctctagggcaagaaaacagcatatcactggtgaaagcttactggaatgaactttttactcttggtcttgcccagtgctggcaagtgatgaatgtagcaactatattagcaacatttgtcaattgtcttcacaatagtcttcaacaagataaaatgtcaacagaaagaagaaaattattgatggagcacatcttcaaactacaggagttttgtaacagcatggttaaactctgcattgatggatacgaatatgcctacctgaaggcaatagtactcttcagtccagatcatccaagcctagaaaacatggaactgatagagaaatttcaggaaaaggcttatgtggaattccaagattatataaccaaaacatatccagatgacacctacaggttatccagactactactcagattgccagctttaagactgatgaatgctaccatcactgaagaattgtttttcaaaggtctcattggcaatatacgaattgacagtgttatcccacatattttgaaaatggagcctgcagattataactctcaaataattggtcacagcatttgaaaactgtgactgcagtgctgtaaacttaactgttctttgccagaacacaagacaccaaattgaactcactgcttttgaggcatctggaaatttttactttaaaaagtaaccagaatccaaggtatttttattttagcttcccttaagaatttttgaagtgactgggcaggcagcagaaattaaatgaatttttcttcctgattcctttaaatgaatatgaaacactacaaatttattcttggtgaagatgatacctgaagctgtcacctcttgattatctaaactaagcgctcattctattttataaaacaaataaattagtctcttttttctg

SEQ ID NO: 10 - protein, 483 aa, Artificial Sequence; identical to SEQ ID NO: 1
  Description of Artificial Sequence/note = synthetic construct

SEQ ID NO: 11 - DNA, 2029 bp, Artificial Sequence
  Description of Artificial Sequence/note = synthetic construct
gaattcgggcccgtcggctttcttcaaccctctcttcccggagcgcccccaatccacgagtggcagccgcgggactgtcgcgtcggcgcccgacgcggagtcagcaggggcgaaaagcggtagatcatggcaaccatagaagaaattgcacatcaaattattgaacaacagatgggagagattgttacagagcagcaaactgggcagaaaatccagattgtgacagcacttgatcataatacccaaggcaagcagttcattctgacaaatcacgacggctctactccaagcaaagtcattctggccaggcaagattccactccgggaaaagttttccttacaactccagatgcagcaggtgtcaaccagttattttttaccactcctgatctgtctgcacaacacctgcagctcctaacagataattctccagaccaaggaccaaataaggtttttgatctttgcgtagtatgtggagacaaagcatcaggacgtcattatggagcagtaacttgtgaaggctgcaaaggattttttaaaagaagcatccgaaaaaatttagtatattcatgtcgaggatcaaaggattgtattattaataagcaccaccgaaaccgctgtcaatactgcaggttacagagatgtattgcgtttggaatgaagcaagactctgtccaatgtgaaagaaaacccattgaagtatcacgagaaaaatcttccaactgtgccgcttcaacagaaaaaatctatatccgaaaggaccttcgtagcccattaactgcaactccaacttttgtaacagatagtgaaagtacaaggtcaacaggactgttagattcaggaatgttcatgaatattcatccatctggagtaaaaactgagtcagctgtgctgatgacatcagataaggctgaatcatgtcagggagatttaagtacattggccaatgtggttacatcattagcgaatcttggaaaaactaaagatctttctcaaaatagtaatgaaatgtctatgattgaaagcttaagcaatgatgatacctctttgtgtgaatttcaagaaatgcagaccaacggtgatgtttcaagggcatttgacactcttgcaaaagcattgaatcctggagagagcacagcctgccagagctcagtagcgggcatggaaggaagtgtacacctaatcactggagattcaagcataaattacaccgaaaaagaggggccacttctcagcgattcacatgtagctttcaggctcaccatgccttctcctatgcctgagtacctgaatgtgcactacattggggagtctgcctccagactgctgttcttatcaatgcactgggcactttcgattccttctttccaggctctagggcaagaaaacagcatatcactggtgaaagcttactggaatgaactttttactcttggtcttgcccagtgctggcaagtgatgaatgtagcaactatattagcaacatttgtcaattgtcttcacaatagtcttcaacaagatgccaaggtaattgcagccctcattcatttcacaagacgagcaatcactgatttataaatgcttaactatagaatggcttatgactacccaaaacagtgccccatcaacaaatggggaaaattgccttttgagctcaggaataatttataaattggggactaccttttagttctttagcatattctatttcttattgttttatataatttttaaatcatttgcttcctccttatgtttaacagcagaggggtaatcaccttaaaatgtcatcaaaaatagatctactagaaggcagcatcacattcccatcttacttatggactcctacccctggttcatgtcttatatgcctgtaatggttataaagcctaccttcaggaaagctatggttgactaattactaatggatgggttttaaacatgtccctctacaataaattaaaatctttcaatgtttgaatataatgtggaggtgtttacctgagggcctctctatctccccgaattc

SEQ ID NO: 12 - protein, 596 aa, Artificial Sequence
  Description of Artificial Sequence/note = synthetic construct
MTSPSPRIQIISTDSAVASPQRIQIVTDQQTGQKIQIVTAVDASGSPKQQFILTSPDGAGTGKVILASPETSSAKQLIFTTSDNLVPGRIQIVTDSASVERLLGKTDVQRPQVVEYCVVCGDKASGRHYGAVSCEGCKGFFKRSVRKNLTYSCRSNQDCIINKHHRNRCQFCRLKKCLEMGMKMESVQSERKPFDVQREKPSNCAASTEKIYIRKDLRSPLIATPTFVADKDGARQTGLLDPGMLVNIQQPLIREDGTVLLATDSKAETSQGALGTLANVVTSLANLSESLNNGDTSEIQPEDQSASEITRAFDTLAKALNTTDSSSSPSLADGIDTSGGGSIHVISRDQSTPIIEVEGPLLSDTHVTFKLTMPSPMPEYLNVHYICESASRLLFLSMHWARSIPAFQALGQDCNTSLVRACWNELFTLGLAQCAQVMSLSTILAAIVNHLQNSIQEDKLSGDRIKQVMEHIWKLQEFCNSMAKLDIDGYEYAYLKAIVLFSPDHPGLTSTSQIEKFQEKAQMELQDYVQKTYSEDTYRLARILVRLPALRLMSSNITEELFFTGLIGNVSIDSIIPYILKMETAEYNGQITGASL

SEQ ID NO: 13 - DNA, 13 bp, Artificial Sequence
  Description of Artificial Sequence/note = synthetic construct
aggtcanaggtca

SEQ ID NO: 14 - DNA, 21 bp, Artificial Sequence
  Description of Artificial Sequence/note = synthetic construct
ccctgactagtttctgcgatc

SEQ ID NO: 15 - DNA, 22 bp, Artificial Sequence
  Description of Artificial Sequence/note = synthetic construct
gcctactcatggaaatataacc

SEQ ID NO: 16 - DNA, 21 bp, Artificial Sequence
  Description of Artificial Sequence/note = synthetic construct
gctgatgctaccaagtccacg

SEQ ID NO: 17 - DNA, 20 bp, Artificial Sequence
  Description of Artificial Sequence/note = synthetic construct
gcagcgcatcgccttctatc

SEQ ID NO: 18 - DNA, 21 bp, Artificial Sequence
  Description of Artificial Sequence/note = synthetic construct
tgcaagcatacttcttgttcc

SEQ ID NO: 19 - DNA, 20 bp, Artificial Sequence
  Description of Artificial Sequence/note = synthetic construct
gcagcgcatcgccttctatc

SEQ ID NO: 20 - DNA, 27 bp, Artificial Sequence
  Description of Artificial Sequence/note = synthetic construct
ggagacacactgcacatgttcgaatac

SEQ ID NO: 21 - DNA, 28 bp, Artificial Sequence
  Description of Artificial Sequence/note = synthetic construct
cacagctcatttctctgctcacttactc

SEQ ID NO: 22 - DNA, 27 bp, Artificial Sequence
  Description of Artificial Sequence/note = synthetic construct
ggagacacactgcagatgtccgaatac

SEQ ID NO: 23 - DNA, 28 bp, Artificial Sequence
  Description of Artificial Sequence/note = synthetic construct
cacagctcatttctctgctcacttactc

SEQ ID NO: 24 - DNA, 21 bp, Artificial Sequence
  Description of Artificial Sequence/note = synthetic construct
tgcaagcatacttcttgttcc

SEQ ID NO: 25 - DNA, 20 bp, Artificial Sequence
  Description of Artificial Sequence/note = synthetic construct
gcagcgcatcgccttctatc

SEQ ID NO: 26 - DNA, 27 bp, Artificial Sequence
  Description of Artificial Sequence/note = synthetic construct
ggagacacactgcacatgttcgaatac

SEQ ID NO: 27 - DNA, 28 bp, Artificial Sequence
  Description of Artificial Sequence/note = synthetic construct
cacagctcatttctctgctcacttactc

SEQ ID NO: 28 - DNA, 20 bp, Artificial Sequence
  Description of Artificial Sequence/note = synthetic construct
gcagcgcatcgccttctatc

SEQ ID NO: 29 - DNA, 21 bp, Artificial Sequence
  Description of Artificial Sequence/note = synthetic construct
tgcaagcatacttcttgttcc

SEQ ID NO: 30 - DNA, 22 bp, Artificial Sequence
  Description of Artificial Sequence/note = synthetic construct
catattcaccacctcggacaac

SEQ ID NO: 31 - DNA, 18 bp, Artificial Sequence
  Description of Artificial Sequence/note = synthetic construct
tgacgccacagaccacac

SEQ ID NO: 32 - DNA, 21 bp, Artificial Sequence
  Description of Artificial Sequence/note = synthetic construct
ccgcatctaatcgctggagag

SEQ ID NO: 33 - DNA, 22 bp, Artificial Sequence
  Description of Artificial Sequence/note = synthetic construct
gcataggagaaggcatggtgag

SEQ ID NO: 34 - DNA, 24 bp, Artificial Sequence
  Description of Artificial Sequence/note = synthetic construct
tgtgcccatctacgaggggtatgc

SEQ ID NO: 35 - DNA, 24 bp, Artificial Sequence
  Description of Artificial Sequence/note = synthetic construct
ggtacatggtggtgccgccagaca

SEQ ID NO: 36 - DNA, 20 bp, Artificial Sequence
  Description of Artificial Sequence/note = synthetic construct
gcagcagcagcaagagacta

SEQ ID NO: 37 - DNA, 20 bp, Artificial Sequence
  Description of Artificial Sequence/note = synthetic construct
dcatccaggaccaggtagcc

SEQ ID NO: 38 - DNA, 18 bp, Artificial Sequence
  Description of Artificial Sequence/note = synthetic construct
cctggagatgttggatgc

SEQ ID NO: 39 - DNA, 20 bp, Artificial Sequence
  Description of Artificial Sequence/note = synthetic construct
gtaaggaatgtgctgaagtg

SEQ ID NO: 40 - DNA, 18 bp, Artificial Sequence
  Description of Artificial Sequence/note = synthetic construct
gcgacgacggcacggttc

SEQ ID NO: 41 - DNA, 21 bp, Artificial Sequence
  Description of Artificial Sequence/note = synthetic construct
ctgcttcctggcttgcggtag

SEQ ID NO: 42 - DNA, 20 bp, Artificial Sequence
  Description of Artificial Sequence/note = synthetic construct
tgtgctggacctggatatgc

SEQ ID NO: 43 - DNA, 19 bp, Artificial Sequence
  Description of Artificial Sequence/note = synthetic construct
agtgtgcgggaatgctctc

SEQ ID NO: 44 - DNA, 18 bp, Artificial Sequence
  Description of Artificial Sequence/note = synthetic construct
cttggcttactggctctg

SEQ ID NO: 45 - DNA, 18 bp, Artificial Sequence
  Description of Artificial Sequence/note = synthetic construct
cagatgcttggtccgaag

SEQ ID NO: 46 - DNA, 21 bp, Artificial Sequence
  Description of Artificial Sequence/note = synthetic construct
ctggtcaaccttgacttcctt

SEQ ID NO: 47 - DNA, 6450 bp, Artificial Sequence
  Description of Artificial Sequence/note = synthetic construct
gagttgtgcctggagtgatgtttaagccaatgtcagggcaaggcaacagtccctggccgtcctccagcacctttgtaatgcatatgagctcgggagaccagtacttaaagttggaggcccgggagcccaggagctggcggagggcgttcgtcctgggagctgcacttgctccgtcgggtcgccggcttcaccggaccgcaggctcccggggcagggccggggccagagctcgcgtgtcggcgggacatgcgctgcgtcgcctctaacctcgggctgtgctctttttccaggtggcccgccggtttctgagccttctgccctgcggggacacggtctgcaccctgcccgcggccacggaccatgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggacataacgactatatgtgtccagccaccaaccagtgcaccattgataaaaacaggaggaagagctgccaggcctgccggctccgcaaatgctacgaagtgggaatgatgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagtctgagagctccctggctcccacacggttcagataatccctgctgcattttaccctcatcatgcaccactttagccaaattctgtctcctgcatacactccggcatgcatccaacaccaatggctttctagatgagtggccattcatttgcttgctcagttcttagtggcacatcttctgtcttctgttgggaacagccaaagggattccaaggctaaatctttgtaacagctctctttcccccttgctatgttactaagcgtgaggattcccgtagctcttcacagctgaactcagtctatgggttggggctcagataactctgtgcatttaagctacttgtagagacccaggcctggagagtagacattttgcctctgataagcactttttaaatggctctaagaataagccacagcaaagaatttaaagtggctcctttaattggtgacttggagaaagctaggtcaagggtttattatagcaccctcttgtattcctatggcaatgcatccttttatgaaagtggtacaccttaaagcttttatatgactgtagcagagtatctggtgattgtcaattcacttccccctataggaatacaaggggccacacagggaaggcagatcccctagttggccaagacttattttaacttgatacactgcagattcagagtgtcctgaagctctgcctctggctttccggtcatgggttccagttaattcatgcctcccatggacctatggagagcaacaagttgatcttagttaagtctccctatatgagggataagttcctgatttttgtttttatttttgtgttacaaaagaaagccctccctccctgaacttgcagtaaggtcagcttcaggacctgttccagtgggcactgtacttggatcttcccggcgtgtgtgtgccttacacaggggtgaactgttcactgtggtgatgcatgatgagggtaaatggtagttgaaaggagcaggggccctggtgttgcatttagccctggggcatggagctgaacagtacttgtgcaggattgttgtggctactagagaacaagagggaaagtagggcagaaactggatacagttctgagcacagccagacttgctcaggtggccctgcacaggctgcagctacctaggaacattccttgcagaccccgcattgcctttgggggtgccctgggatccctggggtagtccagctcttattcatttcccagcgtggccctggttggaagaagcagctgtcaagttgtagacagctgtgttcctacaattggcccagcaccctggggcacgggagaagggtggggaccgttgctgtcactactcaggctgactggggcctggtcagattacgtatgcccttggtggtttagagataatccaaaatcagggtttggtttggggaagaaaatcctcccccttcctcccccgccccgttccctaccgcctccactcctgccagctcatttccttcaatttcctttgacctataggctaaaaaagaaaggctcattccagccacagggcagccttccctgggcctttgcttctctagcacaattatgggttacttcctttttcttaacaaaaaagaatgtttgatttcctctgggtgaccttattgtctgtaattgaaaccctattgagaggtgatgtctgtgttagccaatgacccaggtagctgctcgggcttctcttggtatgtcttgtttggaaaagtggatttcattcatttctgattgtccagttaagtgatcaccaaaggactgagaatctgggagggcaaaaaaaaaaaaaaaagtttttatgtgcacttaaatttggggacaattttatgtatctgtgttaaggatatgcttaagaacataattcttttgttgctgtttgtttaagaagcaccttagtttgtttaagaagcaccttatatagtataatatatatttttttgaaattacattgcttgtttatcagacaattgaatgtagtaattctgttctggatttaatttgactgggttaacatgcaaaaaccaaggaaaaatatttagtttttttttttttttttgtatacttttcaagctaccttgtcatgtatacagtcatttatgcctaaagcctggtgattattcatttaaatgaagatcacatttcatatcaacttttgtatccacagtagacaaaatagcactaatccagatgcctattgttggatattgaatgacagacaatcttatgtagcaaagattatgcctgaaaaggaaaattattcagggcagctaattttgcttttaccaaaatatcagtagtaatatttttggacagtagctaatgggtcagtgggttctttttaatgtttatacttagattttcttttaaaaaaattaaaataaaacaaaaaaaatttctaggactagacgatgtaataccagctaaagccaaacaattatacagtggaaggttttacattattcatccaatgtgtttctattcatgttaagatactactacatttgaagtgggcagagaacatcagatgattgaaatgttcgcccaggggtctccagcaactttggaaatctctttgtatttttacttgaagtgccactaatggacagcagatattttctggctgatgttggtattgggtgtaggaacatgatttaaaaaaaaaactcttgcctctgctttcccccactctgaggcaagttaaaatgtaaaagatgtgatttatctggggggctcaggtatggtggggaagtggattcaggaatctggggaatggcaaatatattaagaagagtattgaaagtatttggaggaaaatggttaattctgggtgtgcaccaaggttcagtagagtccacttctgccctggagaccacaaatcaactagctccatttacagccatttctaaaatggcagcttcagttctagagaagaaagaacaacatcagcagtaaagtccatggaatagctagtggtctgtgtttcttttcgccattgcctagcttgccgtaatgattctataatgccatcatgcagcaattatgagaggctaggtcatccaaagagaagaccctatcaatgtaggttgcaaaatctaacccctaaggaagtgcagtctttgatttgatttccctagtaaccttgcagatatgtttaaccaagccatagcccatgccttttgagggctgaacaaataagggacttactgataatttacttttgatcacattaaggtgttctcaccttgaaatcttatacactgaaatggccattgatttaggccactggcttagagtactccttcccctgcatgacactgattacaaatactttcctattcatactttccaattatgagatggactgtgggtactgggagtgatcactaacaccatagtaatgtctaatattcacaggcagatctgcttggggaagctagttatgtgaaaggcaaataaagtcatacagtagctcaaaaggcaaccataattctctttggtgcaagtcttgggagcgtgatctagattacactgcaccattcccaagttaatcccctgaaaacttactctcaactggagcaaatgaactttggtcccaaatatccatcttttcagtagcgttaattatgctctgtttccaactgcatttcctttccaattgaattaaagtgtggcctcgtttttagtcatttaaaattgttttctaagtaattgctgcctctattatggcacttcaattttgcactgtcttttgagattcaagaaaaatttctattcatttttttgcatccaattgtgcctgaacttttaaaatatgtaaatgctgccatgttccaaacccatcgtcagtgtgtgtgtttagagctgtgcaccctagaaacaacatacttgtcccatgagcaggtgcctgagacacagacccctttgcattcacagagaggtcattggttatagagacttgaattaataagtgacattatgccagtttctgttctctcacaggtgataaacaatgctttttgtgcactacatactcttcagtgtagagctcttgttttatgggaaaaggctcaaatgccaaattgtgtttgatggattaatatgcccttttgccgatgcatactattactgatgtgactcggttttgtcgcagctttgctttgtttaatgaaacacacttgtaaacctcttttgcactttgaaaaagaatccagcgggatgctcgagcacctgtaaacaattttctcaacctatttgatgttcaaataaagaattaaact

(19) United States
(12) Patent Application Publication
(10) Pub. No.: US 2006/0212953 A1
(43) Pub. Date: Sep. 21, 2006

(54) Tr2, tr4, tr2/tr4 double knockouts and uses thereof

(76) Inventor: Chawnshang Chang, Pittford, NY (US)

Correspondence Address: NEEDLE & ROSENBERG, P.C., SUITE 1000, 999 PEACHTREE STREET, ATLANTA, GA, 30309-3915, US

(21) Appl. No.: 10/515,675

(22) PCT Filed: May 28, 2003
(86) PCT No.: PCT/US03/16901

§ 371 Date: Jan. 18, 2005

Related U.S. Application Data
(60) Provisional application No. 60/383,844, filed on May 28, 2002.
(60) Provisional application No. 60/426,519, filed on Nov. 14, 2002.
(60) Provisional application No. 60/426,582, filed on Nov. 14, 2002.

(51) Int. Cl. A01K 67/027; C12N 5/06

(52) U.S. Cl. 800/18; 435/354

(57) ABSTRACT

Disclosed are compositions and methods for disrupting an testicular orphan nuclear receptor 2.

This application claims benefit of U.S. Provisional Application 60/383,844, filed on May 28, 2002, for “Testicular Orphan Nuclear Receptor-2 Knock-out Animals,” 60/426,519, filed on Nov. 14, 2002, for TR4 Knockout Animals,” and 60/426,582, filed on Nov. 14, 2002 for TR2/TR4 Double Knockouts and Their Uses.” All three of these applications are specifically incorporated herein by reference in their entireties.

### I. ACKNOWLEDGEMENTS

This work was funded in part by National Institutes of Health Grant Number 47258, and the United States may have certain rights in this work.

### II. BACKGROUND OF THE INVENTION

The nuclear receptors are ligand-inducible transcription factors that regulate the expression of target genes by binding to their specific hormone response elements (HREs). They are characterized by a central DNA binding domain (DBD), which binds to HRE. The C-terminal half of the receptor encompasses the ligand-binding domain (LBD), which mediates ligand binding, dimerization, and transactivation function. Nuclear receptors play roles in various aspects of physiology including metabolism, development, homeostasis, and reproduction (Evans, R. M. (1988) Science 240:889-95, Mangelsdorf, D. J., et al. (1995) Cell 83:835-9). Orphan nuclear receptors embody structures of nuclear receptors but are without identified ligands, and make up the vast majority of the nuclear receptor superfamily (Enmark, E., and J. A. Gustafsson (1996) Mol Endocrinol 10: 1293-307). With genetic knockout approaches, several orphan nuclear receptors have been demonstrated to have important physiological functions (Chen, W. S., et al., (1994) Genes Dev 8:2466-7); (Ingraham, H. A., et al. (1994) Genes Dev 8:2302-12); (Pereira, F. A., et al. (1999) Genes Dev 13:1037-49); (Qiu, Y., et al. (1997) Genes Dev 11:1925-37); (Steinmayr, M., et al. (1998) Proc Natl Acad Sci USA 95:3960-5.) Orphan nuclear receptors testicular orphan nuclear receptor-2 (TR2) (Chang, C., and J. Kokontis (1988) Biophys Res Commun 155:971-7) and testicular orphan nuclear receptor-4 (TR4) (Chang, C., S. et al. (1994) Proc Natl Acad Sci USA 91:60404) constitute a subfamily of nuclear receptors. TR2 was isolated from testes and prostate cDNA libraries and its cDNA encodes a protein of 603 amino acids with a calculated molecular mass of 67 kilodaltons (Chang, C., and J. Kokontis (1988) Biophys Res Commun 155:971-7); (Chang, C. et al. (1989) Biophys Res Commun 165:73541).

An abundance of TR2 mRNA detected in developing mouse embryos and in situ hybridization revealed that TR2 is highly expressed in the active proliferating zone of the developing nervous system and other developing organs (Lee, C. H., et al., (1996) Mol Reprod Dev 44:305-14, Young, W. J., et al., (1998) J Biol Chem 273:20877-85). TR2 has been shown to be specifically expressed in adult mice testes and is confined to advanced germ cells (Lee, C. H., et al. (1996) Mol Reprod Dev 44:305-14). The role of TR2 has been demonstrated in the regulation of several signaling pathways included retinoic acid (Lin, T. M. et al. (1995) J Biol Chem 270:30121-8), thyroid hormone (Chang, C., and H. J. Pan (1998) Mol Cell Biochem 189:195-200), and ciliary neurotrophic factor (Young, W. et al. (1998) J Biol Chem 273:20877-85) TR2-mediated repression has been shown to be, in part, a direct interaction with both class I and class II histone deacetylase proteins (Franco, P. J. et al. (2001) Mol Endocrinol 15:1318-28). The expression of TR2 was completely repressed in the surgery-induced cryptorchidism of the rhesus monkey through a pathway which involves p53 and the retinoblastoma gene product (Rb). Such dramatic repression of TR2 in cryptorchidism suggests that TR2 may play important roles in male infertility associated with cryptorchidism (Mu, X. et al. (2000) J Biol Chem 275:23877-83). In vitamin A-depleted mice, the spermatogenesis was blocked at an early stage. In testes losing advanced germ cells, the expression of TR2 could not be detected, suggesting a biological relationship between TR2 and male germ-cell differentiation (Lee, C. H., et al. (1996) Mol Reprod Dev 44:305-14). These studies strongly indicate that TR2 may play a role in the embryogenesis and male germ-cell differentiation.

### III. SUMMARY OF THE INVENTION

In accordance with the purposes of this invention, as embodied and broadly described herein, this invention, in one aspect, relates to compositions and methods related to testicular orphan nuclear receptor.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### IV. BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 shows targeted disruption of the murine TR2 gene. (A) Structure of wild-type allele, targeting construct, and recombinant locus. Dark boxes represent exons of TR2. The expected fragments after EcoRV digestion are 7.4 kb for the wild-type allele and 6.5 kb for the mutant allele. Arrows indicate the primers used for PCR genotyping and the bar represents the region used for probe. RV, EcoRV. (B) Southern blot analysis of mouse tail DNA isolated from the progeny of a mating between heterozygous parents. DNA was digested with EcoRV and hybridized with the probe indicated in panel (C)PCR analysis of mouse genomic DNA. The wild-type and targeted alleles give 498 and 774-bp PCR products, respectively. (D). Northern blot analysis of total RNA isolated from testis tissue from 6 weeks old mice. The same membrane was sequentially hybridized with ³²P-labeled TR2 and β-actin cDNA probes. +/+, wild-type mouse; +/−, heterozygous mutant mouse; −/−, homozygous mutant mouse.

FIG. 2 shows histological analysis of testes from 6 week old wild type (A) and mutant mice (B). Testes were fixed in neutral buffered formalin, sectioned, and stained with H&E. Magnification, 100×. (C) The selected stages of the seminiferous cycle of 6 week old TR2 homozygous mutant mice. Testes sections were stained with Periodic Acid Schiff (PAS) and counterstained with Hematoxylin. Magnification, 100×. (D-F) The testis weight, sperm count and sperm motility assays of 6 week old wild-type mice (+/+), heterozygous mutant mice (+/−) and homozygous mutant mice(−/−). (n=6 from each genotype). Error bars represent the standard errors of the means.

FIG. 3 shows the Design of the TR4 Knockout, βgal Knockin Targeting Construct/Knockout Screening Strategy. A segment of TR4 genomic DNA is shown, including exons 4-9 and their associated introns. The IRES βgal MC1-Neo selection cassette is inserted between introns 3 and 5, taking the place of exons 4 and 5, as well as the intervening intron 4. PCR primers designed for mouse genotyping include TR4-34 and Neo-3a, to screen for the presence of the selection cassette, as well as LC-7 and LC-11 to screen for the presence of the wildtype gene. Animal genotypes were also confirmed using a Southern blot/restriction enzyme digestion strategy. Genomic DNA isolated from mice was digested with Eco RI and then probed with a 5′ external probe, yielding an 8.0 kb fragment in the case of the wildtype or a 4.9 kb fragment if the selection cassette was present. Genomic DNA was also digested with Nsi I and then probed with a 3′ eternal probe, yielding an 8.2 kb fragment of the wildtype gene or a 12.5 kb fragment if the selection cassette was present.

FIG. 4 shows design of the TR2 Knockout, βgal Knockin Targeting Construct/Knockout Screening Strategy A segment of TR2 genomic DNA is shown, including exons 3-7 and their associated introns. The IRES βgal MC1-Neo selection cassette is inserted between introns 3 and 5, taking the place of exons 4 and 5, as well as the intervening intron 4. Animal genotypes will be confirmed using a Southern blot/restriction enzyme digestion strategy. Genomic DNA isolated from mice will be digested with Eco RV and then probed with a 5′ external probe, yielding an 7.4 kb fragment in the case of the wildtype or a 6.5 kb fragment if the selection cassette is present. Genomic DNA will also be digested with Xba I and then probed with a 3′ internal probe, yielding an 4.5 kb fragment of the wildtype gene or a 8.5 kb fragment if the selection cassette is present.

FIG. 5 shows growth curves of TR4−/−, TR4+/−, and wildtype mice. Left panel (male animlas)Pups resulting from heterozygous matings were weighed every other day starting at day 2, until day 30. Pups were then weighed once per week for 5 additional weeks. Male homozygous KO pups display a range of significant weight reduction (p<0.05) between 24% and 56% less than their WT and heterozygous counterparts at all time points except day 4 (16% reduction, p<0.1). The KO animals display an approximately 30% growth reduction by day 10, which increases to approximately 50% in weeks 4 and 5, and then returns to 30% and is maintained through week 12. +/+n=15; +/−n 15; +/−n=6. right panel (female animals) Mice were weighed using the same timepoints as described for males. Female homozygous KO pups display a range of significant weight reduction (p<0.05) between 20% and 54% less than their WT and heterozygous counterparts at all time points. The KO animals display an approximately 30% growth reduction at the first time point, day 2, which increases to approximately 50% in week 3. The reduction in KO weight then drops to 20% by week 5, a level which is maintained through week 12. +/+n=15; +/−n=15; −/−n=5 up to day 22, n=3 from day 24 to day 86.

FIG. 6 shows the up-regulation of iNOS transactivation and NO releasing by TR4. (A) Two week old astrocyte primary culture from either wild type, heterozygous, and knockout mice cerebella were treated with 10 nM DHT, 10 μM of LPS for 24 hrs and assayed for amount NO releasing in conditional medium by Griess reagents (Sigma, G-4410). (B) COS-1 cells were transfected with luciferase reporter plasmid that contains human iNOS promoter together with either TR4 or TR2 mammalian expression plasmids (pCMX-TR4, or pCMV-TR2) by SuperFect (Qiagen) and then 2 days later cells were harvested and assayed for luciferase acitivity.

FIG. 7 shows a flow chart related to growth retardation.

FIG. 8 shows a flow chart related to fertility.

FIG. 9 shows a time course of expression of TR4 during testis development A: Total RNA was isolated from testes of mice at different ages as indicated. RT-PCR was performed. β-actin served as an internal control. B: Total RNA was isolated from testes of mice at different ages as indicated in the same manner as FIG. 2A and real-time quantitative RT-PCR were performed. C: Timetable of first wave of spermatogenesis including the preleptene (PL), leptene (L), Zygotene (Z), pachytene and diplotene stages (Di) of germ cell differentiation.

FIG. 10 shows testes weight and sperm production in TR4^{+/+} and TR4^{-/-} mice. (A) The comparison of testes weight from TR4^{+/+} and TR4^{-/-} mice. Testes from TR4^{+/+} mice and TR4^{-/-} mice were removed and the weights of testes were measured. (B) The comparison of sperm count from cauda epididymis between TR4^{+/+} and TR4^{-/-} mice. Sperm from cauda epididymis of 2-3 month old TR4^{+/+} and TR4^{-/-} mice were counted. The cauda epididymis from more than five TR4^{+/+} and TR4^{-/-} mice were counted by hemocytotometer under phase-contrast microscopy.

FIG. 11 shows the numbers of stage X-XII tubules and total tubules from each of 6 testis sections from TR4^{+/+} and TR4^{-/-} mice stained with PAS and hematoxylin were counted and the ratio between stage X-XII and total tubules were calculated.

FIG. 12 shows analysis of testis specific gene expression in TR44 mice. RT-PCR and real-time quantitative RT-PCR of testis specific gene were performed A: premeiosis expressed genes proacrosin, HSP 70 and Histone 1 expression pattern in TR4^{-/-} and TR4^{+/+} mice at indicated ages. B: Postmeiosis expressed gene protamine 1 and 2, transition protein 1 and 2 expression pattern in TR4^{-/-} and TR4^{+/+} mice at indicated ages. C: late meiotic prophase expressed genes sperm-1 and cyclin A1 expression pattern in TR4^{+/+} and TR4^{-/-} mice at indicated ages. D: Quantitative analysis of sperm-1 and cyclin A1 in TR4^{+/+}, TR4^{+/-}, and TR4^{-/-} mice at indicated ages. E: Comparison of sperm-1 and cyclin A1 expression pattern between TR4^{+/+} and TR4^{-/-} mice at various developing and adult stages by RT PCR. F; Quantitative analysis of sperm-1 expression pattern in TR4^{+/+} and TR4^{-/-} mice at various indicated developing and adult stages by real-time PCR. G: Quantitative analysis of cyclin A1 expression pattern in TR4^{+/+} and TR4^{-/-} mice at various indicated developing and adult stages by real-time RT-PCR. A-G: All RT-PCR experiments were repeated three times with RNA samples from three different mice. One representative experiment was shown, and β-actin mRNA are indicated as internal controls. All real-time RT-PCR reactions were triplicated and repeated two times, and all results are normalized with β-actin.

FIG. 13 shows cerebral and cerebellar area and neuronal composition (A) The surface area of the cerebral hemisphere is significantly reduced in TR4KO animals at all ages shown. Data is expressed as the mean of samples of the same age and genotype. ^{a}N=3-5 for WT at each age, N=3-4 for TR4KO at each age; ^{b}Number of cortical pyramidal neurons in a 0.106 mm² area of the front parietal cortex, N=2 for each genotype at each age; ^{c} TR4KO neuron number per area is significantly higher than WT. (B) The area of the cerebellar mid-sagittal section is significantly reduced in 6 mo. old TR4KO female mice, but not in TR4KO males at 3-3.5 months of age. Significant reduction of granule neuron number per area is observed in TR4KO males at 3-3.5 mo. of age. Data is expressed as the mean of 2-3 samples of the same age and genotype. * p=0.05, ** p<0.05 (A, B).

FIG. 14 shows reduced Purkinje cell number and larger parallel fiber synaptic boutons in TR4KO mice (A) A 15% reduction in the number of Purkinje cells present in the adult TR4KO cerebellar cortex was observed; N=9-10 for each genotype, **p<0.01. (B) Granule cell axons form parallel fibers in the molecular layer of the cerebellar cortex where synapses with Purkinje cell dendrites are made in the form of synaptic boutons. (C) Electron micrographs of cerebellar granule cell synaptic endings demonstrate increased size of parallel fiber synaptic boutons in TR4KO (right, KO) mice compared to WT (left) animals.

FIG. 15 shows granule cell parallel fiber synaptic terminal boutons are fewer and larger in TR4KO mice EM morphometric analysis demonstrated that (A) the number of parallel fiber synaptic ending boutons were reduced by 45% in TR4KO mice (p<0.0001), and (B) the size of individual boutons are 40% larger in TR4KO animals (p<0.001).

FIG. 16 shows a model of inhibitory neurotransmission in normal and TR4KO mice pathways of normal inhibitory neurotransmission are depicted on the left. The defects in cerebellar structure observed in TR4KO mice, and the hypothesized consequences of those defects, are depicted on the right. Granule cells secrete the excitatory neurotransmitter glutamate, which stimulates the inhibitory (GABAergic) Puridnje cells. Interneurons (basket cells), as well as climbing and mossy fibers, that innervate the cerebellum are GABAergic and function to inhibit Purkinje cell activity. The downstream targets of Purkinje cells include dentate neurons, which ultimately affect muscle cell stimulation. In the case of the TR4KO mouse, fewer granule cells are present in the cerebellum, likely providing less stimulatory signal to activate Purkinje cells. With nembutal (pentobarbital) anesthesia, a known GABA agonist at high concentrations, Purkinje cells are inhibited to a greater degree by GABAergic interneurons, climbing fibers, and mossy fibers. The combination of less Purkinje cell activation and increased Purkinje cell inhibition results in reduced inhibition of downstream targets (dentate neurons), leading to increased motor activity.

FIG. 17 shows a TR4KO targeting construct, genotype confirmation, and mouse production and mortality rates (A) A segment of TR4 genomic DNA is shown, including exons 4-9 and their associated introns. The IRES β-gal MC1-Neo selection cassette is inserted between introns 3 and 5, taking the place of exons 4 and 5, as well as the intervening intron 4. PCR primers designed for mouse genotyping include Neo-3a and TR4-34, to screen for the presence of the selection cassette, as well as TR4-107 and TR4-111 to screen for the presence of the wildtype gene. (B) Genotypes were confirmed by PCR analysis which yielded a 495 bp fragment in the wildtype (+/+) and a 760 bp fragment if the knockout allele was present (−/−). Heterozygous animals display one copy of each allele (+/−). (C) RT-PCR analysis of TR4 and TR2 mRNA expression levels in cerebellum and testis tissue from WT and TR4KO mice. Expression of TR4 is absent in tissue from TR4KO mice, and no increase in TR2 expression is observed in either cerebellum or testis tissue from TR4KO animals. Pactin levels were determined as a control for template amount in PCR reactions. (D) Ratios of genotypes generated from heterozygous pairings, and pup mortality rates. TR4+/− breeding pairs (110 total) generated 751 pups. Genotype ratios are significantly different from those expected among all mice, or among females and males considered independently. TR4−/− female mice are generated at a significantly lower rate than are male TR4−/− mice^{a} (p<0.005). TR4−/− pups showed an increase in mortality near the age of weaning (3-5 weeks). ***p<0.001

FIG. 18 shows a TR4KO males display priapism (A) WT male (left), at 7 months of age, without priapism; TR4KO male (right), at 7 months of age, showing priapism. (B) Histological staining of penis sections from 16 week old WT and TR4KO mice. The penis of a TR4KO mouse that had displayed priapism, in section and stained with hematoxylin and eosin, is compared with a similarly stained penis section from a WT mouse. The TR4KO tissue exhibits blood trapped within the corpus cavemosum (CC), causing swelling and thus reduction of the preputial cavity (PC), and epithelial evidence of external trauma, 40× magnification. E, keratinized epithelial cells; S, penile sheath. (C) Number and percentage of TR4KO male mice showing priapism at different ages.

FIG. 19 shows a immunohistochemical staining for nNOS and S100 in WT and TR4KO penis sections, and regulation of the nNOS promoter by TR4 (A) Penis sections from 16 week old TR4KO and WT mice were stained using an antibody recognizing neuronal nitric oxide synthase (nNOS), 400× magnification. Magnified regions of each stained section are shown adjacent to the original photomicrographs. Increased space between cells in the sections from TR4KO mice suggests edema, possibly resulting from trauma-induced penile inflammation. nNOS expression is observed in WT penis tissue (brown color indicates positive staining), and is reduced significantly in TR4KO tissue. (B) Penis sections from 16 week old TR4KO and WT mice were stained using an antibody recognizing the neuronal marker S100, 400× magnification. Expression of S100 is present at similar levels in both WT and TR4KO penis tissue. (C) p4.3nNOSLUC, p2.3nNOSLUC, or pnNOS(1880/2187)LUC (0.8 μg) was transiently co-transfected into COS1 cells with pCMX-TR4 (0.2 μg) or its parental vector. Forty-eight hours after transfection, cells were harvested, lysed and assayed for luciferase reporter activity. Results were obtained from three independent experiments performed in triplicate.

FIG. 20 shows that TR4 binds to the nuclear hormone receptor binding site (nNOS-NHR) of the mouse nNOS exon 2 promoter In vitro synthesized TR4 protein was incubated with a radiolabeled nNOS-NHR probe for 15 min. at room temperature, in the presence or absence of a mouse monoclonal anti-TR4 antibody. The reaction mixtures were analyzed on a 5% native polyacrylamide/0.25×TBE gel. The result was visualized by autoradiography. TR4 protein was found to bind to the nNOS-NHR probe (TR4/nNOS-NHR). The presence of TR4 in the complex was confirmed by supershift of the complex using a TR4-specific antibody (TR4/nNOS-NHR/Ab). n.s., nonspecific binding.

FIG. 21 shows reproductive rates, testis weight, epididymis weight, and sperm counts reduced in TR4KO mice (A) Continuous mating of 5 month old males, each with one WT female for 4 months; four TR4KO (−/−) males produced no litters. TR4KO males produced significantly fewer litters than WT males. ***p<0.001 (B) TR4KO mice showed significantly reduced body, testis, and epididymal weights when compared to WT males of the same age (7 months). The testis to body weight ratio of TR4KO males is significantly increased, while there is no change in the TR4KO epididymis to body weight ratio compared to WT males, *p<0.01, ** p<0.001; WT N=8, TR4KO N=9. (C) Epididymal sperm counts were taken using a hemacytometer. Data shown is the mean±s.d. of 3-9 samples, p<0.05 between genotypes at each age, except at 44-56 weeks (p<0.1).

FIG. 22 shows RT-PCR and Real Time PCR analysis of sexual behavior/function-related gene expression in the hypothalamus (A) RT-PCR analysis of AR, ERα, ERβ, VP and OT mRNA expression. Pactin levels were determined as a control for template amount in PCR reactions. (B) Real Time PCR quantitation of AR, ERα, ERβ, OT, and VP gene expression. Relative gene expression levels were calculated using the −2^{ΔΔC}_{T} method. (C) Graphical representation of the relative expression (from above Real Time data) of the ERα, ERβ, and OT genes in the hypothalami of WT and TR4KO mice.

FIG. 23 shows reproductive deficiencies and lack of maternal behavior among TR4KO females (A) Age-matched adult WT and TR4KO female mice were each paired with a sexually mature WT male for 2.5 weeks and then separated. Only 1 out of 5 TR4KO female produced a litter, whereas all WT females paired produced litters. (B) Observations of TR4KO female mothers suggest defects in maternal behavior. TR4KO mothers do not build nests, collect pups to a single location, crouch over pups, or nurse their offspring. (C) Pups of TR4KO mothers die within 24-36 hours after birth with no milk in their stomachs. (D) Histology of mammary gland tissue from a mouse heterozygous (Het) for TR4 (heterozygous females show normal reproductive capacity and maternal behavior) and from a TR4KO female, on postpartum day 1, demonstrate no obvious defect in milk production in the mutant animal. The magnified mammary gland structures (lower panels) show milk (pink staining) within the glandular lumen (GL). GE, glandular epithelium.

FIG. 24 shows growth retardation in TR4KO mice (A) Male mice at 7 months of age are pictured. From left to right: wildtype (32.2 g), TR4+/−(33.7 g), and TR4−/− (18.4 g). Wildtype and TR4+/− animals are comparable in size, whereas TR4−/− animals show an approximately 40% reduction in body weight. (B) Pups resulting from heterozygous matings were weighed every other day starting at day 2, until day 30. Pups were then weighed once per week for 8 additional weeks. Male homozygous KO pups (left panel) display a range of significant weight reduction (p<0.05) between 24% and 56% less than their WT and heterozygous counterparts at all time points except day 4 (16% reduction, p<0.1). TR4+/+N=15; TR4+/−N=15; TR4^{-/-} N=6. Female homozygous KO pups (right panel) display a range of significant weight reduction (p<0.05) between 20% and 54% less than their wildtype and heterozygous counterparts at all time points. TR4+/+N=15; TR4+/−N=15; TR4−/− N=5 up to day 22, N=3 from day 24 to day 86. (C) Reduced IGF-1 staining was observed in liver tissue from 4 month old KO male mice compared with liver samples from WT male mice of the same age. Immunostaining was carried out with an antibody recognizing IGF-1 (Upstate Biotechnology), and the tissues were counterstained with hematoxylin, 400× magnification. (D) Serum levels of IGF-1 in 7 month old male wildtype (WT) and TR4KO (KO) mice were determined via radioimmunoassay. A significantly lower serum IGF-1 level was observed in KO mice compared to WT animals (p=0.05, N=3 for each genotype).

### V. DETAILED DESCRIPTION

The present invention may be understood more readily by reference to the following detailed description of preferred embodiments of the invention and the Examples included therein and to the Figures and their previous and following description.

Before the present compounds, compositions, articles, devices, and/or methods are disclosed and described, it is to be understood that this invention is not limited to specific synthetic methods, specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this invention pertains. The references disclosed are also individually and specifically incorporated by reference herein for the material contained in them that is discussed in the sentence in which the reference is relied upon.

B. Compositions and Methods

1. Nuclear Receptors

The nuclear receptor superfamily is comprised of transcription factors that are related by sequence and structure, yet are specifically induced or repressed by a wide variety of chemical compounds. As transcription factors, nuclear receptors control the expression of target genes and thereby direct developmental, physiological, and behavioral responses from the cellular level to that of the whole organism (Evans, R M. (1988) Science 240, 889-895). The structural features common to nuclear receptors include those required for ligand binding, dimerization, DNA binding, and transactivation (Mangelsdorf, D. J. et al. (1995) Cell 83, 835-839). Binding of a particular receptor to a specific DNA sequence, or response element (RE), within the promoter of one of its target genes is mediated by a region of the receptor containing two zinc finger motifs (Freedman, L. P. (1992) Endocrine Reviews 13, 129-145). This DNA binding domain (DBD) displays a high level of amino acid homology between nuclear receptors and has been used as a template when developing probes with which to screen for new members of the nuclear receptor family. Using this strategy, many structurally related receptors have been identified, yet remain a mystery in terms of their specific ligands and/or their physiological functions, and are therefore referred to as orphan receptors (Laudet, V. (1997) J. Mol. Endocrinol. 19, 207-226).

More specifically, the overall structure shared among members of the nuclear receptor superfamily is highly conserved and is made up of four general domains termed A/B, C, D, and E, in order from the amino to carboxy terminus of the receptor (Laudet, V. (1997) J. Mol. Endocrinol 19,207-226). The most highly conserved regions are the C and E domains. The C domain is the DNA binding region of a nuclear receptor, and a key feature of this family of transcription factors. Additionally, the C domain has been found to be important for selection of a partner with which a receptor may interact to form a hetero- or homodimeric molecule (Gronmeyer, H., and Laudet, V. (1995) Protein Profile 2(11), 1173-1308). The D domain of a nuclear receptor is a hinge region between domains C and D, often contains nuclear localization signals (Guiochon-Mantel, A. et al. (1994) Proc. Natl. Acad. Sci. USA 91, 7179-7183), and the E domain is a large region that functions in ligand binding, dimerization, and transactivation (Gronmeyer, H., and Laudet, V. (1995) Protein Profile 2(11), 1173-1308). Within the E domain, near its C-terminus, there exists an activation function 2-activation domain (AF2-AD), which displays a ligand-dependent activation function (Durand, B. et al. (1994) EMBO J. 13, 5370-5382). The A/B domain, at the amino terminus of a nuclear receptor, contains a ligand-independent activation function domain (Beato, M. et al. (1995) Cell 83, 851-857). The highly conserved, complex structure of nuclear receptors make them well suited to their function as DNA-binding transcription factors, but it is now known that nuclear receptors mediate cellular signaling via non-genomic pathways as well (Farhat, M. et al. (1995) Biochem. Pharmacal. 51,571-576).

Using probes specific for the DBDs of various steroid receptors, two highly homologous orphan receptors, Testicular Receptor 2 (TR2) and Testicular Receptor 4 (TR4), were isolated (Chang, C., and Kokontis, J. (1988) Biochem. Biophys. Res. Comm. 155,971-977); Chang, C. et al. (1994) Proc. Natl. Acad. Sci. USA 1994, 6040-6044). TR2 and TR4 are closely related to the retinoid X receptor (RXR), COUP-TF, and HNF4 in sequence and structure (Laudet, V. (1997) J. Mol. Endocrinol. 19, 207-226), and bind to AGGTCA DNA sequence motifs in direct repeat orientation, with variable spacing, in the promoters of their target genes (Lin, D. L. et al. (1998) Endocrine 8, 123-134). Both embryonic and adult tissue distribution of TR2 and TR4, as well as the substantial number of target genes known to be regulated by these orphan receptors, suggest that significant developmental and physiological pathways are affected by TR2 and TR4. Both TR2 and TR4 are expressed in neural and non-neural tissues during embryonic development (Lee, Y. F. et al. (1998) J. Bioi. Chern. 273, 13437-13443); (Young, W. J. et al. (1997); J. Bioi. Chern. 272, 3109-3116); (Young, W. J. et al. (1998) J. BioI. Chern. 273, 20877-20885). In situ hybridization experiments using probes specific for TR2 or TR4 have shown transcripts present in actively proliferating cell populations of the brain and peripheral organs, during embryonic development. The expression of TR2 and TR4 at sites of sensory innervation and in sensory organs throughout embryogenesis indicate an important role for these receptors in this critical aspect of nervous system development. Additionally, high expression of TR2 and TR4 in the developing brain and spinal cord, including specific expression in motor neurons, suggest that these receptors may be involved in the proper development of movement and limb coordination.

In studies of the genomic structure and chromosomal location of TR2 and TR4, it was found that TR4 mapped to human chromosome 3q24.3, and TR2 to human chromosome 12q22 (Lin,. D. L. et al. (1998) Endocrine 8, 123-134). From cytogenetic analysis of human germ cell tumors, two common abnormalities were found in chromosome 12, one on the short arm, 12p, and one on the long arm, 12q (Murty, V. V. V. S. et al. (1992) FroG. Natl. Acad. Sci. USA 89, 11006-11010). As part of the same study, loss of heterozygosity analysis revealed two regions of frequent loss, one at 12q13 and the other at 12q22. These sites were then postulated to be the locations of potential tumor suppressor genes. As TR2 is mapped to one of the regions thought to be the location of a tumor suppressor gene, and has an expression pattern suggesting a role in germ cell development, TR2 may be a candidate. Further studies have been carried out in attempts to specify the region, and ultimately the gene, responsible for the observed tumor-suppressive activity (Murty, V. V. V. S. et al. (1996) Genomics 35, 562-570).

2. TR2 and TR4 Gene Regulation

As TR2 and TR4 function as transcription factors, a characteristic shared by members of the nuclear receptor superfamily, there are several genes known to be regulated by either TR2, TR4, or similarly by both receptors (Table 1). The functions of these target genes range from maintenance of erythrocyte progenitor populations, in the case of the human erythropoietin gene (EPO), through roles in the process of neurogenesis, in the case of the ciliary neurotrophic factor alpha (CNTFRα), and facilitation of viral infection and propagation, in the case of HPV-16 and SV40.

TABLE 1
Table 1. TR2 and TR4 target genes

| Target Gene | RE Motif | Direction of Regulation |
|---|---|---|
| TR2 Target Genes | | |
| EPO | DR2 | repression |
| histamine H1 receptor | DR3/DR4 | repression |
| aldolase A | DR1 | activation |
| TR4 Target Genes | | |
| VDR target gene (p450cc24) | DR3 | repression |
| HIV 1 | indirect | activation |
| TR2/TR4 Target Genes | | |
| CRBPII | DR1 | repression |
| RARbeta | DR5 | repression |
| SV40 | DR2 | repression |
| T3Ralpha target genes | DR4 | activation |
| CNTFRalpha | DR1 | activation |
| HPV-16 | DR4 | activation |

Abbreviations used: EPO, erythropoietin; VDR, vitamin D receptor; p450cc24, 25-hydroxyvitamin D₃ 24-hydroxylase; RAR, retinoic acid receptor; SV40, Simian virus 40; T3R, thyroid hormone receptor; CNTFR, ciliary neurotrophic factor; HPV-16, human papilloma virus type 16; and DR, direct repeat.

Disclosed herein the genomic structure of TR2 and TR4 has been identified, cloned and analyzed. The DNA response elements to which these receptors bind, and through binding, carry out their function as transcription factors, have also been identified and characterized. In addition several target genes of these receptors have been identified through which TR2 and TR4 are likely to affect such diverse physiological functions as neurogenesis, erythrocyte development and maturation, muscle physiology, growth, and bone development. Adding more support for the potential roles of TR2 and TR4 is the pattern of expression in embryonic and adult tissue. The distribution of both TR2 and TR4 in the male germ cell lineage, as well as the extensive expression of the receptors in the developing and mature nervous system. Also disclosed are animals, such as mice that have functionally ablated copies of either TR2 or TR4, or of both genes.

A distinct feature of members of the nuclear receptor superfamily is the DNA binding domain, containing two zinc finger motifs (Evans, R. M. (1988) Science 240, 889-895). Various steroid receptors bind to different cognate response elements (Beato, M. (1989) Cell 56, 335-344). The response element recognized by the TR2 and TR4 is made up of the AGGTCA half-site in direct repeat (DR) orientation, with variable nucleotide spacing between half-sites (Young, W. J. et al. (1998) J. BioI. Chern. 273, 20877-20885). Considering the high homology between TR2 and TR4 and their recognition of similar response elements, it is not surprising that these orphan receptors modulate some of the same target genes or are involved in some of the same signaling pathways. Examples of shared affinity of TR2 and TR4 for certain target genes include the positive regulation of thyroid hormone receptor target genes via competition with T₃Rα for the DR4RE recognized by all three receptors (Chang, C., and Pan, H. (1998) Mol. Cell. Biochem. 189, 195-200); (Lee, Y. F. et al. (1997) J. BioI. Chern. 272, 12215-12220). Likewise, a response element for both TR2 and TR4 has been identified in intron 5 of the ciliary neurotrophic factor receptor alpha (CNTFRα) gene, a region demonstrated to have enhancer activity. Binding of TR2 or TR4 to the DR1 element of CNTFRα leads to induction of CNTFRα transcription in a reporter gene assay. These data describe the first neural-specific gene regulated by TR2 or TR4, and suggest important roles for these receptors in the process of neurogenesis (Young, W. J. et al. (1997) J. Bioi. Chern. 272, 3109-3116); (Young, W. J. et al. (1998) J. Biol. Chern. 273, 20877-20885). An additional target for positive regulation by both TR2 and TR4 is the pathogenic human papilloma virus. A DR4 response element has been identified in the long control region of the human papilloma virus type 16 (HPV-16), and induction of viral gene expression by the two receptors has been demonstrated via reporter gene assay. Combined with data demonstrating expression of TR2 in the stratified squamous epithelium of the cervix and vagina, the predominant site of genital papilloma virus infection, these results suggest possible involvement of TR2 and TR4 in maintenance of papilloma virus infection as well as in the development of HPV-16 associated cervical cancer (Collins, L. L. et al. (2001) J. BioI. Chern. 276, 27316-27321). TR2 and TR4 down-regulate some of the same target genes as well. Examples of these include genes containing response elements recognized by RAR/RXR heterodimers, such as CRBPII (containing a DR1) and RARE (containing a DR5). Previous studies have demonstrated that TR2 and TR4 bind to DR1 and DR5 response elements with higher affinity than do RAR/RXR heterodimers. Through competition for binding, TR2 and TR4 may modulate the retinoic acid signaling pathway (Lee, Y. F. et al. (1998) J Bioi. Chern. 273, 13437-13443); (Lin, D. et al. (1995) J BioI. Chern. 270, 30121-30128). Finally, both TR2 and TR4 also down-regulate expression of Simian Virus 40 (SV40) genes via binding to a DR2 response element in the +55 region of SV40 (Lee, H., and Chang, C. (1995) J Biol. Chem. 270, 5434-5440); (Lee, H. et al. (1995) J. Biol. Chem. 270, 30129-30133).

Although TR2 and TR4 are extremely similar in sequence and expression pattern, recognize many of the same hormone response elements, and modulate transcription of several of the same target genes, there are particular targets unique to each. A DR2 response element, specifically recognized by TR2, has been identified in the hypoxia-inducible enhancer of the human erythropoietin gene (EPO). Based on a reporter gene assay, it has been demonstrated that TR2 suppresses expression of EPO, suggesting a role for TR2 in modulation of the pathways important for survival and growth of erythrocyte progenitor cells of the bone marrow (Lee, H. J. et al. (1996) J BioI. Chern. 271, 10405-10412); (Krantz, S. B. (1991) Blood 77,419-434). An additional target negatively regulated by TR2 is the histamine H1 receptor gene, which contains a DR4 as well as two DR3 elements in its 3′ flanking region (Lee, H. et al. (1999) Mol. Cell. Biochem. 194, 199-207). Histamine is a neuromodulator in the mammalian central nervous system, acting through three receptors including the H1 receptor (Lee, H. et al. (1999) Mol. Cell. Biochem. 194, 199-207). The histamine H1 receptor plays a role in smooth muscle and terminal venule contraction, as well as in the release of catecholamine from the adrenal medulla (Yamashita, M. et al. (1991) FroG. Natl. A cad. Sci. USA 88, 11515-11519), and has also been implicated in sex hormone signaling pathways (Off, E. L., and Quay, W. B. (1975) Endocrinology 96, 941-945); (Sawyer, C. H. (1955) Am. J Physiol. 180, 3746); (Donoso, A. O. et al. (1976) J Endocrinol. 68, 171-172). The known functions of this TR2 target gene, the histamine H1 receptor, suggest significant roles for TR2 in both the nervous and reproductive systems. Finally, TR2 is also a positive regulator of the muscle-specific pM promoter of the human aldolase A gene which contains a DR1 response element. Regulation of muscle-specific expression of this gene suggests a potential role for TR2 in muscle development and/or physiology (Chang, C. et al. (1997) Biochem. Biophys. Res. Commun. 235,205-211).

Another gene that is regulated by TR4 is the vitamin-D receptor (VDR) target gene, 25-hydroxyvitamin D₃ 24-hydroxylase (p450 cc24), which is repressed by TR4 via binding of the receptor to a DR3 response element within the gene (Chang, C. et al., (1997) Biochem. Biophys. Res. Commun. 235,205-211). These data, combined with expression pattern of TR4 showing expression in vitamin D₃ target organs such as kidney, intestine, and bone (Chang, C. et al. (1997) Biochem. Biophys. Res. Commun. 235,205-211), suggest a role for TR4 in the vitamin D₃-VDR signaling pathway. An additional TR4-specific target gene is the human immunodeficiency virus type 1 (HIV1), although not a direct target. Through crosstalk with the chicken ovalbumin upstream protein-transcription factor (COUP-TF1) and the thyroid hormone receptor, TR4 was able to increase transcriptional activity of the HIV1 long terminal repeat region (Hwang, S. et al. (1998) Endocrine 8, 169-175).

3. Compositions and Methods for Disrupting a TR2 loci

Disclosed are animals, such as mouse, that have had the TR2 loci or portion thereof knocked out. By “knocked out” it is met that the endogenous TR2 loci no longer produces a functional TR2 protein. As discussed herein, these knockouts can be made in many ways, by for example, disrupting one or more of the exons of TR2. This disruption can take place in a variety of ways, through for example, homologous recombination events, which substitute non-TR2 coding sequence, such as a marker gene, such as the neo gene, for TR2 coding sequence, or the lacZ gene. The knockouts could also be made with inducible expression systems, such as a Cre/lox system, so that the disruption of the TR2 gene is inducible, for example, through tissue specific promoters of Cre. It is understood that the knockouts can be made by disrupting any exon or multiple exons of the TR2 gene. The disruption can include for example, a point mutation, which alters the protein sequence or a point deletion which causes a mis-sense polypeptide to be produced, or deletions or alterations so any fragment of the TR2 gene is disrupted which disrupts TR2 protein production.

The disclosed animals can be used in a variety of ways. For example, they can be used as tools to study drugs related to TR2 and molecules involved in TR2 signaling in vivo. Thus, the disclosed animals can be used for drug discovery and for drug validation. The disclosed animals can also be used reagents to produce other beneficial knockout animals, by for example, breeding the disclosed knockout animals with other knockout animals, producing double or even mupltiple gene knockouts. These animals are useful as model systems for drug discovery and validation.

Disclosed are methods of generating a cell line wherein the TR2 loci has been disrupted. For example, the TR2 loci can be disrupted by, for example, disrupting one of the exons, such that a stop codon terminates translation of the TR2 peptide early or where the exon is completely taken out. The TR2 loci would include any exon or intron associated with the TR2 gene.

The TR2 gene is considered any sequence associated with the TR2 locus. Thus, it would at least include the chromosomal nucleic acid contained within any organism that expresses a TR2, such as, the introns, exons, 5′ upstream sequence involved with the TR2 coding and non-coding sequence, and 3′ downstream sequence involved with the TR2 coding and non coding sequence. It is also understood that fragments of the TR2 locus are disclosed.

A disrupted TR2 loci can be any TR2 loci that does not produce a native TR2 protein. A disrupted TR2 loci would also include any TR2 loci wherein the nucleic acid of the natural TR2 gene, including exons and introns has been altered. Typically the altering of the TR2 gene will cause a disruption in TR2 function, by for example, preventing DNA binding in the TR2 gene product or ligand binding in the TR2 gene product or transactivating activity in the TR2 gene product. The disrupted TR2 loci can be made using any known technique, including homologous recombination techniques. The disrupted loci can be an alteration of any exon to produce a non-functional TR2 protein. Furthermore, disclosed are constructs and methods to mutate any exon in the TR2 through homologous recombination via the surrounding introns.

The disrupted TR2 gene can be in any cell that contains a TR2 gene, such as an embryonic stem cell, an embryonic germ cell, a breast cell, a breast cancer cell, an ovary cell, an ovary cancer cell, and any cell line of cells that contain TR2 genes which are expressed, such as prostate cells, testis, bone, brain, neural, and muscle.

Disclosed are methods of determining the effect of steroids on TR2 using a TR2 disrupted cell line, comprising administering a steroid to a any of the cells or cell lines disclosed herein containing a disrupted TR2.

Disclosed are methods of generating an animal wherein the TR2 loci has been disrupted.

Disclosed are methods of generating an animal wherein the TR2 loci has been disrupted and wherein the disruption is inducible.

Disclosed are methods of generating an animal wherein the TR2 loci has been disrupted a) wherein the disruption is inducible and b) wherein the inducible gene is flanked by sites which can be acted upon by a recombinase, such as loxP sites.

Disclosed are methods of generating an animal wherein the TR2 loci has been disrupted a) wherein the disruption is inducible, b) wherein sequence associated with the TR2 loci is flanked by sites which can be acted upon a recombinase, such as loxP sites, and c) wherein the sites can be cleaved by a recombinase, such as cre recombinase, under the control of an inducible promoter or a constitutive promoter, such as, the CMV promoter.

Disclosed are inducible expression systems to generate mice without a functional testicular orphan nuclear receptor 2. It is understood that many inducible expression systems exist in the art and may be used as disclosed herein. Inducible expression systems can include, but are not limited to the Cre-lox system, Flp recombinase, and tetracycline responsive promoters. Any recombinase system can be used. The Cre recombinase system which when used will execute a site-specific recombination event at loxP sites. A gene that is flanked by the loxP sites, floxed, is excised from the transcript. To create null mice using the Cre-lox system, two types of transgenic mice are created. The first is a mouse transgenic for Cre recombinase under control of a known inducible and/or tissue-specific promoter. The second is a mouse that contains the floxed gene. These two transgenic mouse strains are then crossed to create one strain comprising both mutations. Disclosed are constructs and mice that place the Testicular Orphan receptor 2 (TR2) gene in the floxed position such that upon recombination an TR2 null mutation is corrected. Control of the recombination event, via the Cre Recombinase, can be constitutive or inducible, as well as ubiquitous or tissue specific, depending on the promoter used to control Cre expression. Disclosed is a constitutive system in which the Cre recombinase is expressed from a β-actin promoter. Other inducible expression systems exist and can be used as disclosed herein. It is understood that the promoter region of TR4 could be flanked by recombinase sites, such as flox sites, as well, to produce a knockout.

Disclosed are vectors for making TR2 knockout animals, such as mice. Disclosed are vectors comprising a region 1 for homologous recombination with a region of the TR2 gene, for example, an intron, and a region of one or more exons, such as exon 1, of the testicular orphan nuclear receptor 2 gene, a region encoding a selectable marker, and a region 2 for homologous recombination with for example, intron 1, of the testicular orphan nuclear receptor 2 gene.

Disclosed are vectors, wherein the homologous recombination regions, such as a region 1 can be at least 300 nucleotides long, at least 750 nucleotides long, at least 1000 nucleotides long, or at least 1100 nucleotides long.

Also disclosed are vectors, wherein the homologous recombination regions comprise sequence that has at least 70%, 80%, 90%, or 95% homology to one or more regions of the TR2 gene, such as exons 3-7 or exon 4 or exon 5.

Also disclosed are vectors, comprising selectable markers, for example, wherein the selectable marker is a Neo marker.

Also disclosed are vectors, wherein the selectable marker is a negative selection marker or wherein the selectable marker is a positive selection marker.

Also disclosed are vectors comprising a region 1 for homologous recombination exon 3 or fragment thereof of the TR2 loci, a region encoding one or more selectable markers, such as a B-gal and/or a neo marker, and a region 2 for homologous recombination with for example exon 6 or exon 7 or any intervening sequence.

Disclosed are vectors, comprising a region of exons 3-7 or exon 4 and 5, for example, of the TR2 gene.

Disclosed are cells comprising any of the vectors or nucleic acid molecules disclosed herein.

Disclosed are cells, wherein the cell is a cell which can be cultured, wherein the cell is an ES cell, and/or wherein the ES cell is a mouse ES cell.

Also disclosed are cells comprising a disrupted TR2 gene.

Disclosed are cells, wherein the disrupted TR2 gene comprises sites for recombination by a recombinase, wherein the sites are lox sites, wherein the recombinase is cre recombinase, and/or wherein the disrupted TR2 gene comprises a variant of the TR2 gene.

Disclosed are mammals comprising the vector and/or cells disclosed herein.

Disclosed are mammals, wherein the mammal is bovine, ovine, porcine, primate, mouse, rat, hamster, or rabbit.

Disclosed are mammals, wherein the disclosed vector has integrated into the mammals genome, comprising an integrated nucleic acid.

4. Compositions and Methods for Disrupting a TR4 loci

Disclosed are animals, such as mouse, that have had the TR4 loci or portion thereof knocked out. By “knocked out” it is met that the endogenous TR4 loci no longer produces a functional TR4 protein. As discussed herein, these knockouts can be made in many ways, by for example, disrupting one or more of the exons of TR4. This disruption can take place in a variety of ways, through for example, homologous recombination events, which substitute non-TR4 coding sequence, such as a marker gene, such as the neo gene, for TR4 coding sequence or the lacZ gene. The knockouts could also be made with inducible expression systems, such as a Cre/lox system, so that the disruption of the TR4 gene is inducible, for example, through tissue specific promoters of Cre. It is understood that the knockouts can be made by disrupting any exon or multiple exons of the TR4 gene. The disruption can include for example, a point mutation, which alters the protein sequence or a point deletion which causes a mis-sense polypeptide to be produced, or deletions or alterations so any fragment of the TR4 gene is disrupted which disrupts TR4 protein production.

The disclosed animals can be used in a variety of ways. For example, they can be used as tools to study drugs related to TR4 and molecules involved in TR4 signaling in vivo. Thus, the disclosed animals can be used for drug discovery and for drug validation. The disclosed animals can also be used reagents to produce other beneficial knockout animals, by for example, breeding the disclosed knockout animals with other knockout animals, producing double or even mupltiple gene knockouts. These animals are useful as model systems for drug discovery and validation.

Disclosed are methods of generating a cell line wherein the TR4 loci has been disrupted. For example, the TR4 loci can be disrupted by, for example, disrupting one of the exons, such that a stop codon terminates translation of the TR4 peptide early or where the exon is completely taken out. The TR4 loci would include any exon or intron associated with the TR4 gene.

The TR4 gene is considered any sequence associated with the TR4 locus. Thus, it would at least include the chromosomal nucleic acid contained within any organism that expresses a TR4, such as, the introns, exons, 5′ upstream sequence involved with the TR4 coding and non-coding sequence, and 3′ downstream sequence involved with the TR4 coding and non coding sequence. It is also understood that fragments of the TR4 locus are disclosed.

A disrupted TR4 loci can be any TR4 loci that does not produce a native TR4 protein. A disrupted TR4 loci would also include any TR4 loci wherein the nucleic acid of the natural TR4 gene, including exons and introns has been altered. Typically the altering of the TR4 gene will cause a disruption in TR4 function, by for example, preventing DNA binding in the TR4 gene product or ligand binding in the TR4 gene product or transactivating activity in the TR4 gene product. The disrupted TR4 loci can be made using any known technique, including homologous recombination techniques. The disrupted loci can be an alteration of any exon to produce a non-functional TR4 protein. Furthermore, disclosed are constructs and methods to mutate any exon in the TR2 through homologous recombination via the surrounding introns.

The disrupted TR4 gene can be in any cell that contains a TR4 gene, such as an embryonic stem cell, an embryonic germ cell, a breast cell, a breast cancer cell, an ovary cell, an ovary cancer cell, and any cell line of cells that contain TR4 genes which are expressed, such as prostate cells, testis, bone, brain, neural, and muscle.

Disclosed are methods of determining the effect of steroids on TR4 using a TR4 disrupted cell line, comprising administering a steroid to a any of the cells or cell lines disclosed herein containing a disrupted TR4.

Disclosed are methods of generating an animal wherein the TR4 loci has been disrupted.

Disclosed are methods of generating an animal wherein the TR4 loci has been disrupted and wherein the disruption is inducible.

Disclosed are methods of generating an animal wherein the TR4 loci has been disrupted a) wherein the disruption is inducible and b) wherein the inducible gene is flanked by sites which can be acted upon by a recombinase, such as loxP sites.

Disclosed are methods of generating an animal wherein the TR4 loci has been disrupted a) wherein the disruption is inducible, b) wherein sequence associated with the TR4 loci is flanked by sites which can be acted upon a recombinase, such as loxP sites, and c) wherein the sites can be cleaved by a recombinase, such as cre recombinase, under the control of an inducible promoter or a constitutive promoter, such as, the CMV promoter.

Disclosed are inducible expression systems to generate mice without a functional testicular orphan nuclear receptor 4. It is understood that many inducible expression systems exist in the art and may be used as disclosed herein. Inducible expression systems can include, but are not limited to the Cre-lox system, Flp recombinase, and tetracycline responsive promoters. Any recombinase system can be used. The Cre recombinase system which when used will execute a site-specific recombination event at loxP sites. A gene that is flanked by the loxP sites, floxed, is excised from the transcript. To create null mice using the Cre-lox system, two types of transgenic mice are created. The first is a mouse transgenic for Cre recombinase under control of a known inducible and/or tissue-specific promoter. The second is a mouse that contains the floxed gene. These two transgenic mouse strains are then crossed to create one strain comprising both mutations. Disclosed are constructs and mice that place the Testicular Orphan receptor 4 (TR4) gene in the floxed position such that upon recombination an TR4 null mutation is corrected. Control of the recombination event, via the Cre Recombinase, can be constitutive or inducible, as well as ubiquitous or tissue specific, depending on the promoter used to control Cre expression. Disclosed is a constitutive system in which the Cre recombinase is expressed from a β-actin promoter. Other inducible expression systems exist and can be used as disclosed herein. It is understood that the promoter region of TR4 could be flanked by recombinase sites, such as flox sites, as well, to produce a knockout.

Disclosed are vectors for making TR4 knockout animals that delete the DNA binding domain, for example, exons 4 and 5.

Disclosed are vectors for making TR4 knockout animals, such as mice. Disclosed are vectors comprising a region 1 for homologous recombination with a region of the TR4 gene, for example, an intron, and a region of one or more exons, such as exon 1, exon 2, exon 3, exon 4, exon 5, exon 6, exon 7, exon 8, exon 9 or some combination of these, of the testicular orphan nuclear receptor 4 gene, a region encoding a selectable marker, and a region 2 for homologous recombination with for example, intron 1, intron 2, intron 3, intron 4, intron 5, intron 6, intron 7, intron 8, intron 9, or intron 10 of the testicular orphan nuclear receptor 4 gene.

Disclosed are vectors, wherein the homologous recombination regions, such as a region 1 of the TR4 gene can be at least 300 nucleotides long, at least 750 nucleotides long, at least 1000 nucleotides long, or at least 1100 nucleotides long.

Also disclosed are vectors, wherein the homologous recombination regions comprise sequence that has at least 70%, 80%, 90%, or 95% homology to one or more regions of the TR4 gene, such as exons 3-7 or exon 4 or exon 5.

Also disclosed are vectors, comprising selectable markers, for example, wherein the selectable marker is a Neo marker.

Also disclosed are vectors, wherein the selectable marker is a negative selection marker or wherein the selectable marker is a positive selection marker.

Also disclosed are vectors comprising a region 1 for homologous recombination exon 3 or fragment thereof of the TR4 loci, a region encoding one or more selectable markers, such as a B-gal and/or a neo marker, and a region 2 for homologous recombination with for example exon 6 or exon 7 or any intervening sequence.

Disclosed are vectors, comprising a region of exons 3-7 or exon 4 and 5, for example, of the TR4 gene.

Disclosed are cells comprising any of the vectors or nucleic acid molecules disclosed herein.

Disclosed are cells, wherein the cell is a cell which can be cultured, wherein the cell is an ES cell, and/or wherein the ES cell is a mouse ES cell.

Also disclosed are cells comprising a disrupted TR4 gene.

Disclosed are cells, wherein the disrupted TR4 gene comprises sites for recombination by a recombinase, wherein the sites are lox sites, wherein the recombinase is cre recombinase, and/or wherein the disrupted TR4 gene comprises a variant of the TR4 gene.

Disclosed are mammals comprising the vector and/or cells disclosed herein.

Disclosed are mammals, wherein the mammal is bovine, ovine, porcine, primate, mouse, rat, hamster, or rabbit.

Disclosed are mammals, wherein the disclosed vector has integrated into the mammals genome, comprising an integrated nucleic acid.

Disclosed are mice that are made by mating the TR4 knockout phenotype together with any other phentype or genotype mouse, for example, other knockout animals to produce double knockouts.

Disclosed are animals including mammals including bovine, ovine, porcine, primate, mouse, rat, hamster, or rabbit, which have had their TR4 gene disrupted. The TR4 gene can be disrupted in any way that interferes with the function of the TR4 gene product. The TR4 gene can be disrupted such that the resulting animal has anyt of the phenotypes disclosed herein.

C. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular TR2 is disclosed and discussed and a number of modifications that can be made to a number of molecules including the TR2 are discussed, specifically contemplated is each and every combination and permutation of TR2 and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

1. TR2/TR4

TR2 and TR4 are transcription factors that are able to modulate expression of a diverse panel of target genes. Through information gained from analysis of tissue expression of these orphan receptors throughout development and in the adult mouse, TR2 and TR4 play roles in the regulation of various aspects of developmental, physiological and behavioral systems. Disclosed are mouse models, having either TR2 or TR4 or both ablated, that can be used to determine the specific roles of TR2 and TR4 in vivo as well as used to identify and characterize molecules that interact with TR2 and TR4 in vivo. These disclosed models can also be used for the study of physiologically relevant information regarding the spatial and temporal expression patterns of TR2 and TR4, in addition to information regarding the consequences of lack of expression of the receptors. Disclosed, is data indicating that TR2 may be important in early developmental stages, whereas TR4 may play a more significant role later in development. The double knockouts have been made through mating of the single TR2 and TR4 knockouts and they are viable. Furthermore, in TR4 knockout, β-gal knockin animals, growth abnormalities and indications of infertility were observed.

a) Characterization of TR2 knockout/β-gal Knockin (TR2−/−) Mice

To explore the role of TR2 in mouse development, physiology, and behavior, mice with a targeted disruption of the receptor have been generated. These mice can be used as model systems for regulation of a variety of genes in vivo. Additionally, the mice are viable and fertile.

b) Characterization of TR4 knockout/β-gal Knockin (TR4−/−) Mice

To explore the role of TR4 in mouse development, physiology, and behavior, mice with a targeted disruption of the receptor have been generated. These mice can be used as model systems for regulation of a variety of genes in vivo. TR4−/− animals exhibit post-weaning size and weight reduction, and an abnormal gait, with the most difficulty in movement occurring with the hind limbs. Additionally, there are indications of infertility, as well as behavioral abnormalities characterized by general inactivity and reduced exploratory behavior.

Disclosed herein TR4 was specifically highly expressed in the primary spermatocytes at meiotic prophase, and TR4 expression dramatically increases and reach the highest level at this phase during the first wave of spermatogenesis in normal mice. In developing TR4^{-/-} mice, meiotic prophase and subsequent meiotic divisions were significant delayed and interrupted resulting in seriously delayed and disrupted first wave spermatogenesis. In TR4^{-/-} adult mice, stages XI-XII were prolonged and disrupted, where late meiotic prophase and subsequent meiotic divisions take place, resulting in the increased and prolonged metaphase cells and appearance of abnormal cells. Delayed and disrupted meiotic prophase and subsequent meiotic divisions can be further confirmed at the molecular level since two late meiotic prophase specific genes, sperm-1 and cyclin A1, expression were significantly delayed at the first wave of spermatogenesis and decreased in most developing and adult stages in TR4^{-/-} mice. Cyclin A1 has been shown to be a potential new molecular diagnostic marker for male infertile patients (Schrader et al., 200). Sperm-1 knockout mice show reduced fertility (Pearse et al., 1997). Delayed and decreased expression in TR4^{-/-} mice indicates the role of TR4 in late meiotic prophase and subsequent meiotic divisions. Sperm production in TR4^{-/-} mice is significantly reduced. Together, these data indicate that TR4 plays an important role during late meiotic prophase and subsequent meiotic divisions and TR4 is essential for normal spermatogenesis.

It has been known that there is close communication and interaction among testis cell types (Skinner, 1991). In TR4^{-/-} mice, the late stage pachytene spermatocytes and diplotene spermatocytes in some seminiferous tubules could not progress and complete the meiotic divisions, due to disrupted meiotic prophase. This could result in degeneration in other primary spermatocytes in these tubules, which will eventually spread into other testis cells and result in necrosis of these tubules. Necrotic tubules were observed in most testis sections that were examined from TR4^{-/-} mice, which explain why sperm production in TR4^{-/-} mice is significantly decreased.

In juvenile TR4^{-/-} mice, the first wave of spermatogenesis can be finished with several weeks delay, and in adult TR4^{-/-} mice, although stage XI-XII was prolonged, most tubules can eventually complete meiotic divisions and produce sperm. It has been known that many genes play important roles in spermatogenesis. The knockout of some these genes, like AR, could result in arrest of spermatogenesis (Yeh et al., 2002; Martianov et al., 2001). Knockout of other genes could result in significantly reduced spermatogenesis (Pearse et al., 1997). The latter genes or molecules have a potential to be developed as contraceptive methods. The ligand for TR4 so far is not known, but it is believed that this ligand could be a metabolite with a small molecular weight (Lee et al., 2002).

The disclosed data indicate that interruption or inhibition of TR4 can function as a male contraceptive composition. Thus disclosed are contraceptives comprising inhibitors of TR4 function.

It was observed that the fertility of TR4^{-/-} mice is significantly reduced, as well as quite a few abnormal sperm from TR4 mice. The disclosed data indicate that the many infertile human males that exhibit reduced sperm production but relatively normal mobile spermatozoa raises is consistent with impairment of TR4 function. The reduction of sperm production and fertility of the TR4^{-/-} mice make them useful models for studying the subtle events in mammalian reproduction, to identify cures, for example, for infertility. Furthermore, the addition of or administration of TR4 or TR4 enhancers, can aid in treating male infertility.

c) Characterization of TR2/TR4 Double Knockout Mice

Due to the high degree of structural and potential functional homology between TR2 and TR4, it is possible that these receptors are, to some degree, functionally redundant. TR2/TR4 double knockout animals from animals heterozygous for each targeted locus were generated. Analysis of the double knockout animals can be performed as for the single kockouts and can be compared to the phenotypes will be analyzed in comparison to TR2 knockout, TR4 knockout, and wildtype mice.

d) Analysis of the Effects of TR4 and/or TR2 Ablation on Target Gene Regulation

Although numerous genes have been identified as targets of TR4- and TR2-mediated regulation, the data have predominately been products of cell line-based transient transfection experiments. TR4, TR2, and TR2/TR4 knockout animals are excellent tools with which to study the known target genes of these orphan receptors in an in vivo system, and to confirm the physiological significance of the identified regulatory pathways. Additionally, the knockout animals provide excellent sources of material for the screening of novel TR2/TR4 target genes. Analysis of gene expression can be performed with the disclosed animals. All permutations of expression can be compared.

To determine the effects of loss of either TR4 or TR2, or the loss of both receptors on target gene expression, endogenous gene expression, and protein levels of TR4 and TR2 downstream targets in knockout animals versus wildtype controls can be compared. This type of data can validate prior in vitro data characterizing genes regulated by TR4, TR2, or both receptors. As most of the target genes of TR4 and TR2 identified to date are regulated similarly by both receptors, the disclosed TR4, TR2, and TR4/TR2 knockout mice can be used to screen for genes which are regulated by only one of the two receptors, or that are differentially regulated by TR4 and TR2. Gene microarray technology, can be used to dissect the differences in target gene regulation mediated by TR4 and TR2.

2. TR2

The human TR2 orphan receptor (TR2), a member of the nuclear hormone receptor superfamily, was cloned from human testis and prostate cDNA libraries and has no previously identified ligand(s) (Chang, C. et al. (1994) Proc Natl Acad Sci USA 91:6040-4); (Chang, C., and J. Kokontis (1988) Biophys Res Commun 155:971-7). TR2 is mapped to locate on chromosome 12q22 (Chang, C. et al. (1989) Biophys Res Commun 165:735-41), known to be frequently deleted in various tumors, including testicular and ovarian germ cell tumors (Chang, C., and H. J. Pan (1998) Mol Cell Biochem 189:195-200); Chen, W. S., et al. (1994) Genes Dev 8:2466-77). Four RNA isoforms, TR2-5, -7, -9, and -11, have been identified. While TR2-11 encodes the full-length receptor, TR2-5, -7, and -9 encode truncated receptors with distinct deletions of ligand-binding domains (LBD) (Chang, C. et al. (1994) Proc Natl Acad Sci USA 91:6040-4) TR2 has high homology with TR4, which places them in a unique subfamily within the nuclear hormone receptor superfamily (DeChiara, T. M. et al. (1995) Cell 83:313-22). TR2 is evolutionarily conserved among species from primitive creatures to mammalians, including sea urchin, rainbow trout, axolotl, xenopus, drosophila, mouse, and human (Chang, C., et al. (1994) Proc Natl Acad Sci USA 91:6040-4); (Chang, C., and J. Kokontis (1988) Biophys Res Commun 155:971-7); (Enmark, E., and J. A. Gustafsson (1996) Mol Endocrinol 10: 1293-307); (Evans, R. M. (1988) Science 240:889-95); (Franco, P. J. et al. (2001) Mol Endocrinol 15:1318-28); (Ingraham, H. A. et al. (1994) Genes Dev 8:2302-12); (Lee, C. H. et al. 1996.) Mol Reprod Dev 44:305-14).

The facts that TR2 is broadly expressed in many tissues throughout development starting at as early as midgestation stage (Lee, C. H. et al. (1995) Genomics 30:46-52); (Lee, Y. F. et al. (1997) J Biol Chem 272:12215-20); (Lee, Y. F. et al. (1998) J Biol Chem 273:13437-43); Lee, Y. F. et al. (1999) J Biol Chem 274:16198-205) and that drosophila with null mutations of DHR78 nuclear receptor, a homolog of human TR2, is lethal at the third-instar larval stage with severe defects in ecdysteroid-triggered metamorphosis (Lin, D. L. et al. (1998) Endocrine 8:123-34) are consistent with the biological importance of TR2 being involved in the development process. It has been emphasized that with prominent expression throughout the active proliferating zones of the neural areas and the sensory nerve-targeted organs and the testes during development, TR2 may exert an important role in the early development of the nervous system and the male reproductive system (Lee, C. H. et al. (1995) Genomics 30:46-52); (Lee, Y. F. et al. (1997) J Biol Chem 272:12215-20); (Lee, Y. F. et al. (1998) J Biol Chem 273:13437-43); (Lee, Y. F. et al. (1999) J Biol Chem 274:16198-205). Also, it has been shown that TR2 is primarily expressed in the mouse testis, particularly in the developing germ cells, indicating a role of TR2 in spermatogenesis (Lee, C. H. et al. (1995) Genomics 30:4&52); (Lin, T. M. et al. (1995) J Biol Chem 270:30121-8).

In cell line models, information regarding TR2 function, in terms of transcription activity, has been demonstrated by many studies. TR2 functions as a transcription factor that binds to its consensus response element (AGGTCA) in a direct repeat (DR) orientation (AGGTCA(n)ₓAGGTCA, x=1-6) (Lee, Y. F. et al. (1999) J Biol Chem 274:16198-205). New TR2 target genes are continually being discovered, such as cellular retinol-binding protein II (CRBPII), retinoic acid receptor β (RARβ), SV40, erythropoietin, histamine H1 receptor, muscle-specific aldolase A, and ciliary neurotrophic factor receptor (CNTFR) (Lee, Y. F. et al. (1997) J Biol Chem 272:12215-20); (Lee, Y. F. et al. (1998) J Biol Chem 273:13437-43); (Lee, Y. F. et al. (1999) J Biol Chem 274:16198-205), (Mangelsdorf, D. J. et al. (1995) Cell 83:835-9); (Masu, Y. et al. (1993) Nature 365:27-32); (Mu, X. et al. (2000) J Biol Chem 275:23877-83); (Pereira, F. A. et al. (1999) Genes Dev 13:1037-49.), suggesting that TR2 has a broad range of biological functions. In terms of the regulation of TR2 expression, TR2 can be induced during neuronal differentiation in P19 embryonic carcinoma cells stimulated by ciliary neurotrophic factor (CNTF). In return, TR2 activates its target gene, CNTFR, expression which mediates CNTF signaling and is required for the motor neuron development (Lee, Y. F. et al. (1997) J Biol Chem 272:12215-20); (Qiu, Y. et al. (1997) Genes Dev 11:1925-37). These may provide a linkage between TR2 and neurogenesis. The tumor suppressor genes, p53 and Rb, that induce cell cycle arrest can down-regulate TR2 expression in cells after ionizing radiation and in cells overexpressing p53 or Rb (Steinmayr, M. E. et al. (1998) Proc Natl Acad Sci USA 95:3960-5); (Wattler, S. et al. (1999) Biotechniques 26:1150-6, 1158, 1160). TR2 can then go through a feed-back control mechanism to induce HPV-16 E6 and E7 target gene expression that are known to enhance the P53 protein degradation and inactivate the Rb function, respectively (Steinmayr, M. et al. (1998) Proc Natl Acad Sci USA 95:3960-5); (Young, W. J., et al. (1998) J Biol Chem 273:20877-85). TR2 is, therefore, thought to be involved in cell cycle regulation.

In addition to functioning as a transcription regulator, TR2 can modulate other signaling via different mechanisms. For example, TR2 suppresses RXR- and RXR/RAR-mediated transcription by binding to the same DNA response element (DRE) with a higher binding affinity (Lee, Y. F. et al. (1999) J Biol Chem 274:16198-205) and represses thyroid receptor α/RXR signaling by competing for limited amounts of DREs (Mu, X. et al. (2000) J Biol Chem 275:23877-83).

TR2 can also exert its suppressive effects via the recruitment of class I and class II histone deacetylases (HDAC).

3. TR2

The human TR2 orphan receptor (TR2), a member of the nuclear hormone receptor superfamily, was cloned from human testis and prostate cDNA libraries and has no previously identified ligand(s) (Chang, C. et al. (1994) Proc Natl Acad Sci USA 91:6040-4); (Chang, C., and J. Kokontis (1988) Biophys Res Commun 155:971-7). TR2 is mapped to locate on chromosome 12q22 (Chang, C. et al. (1989) Biophys Res Commun 165:735-41), known to be frequently deleted in various tumors, including testicular and ovarian germ cell tumors (Chang, C., and H. J. Pan (1998) Mol Cell Biochem 189:195-200); Chen, W. S., et al. (1994) Genes Dev 8:2466-77). Four RNA isoforms, TR2-5, -7, -9, and -11, have been identified. While TR2-11 encodes the full-length receptor, TR2-5, -7, and -9 encode truncated receptors with distinct deletions of ligand-binding domains (LBD) (Chang, C. et al. (1994) Proc Natl Acad Sci USA 91:60404) TR2 has high homology with TR4, which places them in a unique subfamily within the nuclear hormone receptor superfamily (DeChiara, T. M. et al. (1995) Cell 83:313-22). TR2 is evolutionarily conserved among species from primitive creatures to mammalians, including sea urchin, rainbow trout, axolotl, xenopus, drosophila, mouse, and human (Chang, C., et al. (1994) Proc Natl Acad Sci USA 91:6040-4); (Chang, C., and J. Kokontis (1988) Biophys Res Commun 155:971-7); (Erunark, E., and J. A. Gustafsson (1996) Mol Endocrinol 10:1293-307); (Evans, R M. (1988) Science 240:889-95); (Franco, P. J. et al. (2001) Mol Endocrinol 15:1318-28); (Ingraham, H. A. et al. (1994) Genes Dev 8:2302-12); (Lee, C. H. et al. 1996.) Mol Reprod Dev 44:305-14).

The facts that TR2 is broadly expressed in many tissues throughout development starting at as early as midgestation stage (Lee, C. H. et al. (1995) Genomics 30:46-52); (Lee, Y. F. et al. (1997) J Biol Chem 272:12215-20); (Lee, Y. F. et al. (1998) J Biol Chem 273:13437-43); Lee, Y. F. et al. (1999) J Biol Chem 274: 16198-205) and that drosophila with null mutations of DHR78 nuclear receptor, a homolog of human TR2, is lethal at the third-instar larval stage with severe defects in ecdysteroid-triggered metamorphosis (Lin, D. L. et al. (1998) Endocrine 8:123-34) are consistent with the biological importance of TR2 being involved in the development process. It has been emphasized that with prominent expression throughout the active proliferating zones of the neural areas and the sensory nerve-targeted organs and the testes during development, TR2 may exert an important role in the early development of the nervous system and the male reproductive system (Lee, C. H. et al. (1995) Genomics 30:46-52); (Lee, Y. F. et al. (1997) J Biol Chem 272:12215-20); (Lee, Y. F. et al. (1998) J Biol Chem 273:13437-43); (Lee, Y. F. et al. (1999) J Biol Chem 274:16198-205). Also, it has been shown that TR2 is primarily expressed in the mouse testis, particularly in the developing germ cells, indicating a role of TR2 in spermatogenesis (Lee, C. H. et al. (1995) Genomics 30:46-52); (Lin, T. M. et al. (1995) J Biol Chem 270:30121-8).

In cell line models, information regarding TR2 function, in terms of transcription activity, has been demonstrated by many studies. TR2 functions as a transcription factor that binds to its consensus response element (AGGTCA) in a direct repeat (DR) orientation (AGGTCA(n)ₓAGGTCA, x=1-6) (Lee, Y. F. et al. (1999) J Biol Chem 274:16198-205). New TR2 target genes are continually being discovered, such as cellular retinol-binding protein II (CRBPII), retinoic acid receptor β (RARβ), SV40, erythropoietin, histamine H1 receptor, muscle-specific aldolase A, and ciliary neurotrophic factor receptor (CNTFR) (Lee, Y. F. et al. (1997) J Biol Chem 272:12215-20); (Lee, Y. F. et al. (1998) J Biol Chem 273:13437-43); (Lee, Y. F. et al. (1999) J Biol Chem 274:16198-205), (Mangelsdorf, D. J. et al. (1995) Cell 83:835-9); (Masu, Y. et al. (1993) Nature 365:27-32); (Mu, X. et al. (2000) J Biol Chem 275:23877-83); (Pereira, F. A. et al. (1999) Genes Dev 13:1037-49.), suggesting that TR2 has a broad range of biological functions. In terms of the regulation of TR2 expression, TR2 can be induced during neuronal differentiation in P19 embryonic carcinoma cells stimulated by ciliary neurotrophic factor (CNTF). In return, TR2 activates its target gene, CNTFR, expression which mediates CNTF signaling and is required for the motor neuron development (Lee, Y. F. et al. (1997) J Biol Chem 272:12215-20); (Qiu, Y. et al. (1997) Genes Dev 11: 1925-37). These may provide a linkage between TR2 and neurogenesis. The tumor suppressor genes, p53 and Rb, that induce cell cycle arrest can down-regulate TR2 expression in cells after ionizing radiation and in cells overexpressing p53 or Rb (Steinmayr, M. E. et al. (1998) Proc Natl Acad Sci USA 95:3960-5); (Wattler, S. et al. (1999) Biotechniques 26:1150-6, 1158, 1160). TR2 can then go through a feed-back control mechanism to induce HPV-16 E6 and E7 target gene expression that are known to enhance the P53 protein degradation and inactivate the Rb function, respectively (Steinmayr, M. et al. (1998) Proc Natl Acad Sci USA 95:3960-5); (Young, W. J., et al. (1998) J Biol Chem 273:20877-85). TR2 is, therefore, thought to be involved in cell cycle regulation.

In addition to functioning as a transcription regulator, TR2 can modulate other signaling via different mechanisms. For example, TR2 suppresses RXR- and RXR/RAR-mediated transcription by binding to the same DNA response element (DRE) with a higher binding affinity (Lee, Y. F. et al. (1999) J Biol Chem 274:16198-205) and represses thyroid receptor α/RXR signaling by competing for limited amounts of DREs (Mu, X. et al. (2000) J Biol Chem 275:23877-83).

TR2 can also exert its suppressive effects via the recruitment of class I and class II histone deacetylases (HDAC) (34A).

Prior evidence regarding embryological and adult expression of TR4 (Young, W. J., et al., Journal of Biological Chemistry 272, 3109-3116 (1997), Young, W. J., et al., Journal of Biological Chemistry 273, 20877-20885 (1998).) in the nervous system, as well as the demonstration that TR4 regulates transcription of the neural-specific gene ciliary neurotrophic factor receptor alpha (CNTFRα), provides indication of a role of TR4 in nervous system development and function. The ciliary neurotrophic factor has been shown to be protective against neurodegeneration in Parkinson's disease, and animals lacking CNTFRα display severe motor defects at birth (Young, W. J., et al., Journal of Biological Chemistry 272, 3109-3116 (1997)). Also, considering the function of the cerebellum in control of motor function (Strouboulis, J., et al., Genes and Development 6, 1857-1864 (1992)) and the high expression of TR4 in granule neurons in this brain region, a defect in motor function in the TR4KO might be expected. Such a defect was apparent early in the analysis of TR4-deficient mice, and especially upon study of male sexual behavior. Reported here is an analysis of the effects of loss of TR4 on the gross and cellular morphology and structure of the mouse brain, as well as a description of the resulting behavioral defects observed.

Nucleic Acids that encode various TR2s. There are many variants and allelic and homolog molecules of TR2. Genbank accession numbers for an exemplary set are provided here. Each of these sequences is herein incorporated by reference, at least for material related to the sequence. It is also understood that one of skill in the art would recognize the various TR2 proteins encoded by the nucleic acids, where protein sequence is not provided. A representative list of TR2 genes and related sequences can be found at Genbank Accession Nose: NT_{—}031693, NM 003807, BM313468, BM272414, BM272208, NM_{—}003297, BF476378, BF223014, BF109885, BE856797, 12: AW743650, AU076765, AW299455, AW272476, AW105139, AW073142, AW002180, A1983624, AF171055, AI893903, A1864325, A1686942, A1653325, AI507032, AI431858, AI370806, AI341113, AI203072, AI385609, AI379335, AI3708071, AA884437, AA770397, AI242989, A127957, AI153653, AI089445, AI081737, AI089220, U30482, AI050052, A1005665, AA227068, AA226914, AA656392, AA641155, AA593861, AA558488, AA454474, AA411285, AA381676, AA375076, AA300579, W36063, W39474, W38377, U19026, H75390, H68838, H68990, T27625, R54467, R52304, T95992, T95892, T84513, M29959, M29960, and M21985 and which are herein incorporated by reference at least for sequences related to TR2.

4. TR4

TR4 are transcription factors that are able to modulate expression of a diverse panel of target genes. Through information gained from analysis of tissue expression of TR4 through development TR4 plays a role in the regulation of various aspects of developmental, physiological and behavioral systems. Disclosed are mouse models, having TR4 ablated, that can be used to determine the specific roles of TR4 in vivo as well as used to identify and characterize molecules that interact with TR4 in vivo. These disclosed models can also be used for the study of physiologically relevant information regarding the spatial and temporal expression patterns of TR4, in addition to information regarding the consequences of lack of expression of the receptors. Disclosed is data indicating that TR4 may play a more significant role later in development. Furthermore, in TR4 knockout, β-gal knockin animals, growth abnormalities and indications of infertility were observed.

The human testicular receptor 4 (TR4) was originally isolated from testes, prostate, and brain cDNA libraries by degenerative polymerase chain reaction cloning (Chang, C., et al., (1994) Proc. Natl. Acad. Sci. USA. 91(13), 6040-4). While TR4 shares the structural features of nuclear receptors, no ligand has yet been previously identified and it is therefore considered an orphan receptor.

TR4 directly regulates transcription through binding to a direct repeat (DR) of a AGGTCA core element separated by a variable number of nucleotides. (TR4 can bind to AGGTCA direct repeats (DRx; AGGTCA (n)ₓ AGGTCA SEQ ID NO:13, x=0-6) (5E-13E)).TR4 functions as a transcriptional activator when bound to the DR separated by four nucleotides (a DR-4 element) (Lee, Y. F., et al., (1997) J. Biol. Chem. 272(18), 12215-20). However, TR4 functions as a transcriptional repressor when bound to DR-1, DR-2, DR-3, or DR-5 type (Lee, Y. F., et al., (1998) J. Biol. Chem. 273(22), 13437-43; Lee, H. J., et al., (1995) J. Biol. Chem. 270(50), 30129-33; Lee, Y. F., et al., (1999) J. Biol. Chem. 274(23), 16198-205). The differential spacings between the core elements cause TR4 to adopt different conformations and alter the ability of TR4 to interact with coregulators (Lee, Y. F., et al., (1999) J. Biol. Chem. 274(23), 16198-205). Consistent with its neuronal localization, TR4 also induces the transcription of the cytokine receptor, which is a ciliary neurotrophic factor receptor (Young, W. J., et al., (1997) J. Biol. Chem. 272(5), 3109-16).

In addition to direct transcriptional regulation, TR4 can also modulate other nuclear receptors' transactivation. Previous studies have indicated that TR4 can compete for binding to the hormone response elements of retinoic acid receptor (RAR), retinoid X receptor (RXR) (Lee, Y. F., et al., (1998) J. Biol. Chem. 273(22), 13437-43) and vitamin D receptor (VDR) (Lee, Y. F., et al., (1999) J. Biol. Chem. 274(23), 16198-205) to suppress RAR/RXR- or VDR-mediated transcription. TR4 may also inhibit peroxisome proliferator activated receptor alpha (PPARα) induced transactivation by competitive binding to PPAR response elements and through competition for coactivators such as RIP140 (Yan, Z. H., et al., (1998) J. Biol. Chem. 273(18), 10948-57). The AR-TR4 interaction could then result in the mutual suppression of AR- or TR4-mediated transcription (Lee, Y. F., et al., (1999) Proc. Natl. Acad. Sci. USA. 96(26), 14724-9). Previous reports have linked TR4 function to neurogenesis (Young, W. J., et al., (1997) J. Biol. Chem. 272(5), 3109-16) and spermatogenesis (Lee, C. H., et al., (1998) J. Biol. Chem. 273(39), 25209-15). TR4 has been demonstrated to suppress many other receptors' transactivation, such as VDR, RAR, RXR, and PPAR (Lee, Y. F., et al., (1998) J. Biol. Chem. 273(22), 1343743; Lee, Y. F., et al., (1999) J. Biol. Chem. 274(23), 16198-205; Yan, Z. H., et al. (1998) J. Biol. Chem. 273(18), 10948-57). The suppression mechanism for these receptors' transactivation has been demonstrated through the competition of TR4 with those receptors' ability to bind their hormone response elements.

The human TR4 cDNA shares structural homology with members of the steroid hormone receptor superfamily (Chang, C., et al., (1994) Proc. Natl. Acad. Sci. USA. 91, 6040-6044.). The TR4 is related to a number of steroid hormone receptors and is also named as TAK1 (Hirose, T., et al., (1994) Mol. Endocrinol. 8, 1667-1680), (Chang, C., and Kokontis, J. (1988) Biochem. Biophys. Res. Commun. 155, 971-977; Chang, C., et al., (1989) Biochem. Biophys. Res. Commun. 165, 735-741; Gronemeyer, H., and Laudet, V. (1995) Protein Profile 2, 1173-1308.), and is part of a subfamily within the superfamily of steroid receptors. Recently, the TR4 was designated as the TR2β, (Mangelsdorf, D. J., and Evans, R. M. (1995) Cell 83, 841-850). The mouse TR4 cDNA has been cloned from mouse testis by reverse transcription-PCR (Young, W.-J., et al., (1997) J. Biol. Chem. 272, 3109-3116). Subsequently, the human TR4 gene has been mapped to chromosome 3p24.3 (Lin, D.-L., et al., (1998) Endocrine 8, 123-134).

TR4 encodes a 67 kDa protein (Chang, C., et al., (1994) Proc. Natl. Acad. Sci. USA 91, 6040-6044). The P-box sequence of the DNA binding domain (DBD), TR4 is classified as a member of the estrogen receptor and thyroid hormone receptor subfamily, which can recognize the hormone response elements (HREs) composed of the AGGTCA motif Examples of HREs with this motif include those of the retinoic acid receptor (RARE), retinoid X receptor (RXRE) (Lee, Y.-F., et al., (1998) J. Biol. Chem. 273, 13437-13443), thyroid hormone receptor (T₃RE) (Lee, Y.-F., et al., (1997) J. Biol. Chem. 272, 12215-12220) and vitamin D receptor (VDRE)¹. In this case, TR4 may interfere with other steroid hormone pathways by binding to the same HREs. Several in vitro studies have demonstrated that TR4 acts as a regulator of various steroid/thyroid hormone pathways (Lee, Y.-F., et al., (1997) J. Biol. Chem. 272, 12215-12220; Young, W.-J., et al., (1997) J. Biol. Chem. 272, 3109-3116; Lee, H.-J., et al., (1995) J. Biol. Chem. 270, 30129-30133; Young, W.-J., et al., (1998) J. Biol. Chem. 273, 20877-20885).

In situ hybridization analysis shows that TR4 is highly expressed in adult mouse brain especially in the regions in which cells undergo active proliferation and in the granule cells of the hippocampus and cerebellum (Chang, C., et al., (1994) Proc. Natl. Acad. Sci. USA. 91(13), 6040-4). It has been demonstrated that TR4 inhibits the retinoic acid (RA) pathway that is highly involved in the development of the nervous system (Young, W.-J., et al., (1998) J. Biol. Chem. 273, 20877-20885). In contrast, TR4 enhanced the transactivation activity of the ciliary neurotrophic factor receptor (CNTFR) gene, whose expression pattern is restricted to nervous tissues and is highly similar to that of TR4, via binding to CNTFR-DR1 (Young, W.-J., et al., (1997) J. Biol. Chem. 272, 3109-3116). It was found that treatment of cells with RA would increase TR4 amounts at both RNA and protein levels (Lee, Y.-F., et al., (1998) J. Biol. Chem. 273, 13437-13443). The TR4 increase was also observed in CNTF-treated mouse P19 teratocarcinoma cells (Young, W.-J., et al., (1998) J. Biol. Chem. 273, 20877-20885). The data from both in situ and in vitro studies suggest that TR4 may be involved in the regulation of differentiation of neuron cells. TR4 is relatively highly expressed in several tissues including testis, kidney, and muscle. Northern blot analyses from multiple human and mouse tissues show a 9.4 kilobase and a 2.8 kilobase transcript. The 9.4 kilobase transcript is expressed ubiquitously, while the 2.8 kilobase transcript is largely restricted to the testis. In testis, TR4 is specifically expressed in germ cells (Hirose, et al., 1994; Hirose et al., 1995).

In situ hybridization analysis has demonstrated that TR4 is expressed in a complex spatiotemporal pattern. In the development of neurons, TR4 transcripts were detected throughout the neural tube at early stages of embryo development, and were subsequently restricted to the regions where cells were rapidly proliferating in the later stages of the embryo (Young, W.-J., et al., (1997) J. Biol. Chem. 272, 3109-3116). Consistent with in situ analysis of mouse embryos, the TR4 transcripts were expressed higher in the S-phase than in G1 and G2/M phases, determined by testing of elutriated P19 cell fractions.

In addition, the expression of the TR4 transcripts occurs widely in many mouse tissues, including the central nervous system and peripheral organs such as the adrenal gland, spleen, thyroid gland, and prostate (Yoshikawa, T., et al., (1996) Endocrinol. 137, 1562-1571; Young, W.-J., et al., (1997) J. Biol. Chem. 272, 3109-3116). These data are consistent with TR4 playing a role in neurogenesis and neuronal maturation.

Penile priapism, or persistent erection, is characterized by trapped blood within the corpus cavernosum, a condition leading to reduced tissue oxygenation, increased blood viscosity, disruption of tissue elasticity, fibrosis, and finally irreversible failure of erection (Winter 1978; Hauri et al. 1983; Panteleo-Gandais et al. 1984). The clinical description of partial priapism as well as segmental priapism refers to engorgement of the corpora cavemosum of the penis with stagnant blood, suggesting defects of blood diversion into the venous outflow or in problems with general venous drainage (Donatucci and Lue). Clinically, priapism is classified as primary (idiopathic) or secondary, with numerous potential causes. Such causes include hematologic disorders, traumatic or surgical injury (to the penis or spinal cord), neoplasia, infective toxic allergy, neurologic disorders, and pharmacologic induction (Hashmat and Rehman). Priapism has been associated with sickle cell disease in humans (Hashmat and Rehman), and is found in mouse models of sickle cell disease as well (Trudel et al. 1994; Beuzard 1996). Upon generation of mice lacking the Testicular orphan nuclear receptor 4 (TR4), penile priapism was one of the most striking phenotypes observed.

TR4 is also known to be highly expressed in the testis, with expression beginning at the end of the first wave of spermatogenesis, concomitant with the onset of meiosis. As spermatogenesis proceeds in waves throughout adulthood, the expression levels remain consistently high in spermatocytes, suggestive of a role for TR4 in regulation of proteins that support meiosis and the subsequent steps of spermatogenesis (Hirose et al. 1994; Lee et al. 1998a). Additionally, it was demonstrated that cryptorchidism and infertility-inducing high dose testosterone treatment in Rhesus monkeys resulted in repression of TR4 expression at the levels of both RNA and protein (Mu et al. 2000). Further, TR4 is known to regulate the luteinizing hormone receptor (LHR), as well as modulate the transcriptional activities of both ER and AR (Lee et al. 1999b; Zhang and Dufau 2000; Shyr et al. 2002b), which are all known mediators of male reproductive processes (Lee et al. 1975; Lubahn et al. 1993; Eddy et al. 1996b; Yeh et al. 2002).

A complex set of phenotypic abnormalities were found to exist in the TR4^{-/-} mouse. In addition to penile priapism among TR4^{-/-} males, reduced fertility among both males and females was found. In characterizing the male fertility defects, it was found that TR4^{-/-} mice displayed delayed onset of spermatogenesis, reduced sperm production, and abnormal sexual behavior. Hypothalamic expression of genes involved in behavior, stress response, and erectile function were found to be reduced in expression in TR4^{-/-} animals, and the synthetic enzyme nNOS, which is involved in production of the erectile mediator NO, was also reduced in expression in penis tissue from TR4-deficient males. Additionally, it was discovered that TR4^{-/-} animals are generated at less than expected Mendelian ratios, display abnormal maternal behavior, and show growth defects.

There are many variants and allelic and homolog molecules of TR4. Genbank accession numbers for an exemplary set are provided here. Each of these sequences is herein incorporated by reference, at least for material related to the sequence. It is also understood that one of skill in the art would recognize the various TR4 proteins encoded by the nucleic acids, where protein sequence is not provided. A representative list of TR4 genes and related sequences can be found at Genbank Accession Nos: NM_{—}017323 Rattus norvegicus TR4 orphan receptor (Tr4), mRNA; AV327704 RIKEN full-length enriched, adult male medulla oblongata Mus musculus cDNA clone 6330436D07 3′ similar to L27513 Rat TR4 orphan receptor mRNA, mRNA sequence; BF439121 Soares_NSF_F8_{—}9W_OT_PA_P_S1 Homo sapiens cDNA clone IMAGE:3270267 3′ similar to SW:TR4_HUMAN P49116 ORPHAN NUCLEAR RECEPTOR TR4; mRNA sequence; NM_{—}003298, Homo sapiens nuclear receptor subfamily 2, group C, member 2 (NR2C2), mRNA; U59454 Rattus norvegicus orphan receptor TR4-NS (TR4) gene, unspliced intron, partial sequence; and partial cds; AW169955 Soares_NFL_T_GBC_S1 Homo sapiens cDNA clone IMAGE:2659198 3′ similar to SW:TR4_HUMAN P49116 ORPHAN NUCLEAR RECEPTOR TR4; mRNA sequence; A1924957 wn26c02.x1 NCI_CGAP_Gas4 Homo sapiens cDNA clone IMAGE:2446562 3′ similar to SW:TR4_HUMAN P49116 ORPHAN RECEPTOR TR4; mRNA sequence; AI571166 tn85h11.x1 NCI_CGAP_Ut2 Homo sapiens cDNA clone IMAGE:2176389 3′ similar to SW:TR4_HUMAN P49116 ORPHAN RECEPTOR TR4; mRNA sequence; AA781743 ai60f03.s1 Soares_testis_NHT Homo sapiens cDNA clone 1375229 3′ similar to SW:TR4_HUMAN P49116 ORPHAN RECEPTOR TR4; mRNA sequence; AI221141qg91c11.x1 Soares_NFL_T_GBC_S1 Homo sapiens cDNA clone IMAGE: 1842548 3′ similar to SW:TR4 HUMAN P49116 ORPHAN RECEPTOR TR4; mRNA sequence; AI218732, oo07b04.x1 Soares_NSF_F8_{—}9W_OT_PA_P_S1 Homo sapiens cDNA clone IMAGE: 1565455 3′ similar to SW:TR4_HUMAN P49116 ORPHAN RECEPTOR TR4;, mRNA sequence; U59456 Rattus norvegicus nuclear hormone receptor rTR4alpha1 (TR4) mRNA, exon Q, partial cds; AH006640 Human orphan receptor TR4 (TR4) gene, intron N1, 5′ sequence; U40267 Human orphan receptor TR4 (TR4) gene, intron N3, 3′ sequence; U40266 Human orphan receptor TR4 (TR4) gene, intron N3, 5′ sequence; U40150 Human orphan receptor TR4 (TR4) gene, intron N2, 3′ sequence; U40149 Human orphan receptor TR4 (TR4) gene, intron N2, 5′ sequence; U40148 Human orphan receptor TR4 (TR4) gene, intron N1, 3′ sequence; U39639 Human orphan receptor TR4 (TR4) gene, intron N1, 5′ sequence; AI024815 ov35f10.x1 Soares_testis_NHT Homo sapiens cDNA clone IMAGE: 1639339 3′ similar to SW:TR4_HUMAN P49116 ORPHAN RECEPTOR TR4;, mRNA sequence AI018200, Soares_NFL_T_GBC_S1 Homo sapiens cDNA clone IMAGE:1626410 3′ similar to SW:TR4_HUMAN P49116 ORPHAN RECEPTOR TR4;, mRNA sequence; U32939 Mus musculus TR4 mRNA, partial cds; U59455 Homo sapiens TR4 gene, unspliced intron, 3′sequence; L27513 Rat TR4 orphan receptor mRNA, complete cds; and L27586 Human TR4 orphan receptor mRNA, complete cds. Protein sequences can be found at for example, Genbank Accession Nos: NP_{—}003289, AAB91433, P55094, AAC52777, AAC50677, AAC50676, AAC50675, AAC50674, AAC50673, AAC29502, AAC18408, AAA21475 and AAA21474 which are herein incorporated by reference at least for the disclosed sequences.

5. Androgen Receptor

Androgen receptor belonged to a superfamily of steroid hormone receptors was first subcloned in 1988 (Chang, 1988). It contains a N-terminal transactivation domain, a central DNA binding domain (DBD) and a C-terminal ligand binding domain (LBD) (Umesono, 1995). By forming a homodimer and taking into account of the ligand and coregulators, the androgen receptors interact and regulate the transcription of numerous target genes (Ing, 1992; Schulman, 1995; Beatp, 1996; Yeh, 1996; Glass, 1997, Shibata, 1997). Androgen is the strongest ligand of the androgen receptor. However, it is not the only ligand. Estradiol has been found to activate androgen receptor transactivation through the interaction with androgen receptor (Yeh, 1998). Besides, androgen and androgen receptor do not only act in male. The increasing evidence has displayed that the androgen and androgen receptor (AR) may also play important role in female physiological processes, including the process of folliculogenesis, the bone metabolism and the maintenance of brain functions (Miller, 2001).

Androgen is the most conspicuous amount of steroid hormone in ovary (Risch H A, 1998). The concentrations of testosterone and estradiol in the late-follicular phase when estrogens are at their peak are 0.06-0.10 mg/day and 0.04-0.08 mg.day respectively (Risch H A, 1998). The ratio of androgens versus estrogens in the ovarian veins of postmenopausal women is 15 to 1 (Risch, 1998; Doldi N, 1998). Androgen receptor is expressed dominantly in granulosa cells of ovary (Hiller S G, 1992; Hild-Petito S, 1991). With the overproduction of ovarian androgen, women with polycystic ovarian syndrome suffered from impairment of ovulatory function which is characterized with the increasing number of small antral follicles, but arrest in grafian follicles development (Kase, 1963; Futterweit W, 1986; Pache T D, 1991; Spinder T, 1989; Spinder T, 1989; Hughesdon P E, 1982). This symptom has suggested that AR may play a proliferative role in early folliculogenesis but turn to inhibitory effect in late folliculogenesis. The recent studies conducted in animals have supported this hypothesis (Harlow C R, 1988; Hilllier S, 1988; Weil S, 1998; Vendola K, 1998; Weil S, 1999; Vendola K, 1999). Administration of hihydroxytestosterone (DHT) in rhesus monkeys has increased the number of primary, preantral and small antral follicles. Since DHT is the metabolite of testosterone and cannot be aromatized, the result suggested the proliferative effect was through AR system (Vendola K, 1999).

6. Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

7. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

8. Nucleic Acids

There are a variety of molecules disclosed herein that are nucleic acid based, including for example the nucleic acids that encode, for example TR2, or any of the nucleic acids disclosed herein for making TR2 knockouts, or fragments thereof, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantagous that the antisense molecule be made up of nucleotide analogs that reduce the degradation of the antisense molecule in the cellular environment.

a) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate). There are many varieties of these types of molecules available in the art and available herein.

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties. There are many varieties of these types of molecules available in the art and available herein.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. There are many varieties of these types of molecules available in the art and available herein.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556). There are many varieties of these types of molecules available in the art and available herein.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

b) Sequences

There are a variety of sequences related to the protein molecules involved in the signaling pathways disclosed herein, for example TR2, or any of the nucleic acids disclosed herein for making TR2 knockouts, all of which are encoded by nucleic acids or are nucleic acids. The sequences for the human analogs of these genes, as well as other anlogs, and alleles of these genes, and splice variants and other types of variants, are available in a variety of protein and gene databases, including Genbank. Those sequences available at the time of filing this application at Genbank are herein incorporated by reference in their entireties as well as for individual subsequences contained therein. Genbank can be accessed at http://www.ncbi.nih.gov/entrez/query.fcgi. Those of skill in the art understand how to resolve sequence discrepancies and differences and to adjust the compositions and methods relating to a particular sequence to other related sequences. Primers and/or probes can be designed for any given sequence given the information disclosed herein and known in the art.

c) Primers and Probes

Disclosed are compositions including primers and probes, which are capable of interacting with the disclosed nucleic acids, such as the TR2 gene as disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the disclosed nucleic acids or region of the nucleic acids or they hybridize with the complement of the nucleic acids or complement of a region of the nucleic acids.

The size of the primers or probes for interaction with the nucleic acids in certain embodiments can be any size that supports the desired enzymatic manipulation of the primer, such as DNA amplification or the simple hybridization of the probe or primer. A typical primer or probe would be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments a primer or probe can be less than or equal to 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

The primers for the TR2 gene typically will be used to produce an amplified DNA product that contains the a region of the TR2 gene or the complete gene. In general, typically the size of the product will be such that the size can be accurately determined to within 3, or 2 or 1 nucleotides.

In certain embodiments this product is at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments the product is less than or equal to 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

d) Functional Nucleic Acids

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of any of the disclosed nucleic acids, such as TR2, and the nucleic acids used for the generation of TR2 knockouts, or the genomic DNA of any of the disclosed nucleic acids, such as TR2, and the nucleic acids used for the generation of TR2 knockouts or they can interact with the polypeptide encoded by any of the disclosed nucleic acids, such as TR2, and the nucleic acids used for the generation of TR2 knockouts. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

9. Delivery of the Compositions to Cells

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991) Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed TR2 nucleic acids or vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the nucleic acid or vector of this invention can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

In the methods described above which include the administration and uptake of exogenous DNA into the cells of a subject (i.e., gene transduction or transfection), the nucleic acids of the present invention can be in the form of naked DNA or RNA, or the nucleic acids can be in a vector for delivering the nucleic acids to the cells, whereby the antibody-encoding DNA fragment is under the transcriptional regulation of a promoter, as would be well understood by one of ordinary skill in the art. The vector can be a commercially available preparation, such as an adenovirus vector (Quantum Biotechnologies, Inc. (Laval, Quebec, Canada).

As one example, vector delivery can be via a viral system, such as a retroviral vector system which can package a recombinant retroviral genome (see e.g., Pastan et al., Proc. Natl. Acad. Sci. U.S.A. 85:4486, 1988; Miller et al., Mol. Cell. Biol. 6:2895, 1986). The recombinant retrovirus can then be used to infect and thereby deliver to the infected cells nucleic acid encoding a broadly neutralizing antibody (or active fragment thereof) of the invention. The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naidini et al., Science 272:263-267, 1996), pseudotyped retroviral vectors (Agrawal et al., Exper. Hematol. 24:738-747, 1996). Physical transduction techniques can also be used, such as liposome delivery and receptor-mediated and other endocytosis mechanisms (see, for example, Schwartzenberger et al., Blood 87:472478, 1996). This invention can be used in conjunction with any of these or other commonly used gene transfer methods.

As one example, if a nucleic acid disclosed herein is delivered to the cells of a subject in an adenovirus vector, the dosage for administration of adenovirus to humans can range from about 10⁷ to 10⁹ plaque forming units (pfu) per injection but can be as high as 10¹² pfu per injection (Crystal, Hum. Gene Ther. 8:985-1001, 1997; Alvarez and Curiel, Hum. Gene Ther. 8:597-613, 1997). A subject can receive a single injection, or, if additional injections are necessary, they can be repeated at six month intervals (or other appropriate time intervals, as determined by the skilled practitioner) for an indefinite period and/or until the efficacy of the treatment has been established.

Parenteral administration of the nucleic acid or vector of the present invention, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein. For additional discussion of suitable formulations and various routes of administration of therapeutic compounds, see, e.g., Remington: The Science and Practice of Pharmacy (19th ed.) ed. A. R. Gennaro, Mack Publishing Company, Easton, Pa. 1995.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer. 58:700-703, (1988); Senter, et al., Bioconjugate Chem. 4:3-9, (1993); Battelli, et al., Cancer Immunol. Immunother. 35:421-425, (1992); Pietersz and McKenzie, Immunolog. Reviews, 129:57-80, (1992); and Roffler, et al., Biochem. Pharmacol. 42:2062-2065, (1991)). These techniques can be used for a variety of other speciifc cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research. 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399409 (1991)).

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

a) In Vivo/Ex Vivo

As described above, the compositions can be administered in a pharmaceutically acceptable carrier and can be delivered to the subject's cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

10. Expression Systems

The nucleic acids that are delivered to cells typically contain expression controlling systems. For example, the inserted genes in viral and retroviral systems can contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

a) Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

b) Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

11. Peptides

a) Protein Variants

As discussed herein there are numerous variants of the TR2 protein that are known and herein contemplated. In addition, to the known functional TR2 allelic variants there are derivatives of the TR2 proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 2 and 3 and are referred to as conservative substitutions.

TABLE 2
Amino Acid Abbreviations

| Amino Acid | Abbreviations |
|---|---|
| Alanine | AlaA |
| Allosoleucine | AIle |
| Arginine | ArgR |
| Asparagines | AsnN |
| aspartic acid | AspD |
| Cysteine | CysC |
| glutamic acid | GluE |
| Glutamine | GlnQ |
| Glycine | GlyG |
| Histidine | HisH |
| Isolelucine | IleI |
| Leucine | LeuL |
| Lysine | LysK |
| Phenylalanine | PheF |
| Proline | ProP |
| pyroglutamic acidp | Glu |
| Serine | SerS |
| Threonine | ThrT |
| Tyrosine | TyrY |
| Tryptophan | TrpW |
| Valine | ValV |

TABLE 3
Amino Acid Substitutions

| | Exemplary Conservative |
|---|---|
| | Substitutions, others are |
| Original Residue. | known in the art |
| Ala | Ser |
| Ar | glys, gln |
| Asn | gln; his |
| Asp | Glu |
| Cys | Ser |
| Gln | asn, lys |
| Glu | Asp |
| Gly | Ala |
| His | asn; gln |
| Ile | leu; val |
| Leu | ile; val |
| Lys | arg; gln; |
| Met | Leu; ile |
| Phe | met; leu; tyr |
| Ser | Thr |
| Thr | Ser |
| Trp | Tyr |
| Tyr | trp; phe |
| Val | ile; leu |

Substantial changes in function or immunological identity are made by selecting substitutions that are less conservative than those in Table 3, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. Specifically disclosed are variants of TR2 and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL. Biol. 48: 443 (1970), by the search for similarity method of Pearson and Lipman, Proc. Natl. Acad. Sci. U.S.A. 85: 2444(1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

12. Antibodies

Disclosed are antibodies related to the disclosed compositions. For example, it is understood that the disclosed knockouty mice could be used for generation of a particular antibody, could produce antigens which would be desirable in the generation of antibodies, such as a monoclonal antibody, and could have antibodies administered to them. Those of skill in the art understand how to generate monoclonal antibodies and administer them, for example, see Kohler and Milstein, Nature, 256:495 (1975) which is herein incorporated by reference for material related to antibody production.

13. Pharmaceutical Carriers/Delivery of Pharamceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews. 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

Effective dosages and schedules for administering the compositions may be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

14. Chips and Micro Arrays

Disclosed are chips where at least one address is the sequences or part of the sequences set forth in any of the nucleic acid sequences disclosed herein. Also disclosed are chips where at least one address is the sequences or portion of sequences set forth in any of the peptide sequences disclosed herein.

Also disclosed are chips where at least one address is a variant of the sequences or part of the sequences set forth in any of the nucleic acid sequences disclosed herein. Also disclosed are chips where at least one address is a variant of the sequences or portion of sequences set forth in any of the peptide sequences disclosed herein.

15. Computer Readable Mediums

It is understood that the disclosed nucleic acids and proteins can be represented as a sequence consisting of the nucleotides of amino acids. There are a variety of ways to display these sequences, for example the nucleotide guanosine can be represented by G or g. Likewise the amino acid valine can be represented by Val or V. Those of skill in the art understand how to display and express any nucleic acid or protein sequence in any of the variety of ways that exist, each of which is considered herein disclosed. Specifically contemplated herein is the display of these sequences on computer readable mediums, such as, commercially available floppy disks, tapes, chips, hard drives, compact disks, and video disks, or other computer readable mediums. Also disclosed are the binary code representations of the disclosed sequences. Those of skill in the art understand what computer readable mediums. Thus, computer readable mediums on which the nucleic acids or protein sequences are recorded, stored, or saved.

Disclosed are computer readable mediums comprising the sequences and information regarding the sequences set forth herein.

16. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended. For example, disclosed is a kit for assessing testing compounds related to testicular orphan nuclear receptor 2 comprising the TR2 mouse disclosed herein, and the reagents to aid in the testing.

D. Methods of Making the Compositions

The compositions disclosed herein and the compositions necessary to perform the disclosed methods can be made using any method known to those of skill in the art for that particular reagent or compound unless otherwise specifically noted.

1. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

2. Peptide Synthesis

One method of producing the disclosed proteins is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides may be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

3. Process for Making the Compositions

Disclosed are processes for making the compositions as well as making the intermediates leading to the compositions. There are a variety of methods that can be used for making these compositions, such as synthetic chemical methods and standard molecular biology methods. It is understood that the methods of making these and the other disclosed compositions are specifically disclosed.

Disclosed are nucleic acid molecules produced by the process comprising linking in an operative way a nucleic acid comprising the sequence of a TR2 exon, such as exons 3-7 or exon 4 and 5, for example, and sequence recognized by a recombinase enzyme.

Also disclosed are nucleic acid molecules produced by the process comprising linking in an operative way a nucleic acid molecule comprising a sequence having 80% identity to a sequence of an TR2 exon, such as exons 3-7 or exon 4 and 5, for example, and sequence recognized by a recombinase enzyme.

Disclosed are nucleic acid molecules produced by the process comprising linking in an operative way a nucleic acid molecule comprising a sequence that hybridizes under stringent hybridization conditions to a sequence of an TR2 exon, such as exons 3-7 or exon 4 and 5, for example, and sequence recognized by a recombinase enzyme.

Disclosed are cells produced by the process of transforming the cell with any of the disclosed nucleic acids. Disclosed are cells produced by the process of transforming the cell with any of the non-naturally occurring disclosed nucleic acids.

Disclosed are any of the disclosed peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the non-naturally disclosed nucleic acids.

Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate, such as a human, monkey, ape, chimpanzee, or orangutan.

Also disclose are animals produced by the process of adding to the animal any of the cells disclosed herein.

Disclosed compositions and methods, such as vectors, that can be used for targeted gene disruption and modification in any animal that can undergo these events. Gene modification and gene disruption refer to the methods, techniques, and compositions that surround the selective removal or alteration of a gene or stretch of chromosome in an animal, such as a mammal, in a way that propagates the modification through the germ line of the mammal. In general, a cell is transformed with a vector which is designed to homologously recombine with a region of a particular chromosome contained within the cell, as for example, described herein. This homologous recombination event can produce a chromosome which has exogenous DNA introduced, for example in frame, with the surrounding DNA. This type of protocol allows for very specific mutations, such as point mutations, to be introduced into the genome contained within the cell. Methods for performing this type of homologous recombination are disclosed herein.

One of the preferred characteristics of performing homologous recombination in mammalian cells is that the cells should be able to be cultured, because the desired recombination event occur at a low frequency.

Once the cell is produced through the methods described herein, an animal can be produced from this cell through either stem cell technology or cloning technology. For example, if the cell into which the nucleic acid was transfected was a stem cell for the organism, then this cell, after transfection and culturing, can be used to produce an organism which will contain the gene modification or disruption in germ line cells, which can then in turn be used to produce another animal that possesses the gene modification or disruption in all of its cells. In other methods for production of an animal containing the gene modification or disruption in all of its cells, cloning technologies can be used. These technologies generally take the nucleus of the transfected cell and either through fusion or replacement fuse the transfected nucleus with an oocyte which can then be manipulated to produce an animal. The advantage of procedures that use cloning instead of ES technology is that cells other than ES cells can be transfected. For example, a fibroblast cell, which is very easy to culture can be used as the cell which is transfected and has a gene modification or disruption event take place, and then cells derived from this cell can be used to clone a whole animal.

Disclosed are nucleic acids used to modify a gene of interest that is cloned into a vector designed for example, for homologous recombination.

E. Methods of Using the Compositions

1. Methods of Using the Compositions as Research Tools

The disclosed compositions can be used in a variety of ways as research tools. For example, the disclosed compositions, such as the TR2 mice can be used to study reagents related to TR2 related cancers, as well as for drug discovery for TR2 related diseases. The disclosed compositions can also be used diagnostic tools and any disease related to testicular orphan nuclear receptor 2 function. Furthermore, the disclosed constructs and animals can be used as reagents to produce double and even multimer knockouts of other genes. The disclosed animals can be bred to make other animal lines possessing the disclosed knockout's as well as any other knockout proteins.

F. Examples

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. EXAMPLE 1

a) Results

(1) Generation of TR2-Deficient Mice

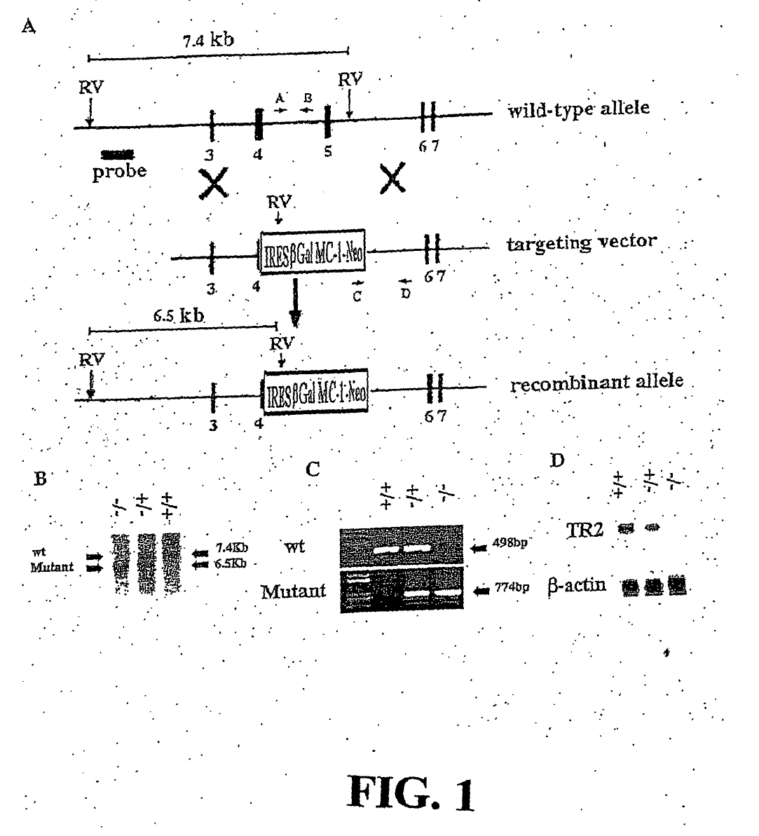

Unlike the human TR2 gene that is located on human chromosome 12 at band q22 (16 A, herein specifically incorporated by reference at least for material related to TR2), the mouse TR2 gene is located in the distal region of chromosome 10 and is organized into 13 exons spanning more than 50 kb (Lee, C. H. et al. (1995) Genomics 30:46-52) herein specifically incorporated by reference at least for material related to TR2)). A replacement vector was designed to delete most of exon 4, which encodes the second zinc finger domain of the TR2 DNA binding domain, and all of exon 5. The deleted sequence was replaced with an IRES LacZ/MC1-Neo selection cassette (FIG. 1A). The targeting vector was electroporated into 129 Sv/Ev^{brd}(LEX1) and colonies were selected in the presence of G418 and FIAU. G418/FIAU resistant ES-cell clones were isolated and analyzed for homologous recombination using Southern blot analyses. Targeted ES-cell clones were injected into C57BL/6(albino) blastocysts and the resulting chimeras were mated to C57BL/6(albino) females to generate heterozygous mice. The heterozygous mice (TR2^{+/-}) were then interbred to produce null mutation (TR2^{-/-}) mice.

(2) Southern Blot, Northern Blot, and PCR Analyses of TR2 Mutant Mice

Genomic DNA isolated from mouse tail biopsies was used for Southern blot analyses to confirm genotypes, with the expectation that TR2^{+/+} animal will have a 7.4 kb DNA fragment after digestion with restriction enzyme EcoRV. The disrupted TR2 gene in TR2^{-/-} animals display a 6.5 kb DNA fragment (FIG. 1B). Genotypes were confirmed by PCR using 2 sets of primers: both primers in the first set (A, B) are located on the TR2 gene between exons 4 and 5, which was replaced by IRES LacZ/MC1-Neo selection cassette (FIG. 1A). For the second set of primers (C, D); primer C is located within the Neo cassette and primer D is outside the Neo cassette and is located on the 3′ end of TR2 gene (FIG. 1A). Using primers A&B, PCR amplification of the TR2 gene without interruption by Neo cassette will produce a 498 bp PCR DNA fragment and indicates a wild-type TR2 allele. While using primers C&D, PCR amplification of the TR2 gene with Neo cassette interruption should produce a 774 bp PCR DNA fragment and indicates the presence of the Neo cassette. As expected, TR2^{+/+} mice produced a single 498 bp DNA fragment, TR2^{-/-} mice produced a single 774 bp fragment, and heterozygous TR2^{+/-} mice produced both 498 bp and 774 bp DNA fragments (FIG. 1C). To confirm the loss of expression of TR2 in the knockout mice, total RNAs from 6-week-old testes of TR2^{+/+}, TR2^{+/-} and TR2^{-/-} mice were analyzed by Northern blotting using a mouse TR2 cDNA fragment as a probe. FIG. 1D clearly demonstrates that TR2^{-/-} mice lack TR2 mRNA which were knocked-out by replacement of Neo cassette. Together, results from FIG. 1B to 1D clearly demonstrate that the TR2 gene was successful disrupted in the TR2^{-/-} mice.

(3) TR2^{-/-} Mice are Viable and Fertile

Mating of TR2^{+/-} mice generates pups of three possible genotypes (TR2^{+/+}, TR2^{+/-}, and TR2^{-/-}) that fit well with normal Mendelian frequencies, suggesting that TR2 is not required for normal embryonic development. The external and internal phenotypes of TR2^{-/-} mice appear normal. Furthermore, breeding studies showed that both male and female TR2^{-/-} mice are fertile and can produce normal offspring. Additionally, the histopathological analyses from necropsy studies of more than 30 tissues including brain and prostate in four adult TR2^{-/-} animals revealed no gross anatomical defects and significant lesions.

(4) Normal Testis Development and Spermatogenesis in TR2^{-/-} Mice

TR2 expression has been demonstrated in mature testes and is mainly present in advanced germ cell population (Lee, C. H. et al. (1995) Genomics 30:46-52). The β-gal activity from the LacZ reporter gene present in the knockout construct and in TR2^{+/-} or TR2^{-/-} mice allows for the analysis of TR2 expression. Testes collected from TR2^{+/-} mice at different ages were examined for the distribution of β-gal activity which represents TR2 protein expression. The results show that TR2 expression was observed in the testis only after the first wave of spermatogenetic cells have completed both meiotic division I and II, which occurs around 3 weeks after birth. β-gal activity localized predominantly in more advanced germ cells (pachytene spermatocytes and round spermatids) and was not found in spermatogonia and elongated spermtids (The data showed expression of TR2 represented by β-gal activity in TR2 (+/−) heterozygous mice. Testes from postnatal mice of different ages were processed through whole-mount X-Gal staining, embedded in paraffin, sectioned and counterstained with H&E. Magnification, 100×(A) 1 week. (B) 2 weeks. (C) 3 weeks. (D) 4 weeks. (E) 5 weeks. (F) 6 weeks. Blue positive staining is indicated by arrows. (G) Northern blot analysis of total RNA from testis tissue of mice at 1, 2, 3, 4, 5 and 6 weeks of age. The same membrane was sequentially hybridized with ³²P-labeled TR2 and β-actin probes.). In contrast, β-gal activity is undetectable in the testes from 1 and 2 week old TR2^{+/-} mice. To further prove this distinct expression pattern of TR2 in testes, total RNAs were isolated from the TR2^{+/+} mice testes at different ages for Northern blotting analysis to compare the TR2 mRNA expression with β-gal expression in TR2^{+/-} mice. The expression of TR2 mRNA in TR2^{+/+} mouse testes is similar to β-gal activity in TR2^{+/-} mouse testes. In both cases, the expression is detected starting at 3 weeks of age and increases as testes develop into maturity.

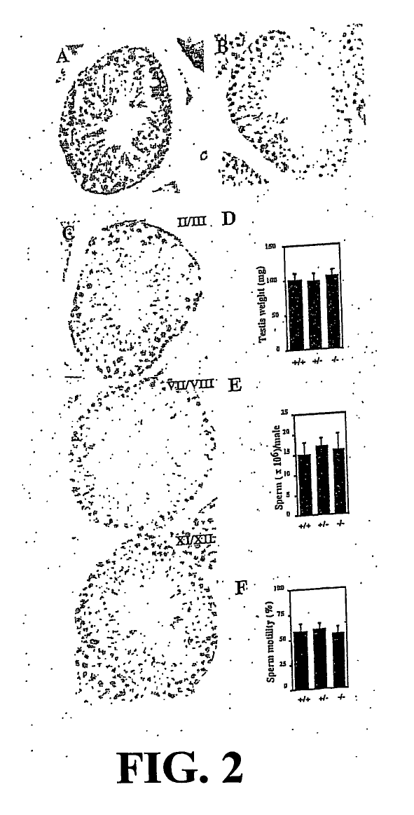

As shown in FIGS. 2A and B, examination of hematoxylin-eosin-stained paraffin-embedded sections from 6-week TR2^{-/-} mice did not reveal any abnormal histological features. The diameter of the seminiferous tubules, the thickness of the seminiferous epithelium, and the size of the lumen in TR2^{-/-} mice appear normal. Spermatogenesis also appears to progress normally in TR2^{-/-} mice.

In mouse spermatogenesis, there are 12 designated cell associations or stages which are defined groupings of germ cell types at particular phases of development in cross-sectioned tubules. The staging of spermatogenesis allows understanding of normal spermatogenesis and what might go wrong with spermatogenesis. FIG. 2C presents selected stages of TR2 homozygous mutant mice. Abnormal cell types were not observed or present in any stage. Additionally, there were no significant differences in testes weight (FIG. 2D), sperm count (FIG. 2E), or sperm motility (FIG. 2F) among TR2^{+/+}, TR2^{+/-}, and TR2^{-/-} mice.

(5) TR4 Expression is Normal in the TR2^{-/-} Mice

One explanation for the absence of abnormality in testes development and spermatogenesis in TR2^{-/-} mice involves functional compensation by another orphan receptor, TR4, which exhibits high homology (65% overall) with TR2 (Chang, C. et al. (1994) Proc Natl Acad Sci USA 91:6040-4). Early studies also indicate that these two orphan receptors share many functions in regulating several signaling pathways. To determine whether TR4 expression is increased in TR2^{+/-}, and/or TR2^{-/-} mice, total RNAs were isolated from 6-week-old testes of TR2^{+/+}, TR2^{+/-}, and TR2^{-/-} mice, and Northern blot analysis was carried out using mouse TR4 cDNA as a probe. The results show that TR4 mRNA is expressed at similar level among TR2^{+/+}, TR2^{+/-} and TR2^{-/-} mice (The data showed an analysis of TR4 expression. Total RNA from 6 week old wild type mice (+/+), heterozygous mutant (+/−), and homozygous mutant mice (−/−) was sequentially hybridized with ³²P-labeled TR4 and β-actin probes.).

(6) The Central Nervous System is normal in TR2^{-/-} Mice

Although TR2 is expressed in neural epithelia, spinal cord, cerebellar primordium, and the periventricular area of the developing brain (Young, W. J. et al. (1998) J Biol Chem 273:20877-85), no gross abnormalities were found with the examination of the serial sections of cerebra, cerebella, and spinal cords in 3 month old wild type mice (+/+), and homozygous mutant mice (−/−). The six cortical layer laminar structure is well-formed and the cerebellar cortex consists of three layers in both mice (The data showed a comparison of the cerebral cortex, cerebellar cortex, and lumbar cord spinal motor neurons in 3 month old wild type mice (+/+), and homozygous mutant mice (−/−). (A) Coronal sections of the cerebral cortex in wild type and mutant mice. Six layer laminar structures are seen in both mice. Magnification, 100×. (B) Coronal sections of cerebellar cortex. Three layers are seen in both mice. Magnification, 100×. (C) Transverse sections of lumbar spinal cord in wild type and mutant mice. Magnification, 400×. Brains and spinal cords were fixed in neutral buffered formalin, sectioned, and stained with H&E. Abbreviations; I-VI, cortical layers I through VI; WM, white matter; ML, molecular later; P, Purkinje cells; and GL, granular cell layer.). TR2 is not only highly expressed in the developing nervous system, but also induces ciliary neurotrophic factor (CNTF) receptor α (CNTFRα) gene transcriptional activity and the expression of TR2 is increased in P19 cells treated with CNTF (Young, W. J. et al. (1998) J Biol Chem 273:20877-85). Mice lacking the CNTF gene exhibit a progressive atrophy and loss of motor neurons in adult mice (Masu, Y. et al. (1993) Nature 365:27-32), Motor neuron number is dramatically reduced in mice homozygous for null mutations in the CNTFRα gene (DeChiara, T. M. et al. (1995) Cell 83:313-22). These studies indicate the importance of CNTF signaling pathway in the maintenance of motor neurons. However, in contrast to mice lacking CNTF and CNTFRα, lumbar motor neurons in TR2^{-/-} mice showed no obvious reduction in number.

b) Materials and Methods

(1) Generation of TR2 Knockout Mice

To generate TR2 deficient mice, the lambda KOS system (Wattler, S. et al. (1999) Biotechniques 26:1150-6, 1158, 1160) herein specifically incorporated by reference at least for material related to knockouts and their generation as well as the nucleic acids related to such)) was used to derive a TR2 targeting vector. Two independent genomic Lambda KOS clones were isolated that spanned exons 3-7. The targeting vector was derived from one clone and contained a 1865 base pair (bp) deletion that included most of exon 4 and all of exon 5. This region was replaced with an IRES LacZ/MC1-Neo selection cassette. The NotI linearized vector was electroporated into 129 Sv/Ev^{brd} (LEX1) ES cells and G418/FIAU resistant ES-cell clones were isolated and analyzed for homologous recombination using Southern blot analysis. Targeted ES-cell clones were injected into C57BL/6(albino) blastocysts and the resulting chimeras were mated to C57BL/6(albino) females to generate animals heterozygous for the mutation.

(2) Southern Blot, Northern Blot, and PCR Analyses of TR2 Knockout Mice

Genomic DNA isolated from mouse tail biopsies was digested with EcoRV, separated by electrophoresis through a 0.8% agarose gel, and transferred to a positively charged nylon membrane. A wild-type 5′ probe external to the target vector sequence was labeled with a random primer labeling kit (Amersham) and used in hybridizations. Wild-type and mutant alleles were identified by predicted restriction fragment size differences.

PCR was also used to screen the genotypes using DNA isolated from mouse tail biopsies. The primers used for the wild-type allele were (A) 5′-CCCTGACTAGTTTCTGCGATC-3′ (SEQ ID NO:14) and (B) 5′-GCCTACTCATGGAAATATAACC-3′ (SEQ ID NO15). Primers (C) 5′-GCTGATGCTACCAAGTCCACG-3′ (SEQ ID NO:16) and (D) 5′-GCAGCGCATCGCCTTCTATC-3′ (SEQ ID NO:17) were used to detect the mutant allele. RNA was extracted from testes of mice at different ages using TRIzol reagent (GIBCO BRL). 30 μg of RNA was loaded into 1.2% agarose gels. Northern blot analysis of TR2 and β-actin transcripts was performed as previously described (Young, W. J. et al. (1998) J Biol Chem 273:20877-85).

(3) Necropsy Studies

Gross and histopathological analyses were performed at the Diagnostic Laboratories, Division of Laboratory animal Medicine, University of Rochester.

(4) Epididymal Sperm Numbers

Epididymides were removed from 6-week-old males under sterile conditions and placed in a dish containing 5 ml of Dulbecco modified Eagle medium (DMEM) with 10% fetal calf serum. Sperm was allowed to disperse into the medium for 1 h at 32° C. Sperm motility was examined by phase-contrast microscopy. Sperm numbers were determined by counting with a hemocytometer.

(5) Histological Analysis

Tissues were fixed in fresh 10% neutral buffered formalin and embedded in paraffin. Tissue sections (5 μm) were stained with hematoxylin and eosin (H&E) and examined by light microscopy. For β-galactosidase (β-gal) activity staining, testes at different ages of mice were dissected and fixed in 0.2% glutaraldehyde for 2 h at room temperature. The tissues were then washed with phosphate buffer and incubated overnight at room temperature in phosphate buffer containing 2 mM MgCl₂, 5 mM K₄Fe(CN)₆, 5 mM K₃Fe(CN)₆, and 1 mg/ml X-Gal. After washing with PBS, testes were dehydrated, embedded in paraffin, and sectioned at 5 μm. The sections were then counterstained with H&E staining and examined by light microscopy.

### 2. EXAMPLE 2

### TR2 and TR4 Expression

a) Tissue Distribution of TR2 and TR4

The expression patterns of these receptors in various tissues, through mouse embryonic development, as well as in the adult has been characterized. High expression of TR2 and TR4 receptors in both the testis and brain are consistent with roles in reproductive function, as well as in learning and behavior.

Both TR2 and TR4 are expressed in the developing nervous system of the mouse embryo. From in situ hybridization analyses probing for either TR2 or TR4 mRNA, transcripts are observed in actively proliferating cell populations of the brain and peripheral organs during embryonic development (The data showed TR4 mRNA expression during mouse embryogenesis. Saggital sections of mouse embryos were probed with either a sense (A) or an antisense (B-J) riboprobe specific for TR4, and photographed under dark-field illumination. A-E, low magnification photographs of TR4 expression in embryos at the indicated developmental stages. Particularly strong expression of TR4 transcripts in the ventricular zones are indicated as arrows (D) and an arrowhead (E). F-G, high magnification photographs of TR4 expression in regions of the developing forebrain (F), inner ear (G), spinal cord (H), eye (I), and the junction between the nasal and oral cavities (J), in an E16 embryo. Ventricular zones are indicated by arrowheads (F). Abbreviations: c, superior cervical ganglion; cp, cerebellar primordium; cx, cerebral cortex; drg, dorsal root ganglia; e, otic epithelium; g, ganglion layer of retina; l, lens; m, motor neuron; ms, muscle; n, nuclear layer of retina; ne, neural epithelium; oe, olfactory epithelium; r, retina; s, sympathetic ganglia; sp, spinal cord; Sr, striatum; t, tongue epithelium; V, trigeminal ganglion. The size bars represent 1 and 100 nm for A-E and F-J, respectively) (Young, W. J. et al. (1997) J. Bioi. Chern. 272,3109-3116). This expression pattern is consistent with roles for TR2 and TR4 in the process of neural development. Specifically, TR4 is expressed in the neural tube of the mouse at embryonic days 9-11 (E9-E11). At E14-16, TR4 expression is particularly strong in ventricular zones of the brain, as well as in the striatum and the cerebellar primordium. Expression of TR4 at E16 also extends to the spinal cord, including spinal motor neurons, suggesting the potential for limb coordination defects in the TR4 knockout animal. Sites of sensory organ development may be affected by TR4 ablation in that neuronal nuclei involved in sensory organ development show high expression of the receptor. The specific areas of expression include the dorsal root ganglia, superior cervical ganglia, sympathetic ganglia, and trigeminal ganglia. Sensory innervation targets including the neuronal epithelium of the inner ear, retina, nasal cavity, and tongue show strong TR4 expression as well. Regions outside the nervous system with significant TR4 expression include the perichondrium, kidney, hair follicle, and tooth bud (The data showed TR4 mRNA expression in non-neural tissues during mouse embryogenesis. Saggital sections of mouse embryos at gestation day 14 (A) and day 16 (B and C) were analyzed by in situ hybridization with a mouse TR4 antisense riboprobe. Autoradiograms were photographed under light field illumination with (C) or without (A and B) hematoxylin staining. Tissues with strong hybridization signals (dark areas) are labeled. High magnification (B and C) shows intensive TR4 signal in the tooth bud (Tb) and whisker follicle (Wf). Other abbreviations: hf, hair follicle; ie, inner ear; k, kidney; pc, perichondrium; r, retina. The bars represent 1 mm for panel A and 0.1 mm for panels B and C.) (Lee, Y. F. et al. (1998) J. Biol. Chern. 273, 13437-13443)). The expression patterns of TR2 and TR4 are very similar throughout development, with subtle differences in timing (The data showed TR2 mRNA expression during mouse embryogenesis. Saggital sections of embryos at gestation days 11-16 (E11-E16) were hybridized with a radiolabeled antisense TR2 riboprobe and photographed under light-field illumination. Tissues and organs with strong hybridization signals (dark areas) are labeled. A-B, low magnification shows the overall expression pattern of TR2 in E1 and E14 embryos. C-F, high magnification shows the neural structures with strong TR2 expression. Abbreviations: cb, cerebellar primordium; di, diencephalon; drg, dorsal root ganglia; e, otic epithelium; ht, hypothalaums; hv, otic vesicle; l, lens; m, motor neuron; mo, dorsal region of the medulla oblongata; nc, neo-cortex; ob, olfactory bulb; oe, olfactory epithelium; tel, telencephalon; V, trigeminal ganglion; and X, vagal ganglion. The size bars represent 1 mm for panel B and 200 mm for panels A and C-F.) (Young, W. J. et al. (1998) J. BioI Chern. 273, 20877-20885)). At E11, TR2 is observed in the developing rhombomeres, retina, lens, and otic vesicle. By E14, TR2 expression is visible in the dorsal region of the medulla oblongata, the dorsal root ganglion, the cerebellar primordium, and the hypothalamus. As development continues, TR2 is expressed in the sympathetic and trigeminal ganglia by E15, and similar to TR4, regions important to sensory organ development begin to show TR2 expression by E15-16. Overall, both TR4 and TR2 are expressed in many regions of active cell proliferation, in the brain as well as peripheral organs, during embryonic development. Although similar in regions of expression, TR2 is generally expressed earlier in development, followed by the onset of TR4 expression. This variation in temporal expression displayed by these highly homologous nuclear receptors is consistent with distinct physiological roles. TR2 may be more important early in development, whereas TR4 may function in the process of differentiation and maintenance of fully developed physiological systems. In the adult mouse brain, TR4 is found in the hippocampus, cortex, habenular nuclei, and piriform cortex. (The data showed Localization of TR4 mRNA in adult mouse brain by in situ hybridization. Coronal sections of an adult mouse brain, at the level of the hippocampus, were hybridized with an antisense TR4 riboprobe. Dark field (A, C, and E) and bright field (B and D) photomicrographs were taken. A, low magnification shows that TR4 transcripts are detected in the cortex (cx), dentate gyrus (dg), habenular nuclei (hn), piriform cortex (p), and the CA1, CA2, and CA3 regions of Ammon's horn in the hippocampus (1, 2, CA3). High magnification reveals intensive TR4 signal in the neuronal population of the piriform cortex (arrowhead, p) (B and C), and in the granule cells of the dentate gyrus (arrow, g) (D and E). The size bars represent 1 nm for panel A, and 100 nm for panels B-E.) (Young, W. J. et al. (1998) J. BioI Chern. 273, 20877-20885). In the hippocampus, expression is restricted to the CA1, CA2 and CA3 regions, as well as the granule cells of the dentate gyrus. From earlier in situ hybridization analysis of the rat brain, TR4 was found to be highly expressed in the hypothalamus, thalamus, and cerebellum, in addition to the hippocampus (Chang, C. et al. (1994) Proc. Natl. Acad. Sci. USA 1994, 6040-6044). Through Northern analysis of tissue from the adult rat, significant TR4 expression in the prostate, adrenal gland, spleen, thyroid gland, and pituitary gland (Chang, C. et al. (1994) Proc. Natl. Acad. Sci. USA 1994, 6040-6044) were also confirmed. In the case of TR2, immunohistochemical analysis with both a monoclonal antibody specific to the receptor has yielded information regarding the expression pattern of TR2 in the testis (Young W J, Collins L L). In the adult mouse testis (The data showed expression of TR2 protein in the adult mouse testis. Immunostaining of testis sections was performed using a TR2-specific monoclonal antibody with (A), or without (B-L) antigen neutralization. Seminiferous tubules were classified by stage through hematoxylin staining and determination of germ cell type composition of each tubule), TR2 protein expression is restricted in timing, as it is expressed in the germ cell lineage, predominately at immature stages in germ cell development. Strong TR2 protein staining was observed in spermatogonia, spermatocytes, and round spermatids. Additionally, TR2 expression is conspicuously absent from leydig and sertoli cells, consistent with a role for TR2 in germ cell development but not in steroid synthesis, or initial steps in the androgen signaling pathway active in the testis.

Additional evidence that both TR2 and TR4 play significant roles in spermatogenesis comes from recent studies of expression of these receptors in the testes of cryptorchid and high dose testosterone-treated Rhesus monkeys (Mu, X. et al. (2000) J BioI. Chern. 275, 23877-23883). Surgically-induced cryptorchidism resulted in repression of both TR2 and TR4 expression in the abdominal testis, compared with the sham-operated scrotal testis of the same animal. Also, high dose testosterone treatment, resulting in infertility, repressed testicular TR4 expression at the levels of both RNA and protein, suggesting repression of TR4 gene transactivation. Furthermore, analysis of TR4 in the testes of normal adult Rhesus monkeys, via in situ hybridization, demonstrated especially high expression of the receptor in germ cells undergoing the process of meiosis. This germ cell stage-specific expression pattern has been observed in adult mouse testes as well (Hirose, T. et al. (1994) Mol. Endo. 8, 1667-1680). These data indicate a role for TR4 in spermatogenesis that is of particular significance in the meiotic stage of germ cell development.

3. Example Generation of TR4 Knockout, β-gal Knockin Mice

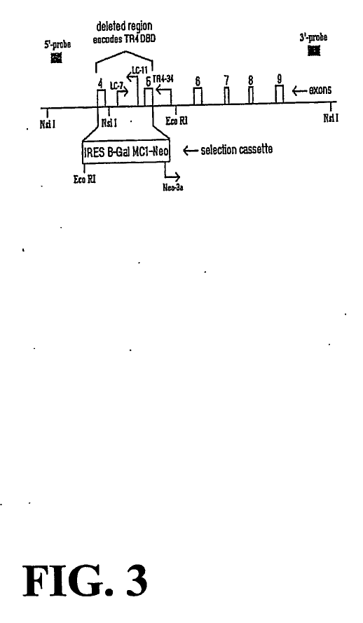

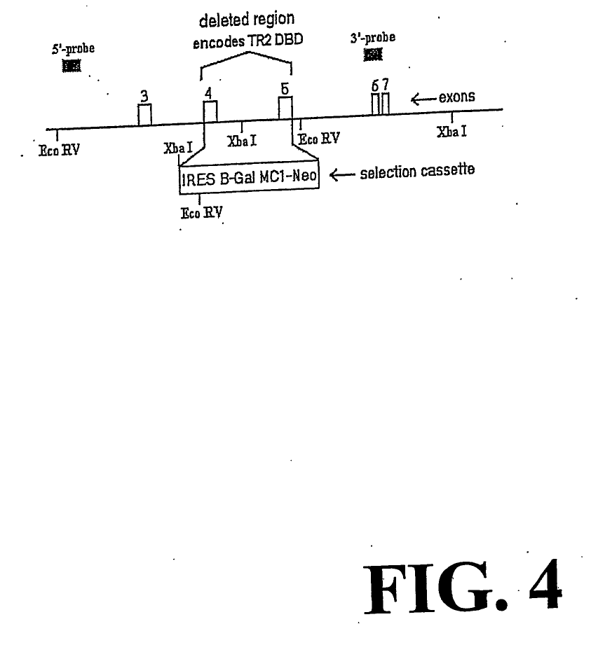

TR4 knockout, β-gal knockin mice have been generated. A vector was constructed to disrupt expression of the TR4 gene (FIG. 3). An IRES β-gal MC1-Neo selection cassette was inserted into the TR4 gene such that it replaced exons 4 and 5, as well as the intervening intron 4. The targeting vector has significant 5′ and 3′ stretches of TR4 genomic DNA to serve as sites of homologous recombination, a neo expression cassette for selection of embryonic stem cell clones, and the DNA sequence encoding Lac-Z (β-galactosidase). After homologous recombination, β-gal expression is driven by the endogenous TR4 promoter and therefore represents the spatial and temporal expression of TR4 in vivo. Additionally, the deleted region of TR4, exons 4 and 5, encode the DBD of the receptor. Elimination of the DBD renders TR4 functionally inactive, as it can no longer act as a transcription factor and regulate its target genes.

Embryonic stem (ES) cells, from a 129/SvEv line derivative that carries the agouti coat color marker, were transfected with the TR4 knockout, β-gal knockin targeting vector. Targeted ES cell clones were then injected into blastocysts from a C57BL/6 albino mouse strain (Baylor College of Medicine, Lexicon Genetics, Inc.). Chimeric animals were bred against the albino C57BL/6 line, allowing the monitoring of coat color as an indicator of germ line transmission. Both Southern blot and PCR analyses were used to screen for animals heterozygous for the disrupted TR4 gene (FIG. 3). Six of each male and female heterozygous mice were then used as founders for colony expansion and breeding to homozygosity, allowing initiation of characterization of the TR4 knockout mice. The colony is maintained as a recombinant inbred strain.

Animals in the colony are identified by ear punches. A combination of holes and notches are punched into the ears of pups at the time of weaning. Also at weaning, a tail snip is taken for the purpose of genotyping the animals. The tail DNA isolation procedure used is described (Hogan, B. et al. (1994) Manipulating the mouse embryo, second Ed., Cold Spring Harbor Laboratory Press, Cold Spring Harbor). Once DNA has been isolated, PCR genotype screening is carried out with two sets of primers. One set is specific to a region including some of both the knockout selection cassette as well as bordering genomic DNA, and the other to a region of the TR4 genomic DNA known to be absent when the selection cassette has integrated (FIG. 3). The knockout primers are TR4-34, a 3′ primer (TGCAAGCATACTTCTTGTTCC SEQ ID NO:18) specific to a region of genomic DNA 3′ of the selection cassette, and Neo-3a, a 5′ primer (GCAGCGCATCGCCTTCTATC SEQ ID NO:19) specific to a sequence within the selection cassette. The PCR product generated using TR4-34 and Neo-3a is 760 bp in length (The data showed PCR-based genotype screening of TR4KO mice. Primers TR4-34 and Neo-3a were used to screen for integration of the targeting construct, yielding a 760 bp PCR product; upper band. Primers LC-7 and LC-11 were used to screen for the wildtype TR4 gene, yielding a 455 bp PCR product; lower band. Symbols: M, DNA size marker; +/+, wildtype; +/−, TR4 heterozygous knockout; −/−, TR4 homozygous knockout.). The wildtype primers are LC-7, a 5′ primer (GGAGACACACTGCACATGTTCGAATAC SEQ ID NO:20), and LC-11, a 3′ primer (CACAGCTCATTTCTCTGCTCACTTACTC SEQ ID NO:21), both specific for sequences of genomic DNA within intron 4, which is absent when the targeting construct has been inserted. The PCR product generated using primers LC-7 and LC-11 is 455 bp in length.

a) TR4 Knockout, β-gal Knockin Phenotype Analysis

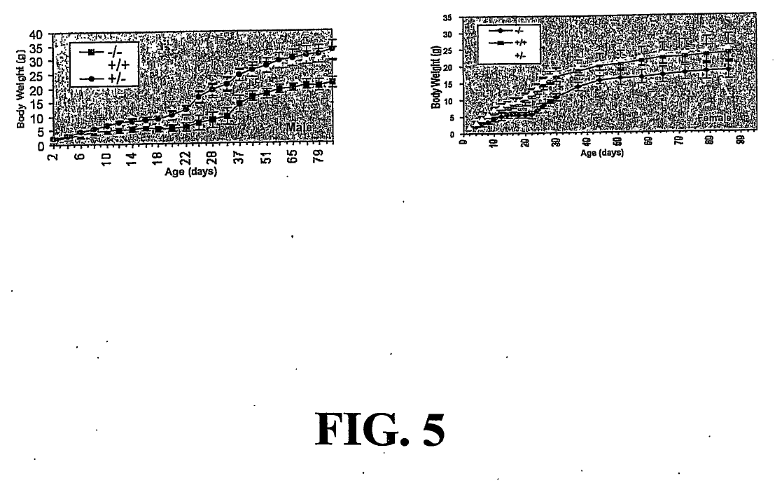

TR4 knockout, β-gal knockin animals, have specific phenotypes involving growth, fertility, and behavior. Multiple litters from pairing the heterozygous founder animals have been produced, and at the time the pups from the first litters were old enough to be weaned and genotyped, it was noticed that several animals were visibly smaller than their littermates. To confirm this qualitative observation, several pups of similar ages were weighed, and the genotype of each animal was determined. It was found that the TR4−/− mice weighed approximately 30% less than wildtype and TR4+/− animals of the same age. As a visible representation, a male mouse of each genotype TR4−/− and TR4+/− were photographed side by side (The data showed the size discrepancy between TR4−/− and TR4+/− or wildtype mice Both mice shown are male at 5.5 months of age. The animal on the left is TR4−/−, weighs 15.7 g, and is 6.8 cm in length. The animal on the right is TR4+/−, weighs 41.1 g, and is 9.8 cm in length. Wildtype mice (not shown) are comparable in size to TR4+/− animals of the same age.). As shown in FIG. 5, TR4−/−, TR4+/−, and wildtype littermates were weighed from two days of age to determine when the growth defect first became apparent. A 30% reduction in weight was seen by 10 days in TR4−/− pups compared to TR4+/− and wildtype mice. No significant difference was observed between TR4+/− and wildtype animals at all ages examined. The growth retardation observed in the TR4−/− animals persisted for the duration of the experiment (12 weeks).

Additionally, although TR4+/− animals show no defect in fertility, initial pairings of TR4−/− mice yielded no pregnancies. To determine if the problem is with the male, female or both, TR4−/− mice of each gender were paired with a known fertile animal of the opposite sex. Each morning for one week after the pairings, the female mouse in each cage was examined for the presence of a vaginal plug. The known fertile female mice that had been paired with TR4−/− males had no sign of having plugs, whereas the TR4−/− female animal mated with a known fertile male was found to have a plug. Again, there have been too few animals tested to make conclusive claims, but the evidence so far suggests either a physiological or behavioral defect causing the TR4−/− animals to be, or appear to be, infertile.

An analysis of spermatogenesis in male TR4−/− compared to wildtype mice has been performed. At 7 weeks of age, TR4−/− mice show a 66% reduction in epididymal sperm number compared to wildtype littermates of the same age. At 12-14 weeks of age TR4−/− mice show a 32-39% reduction in epididymal sperm counts (Table 4). It is not expected that a 30-40% decrease in sperm number would result in complete sterility but implies that a defect in spermatogenesis occurs in the TR4−/− males resulting in a decrease in the numbers of mature sperm. Analysis of sperm motility can be performed and a defect in sperm motility of the TR4−/− males identified and morphological defects present in TR4−/− sperm can also be analyzed.

TABLE 4
Epididymal sperm counts from TR4 −/− and wildtype males.

| | Age | | |
|---|---|---|---|
| Genotype | (weeks) | Mean Sperm Number (10⁶) | Std. Dev. |
| Wildtype | 7 | 3.28 | 0.5 |
| TR4 −/− | 7 | 1.15 | 0.61 |
| Wildtype | 12 | 5.4 | 0.26 |
| TR4 −/− | 12 | 3.3 | 0.96 |
| Wildtype | 13 | 6.03 | 0.31 |
| TR4 −/− | 13 | 4.02 | 0.81 |
| Wildtype | 14 | 7.43 | 0.81 |
| TR4 −/− | 14 | 4.5 | 0.67 |

Epididymi were removed into 0.4% NaCl and minced. After incubation at 37° C. for 20 min., samples were counted using a hemocytometer.

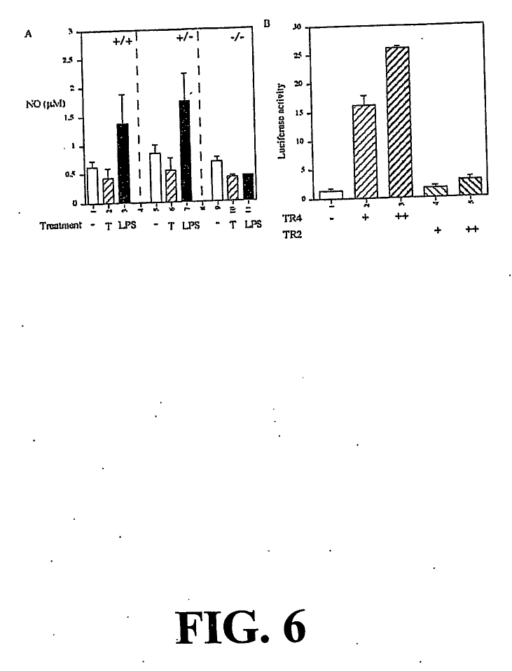

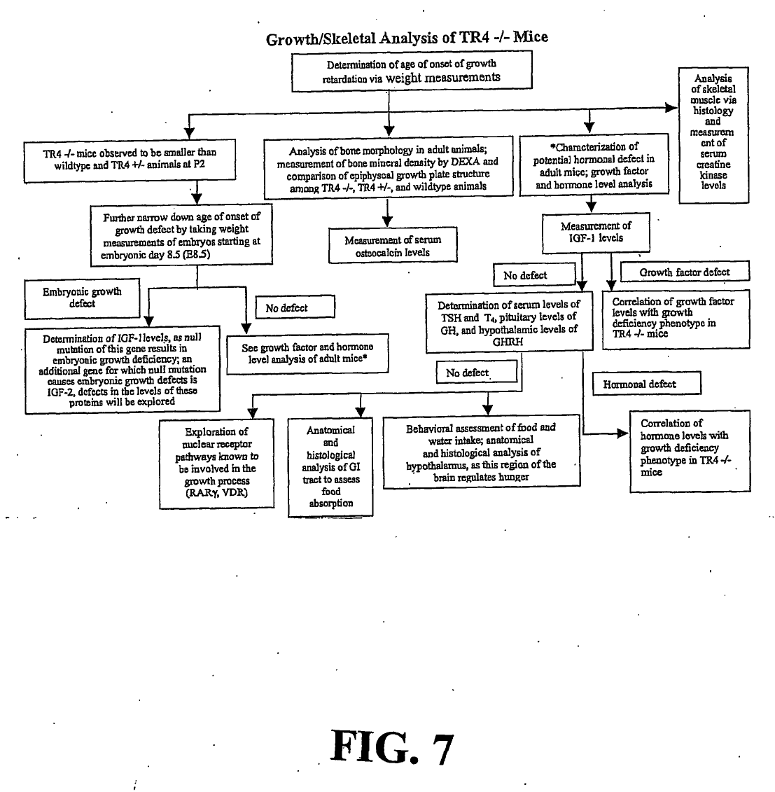

Through observation of general outward physical appearance and behavior of TR4−/− animals, a general inactivity among the knockout animals as compared to their wildtype or heterozygous cagemates was noticed. Also, it was observed that TR4−/− animals display an abnormal gait characterized by what seems to be a lack of coordination, especially in the hind limbs. Such observations are suggestive of either potential limb structure abnormalities or a defect in lumbar spinal cord development. Macroscopic analysis of brain structures, comparing adult TR4−/− animals with adult widltype animals of the same sex, demonstrated the presence of size differences in both the cerebrum and the cerebellum (the data showed a gross morphological comparison of wildtype and TR4−/− mouse brain. Upper panel represents the brain of a 14 week old wild type male. Lower panel shows the brain of a 14 week old TR4−/− male. Mice were perfused with 10% neutral buffered formalin through the left ventricle. Images were taken with a digital camera and morphometric analysis performed using the NIH image program 1.62. Morphometric analysis was performed by manually tracing the hemisphere to be measured and the number of pixels within the demarcated area was counted by the program.). TR4KO male mice, between 3.5 and 13 months of age, have an 8.5% reduction in surface area of the cerebrum (excluding the olfactory bulbs and the inferior colliculus). Similar analysis of the brains of female mice between 6 and 13 months of age resulted in 15.3% reduction of cerebrum size in TR4KO samples compared to those from wildtype mice. Similarly, when considering the surface area of the cerebellum in mid-saggital section, it was found that the measurement was reduced 34.4% in adult TR4KO males and 46.6% in adult TR4KO females when compared to that of wildtype animals of the same sex. The cerebellar granular cell density in TR4−/− mice is reduced compared to wildtype animals. (The data showed granule cell density of the cerebellum Saggital sections of adult wildtype and TR4−/− cerebellum. The cerebellum was embedded in glycol methacrylate and stained using Nissl/toluidine blue.) The TR4−/− mouse shows a 20-30% reduction in granule cell density. In addition, primary astrocytes derived from the cerebellum of two week old TR4−/− mice shows a reduced nitric oxide (NO) production in response to lipopolysaccharide (LPS), an inducer of iNOS (FIG. 6). These observations suggest that multiple defects are present in the brain of TR4−/− mice compared to wildtype or heterozygous littermates. As the wildtype tissue distribution of TR4 suggests a role for the orphan receptor in brain development and function, learning and memory in the knockout animals can be analyzed. The widely used cognitive test for mice is the Morris water maze (Morris, R. G. M. et al. (1982) Nature 297,681-683) and this can be used to test spatial learning ability. As the Morris water maze requires the animals to swim and, based on the abnormal gait of the TR4−/− mice and the potential that a structural defect or loss of coordination may make it difficult or impossible for them to swim, the swimming capability was tested. Both wildtype and TR4−/− animals were able to swim when tested for this ability.

b) Delayed and Disrupted Late Meiotic Prophase and Subsequent Meiotic Divisions of Spermatogenesis in Mice Lacking Testicular Nuclear Orphan Receptor-4 (TR4)

Testicular orphan receptor 4 (TR4) is specifically and stage-dependently expressed in late stage pachytene spermatocytes and round spermatids. In developing mouse testis, the highest expression of TR4 can be detected at day 16-21 when the first wave spermatogenesis progresses to late meiotic prophase. Using knockout strategy to delete TR4 in mice, it was found that sperm production in TR4^{-/-} mice is significantly reduced. The comparison of the testis and epididymis from developing TR4^{+/+} and TR4^{-/-} mice showed that spermatogenesis in TR4^{-/-} mice is seriously delayed. Analysis of the first wave spermatogenesis showed that the delay was caused by the serious delay and disruption of late meiotic prophase and subsequent meiotic divisions. The tubule stage analysis showed stage X to XII, where late meiosis prophase and meiotic divisions take place, was seriously delayed and disrupted in TR4^{-/-} mice. The TUNEL assay and histological examination of testis sections from TR4^{-/-} mice showed apoptotic and degenerated primary spermatocytes, and some necrotic tubules. The testis specific gene expression analysis shows that late meiotic prophase expressed genes, sperm-1 and cyclin A1, were delayed and decreased in TR4^{-/-} mouse testes. Taken together, results from TR4^{+/+} and TR4^{-/-} mice indicate that TR4 is essential for normal mice spermatogenesis.

(1) Results

(a) Cell Specific and Stage-Dependent Expression of TR4

In situ hybridization and PAS and hematoxylin staining using consecutively cut testis sections from TR4+/+ showed TR4 is highly expressed in primary spermatocytes, especially in late stage pachytene spermatocytes (The data showed TR4 cell specifically expresses in pachytene spermatocytes and stage-dependently expresses in late stage spermatids. A. In situ hybridization of normal adult testis sections with antisense TR4 digoxin-labeled probe. Two seminiferous tubules in stage IV and stage VII which show positive signals in pachytene spermatocytes and in both pachytene spermatocytes and spermatids are shown. B. PAS and hematoxylin staining of two seminiferous tubules as in A, in a consecutively cut section. Ps: pachytene spermatocytes. Rs: round spermatids;. PL: preleptene spermatocytes. Magnification: A and B, 400×.). TR4 is also expressed in round spermatids. The expression of TR4 is stage dependent and can only be detected in stage VII.

(b) Time Course of TR4 Expression During Testis Development

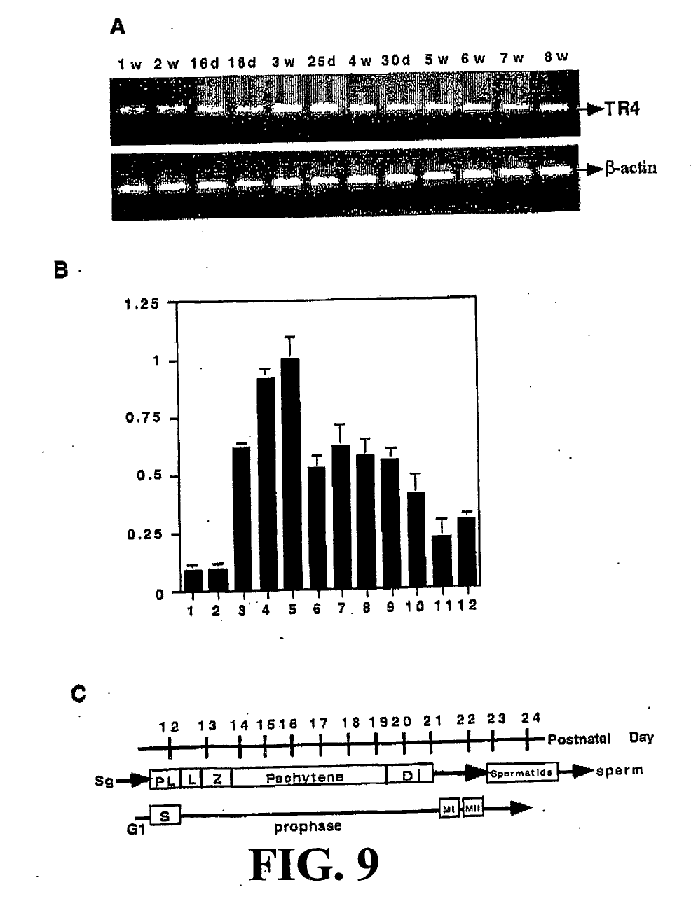

To define the stage where TR4 may play a role, the TR4 expression starting during testicular development and continuous into adult stages was investigated. Total RNA from different ages of mice were prepared and analyzed by RT-PCR (FIG. 9) and real-time quantitative RT-PCR (FIG. 9). As shown in FIG. 9, expression of TR4 mRNA can be detected one week after birth, begins to increase at postnatal day 16, and reaches the highest level at around day 21. At day 25, TR4 expression began to decrease and remained at a moderate level afterwards and throughout the adult stage. Early studies (Bellve et al., 1977) indicate that between days 16-21 the first wave of spermatogenesis progresses at the meiotic prophase and germ cells differentiate at latest pachytene and diplotene stage and then into meiotic divisions (The data showed confirmation of knockout TR4 gene in TR4−/− mice. (A) PCR analysis of mouse genomic DNA. The wild type and target alleles give 454 bp and 760 bp PCR products, respectively. (b) RT-PCR analysis of TR4 in TR4+/+, TR4+/−, and TR4−/− mouse testis. Total RNA from TR4+/+, TR4+/−, and TR4−/− mice were extracted and RT-PCR was performed). TR4 expression in developing testis in FIG. 9 was consistent with its cell specific expression, and the highest expression of TR4 in the late meiotic prophase and advanced pachetene spermatocytes suggests that TR4 may play significant roles in the late meiotic prophase and subsequent meiotic divisions.

(c) Confirmation of Knockout TR4 gene in TR4−/− Mice

Using classic knockout and homologous recombination techniques, most of exon 4 and the complete exon 5 of the TR4 gene was replaced with LAC/MC1-Neo cassette to generate TR4^{-/-} mice. PCR analysis of DNA extracted from mice tails with two sets of primers as described in Materials and Methods, show that TR4^{+/+} mice have an intact TR4 gene, and TR4^{-/-} mice have DNA deleted between exon 4 and exon 5.

RT-PCR analysis of RNA from 3 months-old TR4^{+/+}, TR4^{+/-}, and TR4^{-/-} mice testis indicates TR4 expression is significantly decreased in TR4^{+/-} mice and undetectable in TR4^{-/-} mice. Together, the data clearly demonstrated that the TR4 gene was successfully disrupted in TR4^{-/-} mice.

(d) Morphological Appearance and Weight of Testis and Sperm Production of TR4^{-/-} Mice

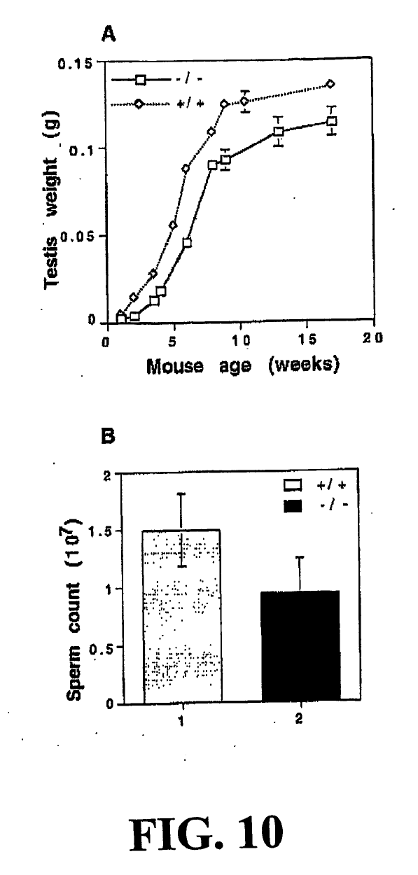

Breeding studies indicated that the TR4^{-/-} male had reduced fertility. The size of adult TR4^{+/+} and TR4^{-/-} mice testes are similar, however, the weight of testes from TR4^{-/-} mice at various developing stages is small as compared to the TR4^{+/+} mice, yet the weight of testes from adult TR4^{-/-} mice is only slightly decreased compared to the TR4^{+/+} mice (FIG. 10). The sperm number is significantly decreased in TR4^{-/-} mice at various ages as compared to TR4^{+/+} mice. FIG. 10 sums up the data from cauda epididymis sperm count from 2-3 month old TR4^{-/-} mice.

(e) Delayed Spermatogenesis

6-wk-old TR4+/+ mice usually can form stage 16 spermatids and have typical stage VII tubules in testis sections. But in 6-wk-old TR4−/− mice, stage 16 spermatids and typical stage VII tubules were hardly observed and the most frequently seen tubule is stage X-XII (The data showed a delayed spermatogenesis in TR4−/− mice. A: Testes morphology of 6-wk-old WT mouse. Note: the stage VII seminiferous tubules were most frequently observed. B: Testes morphology of 6-wk-old TR4−/− mouse. Note: testis lack Stage VII tubule and the tubule at X-XII were mostly frequently observed. C: Morphology of cauda epididymis of 6-wk-old WT mouse. Note: cauda epididymis was full of sperm. Arrows point to the sperm in epididymis. D: Morphology of epididymis from 6-wk-old TR4−/− mice. Note: nearly no sperm can be seen in cauda epididymis. Magnification: A-D, 400×). The typical stage VII tubule, however was more frequently seen until about 10-wk-old (Data not shown). Furthermore, unlike 6-wk-old TR4+/+ mice that have high number of sperm in cauda epididymis, there was almost no sperm in the cauda epididymis in TR4^{-/-} mice, suggesting the first wave of spermatogenesis in TR4−/− mice is delayed.

(f) Delayed and Disrupted Late Meiotic Prophase and Meiotic Divisions of First Wave Speramatogenesis in Juvenile TR4^{-/-} Mice

In order to unveil the mechanism of late spermatogenesis in TR4^{-/-} mice, the progression of the first wave of spermatogenesis was compared in TR4^{+/+} and TR4^{-/-} littermates. At postnatal day 7, the testes histology from both TR4^{+/+} and TR4^{-/-} mice are similar, spermatogenesis arrested at spermatogonia stage, and seminiferous tubules contain Sertoli cells and spermatogonia (Data not shown), suggesting prenatal development in TR4^{-/-} mice was relatively normal. At day 14, when premeiosis phase of spermatogenesis begins, germ cell differentiation in TR4^{-/-} mice is nearly at same stage or is only slightly slowed as compared to the TR4^{+/+} mice. At this stage, germ cell differentiation proceeds to mostly zygotene spermatocytes stage (The data showed delayed and disrupted late meiotic prophase and subsequent meiotic divisions in first wave of spermatogenesis in TR4^{-/-} mice. Morphology of seminiferous tubules from TR4^{+/+} (A, C, E) and TR4^{-/-} (B, D, F, G, and H) mice at day 14 (A, B), 22 (C, D), 28 (E, F), and 31 (G, H) were shown. At day 14, germ cell differentiation progresses to mostly zygotene (Zs) stage in both TR4^{+/+} and TR4^{-/-} mice. At day 22, in TR4^{+/+} mice, meiosis has been completed and many round spermatids (Rs) produced with a few of them differentiated into elongated spermatids (Es). In TR4^{-/-} mice cells are still arrested in pachytene (Ps) or deplotene stages and no round spermatids were produced, and tubules contain multinucleated giant cells (SY) and primary spermatocytes with increased cytosol (Psb). Vaculoes (V) in the cytosol of pachytene spermatocytes can be frequently observed. At day 28, adluminal cells are primarily round spermatids and elongated spermatids in TR4^{+/+} mice, while adluminal cells are still primarily pachytene spermatocytes or deplotene spermatocytes in TR4^{-/-} mice. At day 31, meiosis has been completed and round spermatids appeared in some tubules of TR4^{-/-} mice, but quite a few mutiplenucleated giant cells (i.e., symblast, SY) can be observed. Sections from at least 3 TR4^{+/+} and TR4^{-/-} mice at indicated ages were examined, and a representative section is shown. Magnification: A, B, C, D, and H, 1000x; E, F, and G, 400×). At day 22 in TR4^{+/+} mice some tubules have completed the first and second meiosis, many round spermatids can be seen in the tubules, and a few of them have differentiated into elongated spermatids. In some tubules, meiosis is in process and a few metaphase cells can be observed. But in TR4^{-/-} mice, at day 22, meiosis has not occurred, and cells arrested in meiotic prophase stage, and the most highly differentiated germ cell is still the late pachytene spermatocyte. Some pathological changes such as vacules in the cytosol of pachytene spermatocytes, mutinucleated giant cells (i.e., symblast, SY), and primary spermatocyte with increased cytosol can be observed. At day 28, in TR4^{+/+} mice, most tubules have completed meiosis and postmeiosis morphological changes have taken place. Large numbers of round spermatids and elongated spermatids can be seen. But in TR4^{-/-} mice, meiosis is still arrested and germ cell differentiation is still delayed at prophase of meiosis stage, round spermatids still do not appear, and the highest differentiated germ cell is pachytene or depletene germ cell. Until nearly at day 31, meiosis is completed in some tubules of TR4^{-/-} mice testis and round spermatids can be detected. But, many symblasts (SY) can be frequently observed. These symblasts could result from spermatocytes with meiosis defects. The data indicated that the late spermatogenesis in TR4^{-/-} mice is caused by delayed and disrupted late meiotic prophase and subsequent meiotic divisions.

(g) Disrupted Tubule Stage Characteristics and Prolonged and Disrupted Stage XI-XII in TR4^{-/-} Mice

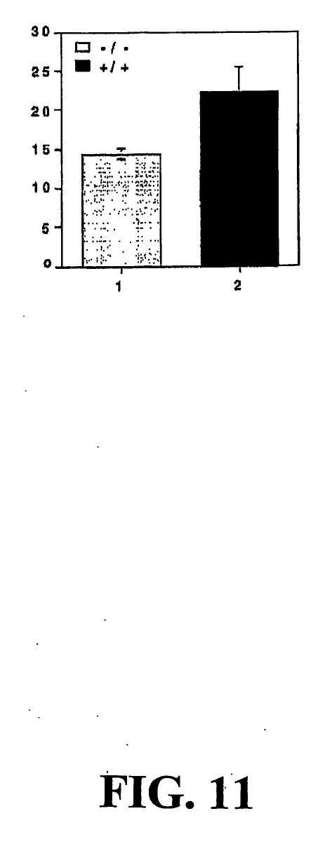

Spermatogenesis is highly organized with germ cells at particular phases of development associated together into 12 distinct stages. To further confirm the role of TR4 in late meiotic prophase and subsequent meiotic divisions, 12 tubule stages between TR4^{+/+} and TR4^{-/-} mice was compared. The characteristics of tubule stage I-IV are the formation of proacrosomic granules in round spermatids, and it was found there is no difference between TR4^{+/+} and TR4^{-/-} mice in these stages. The data showed a typical wild type stage III tubule and a typical TR4^{-/-} stage III tubule respectively. The major events of tubule stage V-VIII are the formation of the acrosome cap and testis mature sperm (stage 16 spermatids) are formed in stage VII which can be released into lumen in stage VIII. No difference between TR4^{-/-} and TR4^{+/+} mice in these stages was found. The data show a typical wild type stage VII tubules and a typical TR4^{-/-} stage VII tubule. The major events of tubule stage IX-XII are the formation of a complete acrosome system in spermatids, late stage pachytene spermatocytes differentiate into diplotene spermatocytes in stage XI, and first and second meiosis shortly takes place in stage XII. It was found there are obvious differences between TR4^{-/-} and TR4^{+/+} mice in stage XI-XII. Meiosis divisions take place quickly in stage XII tubule in TR4+/+, so stage XI-XII in normal and TR4^{+/+} mice were relatively short and only a few metaphase cells are usually observed in Stage XII tubule. In contrast, stage XI-XII tubules can be more frequently observed in tubules from TR4^{-/-} mice and in some sections several surrounding tubules are all in stage XI-XII. Many different stages of metaphase cells can be observed in seminiferous tubules from TR4^{-/-} mice, and the morphology of some of these cells are not typical. Symblasts and primary spermatocytes devoid of chromosome structure can be frequently observed (The data showed disrupted and prolonged tubule stage XI-XII. A. A tubule at stage 3 from TR4^{+/+} mice. B. A tubule at stage 3 from TR4^{-/-} mice. Note: the proacrosome granules in round spermatids stained by PAS both in A and B. C, a tubule at stage VII from TR4^{+/+} mice. D. A tubule at stage VII from TR4^{-/-} mice. Note Stage VII tubules from both TR4^{+/+} mice and TR4^{-/-} mice can produce stage 16 testis maturated spermatids (S 16). No histological difference can be found between A and B, or C and D. E: A tubule at stage XII from TR4^{+/+} mice. F, G, and H. tubules at stage XII from TR4^{-/-} mice. Note: prolonged metaphase cells, symblast (SY). Primary spermatocytes devoid of chromosome structure (Psc) A-H: Sections from at least 3 TR4^{+/+} and TR4^{-/-} mice were examined, and a representative section is shown. Magnification: A-H: 1000×. I.). Furthermore, the numbers of stage X-XII tubules and total tubules from TR4^{-/-} and TR4^{+/+} mice were counted and found that the ratio of stage XII tubules is significantly increased in TR4^{-/-} mice testes (FIG. 11). Taken together, the data clearly indicates that late meiotic prophase and subsequent meiotic divisions are prolonged and disrupted in stage XI-XII tubules in TR4⁴ mice.

(h) Apoptosis, Degeneration and Necrosis in Partial Seminiferous Tubules in TR4^{-/-} Mice

A TUNEL assay was performed to assess possible apoptosis. Apoptotic signals can be detected in some of the tubules from TR4−/− mice, but not in tubules from TR4+/+ mice (The data showed apoptosis and degeneration of primary spermatocytes and necrosis of some tubules. A and B. Apoptosis detection from TR4+/+(A) and TR4^{-/-} (B) mouse sections. Arrows point to apoptotic cells. C. Degeneration of primary spermatocytes. V: vacuoles. D, Necrosis of seminiferous tubules from TR4−/− mice. * represents necrotic tubules. Magnification: A-D, 400×.). According to the location of apoptotic signal in the tubules, most apoptotic signals are from primary spermatocytes. Consistently the histological examination shows some primary spermatocytes going through degeneration with appearance of vacuoles in the cytosol or large vacuoles in the tubules. The degeneration can spread into all the tubules and eventually result in complete necrosis of some tubules. Necrotic seminiferous tubules were observed in most testes sections from TR4−/− mice over 6-wk-old, and necrotic tubules account for 1-33% of total tubules. The data showed a typical testis section with severe necrotic seminiferous tubules.

(i) Testis Specific Gene Expression Pattern in TR4^{-/-} Mice

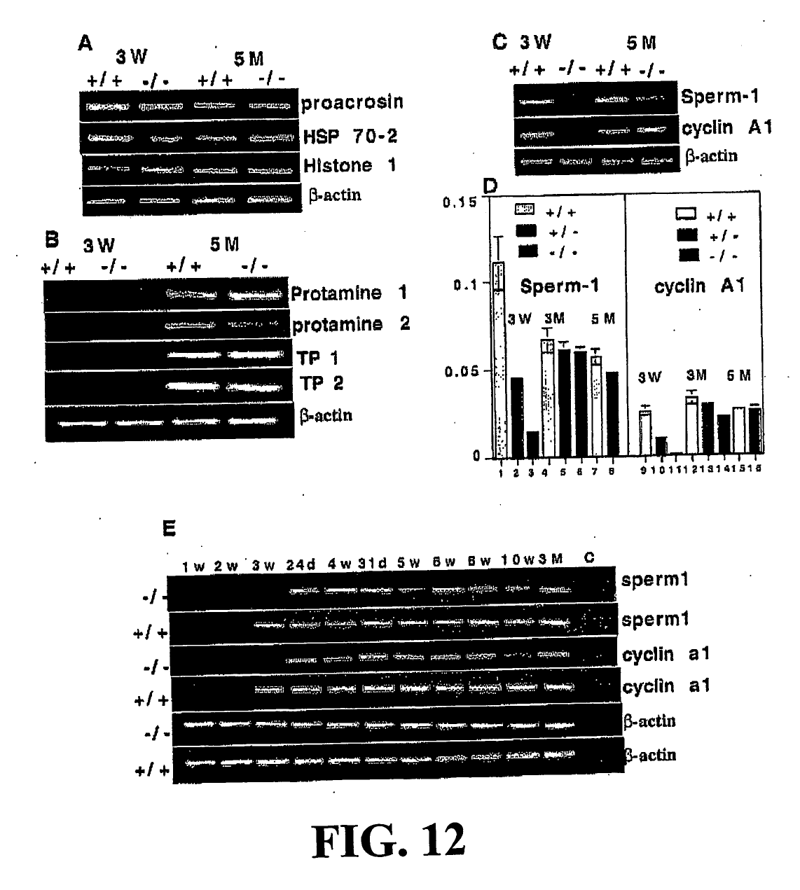

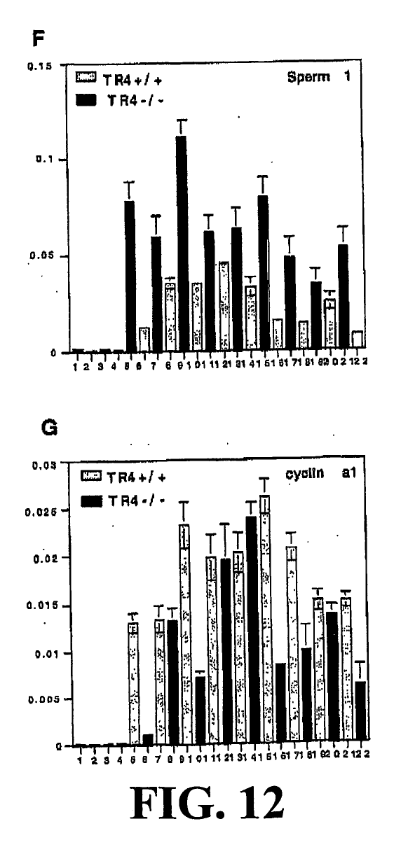

To investigate the effect of TR4 deficiency on testis molecular markers and potential downstream targets, testis RNAs from TR4^{+/+} and TR4^{-/-} mice at different ages were analyzed with three panels of testis-specific genes (Martianov et al., 2001; Zhang et al., 2001). One panel of testis specific genes begins to be transcribed before the first meiotic division and expresses during the pre-meiosis phase, and includes the acrosomal serine protease Proacrosin (Kashiwabara et al., 1990), the heat-shock protein Hsp70-2 (Zakeri et al., 1988), and histone Hlt (Drabent et al., 1996). As shown in FIG. 12A, the expression of this panel of genes is not significantly changed in TR4^{-/-} mice testes compared with that in TR4^{+/+} mice testes. The other panel of testis specific genes is postmeiotic expressed and includes Protamine 1 and 2, and transition protein 1 and 2 (Wouters-Tyrou, et al., 1998), The transitional proteins and protamines are small highly basic proteins that faciliate compaction of the mammalian sperm head during spermatogenesis (Wouters-Tyrou, et al., 1998). As shown in FIG. 12B, the expression of transitional protein 1 was slightly increased, and the expression of transitional protein 2 was slightly decreased. Protamines were not significantly changed in TR4^{-/-} mice testes compared to TR4^{+/+} mice testes. The third panel of genes begins to express at the end of meiotic prophase and plays essential roles in late meiotic prophase and subsequent meiotic division and includes sperm-1 (Anderson et al., 1993), and cyclin A1. As shown in FIG. 12C, Sperm-1 and cyclin A1 are either not detected or only weakly expressed in testes from 3-wk-old TR4^{-/-} mice while they are already highly expressed in 3-wk-old TR4^{+/+} mice testes. In adult TR4⁴ mice testis, sperm-1 expression level was significantly decreased compared with that in TR4^{+/+} mice testis. Results in FIG. 12C are further confirmed by real-time quantitative RT-PCR using more samples from different mice (FIG. 12D). Furthermore, the expression level of sperm-1 and cyclin A1 were detected at various developing stages and several adult stages by both RT-PCR and real-time quantitative RT-PCR, as shown in FIG. 12E, F, and G, both sperm-1 and cyclin A1 expression were significantly delayed in TR4^{-/-} juvenile mice compared with that in TR4^{+/+} juvenile mice, sperm-1 expression level in various developing stages and adult stages were significantly decreased (FIGS. 12E and F), and cyclin A1 expression levels in most developing stages and adult stages were decreased (FIGS. 12E and G).

(2) Materials and Methods

(a) Genotyping of TR4^{-/-} Mice

Mouse genotyping on tail biopsy specimen DNA was performed by PCR. The primers used for wild type allele were: SEQ ID NO:22 5-GGAGACACACTGCAGATGTCCGAATAC-3 (A) and SEQ ID NO:23 5-CACAGCTCATTTCTCTGCTCACTTACTC-3 (B), which locate between exon 4 and exon 5 of TR4 gene The primers used for mutant allele were SEQ ID NO:24 5-TGCAAGCATACTTCTTGTTCC-3 (C) and SEQ ID NO:25 5-GCAGCGCATCGCCTTCTATC-3(D). Primer C is located in the Neo sequence of the IRES LacZ/MC1-Neo selection cassette, and primer D is located on the exon 5 and 6 of TR4 gene.

(b) Histological Analysis

Tissues were fixed in fresh 10% neutral buffered formalin, Bouins fixative, or forman-Zender buffer, and embedded in paraffin. Tissue sections were stained with hematoxylin and eosin (H&E) or periodic acid and Schiff reagent (PAS) and hematoxylin, and examined by light microscopy.

(c) Reverse Transcription (RT)-PCR and Real-Time Quantitative RT-PCR

Mouse testes from TR4^{+/+} and TR4^{-/-} mice at different ages were dissected and total RNA isolated by using a TRIZOL reagent (Invitrogen). cDNA synthesis and PCR were performed using SuperScript™ II RNAse H⁻ Reverse Transcriptase and cDNA cycle kit (Invitrogen) following the manufacturer's protocol. Real-time PCR was performed using icycler real-time PCR amplifier (Bio-Rad). Each PCR reaction contained 1 μl cDNA, 50 μm primers and 12.5 μl IQTM SYBRR green supermix reagent (Bio-Rad) and was triplicated. The results were normalized with β-actin. A list of the primer sequences for RT-PCR and real-time RT-PCR are available upon request.

(d) Detection and Characterization of Apoptotic Germ Cells

Apoptosis detection was performed using Fluorescein-Frag EL™ DNA fragmentation detection kit (Oncogene, QIA 39) following the manufacturer's protocol.

(e) In Situ Hybridization

Digoxigenin-UTP labeled riboprobes were prepared with DIG RNA labeling kit (Roche Molecular Biochemicals) from linearized plasmid DNA templates. Tissues were fixed and embedded in paraffin, 5 μm sections were cut, and mounted on coated slides. Tissues on slides were dehydrated, postfixed, and actylated as described (Mu et al., 2000). After hybridization, slides were washed, exposed to alkaline phosphatase-conjugated anti-digoxigenin antidody and riboprobes detected with nitroblue tetrazolium chloride (NBT) and 5-bromo-4-chloro-3-indolyl-phosphate, 4-toluidine (BCIP) substrate.

(f) Sperm Count

Cauda epididymis were removed from adult males and placed in a dish containing 5 ml of Dulbecco Modified Eagle Medium with 10% fetal calf serum. Sperm was allowed to disperse into the medium for 1 h at 37° C., and numbers were counted with a hemocytotometer under phase-contrast microscopy.

### 4. EXAMPLE

### Determination of Effect of TR4 and/or TR2 Target Gene Ablation on Known Target Gene Expression

To determine endogenous target gene expression and protein levels in TR4 and TR2 target tissues, focusing initially on testis, brain, kidney, and muscle, Northern, Western and immunohistochemical staining analyses will be performed on tissues from TR4 and TR2 various knockout and wildtype mice. Levels of target gene expression will be analyzed in tissues from adult, post-natal, and embryological time points depending on the known function and temporal expression pattern of the target gene in question.

a) Methods

Five animals of each genotype (TR4-I-, TR4+/−, wildtype, TR2-I-, TR2+/−, TR2/TR4-I-), at particular developmental stages, will be sacrificed. mRNA expression levels of known TR4 and TR2 target genes will be determined via Northern blot analysis with probes for the appropriate target genes. To determine protein levels of TR4 and/or TR2 target genes after receptor ablation, five animals of each genotype, at particular developmental stages, will be sacrificed. Histological and Western blot analyses will be performed on various tissues, as discussed herein employing specific antibodies for the proteins produced by the target genes of interest.

b) Identification of Novel Genes Regulated Uniquely or Differentially by TR4 and/or TR2

(1) Methods

(a) Exemplary Tissues

Gene micro array analysis can be used to identify differences in gene regulation mediated by TR4 compared with TR2. Microarry analysis is a good technique for this because it allows detection of low abundance transcripts that exhibit significant alteration between wildtype and knockout mice. A strategy such as differential display has not been selected for this analysis because isolation of sequences from the differentially expressed bands is labor intensive and can be confounded by a large number of cDNAs present in a band. Gene expression between three pairs of conditions will be analyzed: 1) TR4+/+ vs. TR4−/−, 2) TR2+/+ vs. TR2−/−, and 3) TR2/TR4+/+ vs. TR2/TR4−/−, 4) TR4+/+ vs. TR4 +/−, 5) TR4+/− vs. TR4−/−. In each case five animals of each genotype and appropriate gender can be sacrificed, and total RNA can be prepared from tissues of interest. Animals can be age matched for analysis. The analysis of TR4−/− and TR2−/− and double knockout animals can be performed based on intial screening and analysis indicating a possible difference. For example, if an alteration in growth-mediating hormones is detected in TR4−/− mice, RNA derived from the pituitary of TR4−/− mice and TR4 wildtype littermates can be used for micro array analysis. If a skeletal defect in bone remdeling is detected in TR4−/− mice, RNA can be isolated from osteoblasts of neonatal calvariae of TR4−/− and wildtype mice. If differences in skeletat muscle between TR4^{-/-} and wIld type mice are observed, the RNA isolated from skeletal muscle from TR4−/− and wIldtype mice can be compared by microarray. TR4−/− males have a reduced sperm count compared to wildtype males at all ages examined. Therefore in coordination with an analysis of male infertility, RNA can be isolated from the testis and epididymis (combined) of 7 week old TR4−/− and wildtype mice for microarray profiling. If a defect in female infertility is found, the appropriate tissue(s) (e.g. ovary, uterus, or mammary gland) can be isolated from females for microarray analysis. The results discussed herein indicate multiple neurological differences between TR4−/− and wildtype mice. Brain tissue can be used for comparison between TR4−/− and wildtype mice. The determination of whether to isolate RNA from whole brain or isolated brain regions can be made after the analysis of the defect is complete. However, if a hypothallamic defect is detected (hormonal analysis of growth retardation), the hypothallamus of TR4−/− and wild type mice will be analyzed separately. If defects in other organ systems of TR4−/− mice are detected in the analysis, RNA from these organs can be used to compare the microarray results between TR4−/− and wildtype mice. A similar strategy can be used for determining the target tissues to be compared in TR2−/− mice and mice deficient in both TR2 and TR4. After systematic identification of defects in these other two lines, tissues associated with the defect will be used for RNA isolation and micro array analysis. In all cases, RNA derived from the knockout mice will be compared to age and gender matched wildtype controls.

(b) Array Analysis

Control wildtype and knockout RNA tissue can be separately arrayed and analyzed, for example, using the Affymetrix GeneChip murine expression array set (arrays A,B, and C). It is understood that any analogous system can also be used, such as other chip systems or other amplification systems. In the Affymetrix GeneChip, each array of the set contains 12,000 annotated genes or ESTs allowing 36,000 genes to be screened for each tissue. Array screening can be done in triplicate for each tissue and genotype to assure reproducibility of the results. Tissue RNA from wildtype and knockout animals will be isolated and utilized in the chosen gene screening technology. cDNA synthesis, cRNA sythesis and labeling, and array hybridization, washing and scanning can be performed as required and quality controls for probes and other reagents can be performed. For example, a test chip is available for this determination and can be used for all source RNA before probing the full expression chips. After expression chip hybridization, a basic gene expression analysis depicting a fold difference for each RNA between the wildtype and knockout tissue can be performed. Analysis of the results of array data can be done with a variety of software packages including GeneChip Expression Data Mining Tool (EDMT), and other data analysis packages such as Stingray and GeneSpring.

(c) Tissue Isolation

Tissue RNA isolation for microarray analysis will be performed using TRIzol (LifeTechnologies) according to the manufacturers instructions. RNA from each tissue will be isolated from five wildtype and five knockout animals. Three groups of experimental animals will be examined,

1) TR4+/+ vs. TR4−/−, 2) TR2+/+ vs. TR2−/−, and 3) TR2/TR4+/+ vs. TR2/TR4−/−. All mouse strains are maintained as recombinant inbred lines. RNA samples for each genotype and each tissue will be labeled and divided into three identical aliquots for independent hybridization the Affymetrix murine array set. cDNA synthesis, cRNA synthesis and labeling of the RNA samples can be performed as understood in the art. The hybridization, washing and scanning of the arrays can also be performed as understood in the art.

RNA integrity and labeling efficiency are critical to reproducibility in microarray analysis. Prior to probing the expression arrays, each RNA sample to be tested can undergo a quality analysis using, for example, a GeneChip test chip. Initial gene expression analysis can also be performed which allows for a determination of the “fold difference” for each gene between the wildtype and knockout derived tissue. Hybridization data can be scaled as previously described (Tusher, V. G. et al. (2001) Proc. Natl. Acad. Sci. USA 98, 5116-5121) which is herein incorporated by reference at least for material related to analysis of array data). A reference data set can be generated by averaging the expression of each gene from a given tissue from both the wildtype and knockout mice. A cube root scatter plot can be used to compare the data from each individual hybridization to the reference data set. A cube root scatter plot can be used because it is reported to resolve genes expressed at low levels. To calibrate each hybridization, a linear least squares fit will be applied to the cube root scatter plot (Tusher, V. G. et al. (2001) Proc. Natl. Acad. Sci. USA 98, 5116-5121) which is herein incorporated by reference at least for material related to scatter plots). Significance analysis of micro arrays (SAM) (Tusher, V. G. et al. (2001) Proc. Natl. Acad. Sci. USA 98, 5116-5121) which is herein incorporated by reference at least for material related to SAM) will be used to identify statistically significant changes in gene expression between the wildtype and knockout tissue under analysis. SAM functions by incorporating a set of gene specific t tests. This method assigns a score on the basis of the change in gene expression relative to the standard deviation of repeated measurements. Genes are considered potentially significant if their score is greater than a threshold and a false discovery rate is calculated for each data set (Tusher, V. G. et al. (2001) Proc. Natl. Acad. Sci. USA 98, 5116-5121) which is herein incorporated by reference at least for material related to significance).

Genes that are identified as potentially significant can be validated. Initially, the top 20% of genes that show the greatest difference in expression between wildtype and knockout mice for a given tissue will be validated. Annotated genes not in this group that are biologically interesting and show a difference in expression between genotypes can also be validated. Validation can be performed using, for example, quantitative real-time RT-PCR using, for example, TaqMan chemistry. Validation can be performed on the tissue of interest from wildtype and knockout animals. As controls for the real-time RT-PCR, sets of five housekeeping gene primer pairs can be used, such as GAPDH, β-actin, the transferrin receptor, cyclophilin, and elongation factor 1α which are available.

(d) Equipment

The following equipment can be used to perform array analysis: A) Affymetrix GeneChip System. The equipment that comprise the system include a workstation that contains GeneChip suite software for data analysis, a fluidics workstation designed specifically for microarrays, a hybridization oven and a Hewlett Packard GeneArray Scanner and, B) The Laboratory Information Management System (LIMS) and Expression Data Mining Tool (EDMT) running on a Windows NT-based Dell Server that contains Intel Xeon 4×450 MHz processing power, 2 GB RAM, and 110 GB hard drive array. Other equipment includes: C) a Perkin-Elmer 7700 real-time quantitative PCR machine, driven by a dedicated Power Macintosh that also contains Primer Express™ for primer and probe design, Sequence Detector™ and Microsoft Excel for data analysis, and D) Software for array analysis tools (GeneSpring and Stingray). Third party analysis tools consist of additional integrated software for analyzing gene expression, gene function, and for performing gene sequence analysis from microarray data across all platforms. These tools provide methods for clustering, graphing, and predicting the biological function of genes from expression data. Other systems can be used, for example, the PCR-Select subtraction strategy (Clontech).

### 5. EXAMPLE

### Neurological Defects in TR4KO Mice

Several defects in brain structure were discovered in mice lacking the orphan nuclear receptor TR4 (TR4KO). Gross cerebellar size was found to be reduced by 35-50% in TR4KO mice compared to wildtype animals, and a 26% reduction in granule cell number was found in the granule cell layers of cerebella from TR4KO mice. Behaviorally, TR4KO mice display extreme hyperkinesia following injection of pentobarbital anesthesia, as well as an abnormal startle response. Focusing on structural aspects of cerebellar neurotransmission, it was found that there was a 15% reduction in Purkinje cell number, a 45% reduction in granule cell terminal bouton number, and a 40% increase in terminal bouton size in cerebellar cortex tissue of adult TR4KO mice. These cellular and synaptic abnormalities suggest defects in inhibitory neurotransmission in the TR4KO cerebellum. Additionally, evidence of reduced myelination was found in the corpus callosum of TR4KO animals. No abnormalities in cell number, cell type distribution, or structural organization were apparent in either the cortical layers or the hippocampus of TR4KO mice.

a) Materials and Methods

(1) Experimental Animals

TR4KO and WT mice of the same strain were produced through the pairing of animals heterozygous for TR4 ablation. Colony founders were produced using embryonic stem (ES) cells of strain 129SvEv injected into blastocysts of strain C57BL/6. Founders were backcrossed to animals of both C57BL/6 and 129SvEv strains purchased from the Jackson Laboratory (Bar Harbor, Me.). All of the experiments described herein involve descendents of founder animals backcrossed to animals of strain C57BL/6. Mice were housed in the vivarium facility of the University of Rochester Medical Center, in ventilated cages, under conditions of either standard isolation or microisolation. The animals were provided a standard diet with constant access to food and water, and exposed to a 12-hour light/dark cycle (lights on from 06:00 to 18:00). Tail biopsies and ear punching procedures, for genotyping and mouse identification, respectively, were carried out prior to 4 weeks of age. Genomic DNA was isolated from tail samples and extracted DNA was used as template for PCR-based genotyping, as described previously. Mice were routinely monitored for health status by trained vivarium technicians and veterinary staff. All experimental protocols were approved by the University Committee on Animal Resources prior to implementation.

(2) Drawings of TR4KO Hyperkinesia

Video recordings of TR4KO mice were taken, starting just prior to injection of pentobarbital anesthesia for euthanasia. From still videotaped images, Mr. Joel H. Ito, Medical Illustrator (Oregon National Primate Research Center, Beaverton, Oreg.) produced drawings of selected body positions of an anesthesia-injected mouse.

(3) Brain Sample Preparation for Analysis

A total of 24 adult mice, 14 wildtype and 10 TR4KO, were used in the brain morphometry analysis. Animals were anesthetized deeply and perfused through the left ventricle with 20 ml saline (pH 7.3), followed by 20 ml 10% neutral buffered formalin. After fixation, the calvarium was removed from each skull and the exposed brain tissue was post-fixed in 10% neutral buffered formalin. Alternatively, fresh tissues were removed from animals and fixed directly via submersion in 10% formalin. After dissection of the brain from the cranial fossa, photographs of whole brain, as well as of coronal and mid-sagittal sections were taken. Photographic images were used in morphometric analysis to determine the approximate size of the cerebrum and the area of the mid-sagittal cerebellum of each mouse.

(4) Tissue Embedding, Sectioning, and Staining

Brain sections were prepared for microscopy in the laboratories of the University of Wisconsin or of Neuroscience Associates, Knoxville, Tenn. In the case of neuronal counting, tissue was embedded in glycol methacrylate resin, cut into 2 micron thick sections, and stained with toluidine blue.

Analysis of cerebellar development was carried out using samples from 2 wildtype and 2 TR4KO samples from each time point, including P0 and P7. Brains from these animals were exposed through removal of the calvarium, and were fixed for 1-7 days in 10% neutral buffered formalin. The brains were then removed from the cranial fossa and the cerebellum from each sample was processed and embedded in glycol methacrylate resin, cut into 1 micron thick sections, and stained with toluidine blue. For analysis of neuronal myelination in the corpus callosum, tissue samples were embedded in paraffin, cut into 10 micron thick sections, and stained with Luxol fast blue.

(5) Brain Morphometry

Due to the differences in overall brain size between TR4KO and WT mice, thick coronal sections were cut based on markers on the ventral aspect of each brain to obtain comparable brain regions for analysis. A coronal cut was made at the anterior edge of the optic chiasm to obtain comparable regions of the front parietal cortex, and a coronal cut was made at the center of the median eminence (below the hypothalamus and 3^{rd} ventricle) to obtain comparable regions of the dorsal lobes of the hippocampus and the corpus callosum. Cerebellar samples, with associated ponticular region, were cut in mid-sagittal section. The morphometric analysis of both gross and microscopic specimens was carried out on images captured using a digital camera. Images were transferred to a computer and imported into NIH Image software. The area of imported images of cerebral and cerebellar samples was traced manually and measured relative to pixel number. Cortical neuron numbers were determined through counting neurons in microvideo photographs of 0.106 mm² regions of the frontal parietal cortex, in the left and right hemispheres, of each specimen. Hippocampal pyramidal neuron numbers (CA1 and CA3 regions) and granule cell numbers (dentate gyrus) were counted in 0.085 mm² areas. Cerebellar granule cell numbers were counted in areas of 5 nm², and cerebellar Purkinje cell numbers were counted in areas of 0.085 mm². The criteria for inclusion of a cell in the counts was the presence of a complete nuclear halo visible within the cell. For all brain regions, 3-5 areas were counted per sample.

Electron microscopic analysis of granule cell-Purkinje cell synapses were carried out using a JEOL microscope according to previously described methods (Phend, K. D., et al., Journal of Histochemistry and Cytochemistry 40, 1011-1020 (1992), Meshul, C. K., et al., Brain Research 648, 181-195 (1994).). Electron micrographs were imported into ImagePro Plus software, granule cell terminal boutons were traced, and area measurements were obtained via conversion of pixel counts.

(6) Statistics

Differences in mean cerebral surface area, number of cerebral cortical neurons, mean mid-sagittal cerebellar area, mean cerebellar granule neuron number, and mean Purkinje cell number between wildtype and TR4KO mice were analyzed using independent sample 1-tailed t-tests, assuming equal variance. Synaptic bouton number and size data was analyzed via 2-way ANOVA. In many cases in this study extremely small sample sizes have been used to generate the data reported. However, the statistical tests chosen for the analysis of such data is valid in that independent samples are used. In the t-tests, two sample variances are used in calculation of the t value. Each variance is based on squared deviations relative to the sample mean. Each sample then has n₁−1 degrees of freedom. For the two samples there will be (n₁−1)+(n₂−1)=(n₁+n₂−2) degrees of freedom. The t for two independent samples will be based on sample size 1 (n₁)+sample size 2 (n₂)−2 degrees of freedom (Howell, D. C. in Statistical Methods for Psychology 198-205 (Duxbury, Pacific Grove, Calif., 2002)). In this case, samples sizes as small as two for each genotype will allow calculation of the t statistic with 2 degrees of freedom.

b) Results

(1) General Phenotypic Observations and Anesthesia-Induced Hyperkinesia in TR4KO Mice

Observations of both TR4KO and WT animals suggest that the mutant mice have abnormalities of movement as well as behavior. TR4KO males display abnormal sexual behavior, and that increased levels of anxiety or fear, as well as defects in motor coordination or muscle strength, may contribute to the reported behavioral outcomes. Further, TR4KO animals display hypersensitivity to various sensory stimuli, such as sound and touch, and thus hyperactivity in response to such stresses. Typical responses include short bursts of activity (either running or jumping) to avoid a threat Such behavior may be indicative of abnormal startle response (Simon, E. S. et al., Movement Disorders 12, 221-228 (1997).). Although WT animals display threat avoidance behavior as well, the behavior of the TR4KO mice is much more pronounced. Additionally, the mutant mice tend to be more aggressive when handled, compared with WT animals. Other evidence suggesting possible neurological defects include abnormal hindlimb clasping (Simon, E. S. et al., Movement Disorders 12, 221-228 (1997), Labosky, P. A., et al., Development 124, 1263-1274 (1997).) in some TR4KO mice when suspended by the tail, and striking anesthesia-induced hyperkinesia. After being injected with an overdose of pentobarbital anesthesia, as a method of euthanasia, WT animals generally demonstrate exploratory activity until the sedative takes effect. In contrast, TR4KO mice tend to remain in one place, generally a cage corner, (yet appear agitated, likely an effect of the stress of manipulation) for several seconds, after which occurs a period of extremely high activity as the animals jump, run, and flail about the cage (The data showed behavioral defects, and cerebral and cerebellar morphometry in TR4KO mice (A) Hyperkinesia of a 13 week old TR4KO mouse after injection with Pentobarbital anesthesia. Drawings were made from still video images. (B) Pictures of whole brains from adult wildtype (WT) and TR4KO (KO) mice, demonstrating reduced overall brain size as well as further reduction in cerebellar size in TR4KO animals. CX, cortex; CB, cerebellum. (C) Coronal sections of the pre-frontal (a), frontal (b), hypothalamic (c), and thalamic (d) regions of the cerebrum from a TR4KO and a WT mouse at 3.5 months of age are shown. Reduction in the mid-sagittal area of the cerebellum in the TR4KO mouse is apparent (e) when compared with that of the wildtype mouse. C, cerebellum; P, pons; M, medulla.). This hyperkinesia is not, however, observed in all TR4KO mice treated with pentobarbital.

(2) Reduction in Cerebral Size, But not in Neuron Density in TR4KO Mice

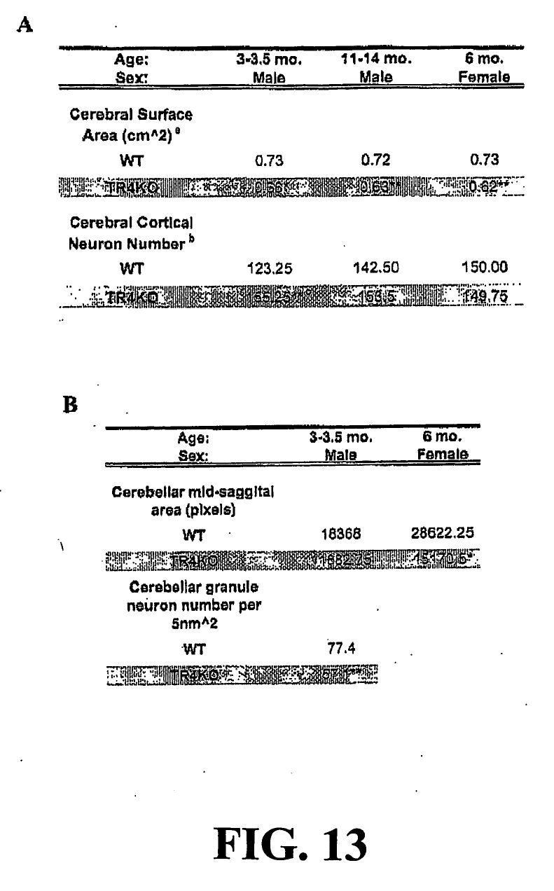

Gross morphometric analysis of the adult TR4KO cerebrum showed reduction in area in comparison to WT animals. The bilateral cerebral hemispheres were included in the measurement, excluding the olfactory bulbs, inferior colliculus, and cerebellum. The cerebral hemisphere area of TR4KO mice is reduced by 9.6% in males at 3-3.5 months of age, 12.5% in males at 11-14 months of age and 14.6% in females at 6 months of age, compared to WT animals at the same time points (FIG. 13A). However, when taking into account an overall 30% reduction in body weight in each of these groups of animals, the reduction in cerebral size in the TR4KO animals may be easily explained.

More specifically, the number of cerebral cortical neurons per 0.106 mm² area was also compared between TR4KO mice and WT controls. Although overall size of the cerebral cortex was diminished in TR4KO animals, there was no significant difference in neuron density in layers III and IV of the front parietal cortex of the right and left hemispheres (FIG. 13A), except among 3-3.5 month old male mice where a 20% increase in neuron density was observed in the TR4KO animals. No differences were seen in males at approximately 1 year of age or in females at 6 months of age, again suggesting that the overall decrease in cerebral size is an effect of smaller overall body size among TR4KO mice. Additionally, no significant defect was observed in the zonal arrangement of neurons of TR4KO cortical grey matter (The data showed the structure of the cerebral cortex and hippocampus is normal, but myelination of the corpus callosum is reduced in TR4KO mice (A) Histological analysis of sections of the cerebral cortex demonstrated no differences in zonal arrangement of neurons between WT and TR4KO (KO) mice. Roman numerals designate layers of the cerebral cortex. (B) Histological analysis of hippocampal sections from 3.5 month old male WT (top) and TR4KO (bottom) mice, after hematoxylin and eosin staining, demonstrated that no obvious differences existed in structure, or in neuron number and zonal arrangement. DG, dentate gyrus. (C) Histological staining with Luxol Fast Blue qualitatively demonstrates reduced myelination of the corpus callosum (CC) in adult TR4KO (KO) mice. HC, hippocampus; Hbn, habenular nucleus.).

The increase in cortical neuron density in 3-3.5 month old TR4KO males compared to WT males at the same age suggests an increased total neuron number in the cerebrum of TR4KO mice. Indeed, when neuronal density is converted to estimated neuron number based on the calculation of the estimated area of the cerebral hemisphere, TR4KO males at 3-3.5 months of age have significantly more total neurons than do WT males, although this effect was not seen among either 11-14 month old males or 6 month old females. The samples sizes from which data regarding neuronal numbers was derived were extremely small, suggesting that, by chance, two 3-3.5 month old TR4KO male mice with particularly high cortical neuron density could have been chosen for this analysis. Adding samples to these data sets will help to determine whether the neuronal densities reported are accurate descriptions of the populations of WT and TR4KO mice at the ages indicated. Indeed, it has been shown that a large degree of heterogeneity in neuronal numbers is present in regions of the cerebral cortex among rats of the same strain (Skoglund, T. S., et al., Neutroscience Letters 208, 97-100 (1996)). Nevertheless, if the total neuron number in the cerebral cortices of TR4KO mice is larger than that of WT animals, there may be functional implications of this phenomenon relative to the various functions of particular cortical regions. For example, increases in neuron number in areas such as the motor cortex, somatosensory cortex, and visual cortex in the TR4KO mice may indicate enhanced function of such regions, leading to increased sensitivity to various stimuli. Further, analysis of sizes of individual neurons in various brain regions suggest reduction in TR4KO cell size compared with WT. Analysis of additional samples can be carried out to confirm differences in cell size, as reduced cell size would reduce the probability of any particular neuron from being included in cell counts, likely causing misinterpretation of neuronal density measurements. Characterization of neuronal processes can also be carried out, as increased process formation may occur in response to reduced neuronal number in the TR4KO, particularly among granule cells of the cerebellum, which is the cell type for which the largest reduction in numbers is observed. Such an increase in neuronal process formation has been demonstrated relative to gender differences in the human cerebral cortex (Rabinowicz, T., et al., Journal of Child Neurology 14, 98-107 (1999)). Mean cortical thickness was found to be comparable between males and females, yet higher neuronal density and number estimates were found in males, suggesting increased neuronal process/neuropil formation in females.

(3) No Defects in Neuron Density, Structure, or Zonal Arrangement in the TR4KO Hippocampus

Based on the distribution of TR4 in the mouse brain, an additional region of interest is the hippocampus. TR4 is known to be highly expressed in granule neurons of the adult mouse hippocampus (Young, W. J., et al., Journal of Biological Chemistry 272, 3109-3116 (1997)). Initial morphometric analysis of the hippocampal regions CA1, CA3 and dentate gyrus suggested no difference between WT and TR4KO mice in neuronal density, structure or zonal arrangement in those areas. Quantitative morphometric analysis can confirm this observation.

(4) Reduced Myelination of the White Matter of the Corpus Callosum in TR4 Knockout Mice

The corpus callosum is composed of myelinated fibers that connect the neocortical hemispheres, defects of which have been associated with the CRASH (corpus callosum hypoplasia, mental retardation, adducted thumbs, spastic paraplegia, and hydrocephalus) syndrome in humans (Freedman, L. P. Endocrine Reviews 13, 129-145 (1992)). Initial analysis of coronal sections of TR4KO and WT brain samples, stained with Luxol fast blue, suggests reduced myelination of neurons of the corpus callosum of adult TR4KO mice. Observations of very few cases in which aged TR4KO animals have been observed to develop posterior paralysis, resulting in dragging of the abdomen and hindlimbs, may be indicative of various levels of progressive demyelination of motor neurons in TR4KO mice. However, further experimental analysis is necessary to support such a claim.

(5) Cerebellar Hypoplasia and Reduced Cerebellar Granule Cell Density in Adult TR4 Knockout Mice

Gross observation of the TR4KO cerebellum (The data showed reduction in width of, and granule cell number in, TR4KO cerebellar granular layers (A) Histology of mid-sagittal cerebellar sections show reduction in overall TR4KO cerebellar size, width of the internal granule cell layer, and folia development. Roman numerals designate cerebellar folia structure. C, cerebellum; P, pons; M, medulla. (B) Toluidine blue-stained cerebellar sections qualitatively demonstrate reduction in width of the internal granule cell layer (IGL), as well as reduced granule cell number in 3 month old male TR4KO mice.), as well as morphometry of cerebellar mid-sagittal sections (FIG. 13B), showed that cerebellar size is significantly reduced in adult TR4KO mice compared to WT mice. TR4KO females at 6 months of age had a mid-sagittal cerebellar area that was 47% less compared to that of WT animals of the same sex and age (p=0.05, N=2 for each genotype). In this case, an approximately 30% reduction in overall body weight cannot explain such a dramatic decrease in cerebellar size. Further, cerebellar lobule development appeared delayed in mid-sagittal sections from TR4KO mice. Initial observations of the microscopic structure of the cerebellum in adult TR4KO animals indicated that there was a reduction in the width of the internal granule cell layer (IGL). By counting cerebellar granular neurons in comparably sized areas of 3 month old male TR4KO and WT mice, it was confirmed that the mutant mice had a 26% reduced population of cerebellar granule cells (FIG. 13B).

(6) Reduction in Cerebellar Granule Cell Number Begins Early in Cerebellar Development of TR4 Knockout Mice

To explore the effects of the loss of TR4 in cerebellar development more closely, TR4KO and WT control mice were sacrificed perinatally (P0) and at postnatal day 7 (P7). After sectioning and staining with toluidine blue, the structure and cellular composition between TR4KO and wildtype animals was compared. At P0, a delay in cerebellar folia formation was observed in the TR4KO (data not shown). At both P0 and P7, there appears to be a reduction in granule cell number in the external granule cell layer (EGL) of the TR4KO (The data showed reduced granule cell density and migration in the developing TR4KO cerebellum The cerebella from newborn (P0) and seven day old (P7) WT and TR4KO mouse pups were sectioned and stained with toluidine blue for structural analysis. (A) At P0, there appears to be a slight reduction in density of granule cells in the TR4KO external granule cell layer (EGL, right), as well as a reduced number of granule cells migrating inward (down). (B) By P7, the Purkinje cell layer (PCL) has formed, and a slight reduction in the density of the EGL as well as in the number of cells migrating inward to form the IGL is again observed in TR4KO mice.), along with reduced numbers of granule cells migrating toward the IGL. Quantitative granule cell counts from WT and TR4KO samples will be necessary to confirm this observation.

(7) Purkinje Cell Number is Reduced in the TR4KO Cerebellum

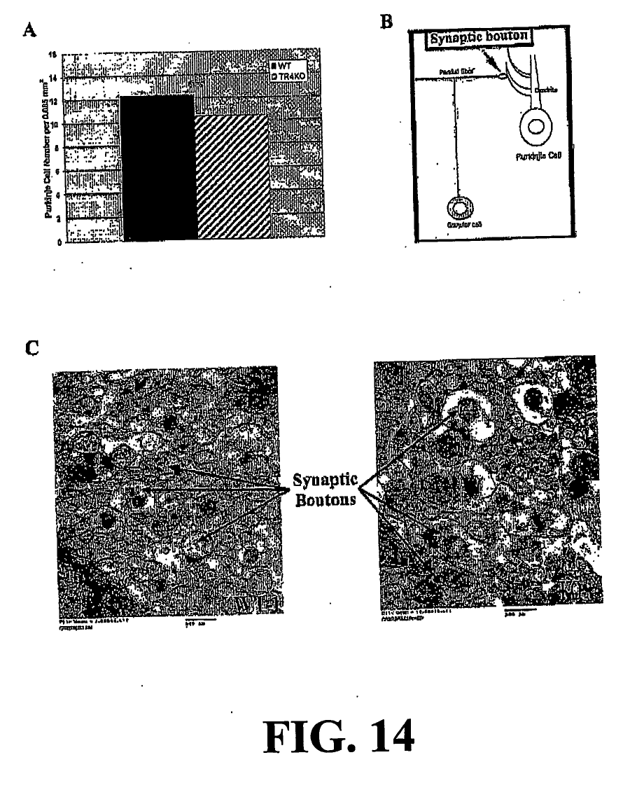

Morphometric analysis focusing on Purkinje cells of the cerebellar cortex revealed a 15% reduction in the number of these neurons per 0.085 mm² area in TR4KO animals compared to the number in WT mice (FIG. 14A). FIG. 14B depicts the interaction between granule cell parallel fibers (axons) and Purkinje cell dendrites, which occurs in the molecular layer of the cerebellar cortex (Voogd, J. & Glickstein, M., Trends in Neuroscience 21, 370-375 (1998), Goldowitz, D. & Hamre, K., Trends in Neuroscience 21, 375-382 (1998)).

(8) Parallel Fiber Synaptic Defects are Present in the TR4 Knockout Mouse Cerebellum

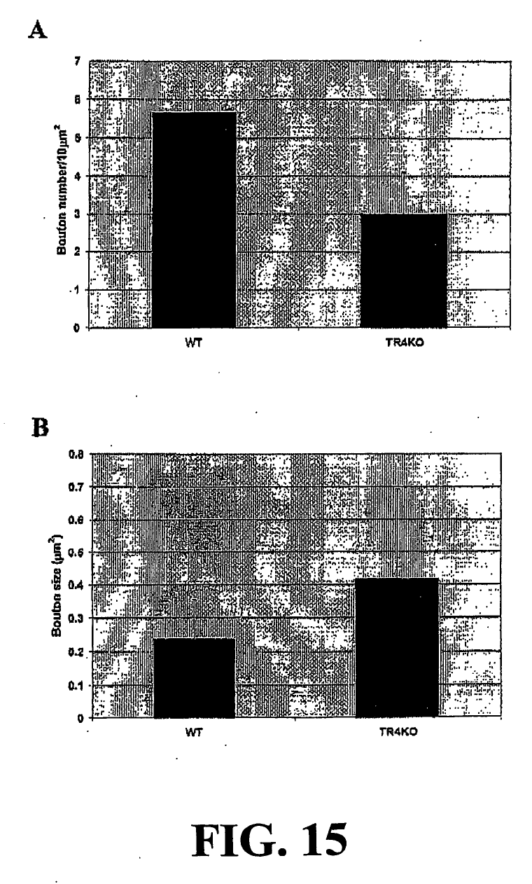

Ultrastructurally, the number of synaptic terminal boutons of granule cell parallel fibers in the cerebella of TR4KO mice was reduced by approximately 45% (FIG. 15A), but the size of individual boutons was increased by 40% (FIG. 14C; FIG. 15B), compared to those found in WT mice. The cell distribution and structural abnormalities reported here are likely to have disruptive effects upon the balance of excitatory and inhibitory signaling pathways present in the mouse cerebellum, and may explain aspects of the behavioral abnormalities, motor defects, and drug sensitivity displayed by TR4-deficient mice.

In the mouse, cerebellar development begins in mid-gestation, with appearance of the cerebellar anlage during embryonic days 10-12 (E10-12), and continues for three weeks postnatally. During this time, development and colonization of the cerebellar cortex with four major types of neurons occurs (Voogd, J. & Glickstein, M., Trends in Neuroscience 21, 370-375 (1998), Goldowitz, D. & Hamre, K., Trends in Neuroscience 21, 375-382 (1998).). These neuronal cell types include the Purkinje cells, which separate the molecular layer from the internal granule layer (IGL) and serve as the sole output from the cerebellar cortex; the granule cells, which are the main component of the IGL of the adult mouse and receive mossy fiber afferents conveying sensory information; the Golgi cells, which are inhibitory interneurons providing feed backward inhibition to granule cells via GABA and glycine neurotransmission; and the stellate/basket cell inhibitory interneurons, which are GABAergic cells providing feed forward inhibition to Purkinje cells (Voogd, J. & Glickstein, M., Trends in Neuroscience 21, 370-375 (1998), Colin, F., R is, L. & Godaux, E. in The Cerebellum and its Disorders (eds. Manto, M. & Pandolfo, M.) 6-29 (Cambridge University Press, Cambridge, UK, 2002).). Important during postnatal cerebellar development in mice is the migration of granule cells from the EGL, where initial granule cell proliferation occurs, to their final residence in the IGL. Also vital to proper cerebellar development are adequate numbers of Purkinje cells, as they determine the size of the granule cell population through control of granule cell mitotic activity in the EGL (Manto, M. in The Cerebellum and its Disorders (eds. Manto, M. & Pandolfo, M.) 1-5 (Cambridge University Press, Cambridge, UK, 2002)).

Hypoplasia and/or degeneration of the cerebellum is often linked to ataxia, hypotonia, tremor, and other defects of motor function. A number of hereditary ataxias, including Friedreich's ataxia and spinocerebellar ataxia type I, which involve degeneration of the cerebellum and associated neural connections, have been described in humans (reviewed in (Klockgether, T. & Evert B., Trends in Neuroscience 21, 413-418 (1998)). Similarly, several mouse models of ataxia have also been described. The weaver mouse mutant displays extensive cell death among post-mitotic granule cells, close to the site of proliferation in the external granule cell layer (EGL) (Rakic, P. & Sidman, R L., Proceedings of the National Academy of Sciences USA 70, 240-244 (1973)), an effect thought to be secondary to migratory failure and mediated by defects in structure and function of supporting Bergmann glia. More interesting relative to the phenotypes observed in TR4KO mice are the cerebellar defects observed in mouse models of other orphan receptor mutants. In the staggerer mutant, severe neurodegeneration of the cerebellum, resulting in ataxia, was observed (Sidman, R. L., et al., Science 136, 610-612 (1962)). More recently, the genetic mutation causing the staggerer phenotype has been defined as affecting the Retinoic acid receptor-related Orphan Receptor α (RORα) gene (Hamilton, B. A., et al., Nature 379, 736-739 (1996)). As a transcription factor, RORα has been suggested to be a constitutive activator of transcription of various target genes; however, additional evidence of association of regions of the gene with nuclear co-repressors indicate two-way regulation (Schrader, M., et al., Journal of Biological Chemistry 271, 19732-19736 (1996), Harding, H. P., et al., Molecular Endocrinology 11, 1737-1746 (1997).). Another orphan receptor, which results in cerebellar abnormalities when disrupted in the mouse, is rev-erbAα. Mice deficient in expression of this gene display Purkinje cell abnormalities, delayed granule cell proliferation and migration, and increased granule cell apoptosis (Chomez, P., et al., Development 127, 1489-1498 (2000).). Further, thyroid hormone has been shown to regulate expression of two genes, reelin and dab1, essential for proper neuronal migration and lamination during brain development (Alvarez-Dolado, et al., Journal of Neuroscience 19, 6979-6993 (1999)). With a nuclear hormone response element (DR2) shared by RORα, rev-erbAα and TR4 (Jarvis, C. I., et al., Molecular and Cellular Endocrinology 186, 1-5 (2002)., Harding, H. P. & Lazar, M. A., Molecular & Cellular Biology. 15, 4791-4802 (1995), Lee, Y. F., et al., Journal of Steroid Biochemistry & Molecular Biology 81, 291-308 (2002).), as well as regulation of known thyroid hormone receptor target genes by TR4 (Lee, Y. F., et al., Journal of Biological Chemistry 272, 12215-12220 (1997)), it is consistent that interaction or crosstalk between these nuclear hormone receptor orphan receptors affects cerebellar development and function. The loss of TR4 may disrupt regulatory pathways through lack of a heterodimeric partner, loss of competition for binding to various response elements, or alternative deregulation of the complex developmental signaling pathways in which these nuclear receptors are involved.

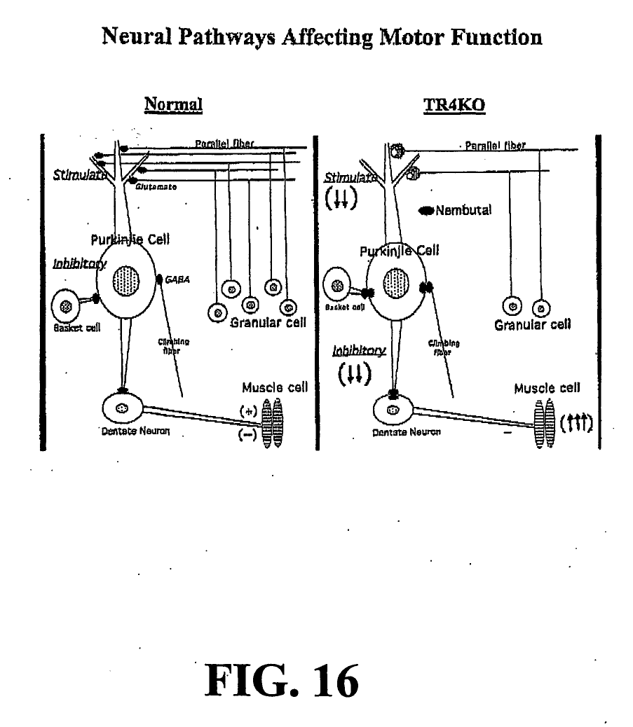

The hyperkinesia abnormal startle response and apparent increased emotionality (anxiety/fear) observed in TR4KO mice suggest functional consequences of the described cerebellar defects. Particularly, the pathways of neurotransmission controlling inhibition of motor function are likely to have been affected by the loss of TR4 expression (FIG. 16). The reduction in Purkinje cell number has potential consequences in both development (support of granule cell proliferation) and function. As a GABAergic efferent projecting from the cerebellar cortex, the inhibitory Purkinje cell synapses with neurons of the cerebellar deep nuclei, which contribute to the control of motor responses. The glutamatergic granule cells are primarily excitatory in function, and, in response to mossy fiber afferents, stimulate inhibitory signaling into the cerebellar deep nuclei by the Purkinje cells (Colin, F., Ris, L. & Godaux, E. in The Cerebellum and its Disorders (eds. Manto, M. & Pandolfo, M.) 6-29 (Cambridge University Press, Cambridge, UK, 2002)). Reduced numbers of Purkinje cells in the TR4KO mouse, combined with reduced granule cell excitatory input for stimulation of inhibitory Purkinje cell signaling (FIG. 15A), are likely to result in over-stimulation of motor pathways. Indeed, such over-stimulation is apparent in TR4KO mice when they are stimulated to move in response to a perceived threat, and is characterized by abrupt bouts of running, jumping, and stereotypical head jerking behavior. Further evidence of deregulation of inhibitory neurotransmission in the TR4KO cerebellum is the response of mutant animals to injection with pentobarbital (Nembutal) anesthesia (FIG. 16). Pentobarbital is a known GABA agonist, and moderate increases in activity are observed in WT animals upon injection, prior to sedation. In the TR4KO condition of reduced Purkinje cell number, enhancement of the function of GABAergic inhibitory interneurons (basket and stellate cells, Golgi cells), which either inhibit Purkinje cell excitation directly or regulate excitation from mossy fiber input (Colin, F., R is, L. & Godaux, E. in The Cerebellum and its Disorders (eds. Manto, M. & Pandolfo, M.) 6-29 (Cambridge University Press, Cambridge, UK, 2002)), likely serves to perpetuate the disinhibition, and results in pronounced hyperkinesia until the sedative properties of the drug take effect.

The qualitative indication of an abnormal startle response among TR4KO mice suggests defects in signaling pathways involving glycine, another inhibitory neurotransmitter. Mutations in the gene encoding glycine receptor subunits have been linked to the clinical disorder hyperekplexia, or abnormal startle response (Andrew, M. & Owen, M. J., British Journal of Psychiatry 170, 106-108 (1997)). Additionally, several mouse models carrying mutations in glycine receptor (GlyR) subunits have been studied (Simon, E. S. et al., Movement Disorders 12, 221-228 (1997)), and comparative levels of severity of particular defects in motor function, such as startle response, tremor, dystonia, righting reflex, gait, and hindlimb clasping, have been reported. Each mutant mouse is described as having a hyperkinetic motor disorder, and similar to TR4, each mutant displays an abnormal startle response, gait defect, and abnormal hindlimb clasping. Dystonia, tremor, and impaired righting reflex were even observed in one severely affected TR4 mouse, subsequent to handling. It is important to note that a range of phenotypes is often observed among TR4KO animals, highlighting the complexity of the physiological defects resulting from disruption of TR4 function. From body weight to drug response, individual TR4KO mice often show significant variance in phenotype, suggesting the interaction of many factors in production of that phenotype and the potential for varying levels of compensation from alternative signaling pathways. However, possible involvement of TR4 in regulation of glycinergic neurotransmission is evident, and clearly worth pursuing. The evidence presented here is consistent with major roles for TR4 in both development and function of the CNS, specifically highlighting its importance in the cerebellum.

### 6. EXAMPLE

### Effect on Fertility Priapism, Reduced Fertility, and Growth Retardation in Mice Lacking Testicular Orphan Nuclear Receptor 4

Testicular orphan nuclear receptor 4 (TR4) is a member of the nuclear hormone receptor superfamily lacking a known ligand. To investigate the physiological roles of TR4, mice deficient in functional TR4 protein (TR4^{-/-}) were generated. The homozygous ablation of TR4 resulted in a complex phenotype with abnormalities in reproduction, growth, and behavior. Varying numbers of male mice lacking TR4 display penile priapism (from 22-65%), with the number of mice showing this phenotype increasing with age. Both male and female TR4^{-/-} mice are subfertile, with significant reduction in offspring generated compared with wildtype (TR4^{+/+}) mice. Male TR4^{-/-} mice produce reduced numbers of sperm, ranging from 33-67% of TR4^{+/+} numbers at different time points from the onset of sexual maturity to adulthood. Further exploration of male TR4^{-/-} reproductive deficiencies via sexual behavior analysis exposed behavioral and motor coordination defects. Specifically, TR4^{-/-} males displayed increased fear or anxiety, and fewer intromissions and ejaculations than TR4^{+/+} males after being paired with hormonally primed females. Upon investigation of the expression levels of genes known to affect sexual behavior, maternal behavior, stress response, and penile erection, it was found that TR4^{-/-} animals express less ERα, ERβ, and oxytocin in the hypothalamus, as well as reduced nNOS in the penis. Additional defects observed among TR4^{-/-} animals include abnormal maternal behavior, with pups born to TR4^{-/-} mothers dying without evidence of milk intake, as well as a 20-50% growth reduction compared to TR4^{+/+} animals. The results provide in vivo evidence that TR4 plays important roles in penile physiology, fertility, behavior, and growth.

a) Materials and Methods

(1) TR4KO Targeting Construct Design and Mutant Mouse Generation

Sequencing of mouse TR4 genomic DNA, targeting construct design and production of genetically manipulated mice was accomplished as disclosed herein. The lambda KOS system (Wattler et al. 1999) was used to derive a TR4 targeting vector. Three independent genomic clones spanning exons 4-10 were isolated. The targeting vector was derived from one clone and contained a 2173 bp deletion that included most of exon 4 and all of exon 5. The genomic sequence encoding the DBD of TR4 was replaced by an internal ribosomal entry site (IRES) Lac-Z/MC1-Neo selection cassette. The Not I linearized vector was electroporated into strain 129SvEv^{brd} (LEX1) embryonic stem (ES) cells, and G418/FIAU-resistant ES cell clones were isolated and screened for homologous recombination of the mutant DNA by Southern blot. One targeted ES cell clone was injected into blastocysts of strain C57BL/6(albino), which were then inserted into pseudopregnant female mice for continuation of fetal development. Resulting chimeric male mice were then mated to C57BL/6(albino) females to generate animals heterozygous for the mutation. Three sexually mature breeding pairs, with each mouse heterozygous for the disrupted TR4 gene, were provided.

(2) Experimental Animals

The mice used in all experiments described were housed in the vivarium facility of the University of Rochester Medical Center, in ventilated cages, under conditions of either standard isolation or microisolation. The animals were provided a standard diet with constant access to food and water, and exposed to a 12-hour light/dark cycle (lights on from 06:00 to 18:00). Due to their small size, TR4KO pups were weaned at approximately 4-5 weeks of age, whereas WT and heterozygous pups were weaned at approximately 3 weeks. Tail biopsies and ear punching procedures, for genotyping and mouse identification, respectively, were carried out prior to 4 weeks of age. To determine pup genotype ratios generated by heterozygous pairings, 751 pups from 110 litters were genotyped via PCR amplification of WT or mutant TR4 alleles. For weight measurements, littermates from heterozygous matings were weighed every other day, starting at postnatal day 2, until day 30. Subsequently, pups were weighed once per week for 8 additional weeks (last time point: 12 weeks). PCR analysis of DNA prepared from tail snips was used to determine pup genotypes upon weaning. Mice were routinely monitored for health status by trained vivarium technicians and veterinary staff. All experimental protocols were approved by the University Committee on Animal Resources and the office of Environmental Health and Safety prior to implementation.

(3) Breeding Colony Establishment

To produce adequate numbers of TR4KO mice and control animals for experimental analysis, the heterozygous founder mice were backcrossed to animals of both C57BL/6 and 129SvEv strains purchased from the Jackson Laboratory (Bar Harbor, Me.). All of the experiments described herein involve descendents of founder animals backcrossed to animals of strain C57BL/6. The ES cells genetically manipulated to carry the TR4KO construct were of the 129SvEv strain, and were subsequently injected into blastocysts of strain C57BL/6. Therefore, a small colony of descendents of founder mice backcrossed to animals of strain 129SvEv have been maintained in the event that strain specific differences are found to exist among the mutant mice and exploration of those differences are necessary.

(4) PCR Genotyping

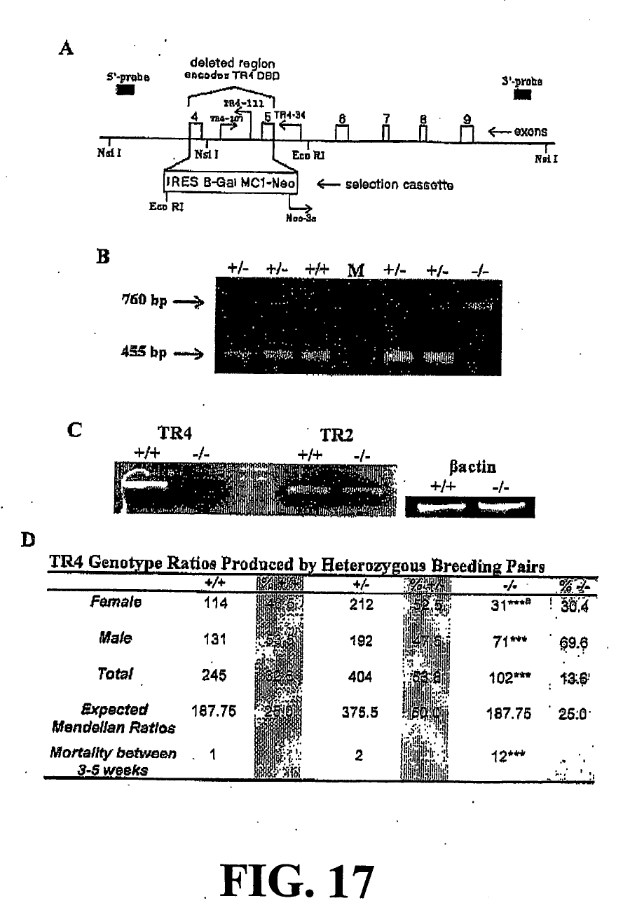

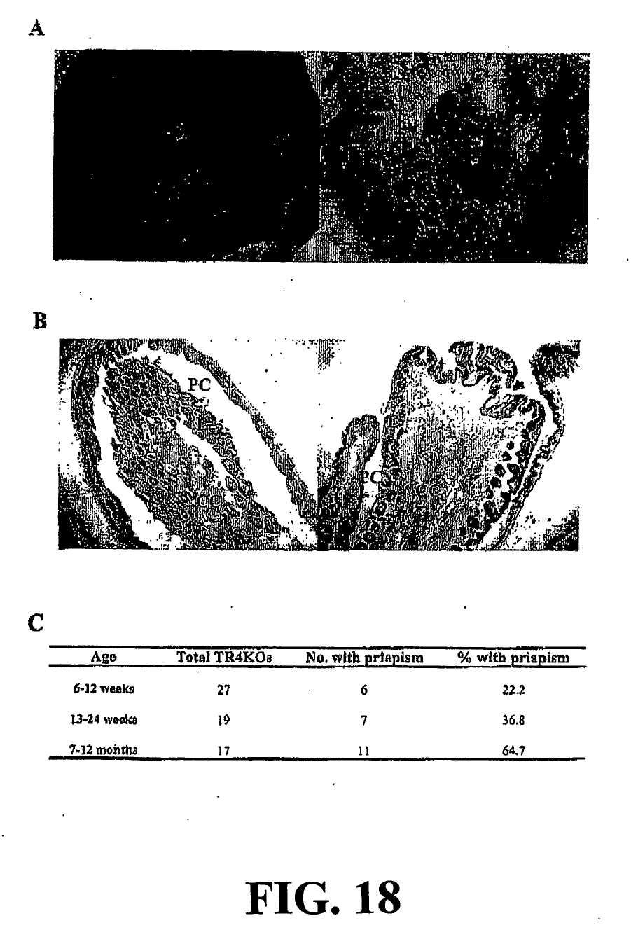

Tail biopsies were collected from mice prior to 4 weeks of age, and genomic DNA was isolated from the tail samples, after proteinase K digestion, via phenol/chloroform extraction (Hogan et al. 1994). Extracted DNA was used as template for PCR-based genotyping. Primers for amplification of the wildtype and mutant alleles were designed based on sequences within intron 4 (deleted in the mutant), or within the selection cassette and external to the deleted region, respectively (FIG. 18-17). The PCR primer sequences are SEQ ID NO:26 TR4-107 (WT, forward): 5′-GGAGACACACTGCACATGTTCGAATAC-3′, SEQ ID NO:27 TR4-111 (WT, reverse): 5′-CACAGCTCATTTCTCTGCTCACTTACTC-3′, SEQ ID NO:28 Neo-3a (TR4KO, forward): 5′-GCAGCGCATCGCCTTCTATC-3′, and SEQ ID NO:29 TR4-34 (TR4KO, reverse): 5′-TGCAAGCATACTTCTTGTTCC-3′. The wildtype and TR4KO primer sets were used to amplify mouse DNA samples in independent 25 μl reactions. DNA in PCR genotyping reactions was amplified in 35 cycles with melting, annealing, and extension temperatures of 94° C., 61° C., and 72° C., respectively.

(5) RT-PCR Analysis of TR4 and TR2 Gene Expression

For RT-PCR analysis of TR4, TR2, and β-actin RNA expression (FIG. 18-17), total RNA was isolated from the cerebella of 7 month old wildtype (+/+) and TR4KO (−/−) mice, using TRIzol® Reagent (GIBCO BRL). First strand cDNA synthesis was achieved using the Superscript™ II RNase H Reverse Transcriptase kit (GIBCO BRL) and 2 μg of total RNA from each sample. PCR primers complimentary to regions of the TR4, TR2 and β-actin genes were used to amplify the gene segments and therefore determine relative expression levels. The sense strand (S) and antisense strand (AS) PCR primer sequences are (m, mouse): SEQ ID NO: 30 mTR4(S): 5′-CATATTCACCACCTCGGACAAC-3′, SEQ ID NO:31 mTR4(AS): 5′ TGACGCCACAGACCACAC-3′, product size: 137 bp, SEQ ID NO:32 mTR2(S): 5′-CCGCATCTAATCGCTGGAGAG-3′, SEQ ID NO:33 mTR2(AS): 5′-GCATAGGAGAAGGCATGGTGAG-3′, product size: 100 bp, SEQ ID NO:34 β-actin(S): 5′-TGTGCCCATCTACGAGGGGTATGC-3′, SEQ ID NO:35 β-actin(AS): 5′-GGTACATGGTGGTGCCGCCAGACA-3′, product size: 448 bp

(6) Radioimmunoassay

Serum levels of insulin-like growth factor-1 (IGF-1) were determined using a commercially available radioimmunoassay kit employing the competitive binding immunoassay format (Diagnostic Systems Laboratories, Webster, Tex.).

(7) Tissue Preparation, Histology, and Immunostaining

Mice were anesthetized with an overdose of pentobarbital and perfused through the left ventricle with 20 ml saline (pH 7.3), followed by 20 ml of 4% paraformaldehyde or 10% neutral buffered formalin. Tissues were then removed and post-fixed by submersion in 10% formalin. Alternatively, fresh tissues were fixed by direct submersion in 10% neutral buffered formalin, prior to processing. Tissue was processed for embedding in paraffin using an RHS Tissue Processing System (Hacker Instruments & Industries, Inc., Fairfield, N.J.). Testis, epididymis, seminal vesicle, coagulating gland, and penis tissues were cut in 5-7 μm sections, deparaffinized, and stained with hematoxylin and eosin following standard procedures. Liver tissues stained via immunohistochemical methods were embedded in paraffin, cut at a thickness of 7 μm, deparaffinized, and stained with a mouse monoclonal anti-human IGF-1 primary antibody (Upstate Biotechnology, Lake Placid, N.Y.), followed by use of a biotinylated anti-mouse secondary antibody (Vector Laboratories, Burlingame, Calif.). Staining was visualized using the Vector ABC staining kit followed by DAB substrate (Vector Laboratories). Penis sections were stained using an antibody against either nNOS (Transduction Laboratories), at a 1/100 dilution, or S100 (DAKO), at a 1/400 dilution. Substrate development was achieved through use of the Vector AEC kit (Vector Laboratories). Hematoxylin was used as a nuclear counterstain following each immunohistochemical procedure. Sperm smears, prepared at the time sperm counts were taken, were fixed with Cytoprep™ cytology smear fixative (Fisher Scientific International, Inc. Hanover Park, Ill.). Histological staining of sperm smears was accomplished using the Jorvet™ Dip Quick Stain kit (Jorgensen Laboratories, Inc., Loveland, Colo.). Stained sections and sperm smears were analyzed via light microscopy (Nikon, Tokyo, Japan) and digital images were obtained using a SPOT-RT™ digital camera (Diagnostic Instruments, Inc., Sterling Heights, Mich.).

(8) Continuous Mating and Male Sexual Behavior Analysis

For the continuous mating study, 5 WT and 5 TR4KO males at 5 months of age were each paired with an adult WT female for 4 months. The heterozygous or WT pups generated from these matings were used for breeding, as control animals in experiments, or were sacrificed. For male sexual behavior analysis, female mice of strain ICR, ovariectomized (OVX) at 8-9 weeks of age, were purchased from the University of Rochester transgenic core facility. To induce estrous in OVX females, the mice were injected with 12 μg estradiol valerate one day before mating, and with 500 μg progesterone 4-7 hours before mating. At the time of mating, one stimulus female was paired with a 7-8 month old WT or TR4KO mouse for either 90 minutes or 6 hours, beginning between 18:00-22:00. Mating behavior was videotaped and subsequently scored for latency to, and number of, particular behaviors. The behaviors of interest included mounting, intromission, and ejaculation. After the 6 hour mating trial, experimental pairs remained together until the following morning. Female mice were then visually examined for the presence of a vaginal plug.

(9) Body and Reproductive Organ Weights, Sperm Counts, Sperm Motility, and Germ Cell DNA Content Measurements

Sperm numbers were counted using a hemacytometer, and visual assessment of motility was made at the time sperm counts were obtained. The number of motile sperm was compared to the total sperm number to determine the percent of motile sperm in each sample. Mice were weighed prior to sacrifice, and, after anesthesia, the left testis and epididymis from each animal was dissected out and weighed.

Germ cell DNA content was determined via flow cytometric analysis of propidium iodide stained cells. Germ cells from testis tissue from TR4KO and WT mice were dispersed through grinding of tissue between two glass microscope slides and suspended in Phosphate-buffered saline (PBS). After centrifugation at 1200 rpm, each cell pellet was resuspended in 0.5 ml PBS. Cells were fixed by adding 4.5 ml cold 70% ethanol and incubating for at least 2 hr. on ice. Fixed cells were centrifuged at 1200 rpm for 5 min., and the supernatant ethanol was decanted. Cells were resuspended in 5 ml PBS, and, after a 60 sec. incubation, were recentrifuged. Supernatant was decanted and cell pellets were resuspended in 1 ml propidium iodide staining solution (100 ml 0.1% v/v Triton-X-100 in PBS with 20 mg DNase-free RNase A and 2 mg propidium iodide (Molecular Probes)) and incubated for 30 min. at room temperature. Cell fluorescence was measured by a flow cytometer with a 488 nm argon-ion laser fluorescence excitation source. Cell DNA content data was analyzed using Multicycle software (Phoenix Flow Systems).

(10) RT-PCR Analysis and Real Time PCR Quantitation of Sexual Behavior-Related Gene Expression

For RT-PCR and Real Time PCR analyses of androgen receptor (AR), estrogen receptor (ER) alpha (α) and beta (β), vasopressin (VP), oxytocin (OT), and β-actin RNA expression (FIG. 18-23), total RNA was isolated from the hypothalamic region of the brains of 7 month old wildtype (+/+) and TR4KO (−/−) mice, using TRIzol®Reagent (GIBCO BRL). First strand cDNA synthesis was achieved using the Superscript™II RNase H Reverse Transcriptase kit (GIBCO BRL) and 2 μg of total RNA from each sample. PCR primers complimentary to regions of AR, ERα, ERβ, VP, OT, and β-actin genes were used to amplify the gene segments and therefore determine relative or quantified expression levels. The sense strand (S) and antisense strand (AS) PCR primer sequences are (m, mouse; h, human): SEQ ID NO: 36 hAR(S): 5′-GCAGCAGCAGCAAGAGACTA-3′, SEQ ID NO:37 hAR(AS): 5′-TCATCCAGGACCAGGTAGCC-3′, product size: 88 bp; SEQ ID NO:38 mERα(S): 5′-CCTGGAGATGTTGGATGC-3′, SEQ ID NO:39 mERα(AS): 5′-GTAAGGAATGTGCTGAAGTG-3′, product size: 119 bp; SEQ ID NO:40 mERβ(S): 5′-GCGACGACGGCACGGTTC-3′, SEQ ID NO:41 mERβ(AS): 5′-CTGCTTCCTGGCTTGCGGTAG-3′, product size: 118 bp; SEQ ID NO:42 mVP (S): 5′-TGTGCTGGACCTGGATATGC-3′, SEQ ID NO:43 mVP(AS): 5′-AGTGTGCGGGAATGCTCTC-3′, product size: 119 bp; SEQ ID NO:44 mOT (S): 5′-CTTGGCTTACTGGCTCTG-3′, SEQ ID NO:45 mOT(AS): 5′-CAGATGCTTGGTCCGAAG-3′, product size: 143 bp.

Real Time quantitative PCR amplification of reversed transcribed first strand DNA samples was carried out using the iCycler iQ™ PCR cycler and detection system (Bio-Rad Laboratories, Inc., Hercules, Calif.). Analysis of data obtained and calculation of relative gene expression was performed using the 2^{-ΔΔC}_{T} method (Livak and Schmittgen 2001). For each gene analyzed, the expression level of the WT was set at 1 and relative expression in TR4KO samples was calculated. Therefore, absolute expression levels are not reported and comparisons between genes cannot be made.

(11) Maternal Behavior Observation and Tests of Female Reproductive Capacity

Age-matched adult WT and TR4KO female mice were each paired with a sexually mature WT male for 2.5 weeks and then separated. Mothers and pups were observed in their home cage after parturition. Images of the mother and pups in the cage, as well as close-up images of pups alone were taken using a digital camera. Mammary tissue stained with hematoxylin and eosin was processed for embedding and embedded in paraffin. Sections were cut at a thickness of 7 μm, dried at 37° C. overnight, and processed for staining. Stained sections were analyzed via light microscopy (Nikon, Tokyo, Japan) and digital images were obtained using a SPOT-RT digital camera (Diagnostic Instruments, Inc., Sterling Heights, Mich.).

(12) Transient Transfection/Reporter Gene Assay

The human nNOS promoter plasmids p4.3nNOS-Luc, p2.3nNOS-Luc, and pnNOS(1880/2187)-Luc were provided by Dr. Anthony P. Young (Ohio State University). COS1 cells were grown in Dulbecco's Modified Eagle's Medium (DMEM) with 10% fetal bovine serum, and 50 units/ml each of streptomycin and penicillin. Cells were cultured in 24-well plates (Corning) at a concentration of 5×10⁴ cells/per well. The NNOS reporters (0.8 μg/well) were co-transfected, with TR4 expression vectors (0.2 μg/well) or parental vectors, into COS1 cells using Superfect™ reagent (Qiagen) according to the manufacturer's instructions. The Renilla Luciferase gene, driven by the thymidine kinase promoter (pRL-TK, 10 ng/well) served as an internal control for transfection efficiency. Cells were harvested 48 hr. after transfection and lysed in passive lysis buffer (Promega). Luciferase activity was measured using a luminometer (TD-20/20, Turner Designs).

(13) Electrophoretic Mobility Shift Assay (EMSA)

TR4 proteins were synthesized in rabbit reticulocyte lysate (Promega), according to the manufacturer's instructions. The oligonucleotide probe (SEQ ID NO:46 nNOS-NHR, bp-198 to -211, 5′-CTGGTCAACCTTGACTTCCTT-3′) was end-labeled with [γ]³²P in a T4 polynucleotide kinase reaction (New England Biolabs). The EMSA reaction was performed with 2 μl TR4-containing lysate in an EMSA buffer (10 mM HEPES, pH 7.9, 6% (v/v) glycerol, 2% (v/v) Ficoll, 100 mM KCl, 0.5 mM EDTA, 2.5 mM MgCl₂, and 1 mM dithiothreitol). The reaction mixtures were incubated for 15 min. at room temperature, in the presence or absence of a mouse monoclonal anti-TR4 antibody (#15). The protein-DNA complexes were analyzed on a 5% native polyacrylamide gel. The results were visualized by autoradiography (Storm PhosphorImaging System, Amersham Pharmacia).

(14) Statistics

The body weight data collected was analyzed by ANOVA. IGF-1 radioimmunoassay data and mortality rates were analyzed using independent sample 1-tailed t-tests, assuming equal variance. Ratios of genotypes generated from heterozygous pairings and differences from expected Mendelian ratios, overall and within sex, were analyzed using the χ² test. Sex ratios within each genotype were also analyzed using the χ² test.

Male reproductive results (continuous mating experiments), sperm count and motility data, as well as body and organ weight differences, were statistically analyzed using one-tailed, independent sample t-tests, assuming equal variances. In the case of male sexual behavior data, means and standard deviations for behavior latencies, and mean number of behavior displays, with associated ranges, are shown. Extremely high variation in both latencies and numbers of specific behavioral displays was observed both within and between groups. Differences in numbers of mice of a particular genotype displaying a particular behavior were analyzed by one-tailed, independent sample t-tests, assuming equal variances, after coding for the presence (1) or absence (0) of the behavior.

b) Results

To study the physiological roles of TR4 in a mammalian system, the expression of TR4 was disrupted in experimental mice. An IRES β-gal MC1-Neo selection cassette was constructed and inserted into the TR4 gene such that it replaced exons 4 and 5, as well as the intervening intron 4 (FIG. 17A). The targeting vector has significant 5′ and 3′ stretches of TR4 genomic DNA to serve as sites of homologous recombination, a neo expression cassette for selection of embryonic stem cell clones, and the DNA sequence encoding Lac-Z (β-galactosidase). Additionally, the deleted region of TR4, exons 4 and 5, encodes the DBD of the receptor. Elimination of the DBD renders TR4 functionally inactive, as it can no longer act as a transcription factor to regulate the expression of target genes.

(1) Screening Strategies for Confirmation of TR4 Ablation

Two pairs of PCR primers were designed for genotyping. The TR4^{+/+} primers, TR4-107 and TR4-111, are specific for sequences of genomic DNA within intron 4, a region of the TR4 gene known to be deleted upon homologous recombination with DNA from the targeting construct. The TR4^{-/-} primers, Neo-3a and TR4-34, are specific to a region of TR4 genomic DNA 3′ of the selection cassette and to a sequence within the selection cassette, respectively (FIG. 17A). PCR amplification of DNA using primers TR4-107 and TR4-111 in the presence of TR4^{+/+} genomic DNA yields a product of 455 bp, and use of primers TR4-34 and Neo-3a in the presence of the targeting construct yields a product of 760 bp (FIG. 17B).

To confirm loss of TR4 gene expression in TR4^{-/-} mice, total RNA prepared from cerebellar tissue from 7 month old TR4^{+/+} and TR4^{-/-} animals was analyzed by RT-PCR amplification using primers complimentary to a region of the TR4 gene 3′ of the selection cassette insertion site. As expected, TR4 transcript was present in total RNA samples prepared from TR4^{+/+} mice, but was not found in TR4^{-/-} samples (FIG. 17C). β-actin was used as a control in the RT-PCR analysis as expression of this gene is not expected to vary in TR4^{+/+} and TR4^{-/-} mice. In contrast, it was thought that the closely related TR2 gene may be upregulated in compensation for the loss of TR4. FIG. 17C demonstrates that TR2 levels are not significantly different in TR4^{-/-} tissue compared to that from TR4^{+/+} animals.

(2) Homozygous TR4^{-/-} Mice are Generated from Heterozygous Pairings (TR4^{+/-}) at Less than Expected Mendelian Ratios

From 110 TR4^{+/-} breeding pairs, 751 pups were produced and subsequently genotyped. The genotyping results demonstrated that the ratios of genotypes produced were significantly different from expected Mendelian ratios (FIG. 17D). Homozygous TR4^{-/-} mice were found to make up only 13.6% of the total pups produced, when TR4^{+/+} and TR4^{-/-} pups would be expected to account for 25% of the total. Interestingly, it was also discovered that of the entire population of TR4^{-/-} mice produced, significantly less were female (30.4%), compared to the equal sex ratio (50% male, 50% female) that was expected.

(3) Unexplored Phenotypic Observations

TR4^{-/-} animals often display unkempt fur that appears greasy. It is possible that abnormal grooming or less frequent grooming behavior can explain this phenomenon. Alternatively, overactive sebaceous glands may result in greasy skin among TR4^{-/-} mice. Additional behavioral studies focusing on grooming behavior will be necessary to test the former hypothesis, and future studies involving histological analysis of skin samples may provide evidence to support or refute the latter possibility. Another yet unexplored phenotype observed among TR4^{-/-} animals is that of apparent increased secretions around the eye. Severely affected animals have been observed to have difficulty in opening their eyes. The skin surrounding the eyes, as well as the lacrimal glands, and eyes of affected mice will be analyzed histologically for signs of pathological changes. The possibility that TR4^{-/-} animals develop conjunctivitis will be explored (ref.).

(4) TR4^{-/-} Males Exhibit Penile Priapism

An additional physical defect that may affect sexual function in TR4^{-/-} mice is that of priapism, or persistent partial penile erection. This condition is observed in a substantial number, but not all of the TR4^{-/-} males, and increases in prevalence with age (FIGS. 18A and C). The cause of this condition is not well characterized in mice or in humans, although links have been made to sickle cell disease and defects in regulatory pathways involving inhibition of male spinal sexual reflexes (Beuzard 1996; Adams et al. 2001). The clinical description of partial priapism as well as segmental priapism refers to engorgement of the corpora cavemosum of the penis with stagnant blood, suggesting defects of blood diversion into the venous outflow or in problems with general venous drainage (Donatucci and Lue). Comparing histological sections of penis tissue from a TR4^{+/+} mouse (no priapism) and a TR4^{-/-} mouse with priapism, blood trapped within the corpora cavernosum and a swollen penile tip are observed in the TR4^{-/-} tissue, along with evidence of external trauma due to the penis existing outside the body (FIG. 18B).

(5) Regulation of nNOS Transcriptional Activation by TR4

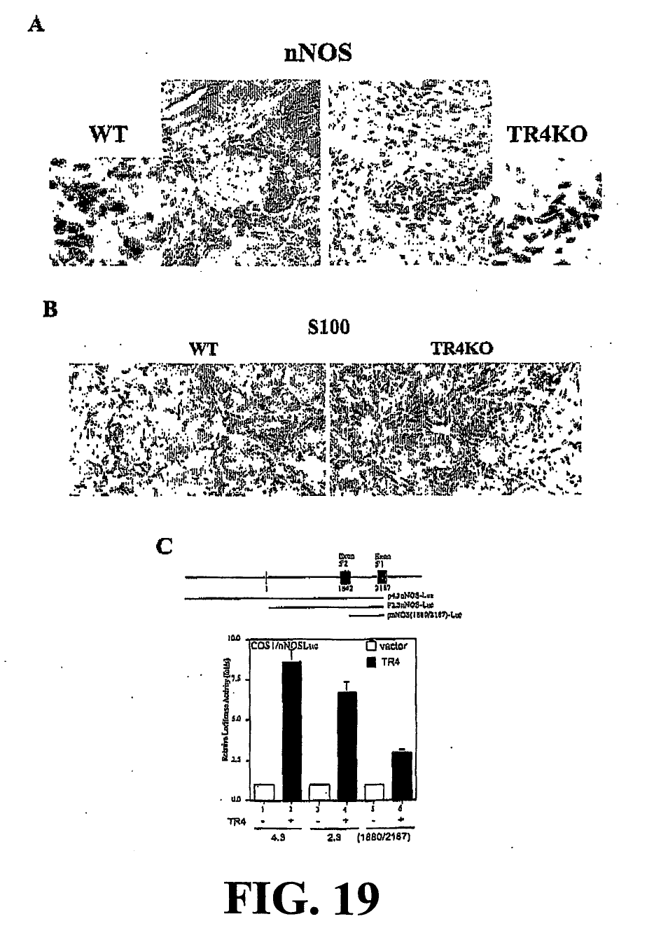

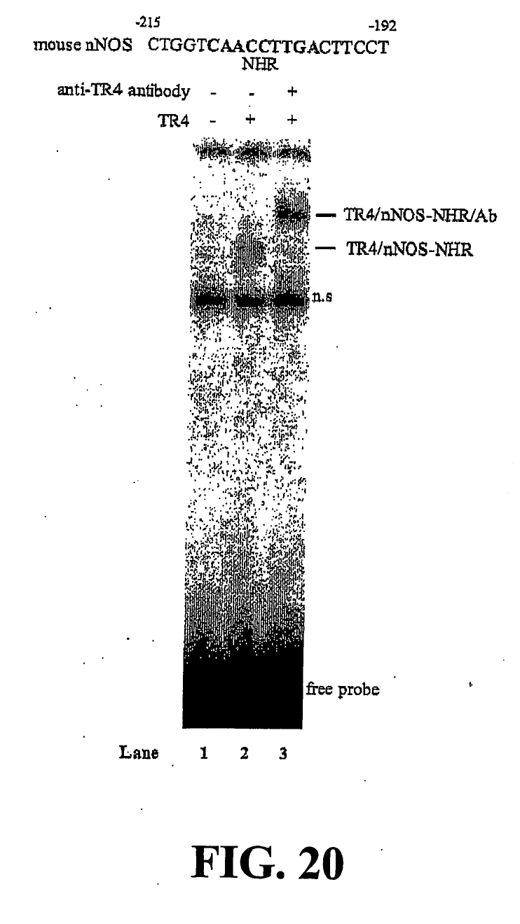

To further dissect the possible mechanism involved in the penile abnormalities observed in TR4^{-/-} mice, expression of a signaling molecule that is known to play a role in regulation of penile erection, neuronal nitric oxide synthase (NNOS) (Marin et al. 1999; Gonzalez-Cadavid et al. 2000; McKenna 2000; Steers 2000; Rampin and Guiliano 2001), was assayed in penis tissue via immunostaining. Sections of penis tissue from TR4^{+/+} and TR4^{-/-} animals were probed with antibodies against either nNOS (FIG. 19A) or S100 (a neuronal marker) (FIG. 19B). The staining results show reduced expression of nNOS in TR4^{-/-} tissue, whereas similar levels of S100 protein are expressed in TR4^{+/+} and TR4^{-/-} sections. A reporter assay, using varying lengths of the nNOS promoter region linked to luciferase reporter genes, was applied to demonstrate the regulation of nNOS gene expression by TR4. As shown in FIG. 19C, addition of TR4 can enhance nNOS reporter gene expression, although the level of induction is lessened with reduction in the length of the nNOS promoter region used. The nNOS exon 2 promoter was found to contain a nuclear hormone receptor response element (NHR) through which the nuclear receptor SF-1 was found to bind and modulate nNOS transcription (Wei et al. 2002). Using the EMSA assay, TR4 protein was shown to bind to the mouse nNOS NHR sequence (FIG. 20, lane 2, TR4/nNOS-NHR), and further retardation of the TR4/nNOS-NHR complex was achieved by addition of a monoclonal TR4-specific antibody (lane 3, TR4/nNOS-NHR/Ab).

(6) Reduced Fertility in TR4^{-/-} Mice

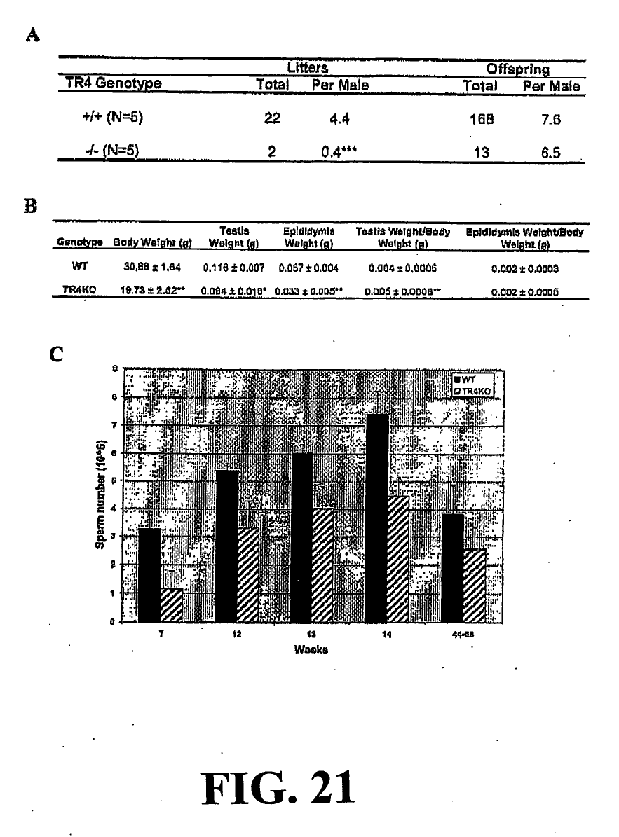

From initial attempts at pairing male TR4^{-/-} mice with TR4^{+/+} females, it was observed that litters were not readily produced. To further characterize the differences in fertility between TR4^{+/+} and TR4^{-/-} males, five mice of each genotype were each paired with a TR4^{+/+} female for 4 months. The results of this continuous mating experiment made it clear that there was a reduction in the ability of TR4^{-/-} male mice to generate offspring (FIG. 21A). Among the five TR4^{+/+} males, 22 litters were produced, with an average of 4.4 litters produced per mouse in 4 months. In contrast, only one of the 5 TR4^{-/-} males produced offspring, with a total of two litters born during the 4 month period. Not only were significantly fewer TR4^{-/-} males able to produce offspring, but the number of litters generated by the only known fertile TR4^{-/-} male was approximately half that of the TR4^{+/+} average. Interestingly, the total number of pups produced by the TR4^{-/-} male per litter was not significantly different from the average pups per litter produced by the TR4^{+/+} males. Clearly, a fertility defect exists among TR4^{-/-} male mice; however, the cause of the defect is not obvious.

(7) Reduced Sperm Count and Delayed Spermatogenesis in TR4^{-/-} Male Mice

The data in FIG. 21A demonstrate that individual males may be able to sire litters, but, as a group, TR4^{-/-} males are subfertile. The data indicating that the number of pups sired, per litter, by TR4^{-/-} animals able to generate offspring does not deviate greatly from that of the TR4^{+/+} group suggests either variability in a physical or physiological defect affecting the capacity of males to reproduce effectively, or behavioral defects of varying levels of severity. A key aspect of male reproductive function in the mouse involves the development and maturation of spermatozoa in the testis and epididymis (Roosen-Runge 1962; Weinbauer and Nieschlag 1993; Weinbauer and Nieschlag 1998). To explore the effects of ablation of TR4 expression with regard to the development and function of male reproductive organs, epididymal sperm counts were determined and histological analysis of the TR4^{-/-} testis and epididymis was carried out. Each male mouse was weighed before sacrifice, and, prior to the isolation of epididymal sperm, the testis and epididymis of each animal was also weighed. FIG. 21B demonstrates that the absolute body, testis and epididymal weights are significantly less in TR4^{-/-} males compared to TR4^{+/+}. After isolation of epididymal sperm from TR4⁴ animals ranging in age from 7-14 weeks, it was found that the number of sperm from epididymi of TR4^{-/-} males was significantly reduced compared with that of TR4^{+/+} males (FIG. 21C). The largest difference was found at 7 weeks (67% reduction in TR4^{-/-}), with the difference diminishing with age. No significant statistical difference is observed in sperm counts from aged mice (44-56 weeks old). The decrease in sperm count among TR4^{-/-} males is most interesting at the earliest time point, as there appears to be a delay in sexual maturation in these animals. The onset of sexual maturation, correlating with the first appearance of sperm in the epididymis and the completion of the first wave of spermatogenesis, occurs at approximately 6 weeks of age in mice. It was observed that TR4^{+/+} males had significant numbers of epididymal sperm by 6 weeks of age, whereas epididymal sperm were absent in most of the TR4^{-/-} epididymi examined. It is not until 7 weeks of age that TR4^{-/-} mice begin to show large numbers of epididymal sperm. Further, visual assessment of sperm motility in samples from mice at 12, 13, and 44-56 weeks of age resulted in no significant differences between TR4^{-/-} and TR4^{+/+} mice (data not shown).

The discovery of lowered sperm numbers in TR4^{-/-} animals supports the hypothesis that TR4 is involved in regulation of genes important for meiosis, and thus proper spermatogenic function. By comparing seminiferous tubule stages between TR4^{-/-} and TR4^{+/+} mice, it was found that spermatogenesis was delayed in TR4^{-/-} mice.

(8) Aberrant Sexual Behavior is Displayed by TR4^{-/-} Male Mice

Based on the known distribution of TR4 in the CNS and previous characterization of TR4 function as a transcription factor to regulate various target genes (Chang et al. 1994; Hirose et al. 1994; Young et al. 1997; Young et al. 1998; Lee et al. 2002), it is possible that the disruption of TR4 signaling affects neurological function, and thus behavior. To study sexual behavior, male mice of each genotype were individually paired with a hormonally-primed stimulus female for 90 min. (Trial 1, Table 5), and 6 hr. (Trial 2, Table 2) intervals.

TABLE 5
Male sexual behavior trial 1, 90 min.

| Behavior | WT (N = 9) | TR4KO (N = 11) |
|---|---|---|
| Mounts | | |
| No. of mice | 9/9⁺ | 0/11* |
| Mean no. of mounts | 30.8 | 0* |
| Range | 3-70 | |
| Intromissions | | |
| No. of mice | 5/9 | 0/11* |
| Mean no. of intromissions | 16.8 | 0* |
| Range | 0-98 | |
| Ejaculation | | |
| No. of mice | 2/9 | 0/11* |

⁺Number of mice showing behavior/number of mice tested;
*p ≦ 0.05

TABLE 2
Male sexual behavior trial 2, 6 hrs.

| Behavior | WT (N = 4) | TR4KO (N = 6) |
|---|---|---|
| Mounts | | |
| No. of mice | 4/4⁺ | 5/6 |
| Latency to behavior⁺⁺ | 21.5 ± 20.0 | 136.8 ± 111.1* |
| Mean no. of mounts | 21.3 | 33.8 |
| Range | 11-24 | 0-58 |
| Intromissions | | |
| No. of mice | 4/4 | 2/6 |
| Latency to behavior | 44.3 ± 32.1 | 89.8 ± 89.5 |
| Mean no. of intromissions | 14 | 7.2 |
| Range | 11-17 | 0-34 |
| Ejaculation | | |
| No. of mice | 4/4 | 1/6* |
| Latency to behavior | 71.2 ± 58.6 | 91 |
| Plug Present | 4/4 | 0/6 |

⁺Number of mice showing behavior/number of mice tested;
⁺⁺Mean ± standard deviation among animals showing behavior; Latencies are reported in minutes;
*p < 0.05

Each mating trial was videotaped and tapes were then scored for latencies to, as well as frequencies and durations of specific sexual behaviors, including mounting, intromission, and ejaculation (Kriegsfeld et al. 1999; Ogawa et al. 1999). For Trial 1 of the sexual behavior experiments, all male and female mice were naïve to the mating situation. The results of Trial 1 confirmed that TR4^{-/-} mice display defects in sexual behavior, as well as in social behavior. It was found that naïve TR4^{-/-} mice do not show defined sexual behavior within 90 minutes of the initial pairing with a primed female mouse. Out of 9 TR4^{+/+} mice tested for sexual behavior, all 9 showed mounting behavior, 5 displayed intromission, and two achieved ejaculation within 90 minutes, whereas none of the same behaviors were observed among the 11 TR4^{-/-} mice tested (Table 5). Interestingly, the TR4^{-/-} mice displayed fear or anxiety in the presence of the hormonally primed female, with an extensive delay in showing any interaction with his potential mate. In the most severe cases, the TR4^{-/-} male spent the entire test session actively avoiding his cagemate (see supplemental video footage).

To determine whether the lack of sexual behavior and abnormal social behavior observed in Trial 1 would continue with extended pairing time, and after gaining experience (Trial 1) with a stimulus female, a subset of the male mice used in Trial 1 were paired for a second, longer behavioral observation session (Trial 2, 6 hr. paring). The results of Trial 2 are displayed in Table 6. It was found that, given time, TR4^{-/-} animals did begin to display sexual behavior (see supplemental video footage. Again, proportionally fewer TR4^{-/-} than TR4^{+/+} males displayed each defined sexual behavior, with increased latencies to each behavior. An unexpected yet logical result is that TR4^{-/-} males show increased numbers of mounts compared to TR4^{+/+} males. The TR4^{-/-} animals were less successful at achieving ejaculation but continued to pursue that goal, thus displaying more mounts than their TR4^{+/+} counterparts. After the videotaped 6 hr. pairing (Trial 2), male and female mice were left paired, and female mice were examined for vaginal plugs the following morning. Vaginal plugs were discovered in each of the female mice mated to TR4^{+/+} males, whereas none of the females mated to TR4^{-/-} males had a vaginal plug (Table 6). It is especially interesting that even the female mouse mated to the TR4^{-/-} male that achieved ejaculation during the 6 hr. trial did not have a vaginal plug. Possible explanations for the lack of vaginal plug include continued attempts by the male to mate with the female, as TR4^{+/+} males commonly ejaculated twice during the 6 hr. trial period, or loss of the plug into the cage bedding before vaginal examination was carried out. Alternatively, a defect in accessory sex organ function could result in the inability of TR4^{-/-} male seminal fluid to form a vaginal plug.

From observing the TR4^{-/-} mice during the sexual behavior trials, it was evident that much of the increased latency to display sexual behaviors could be explained by increased fear or anxiety among TR4^{-/-} mice. A longer time was necessary for these animals to overcome the fear of an intruder and begin to display social or sexual behavior. Additionally, the ability of the TR4^{-/-} males to achieve the appropriate positions necessary for intromission, and the ability to maintain those positions for the lengths of time essential to induce ejaculation, appeared abnormal. Both increased fear and hypersensitivity to various sensory stimuli, demonstrated by an exaggerated startle response, along with possible motor coordination defects demonstrated by abnormalities in gait and posture may also contribute to the abnormalities in sexual behavior and reproductive function observed in male TR4^{-/-} mice.

(9) Analysis of Sexual Behavior-Related Gene Expression

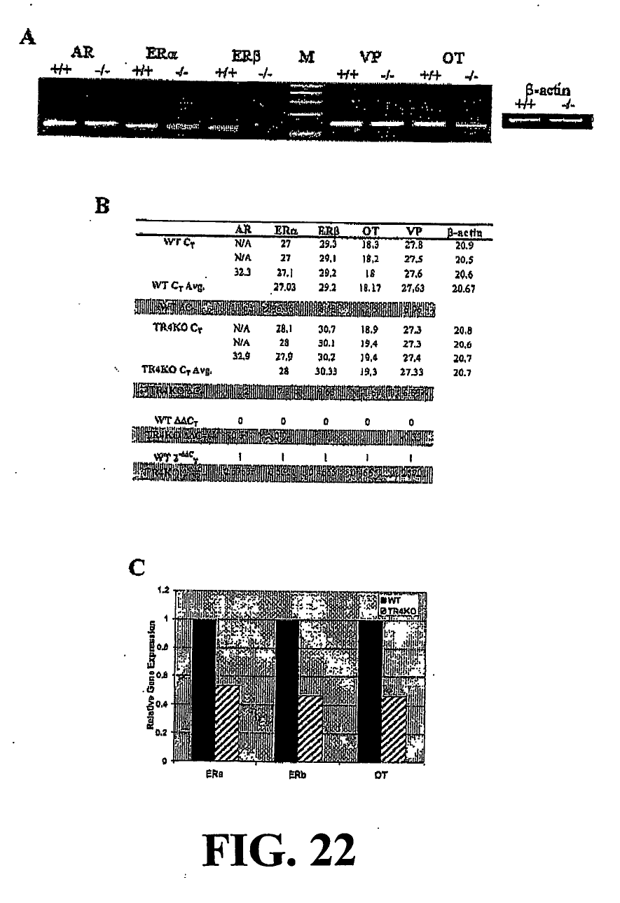

Expression levels of several genes known to affect behavior, and particularly sexual behavior, were determined via semi-quantitative RT-PCR and quantitative Real Time PCR in hypothalamic tissue from TR4^{-/-} and TR4^{+/+} mice. Results from RT-PCR amplification of segments of the androgen receptor (AR), estrogen receptor (ER) alpha (α) and beta (β), vasopressin (VP), and oxytocin (Witt 1995; McGinnis et al. 1996; Ogawa et al. 1997; De Vries and Boyle 1998; Ostrowski 1998; Smock et al. 1998; Coolen and Wood 1999; Ogawa et al. 1999; Gimpl and Fahrenholz 2001; Romeo et al. 2001; McGinnis et al. 2002) suggested that hypothalamic expression of ERα, ERβ, and oxytocin is reduced in TR4^{-/-} mice (FIG. 22A). No differences in levels of AR or VP expression were apparent. After quantitative Real Time PCR analysis was carried out, it was clear that levels of ERα, ERβ, and oxytocin are reduced by approximately 50% in the hypothalamic region of TR4^{-/-} mice compared to TR4^{+/+} mice, and that there is indeed no significant difference in expression of either AR or VP (FIGS. 22B and C). For each gene, TR4^{+/+} expression is set at 1 and relative expression from TR4^{-/-} samples is calculated, thus comparisons of expression levels between genes cannot be made from this data.

(10) Reduced Fertility in Female TR4^{-/-} Mice

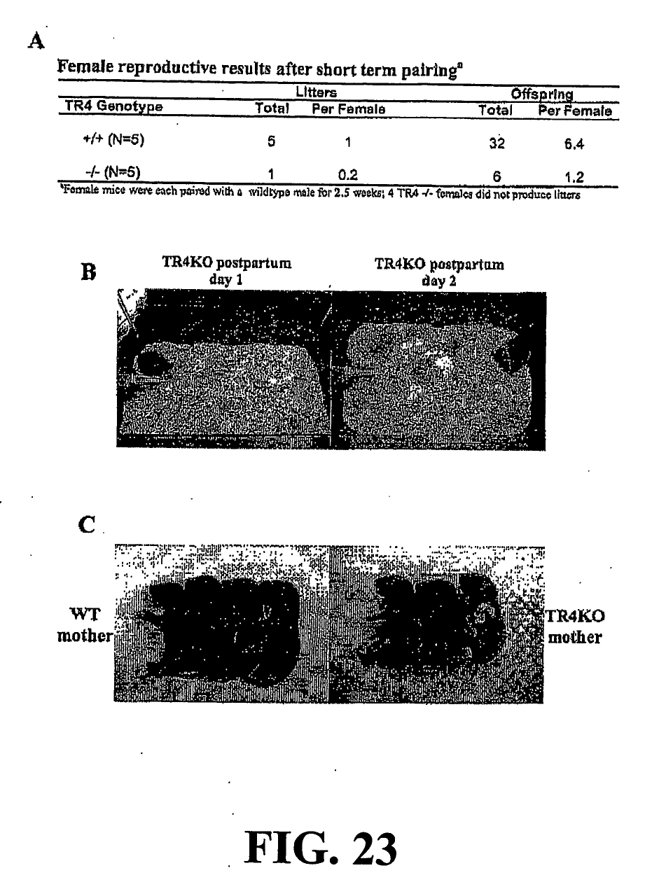

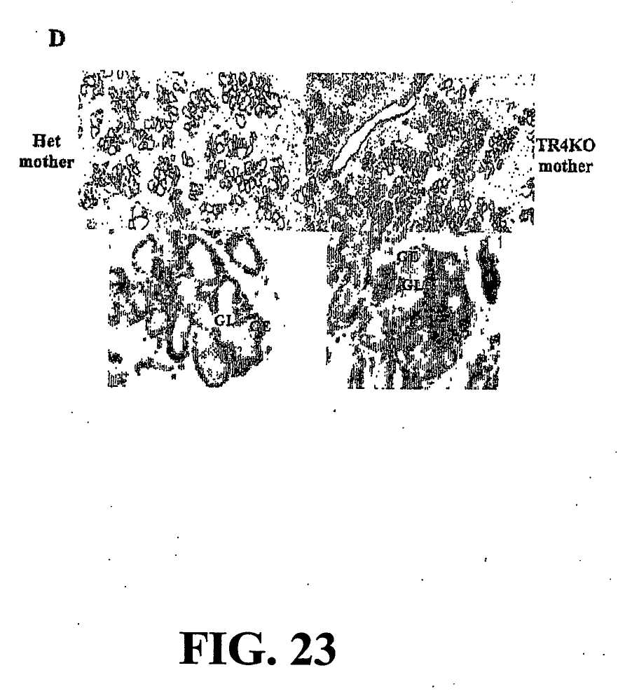

Short term mating experiments to test TR4^{-/-} female reproductive capacity involved the pairing of each of 5 TR4^{+/+} and 5 TR4^{-/-} adult female mice with one sexually mature TR4^{+/+} male for 2.5 weeks. Females were then separated from males and observed daily for the presence of a litter for the next 4 weeks. Upon parturition, the litter size (pup number) was determined and recorded. Only 1 out of 5 TR4^{-/-} female produced a litter, whereas all TR4^{+/+} females paired produced litters (FIG. 23A).

(11) Loss of Maternal Behavior in TR4^{-/-} Mothers

Observations of TR4^{-/-} female mothers suggest defects in maternal behavior. Maternal behavior observations were made in person and via video recordings of the female in her home cage, in the days directly following parturition. TR4^{-/-} mothers do not build nests, collect pups to a single location, crouch over pups, or nurse their offspring, and pups of TR4^{-/-} mothers die within 24-36 hours after birth with no milk in their stomachs (FIG. 23B and C). Histology of mammary gland tissue from a TR4^{+/-} mouse (TR4^{+/-} females show normal reproductive capacity and maternal behavior) and from a TR4^{-/-} female, on postpartum day 1, demonstrate no obvious defect in milk production in the mutant animal (FIG. 23D). The magnified mammary gland structures (lower panels) show milk (pink staining) within the glandular lumen (GL). However, the mammary glands of the TR4^{-/-} mother are abnormally full of milk, which is consistent with the lack of maternal behavior and thus lack of pup nursing observed in these animals. In contrast, the heterozygous mother shows mammary glands with both full and empty lumenal regions, suggesting that milk is drained by pup suckling and restored through continuing milk production (FIG. 23D). Although histological analysis of mammary gland structures supports the observed lack of maternal behavior among TR4^{-/-} mothers, few samples have been analyzed thus far. It is important to note that pups may develop preferences for particular nipples, and with small litters not making use of all available nipples, mammary gland structures may appear histologically similar to those of the TR4^{-/-} mothers, even with normal displays of maternal behavior and pup suckling activity.

(12) Reduced Postnatal Growth of TR4^{-/-} Animals

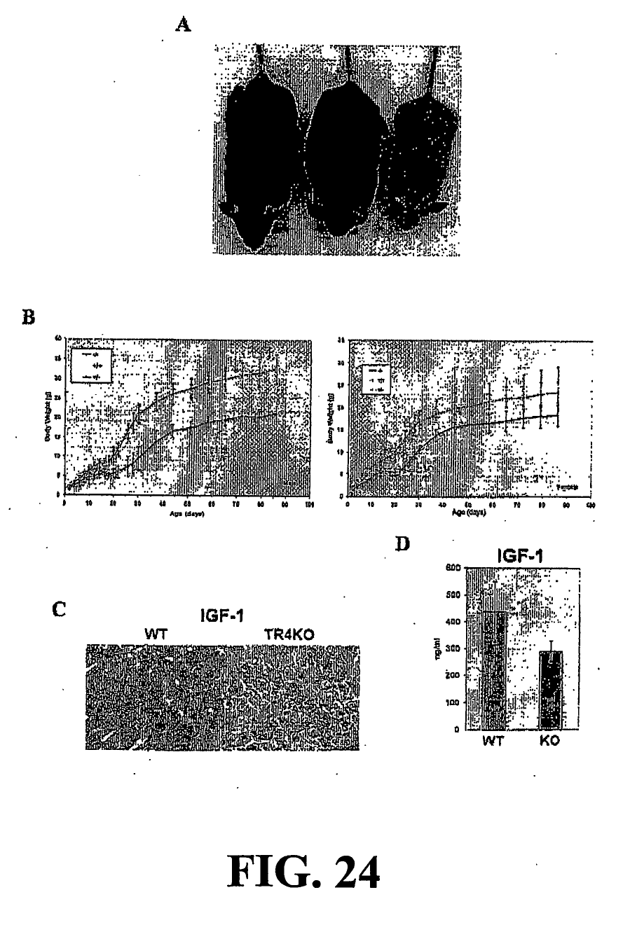

At the time of birth, TR4^{-/-} mice were visibly indistinguishable from TR4^{+/+} or TR4^{+/-} littermates, with no obvious defects in suckling ability. However, it was found that both male and female TR4^{-/-} mice were smaller than TR4^{+/+} or TR4^{+/-} mice at similar ages. The smaller size of TR4^{-/-} mice remains obvious up to 7 months of age (FIGS. 24A and B). Thus, TR4^{-/-} male mice display up to a 40% reduction in weight compared to their TR4^{+/+} and TR4^{+/-} counterparts well into adulthood. To generate growth curves for male and female pups from birth, weight measurements were taken starting from postnatal day 2 until the animals were 12 weeks of age. Female TR4^{-/-} pups displayed a range of significant weight reduction (p<0.05), between 20% and 54%, compared with their TR4^{+/+} and TR4^{+/-} counterparts, at all time points (FIG. 24B, right panel). The TR4^{-/-} mice display an approximately 30% weight reduction at the first time point, day 2, which increases to approximately 50% in week 3. The reduction in weight among TR4^{-/-} animals then drops to 20% by week 5, a level that is maintained through week 12. Male TR4^{-/-} pups display a range of significant weight reduction (p<0.05) between 24% and 56%, compared with their TR4^{+/+} and heterozygous counterparts, at all time points except day 4 (16% reduction, p<0.1) (FIG. 24B, left panel). The TR4^{-/-} mice display an approximately 30% growth reduction by day 10, which increases to about 50% in weeks 4 and 5, and then returns to 30%. The 30% reduction in weight among male TR4^{-/-} mice is maintained through week 12. These data show that weight differences in TR4^{-/-} mice compared to their TR4^{+/+} and TR4^{+/-} littermates are apparent during the first postnatal week, and that a reduction in growth rate is obvious up to 1-2 weeks after the normal time of weaning (3 weeks). Thereafter, the growth curves are nearly parallel, with a 20% to 30% reduction in weight maintained into adulthood. Weaning of TR4^{-/-} mice was routinely delayed for 1-3 weeks, as weaning at 3 weeks often resulted in death of the TR4^{-/-} mouse. Also, as shown in FIG. 17D, there is a higher mortality rate for TR4^{-/-} at 3-5 weeks of age despite delayed weaning.

(13) Analysis of Growth-Related Hormone Levels

Observation of significant growth retardation in the TR4^{-/-} mice prompted further investigation of signaling pathways known to affect postnatal growth. Levels of growth hormone (GH) and insulin-like growth factor-1 (IGF-1), a downstream target of GH, were measured in serum from age-matched TR4^{+/+} and TR4^{-/-} mice or analyzed via immunostaining of organs where the hormone is produced. Overall pituitary structure, as well as somatotroph number and level of GH produced in the anterior pituitary are similar in sections from TR4^{+/+} and TR4^{-/-} mice (data not shown). However, serum levels of IGF-1 were found to be reduced by 34% in TR4^{-/-} mice compared with TR4^{+/+} controls (FIG. 24D), and immunostaining for IGF-1 in liver sections further confirmed a reduced level of IGF-1 in TR4^{-/-} hepatocytes compared with that of the TR4^{+/+} (FIG. 24C).

The results of this study include the successful disruption of TR4 gene expression in mice and the following characterization of several phenotypes observed as a result of the loss of TR4. The number of homozygous TR4^{-/-} pups generated from heterozygous pairings was just over 50% of the expected number based on normal Mendelian ratios of genotypes (FIG. 17D). Without evidence that significant numbers of pups are dying perinatally, the data obtained suggests increased embryologic mortality of TR4^{-/-} mice. Additional analysis of genotypes produced at embryological time points will be necessary to confirm such a conclusion, as well as to determine the time in development at which the loss of TR4 expression is lethal in some animals, and the specific cause of lethality. It is known that TR4 is expressed in the mouse embryo as early as embryonic day 9, with particularly high expression in various regions of the developing nervous system during subsequent prenatal development (Young et al. 1997). This developmental information provides a guide through which to focus further analysis of TR4^{-/-} mouse generation and prenatal survival.

Priapism is observed in many TR4^{-/-} males, with increasing frequency of presentation with age. Trapped blood within the corpus cavemosum of the penis leads to reduced tissue oxygenation, increased blood viscosity, disruption of tissue elasticity, fibrosis, and finally irreversible failure of erection (Winter 1978; Hauri et al. 1983; Panteleo-Gandais et al. 1984). Clinically, priapism is classified as primary (idiopathic) or secondary, with numerous potential causes. Such causes include hematologic disorders, traumatic or surgical injury (to the penis or spinal cord), neoplasia, infective toxic allergy, neurologic disorders, and pharmacologic induction (Hashmat and Rehman). In addition to the obvious inability of the TR4^{-/-} mice with priapism to retract the penis back within the sheath (FIG. 22A), histological evidence of priapism was observed (FIG. 22B). Penis tissue from mice with priapism showed blood trapped within the corpus cavernosum, fibrosis, swelling, and consequently, reduction of the preputial cavity. Priapism has been associated with sickle cell disease in humans (Hashmat and Rehman), and is found in mouse models of sickle cell disease (Trudel et al. 1994; Beuzard 1996). A potential link between sickle cell disease and TR4 is the involvement of a TR2/TR4 heterodimer in the direct repeat erythroid-definitive (DRED) complex, a known repressor element that binds to DR1 sites in the ε- and γ-globin gene promoters (Tanabe et al. 2002). The DRED repressor downregulates expression of the human embryonic and fetal globin gene promoters, but has no effect on the DR1-deficient adult β-globin promoter. Current strategies for the treatment of sickle cell disease focus on increasing the level of γ-globin relative to the mutant β-globin polypeptide (α₂β^{S}₂), a known cause of the disease (Tanabe et al. 2002). It was suggested that disruption of the repressive effect of the DRED complex on γ-globin expression, via prevention of TR2/TR4 binding to response elements in the γ-globin promoter, may be a useful treatment strategy to pursue. However, the presence of priapism in a substantial proportion of TR4^{-/-} males indicates a possible hematologic defect resulting from the ablation of TR4 expression. Further, the nature and complexity of the phenotypic abnormalities displayed by TR4^{-/-} animals suggest that multiple unwanted side-effects may result from disruption of TR4 function, and may eclipse the usefulness of such a strategy. Yet further exploration of TR4 function relative to erythrocyte physiology and hematologic pathology will be an exciting pursuit.

The neurotransmitter nitric oxide (NO) is a known mediator of erectile function that promotes vasodilation and the inflow of blood, resulting in penile tumescence (Burnett 1995; Andersson and Stief 1997). The levels of nNOS, the main synthetic enzyme responsible for production of NO in the penis (Yun et al. 1996; Nelson et al. 1997), were determined in penis tissue from TR4^{-/-} and TR4^{+/+} mice. nNOS expression was reduced dramatically in the TR4^{-/-} mouse penis compared to TR4^{+/+} tissue (FIG. 23A), while levels of the neuronal marker S100 were comparable (FIG. 23B), suggesting normal neuronal innervation of the TR4^{-/-} penis. It was also found that TR4 is able to bind the NHR of exon 2 (FIG. 24), as well as upregulate nNOS gene expression via reporter gene assay (FIG. 23C). These results suggest that erectile function may be negatively affected by the loss of TR4 function in TR4^{-/-} mice, with or without priapism. nNOS, through NO, has also been shown to affect hypothalamic-pituitary function by modulating expression of various hormones produced in the hypothalamus and the pituitary, such as those involved in gonadotroph signaling (LH, FSH, and GnRH) (Ceccatelli et al. 1993; Bhat et al. 1995; Garrel et al. 1998) and stress response pathways (CRH and ACTH) (Kishimoto et al. 1996; Lee et al. 1999a). Furthermore, oxytocin has been shown to be a potent inducer of penile erection as well, and links between oxytocin and NO have been explored, suggesting that oxytocin induces male sexual responses (i.e. penile erection) via increasing nNOS activity in neurons projecting to brain regions outside the hypothalamus (Argiolas and Melis 1998). The reduced expression of both oxytocin and nNOS in TR4^{-/-} mice may then affect these signaling molecules in combination, as well as individually, thereby potentially intensifying defects of male sexual physiology and behavior resulting from the loss of TR4 function.

Although the function of NO in mediation of vasodilation in the penis and the expected reduced production of NO via loss of TR4-promoted nNOS expression in TR4^{-/-} mice seem to be contrary to the observation of priapism among TR4^{-/-} mice, it must be remembered that additional NOS isoforms exist (Ignarro and Jacobs 2000), other factors contribute to penile vasodilation (Simonsen et al. 1997), and NO also functions as a neurotransmitter (Ignarro and Jacobs 2000). Male mice lacking expression of either nNOS (Burnett et al. 1996) or eNOS (Kriegsfeld et al. 1999) retain erectile function and fertility, suggesting that there are factors that compensate for the loss of NOS isoforms in their absence. The retention of vasodilatory activity in nNOS-deficient penis tissue of TR4^{-/-} mice, exemplified by the development of priapism or by reproductive capacity, could be explained by this compensation. As nNOS has been found in most neurons, it is possible that NO, acting as a neurotransmitter, is also involved in the promotion and maintenance of penile erection, as well as in detumescence (Ignarro and Jacobs 2000). The penis receives neuronal afferents from parasympathetic, sympathetic, and somatic nerves, with parasympathetic innervation primarily responsible for vasodilation and erection, and sympathetic innervation thought to mediate detumescence (Steers 2000). Disruption of NO neurotransmission at pre- and/or post-ganglionic aspects of either of these systems may result in defects in the appropriate hemodynamic balance between inflow and outflow, leading to chronic priapism. The molecular defects leading to priapism are not yet characterized and it is not clear whether such defects occur constitutively once presented. Yet due to the likelihood of tissue damage resulting from blood trapped within the corpus cavernosum, irreversible structural defects may develop in an affected penis, leading to chronic priapism regardless of the continuation of the presumably causative molecular abnormalities.

While reduced sperm counts and delayed spermatogenesis in TR4^{-/-} mice may support roles of TR4 in testis function, it may also be argued that the reduced sperm count in adult TR4^{-/-} male mice (FIG. 20A) may be related to the growth defect they display (FIGS. 18A and B, FIG. 19B). The reduced absolute testis size of TR4^{-/-} mice, and thus the proportionally reduced number of sertoli cells present to support sperm cell development may contribute to a reduction in absolute sperm number (Singh and Handelsman 1996; Smith et al. 2002). The presence of large numbers of motile sperm, normal distribution of germ cells in various stages of the cell cycle, typical testis and epididymis morphology and cellularity, normal seminal vesicle and coagulating gland secretory epithelial structure with evidence of appropriate secretions in the luminal regions of these organs (data not shown), as well as gross structural integrity of spermatozoa (data not shown) suggest factors other than defects of the testis or accessory sex organs may contribute to the severe reduction in fertility observed among TR4^{-/-} male mice (FIG. 19A). Abnormalities in social/sexual behavior, as well as in penis function, seem to play a significant role.

For each behavior demonstrated by TR4^{-/-} mice, the latency to behavioral display was increased (although not significantly in the cases of intromission and ejaculation) (Table 6). These results indicate that TR4^{-/-} animals retain sexual motivation, yet take longer to become acclimated to the pairing situation and to begin showing sexual behaviors. Further, TR4^{-/-} males are less successful in achieving ejaculation, the presumed goal toward which reproductive behavior is oriented. In fact, a large proportion of TR4^{-/-} males tested for 6 hr. did not even achieve intromission. From observation of the TR4^{-/-} mice during the mating trials, it became evident that the animals had difficulty in maintaining the appropriate mounting position through which intromission and ejaculation could be achieved. Defects in balance and coordination may explain such difficulty, and therefore additional analyses relative to muscle strength and motor function will help to explore this phenotype further. Likewise, quantitative analysis and characterization of additional behaviors, such as those related to fear and anxiety, will be the subject of future investigation, and assist in further interpretation of the sexual behavior defects reported in this study.

From studies in mice lacking ERα (αER^{-/-}), it was determined that males retained normal motivation to mount hormonally-primed female mice, but achieved fewer intromissions and no ejaculations in 30 min. trial periods (Ogawa et al. 1997). Further similarities to the results of sexual behavior analysis in TR4^{-/-} mice include the report that 1 out of 7 αER^{-/-} mice achieved ejaculation in an extended 3 hr. test period, and that αER^{-/-} male mice had difficulty holding their hind legs tightly against the female during thrusting (Ogawa et al. 1997). Unlike TR4^{-/-} mice, however, the αER^{-/-} males show complete infertility and severe defects in spermatogenesis and sperm function (Eddy et al. 1996a). The similarities in sexual behavior abnormalities of the αER^{-/-} male, combined with reduced expression of ERα in TR4^{-/-} mice, suggests the possibility that TR4 may work partly through ER signaling pathways to control sexual behavior. However, the lesser severity of the TR4^{-/-} male reproductive phenotype compared with that of the αER^{-/-} male indicates that the level to which ERα expression is reduced is insufficient to cause such severe defects as those seen in the αER mouse. In contrast to the mouse model of ERα ablation, the ERβ knockout mouse (βER^{-/-}) showed no defects in male sexual behavior or reproductive function (Ogawa et al. 1999). βER^{-/-} mice did, however, display higher levels of aggression in particular social contexts than did βER^{+/+} control mice. Although aggression was not explored in the current study of TR4^{-/-} mice, the reduced ERβ expression in males suggests that aggressive behavior may be an interesting area to investigate, especially in light of the possible increase in fear-related behavior, and the presumably experience-mediated anxiety attenuation observed throughout the TR4^{-/-} sexual behavior analyses. However, study of aggressive behaviors in TR4^{-/-} mice may be further confounded by the reported reduction in male aggressive behavior, and male-typical offensive attacks in αER^{-/-} male mice (Ogawa et al. 1997).

Oxytocin is a peptide hormone produced in neurons of the paraventricular and supraoptic nuclei of the hypothalamus, as well as in specific tissues and cell types peripherally (Gimpl and Fahrenholz 2001). Oxytocin can affect central and peripheral systems, as well as behavior (reviewed in (Gimpl and Fahrenholz 2001)). Relative to male reproduction, oxytocin is known to promote seminiferous tubule contractility and modulate testicular steroidogenesis. Although oxytocin has no obvious effect on male affiliative or sexual behavior in mice, a pronounced anxiolytic effect has been demonstrated (Uvnas et al. 1994; McCarthy 1995; McCarthy et al. 1996; Windle et al. 1997; Gimpl and Fahrenholz 2001). With a demonstrated anxiety-reducing effect of oxytocin release or exogenous oxytocin treatment, it is likely that the increased fearfulness observed in TR4^{-/-} males involves loss of the anxiolytic effects of oxytocin as a result of reduced expression of the peptide hormone.

The growth defect observed among TR4^{-/-} mice (FIG. 18B) is similar in rate of growth reduction observed among other small mouse mutants (Li et al. 1990; Voss and Rosenfeld 1992; Lin et al. 1993; Sornsen et al. 1996), yet relatively unique in the timing of the onset of growth reduction. Unlike the 2-week postnatal latency to growth reduction in the Snell, Ames and little dwarf mutants (Voss and Rosenfeld 1992), TR4^{-/-} mice display significant growth reduction as early as postnatal day 2 (the first time point at which measurements were collected, FIG. 18). Interestingly, a mutant mouse deficient in the winged helix gene Mf3, which encodes a transcription factor highly expressed in the central nervous system of the developing embryo, shows growth reduction as early as 2-3 days after birth (Labosky et al. 1997). Also similar to TR4, Mf3 is expressed in the neural tube at embryonic day 9.5, as well as in the diencephalon and midbrain. Further, the Mf3^{-/-} mice show variations in phenotype with considerable embryonic lethality, consisting of 30% pre-weaning mortality among those surviving to birth, and surviving adult mutants that, although small in size, can live to over one year of age (Labosky et al. 1997). Many similarities exist between the TR4^{-/-} and the Mf3^{-/-} mice, suggesting that a developmental neural defect may underlie the production, mortality, and growth defects observed in the TR4^{-/-} mice.

Mice with growth retardation often display defects in pituitary structure or function, which may become apparent as an ultimate defect in GH or TSH production or secretion (Li et al. 1990; Lin et al. 1993; Kendall et al. 1995; Somsen et al. 1996). In the TR4^{-/-} animals, pituitary structure, somatotroph number, and somatotroph distribution were all normal, with similar levels of staining for GH in the anterior pituitary among TR4^{-/-} and control mice (data not shown). Despite the lack of structural abnormalities of the pituitaries of TR4^{-/-} animals, it is possible that TR4 may affect pituitary hormone-initiated signaling at points farther downstream. TR4 has been shown to modulate thyroid hormone target genes (Lee et al. 1997; Lee et al. 1999c). Indeed, mice carrying mutations of the thyroid hormone receptors, individually (TRα1^{-/-}, TRβ^{-/-}), or in combination (TRα1^{-/-}β^{-/-}), display growth impairments (Gothe et al. 1999; Kaneshige et al. 2000). Therefore, a disruption of regulation of the thyroid hormone receptor signaling pathway via loss of TR4 may contribute to the growth defect observed in TR4^{-/-} mice. Also, a downstream mediator of GH action, IGF-1, is known to be important in postnatal growth based on IGF-1 gene knockout studies (Baker et al. 1993; Liu et al. 1993). Further, IGF-1 has been shown to be reduced in other mutant mouse models with growth abnormalities, such as steroid receptor coactivator SRC-3^{-/-}, in the absence of corresponding defects in growth hormone secretion (Xu et al. 2000). The discovery of low IGF-1 serum levels (FIG. 18D) and reduced IGF-1 staining in liver sections from TR4^{-/-} mice (FIG. 18C) indicates that an IGF-1 deficiency may contribute to the growth retardation observed in TR4^{-/-} mice.

Collectively, the data reported here describe severe reproductive malfunction in TR4^{-/-} male mice. In addition to reduced sperm counts and delayed spermatogenesis, significant effects on male fertility stem from abnormal sexual behavior among TR4^{-/-} male mice, with TR4^{-/-} mice rarely achieving ejaculation or intromission, despite displaying sexual motivation. In TR4^{-/-} male mice with priapism, erectile function is likely lost, therefore accounting for the lack of intromission or ejaculation. In TR4^{-/-} males without priapism, it is unclear whether erectile function is abnormal, yet disruptions of signaling pathways involving nNOS and oxytocin, proven TR4 target genes (Burbach et al. 1998)), may result in loss of erectile function, inappropriate behavioral responses in mating situations, or both. Furthermore, TR4^{-/-} mice display defects in growth, and female TR4^{-/-} mice show reduced fertility and severe defects in maternal behavior, resulting in neonatal death of their offspring due to starvation. These in vivo data describing TR4^{-/-} mice demonstrate that TR4 plays an essential part in various reproductive functions, particularly involving the testis, penis, and nervous system. Future studies focusing on roles of TR4 in testis, penis, and brain function may lead to the identification of a physiological ligand(s), as well as yet undiscovered physiological roles of TR4.

### 7. EXAMPLE

### Phenotype Analysis of Mice and Characterization of the TR4 Knockout/β-gal Knockin (TR4−/−) Mice

TR4−/− mice differ from heterozygous TR4 knockout animals (TR4+/−) and wildtype animals in several respects. TR4−/− male and female mice are 30% smaller by weight than their TR 4+/− and wildtype littermates by 10 days of age. This size difference persists after weaning, and over the time period monitored to date (86 days), TR4−/− animals remain consistently smaller. The smaller size of the TR4−/− animals can be investigated in terms of potential hormonal defects, skeletal growth defects, and muscular defects. The overall approach can be to analyze the growth defect as diagramed in FIG. 23. Results also indicate that male and female TR4−/− have impaired fertility. This defect can be analyzed as disclosed herein and diagramed in FIG. 24. Behaviorally, the TR4−/− mice are inactive, do not exhibit normal cage exploratory behavior and show a dramatically reduced interaction with cage-mates. When TR4−/− move, they demonstrate an abnormal gait characterized by a lack of coordinated stepping, particularly of the hind limbs. This phenotype could potentially be due to either skeletal malformations or neurological impairment of the spinal cord (assessment of the spinal cord can be performed as disclosed herein. Such malformations may arise developmentally as a neural tube defect. Statistical analysis can be performed as needed.

a) Characterization of Growth Retardation in TR4−/− Mice by Hormonal Analysis

TR4−/− animals are approximately 30% smaller than TR4+/− and wildtype littermates by 10 days of age and remain 30-50% smaller over the period monitored to date (86 days). Other nuclear receptor knockout lines that show a postnatal growth retardation phenotype include those with ablation of the VDR (Yoshizawa, T., Handa, Y., Uematsu, Y., Takeda, S., Sekine, K., Yoshihara, Y., Kawakami, T., Arioka, K, Sato, H., Uchiyama, Y., Masushige, S., Fukamizu, A., Matsumoto, T., and Kato, S. (1997) Nat. Genet. 16, 391-396 I), Thyroid receptor a (T3Ra) (. Fraichard, A., Chassande, O., Plateroti, M., Roux, J. P., Trouillas, J., Dehay, C., Legrand, C., Gauthier, K., Kedinger, M., Malayal, L., Rousset, B., and Samarut, J. (1997) EMBO J. 16, 4412-4420), and RARy (Lohnes, D., Kastner, P., Dierich, A., Mark, M., LeMeur, M., and Chambon, P. (1993) Cell 73, 643-658) genes. In the case of VDR null animals, the reduction in growth rate was manifested after weaning (Yoshizawa, T., Handa, Y., Uematsu, Y., Takeda, S., Sekine, K., Yoshihara, Y., Kawakami, T., Arioka, K., Sato, H., Uchiyama, Y., Masushige, S., Fukamizu, A., Matsumoto, T., and Kato, S. (1997) Nat. Genet. 16, 391-396 I). The T3Ra knockout mice exhibit growth arrest at two weeks of age and were unable to survive beyond weaning (Fraichard, A., Chassande, O., Plateroti, M., Roux, J. P., Trouillas, J., Dehay, C., Legrand, C., Gauthier, K., Kedinger, M., Malayal, L., Rousset, B., and Samarut, J. (1997) EMBO J. 16, 4412-4420). RARy −/− mice were 40-80% smaller than their heterozygous or wildtype littermates at 4 days postpartum, with the smallest homozygous knockout animals displaying 50% mortality by 3 weeks (Lohnes, D., Kastner, P., Dierich, A., Mark, M., LeMeur, M., and Chambon, P. (1993) Cell 73, 643-658). These observations contrast with the TR4−/− phenotype in that the TR4−/− mice are significantly smaller at weaning and do not show an elevated mortality before 3 weeks of age. The TR4 knockout/β-gal knockin animals therefore provide us with a valuable tool for the analysis of the role of TR4 in growth control.

It is possible that the small size of the TR4−/− mice is due to alteration in growth factor production. Mutations in genes that regulate growth factor production (Godfrey, P., Rahal, J. O., Beamer, W. G., Copeland, N. G., Jenkins, N. A., and Mayo, K. E. (1993) Nat. Genet. 4,227-232, Voss, J. W., and Rosenfeld, M. G. (1992) Cell 70, 527-530), IGF-1 (Xu, J., Liao, L., Ning, G., Yoshida-Komiya, H., Deng, C., and O'Malley, B. W. (2000) Proc. Natl. Acad. Sci. USA 97, 6379-6384, Liu, J. P., Baker, J., Perkins, A. S., Robertson, E. J., and Efstratiadis, A. (1993) Cell 75, 59-7), IGF-2 (Baker, J., Liu, J. P., Robertson, E. J., and Efstratiadis, A. (1993) Cell 75, 73-82), or the IGF-I receptor (45. Liu, J. P., Baker, J., Perkins, A. S., Robertson, E. J., and Efstratiadis, A. (1993) Cell 75, 59-72) all result in postnatal growth retardation. Because growth hormone (GH) is secreted in a pulsitile fashion, it can be difficult to obtain consistent serum levels between animals of the same genotype from a single blood draw. IGF-I is considered to be a mediator of GH action postnatally, and shows a less variable production (46. Baker, J., Liu, J. P., Robertson, E. J., and Efstratiadis, A. (1993) Cell 75, 73-82). Therefore, serum IGF-1 levels between TR4−/−, TR4+/−, and wildtype mice at 4 weeks of age can be compared. Reduced levels of IGF-1 are expected to be present in the growth deficient TR4−/− animals, as animals carrying null mutations for IGF-1 display pronounced growth retardation (45. Liu, J. P., Baker, J., Perkins, A. S., Robertson, E. J., and Efstratiadis, A. (1993) Cell 75, 59-72, Baker, J., Liu, J. P., Robertson, E. J., and Efstratiadis, A. (1993) Cell 75, 73-82). Given that defects in IGF-1 levels are found in TR4−/−, as compared to TR4+/− or wildtype animals, the specific level of IGF-1 found in the knockouts can be correlated with the observed growth deficiency. As IGF-1 is a mediator of GH action postnatally and can be studied as an initial indicator of possible involvement of the GH signaling pathway (Baker, J., Liu, J. P., Robertson, E. J., and Efstratiadis, A. (1993) Cell 75, 73-82), the involvement of GH in the cause of the observed TR4−/− growth phenotype can be assessed.

In the event that no difference in IGF-1 levels among mice of each genotype is found, the thyroid hormone pathway, GH, and GHRH as potential effectors of the observed growth defect in TR4−/− animals can be assessed. It is known that animals carrying a null mutation for the T₃Rα gene display postnatal growth arrest, as well as delayed bone development and impaired ossification (Fraichard, A., Chassande, 0., Plateroti, M., Roux, J. P., Trouillas, J., Dehay, C., Legrand, C., Gauthier, K, Kedinger, M., Malayal, L., Rousset, B., and Samarut, J. (1997) EMBO J. 16, 4412-4420). Additionally, disruption of the α subunit of pituitary hormones TSH, LH and FSH resulted in hypothyroidism, hypogonadism and dwarfism (Kendall, S. K., Samuelson, L. C., Saunders, T. L., Wood, R. I., and Camper, S. A. (1995) Genes Dev. 9, 2007-2019). Likewise, dwarf and little mice, with absence of, or reduced GH secretion respectively, exhibit growth retardation (Godfrey, P., Rahal, J. 0., Beamer, W. G., Copeland, N. G., Jenkins, N. A., and Mayo, K. E. (1993) Nat Genet. 4,227-232, Slabaugh, M. B., Lieberman, M. E., Rutledge, J. J., and Gorsky, J. (1981) Endocrinology 109, 1040-1046), and mice carrying the dw/dw and hyt/hyt mutations exhibit hypothyroidism as well as small size (Bouchon, R., and Ropartz, P. (1990) Physiology and Behavior 48,501-505, 50. Beamer, W. G., Eicher, E. M., Maltais, L. J., and Southard, J. L. (1981) Science 212, 61-63). In the case of hyt/hyt mice, the animals have very low serum thyroxine (T₄), and elevated serum TSH (thyroid stimulating hormone) (Bouchon, R., and Ropartz, P. (1990) Physiology and Behavior 48,501-505, Beamer, W. G., Eicher, E. M., Maltais, L. J., and Southard, J. L. (1981) Science 212, 61-63), whereas dw/dw display low levels of TSH (50. Beamer, W. G., Eicher, E. M., Maltais, L. J., and Southard, J. L. (1981) Science 212, 61-63).

The markers of proper function of the thyroid hormone signaling pathway can be measured by looking at levels of TSH and thyroxin in TR4−/−, TR4+/− and wildtype mice at 4 weeks of age. To determine whether defects in the GH pathway are present, growth hormone releasing hormone (GHRH) and GH levels in mice of each genotype, at 4 weeks of age can be assayed. It is expected that low levels of these hormones in TR4−/− mice as compared with TR4+/− and wildtype mice, as part of the explanation for the growth deficit observed in the knockouts. Upon recognizing a defect in the levels of any of these hormones that affect growth regulation, the level of the abnormal hormone levels with the observed growth deficiency phenotype in TR4 knockout mice can be correlated (FIG. 23).

In adult mice, serum IGF-I levels between TR4−/−, TR4+/−, and wildtype mice at 4 weeks of age can be compared. To assay serum levels of IGF-1, the ACTIVE™ Rat IGF-1 RIA kit (Diagnostic Systems Labs, Inc., Webster, Tex.) can be used. Ten mice of each genotype can be used to determine IGF-I serum levels. Based on the standard deviation between duplicate samples using the Diagnostic Systems Labs RIA kit, two mice per genotype can be used to detect a 20% difference with 80 power to achieve p<0.05. Therefore, 10 mice can be used to accurately detect small differences between mice of each genotype. Only 50 μl of serum sample is necessary for the assay, and the rat anti-IGF-1 antibody used is able to detect the mouse protein at a sensitivity of 21 ng/ml. Serum T₄ levels can also be assayed in samples from 10 mice of each genotype when the animals are 4 weeks of age. The ACTIVE™ Free T₄ RIA kit (Diagnostic Systems Labs) can be used. To assay pituitary levels of GH and TSH, 4 week old TR4−/−, TR4+/−, and wildtype mice can be sacrificed. The pituitary from each animal can be removed, and total protein extract can be prepared (Sambrook, J., Fritsch, E. F., and Maniatis, T. (1989) Molecular Cloning: A Laboratory Manual, 2 Ed., 3, Cold Spring Harbor Laboratory Press, Cold Spring Harbor, N.Y.). Antibodies to GH (Ingleton, P. M., Rodgers, M. F., and Parsons, M. A. (1992) Exp. Mol. Pathol. 56, 119-131) and TSH (Santa Cruz Biotech, Inc., Santa Cruz, Calif.) can be used in Western blot analysis (Sambrook, J., Fritsch, E. F., and Maniatis, T. (1989) Molecular Cloning: A Laboratory Manual, 2 Ed., 3, Cold Spring Harbor Laboratory Press, Cold Spring Harbor, N.Y.) of the isolated protein samples. To assay hypothalamic levels of GHRH in adult mice, 10 animals of each genotype can be sacrificed, and the hypothalamus of each animal can be removed. Total RNA can be isolated for Northern blot analysis (42B, Sambrook, J., Fritsch, E. F., and Maniatis, T. (1989) Molecular Cloning: A Laboratory Manual, 2 Ed., 3, Cold Spring Harbor Laboratory Press, Cold Spring Harbor, N.Y.), and samples can be blotted and probed for GHRH mRNA.

If there is no evidence for abnormalities in either IGF-1, TSH, T₄, GH, or GHRH levels, other nuclear, as well as non-nuclear receptors involved in growth processes can be assessed. Mutations eliminating the functions of either RARy, or VDR have resulted in significant growth retardation (Yoshizawa, T., Handa, Y., Uematsu, Y., Takeda, S., Sekine, K, Yoshihara, Y., Kawakami, T., Arioka, K., Sato, H., Uchiyama, Y., Masushige, S., Fukarnizu, A., Matsumoto, T., and Kato, S. (1997) Nat. Genet. 16, 391-396 I), and it is likely that TR4 may play a role in the normal function of the signaling pathways involving these nuclear receptors. It is known that TR4 is able to compete with VDR for binding to VDRE elements in VDR target genes (Lee, Y. F., Young, W. J., Lin, W. J., Shyr, C. R., and Chang, C. (1999) J. Biol. Chern. 274, 16198-16205). Additionally, through recognition of DR4RE-like motifs, those similar to response elements recognized by the thyroid hormone receptor, TR4 has been implicated as playing a role in the thyroid hormone signaling pathway (Lee, Y. F., Pan, H. J., Burbach, P. H., Morkin, E., and Chang, C. (1997) J. BioI. Chem. 272, 12215-12220). These data suggest the potential for the lack of TR4 to affect these and/or other related pathways and result in the observed TR4−/− growth defect. Finally, given the link of growth to food intake and nutritional absorption, anatomical and histological analysis of the gastrointestinal tract can be considered to assess potential defects in nutritional absorption. In related experiments, behavioral assessments of food and water intake can beP performed, making use of metabolic caging devices, as well as histological analysis of the hypothalamus, considering the role of this brain region in maintaining energy balance via regulation of hunger, thirst, and endocrine functions (Ruffin, M., and Nicolaidis, S. (1999) Brain. Res. 846, 23-29).

b) Characterization of Growth Retardation in TR4−/− Mice by Skeletal Analysis.

One of the major mechanisms in which serum growth hormones influence growth is through regulation of skeletal growth. However, skeletal abnormalities can result from a direct effect on bone formation or remodeling (Delany, A. M., Amling, M., Priemel, M., Howe, C., Baron, R., and Canalis, E. (2000) J. Clin. Invest. 105, 915-923, Lecanda, F., Warlow, P. M., Sheikh, S., Furlan, F., Steinberg, T. H., and Civitelli, R. (2000) J. Cell. Biol. 151, 931-943). An initial skeletal analysis of TR4−/− mice can be compared to TR4+/− and wildtype animals in terms of overall skeletal structure, bone mineral density, and bone turnover.

Skeletal analyses can determine structural anomalies that may contribute to the small size and impaired movement of the TR4−/− animals. Gross skeletal structure and ossification analyses can be performed on TR4−/− and wildtype animals of more than 3 weeks of age. Tibial sections can also be assessed for epiphyseal development. Possible defects in bone turnover can be investigated by estimating bone mineral density and bone mineral content using DEXA analysis (Vidal, 0., Lindberg, M. K., Hollberg, K., Baylink, D. J., Andersson, G., Lubahn, D. B., Mohan, S., Gustafsson, J.-A., and Ohlsson, C. (2000) Proc. Natl. Acad. Sci. USA 97, 5474-5479). Serum osteocalcin levels can also be assayed, as osteocalcin is a marker associated with bone metabolism (Richman, C., Baylink, D. J., Lang, K., Dony, C., and Mohan, S. (1999) Endocrinology 140, 4699-4705). If an abnormal skeletal structure is observed, which can account for the movement difficulties displayed by TR4−/− mice, the skeleton can be examined in animals prior to weaning, and at embryological stages, if necessary (FIG. 23).

The analysis of skeletal structure and ossification, can determine bone mineral density via DEXA scans of anesthetized TR4−/−, TR4+/−, and wildtype mice (Vidal, 0., Lindberg, M. K., Hollberg, K., Baylink, D. J., Andersson, G., Lubahn, D. B., Mohan, S., Gustafsson, J.-A., and Ohlsson, C. (2000) Proc. Natl. Acad. Sci. USA 97, 5474-5479). DEXA scans, skeletal staining, and long bone measurement can initially be performed on mice that are 4 weeks of age. Skeletal staining can be carried out as described previously (Yamaguchi, M., Nakamoto, M., Honda, H., Nakagawa, T., Fujita, H., Nakamura, T., Hirai, H., Narumiya, S., and Kakizuka, A. (1998) Proc. Natl. Acad. Sci. USA 95, 7491-7496). Briefly, animals can be skinned, eviscerated and skeletons can be fixed in ethanol. Skeletons can be stained first with alcian blue, then with alizarin red S, and finally cleared with graded glycerol solutions. Bone sizes can be measured and compared between adult TR4−/−, TR4+/−, and wildtype animals; if necessary, similar skeletal analysis of animals at embryonic stages can be performed. Also, serum osteocalcin levels can be assayed via radioimmunoassay (Richman, C., Baylink, D. J., Lang, K., Dony, C., and Mohan, S. (1999) Endocrinology 140, 4699-4705). As serum osteocalcin is known to increase with increases in osteoblast activity, reduced levels of this marker in TR4−/− mice compared with levels in TR4+/− or wildtype mice can be expected, given a defect in bone formation is part of the cause of the observed growth abnormality in knockout animals.

c) Characterization of Growth Retardation in TR4−/− Mice by Analysis of Skeletal Muscle.

Several murine models of muscular dystrophy are associated with growth retardation. Mice with a combined loss of utrophin and dystrophin show growth retardation at approximately 3 weeks of age (Deconinck, A. E., Rafael, J. A., Skinner, J. A., Brown, S. C., Potter, A. C., Metzinger, L., Watt, D. J., Dickson, G., Tinsley, J. M., and Davies, K. E. (1997) Cell 901, 717-727, 61. Grady, R. M., Teng, H., Nichol, M. C., Cunningham, J. C., Wilkenson, R. S., and Sanes, J. R. (1997) Cell 90, 729-738). Laminin α2 deficient mice are visably growth retarded by 14 days of age (Miyagoe, Y., Hanaoka, K., Nonaka, I., Hayasaka, M., Nabeshima, Y., Arahata, K., Nabeshima, Y., and Takeda, S. (1997) FEBS Lett. 415, 33-39, Kuang, W., Xu, H., Vachon, P. H., Liu, L., Loechel, F., Wewer, U. M., and Engvall, E. (1998)J. Clin. Invest. 102, 844-852). Laminin α2 and utrophin-dystrophin deficient mice show gait abnormalities (Deconinck, A. E., Rafael, J. A., Skinner, J. A., Brown, S.C., Potter, A. C., Metzinger, L., Watt, D. J., Dickson, G., Tinsley, J. M., and Davies, K. E. (1997) Cell 901, 717-727, Grady, R. M., Teng, H., Nichol, M. C., Cunningham, J. C., Wilkenson, R. S., and Sanes, J. R. (1997) Cell 90, 729-738, Miyagoe, Y., Hanaoka, K., Nonaka, I., Hayasaka, M., Nabeshima, Y., Arahata, K., Nabeshima, Y., and Takeda, S. (1997) FEBS Lett. 415, 33-39, Kuang, W., Xu, H., Vachon, P. H., Liu, L., Loechel, F., Wewer, U. M., and Engvall, E. (1998)J. Clin. Invest. 102, 844-852). Therefore, muscular pathology can contribute to the growth retardation and abnormal gait observed in the TR4−/− mice.

Initial muscle characterization can be performed using hematoxylin/eosin staining of the quadraceps and tibia anterior muscles from at least 6 mice each of TR4−/−, TR4+/−, and wildtype genotypes at 4 weeks of age. If degeneration or necrosis is observed in the TR4−/− and/or TR4+/− samples, The morphology of the neuromuscular junctions can be examined to determine if they resemble the abnormalities reported in other mouse models of muscular dystrophy (Deconinck, A. E., Rafael, J. A., Skinner, J. A., Brown, S.C., Potter, A. C., Metzinger, L., Watt, D. J., Dickson, G., Tinsley, J. M., and Davies, K E. (1997) Cell 901, 717-727, Grady, R. M., Teng, H., Nichol, M. C., Cunningham, J. C., Wilkenson, R. S., and Sanes, J. R. (1997) Cell 90, 729-738). This can be performed using rhodinated α-bungarotoxin, a selective stain for acetylcholine receptors, on whole muscles from wildtype, TR4−/−, and TR4+/− mice at 4 weeks of age. Any abnormalities detected can be confirmed by electron microscopy of the diaphragm, comparing 4 week old animals of all three genotypes. Additionally, serum creatine kinase levels can be assayed, as this enzyme is a marker for muscle stability (Kuang, W., Xu, H., Vachon, P. H., Liu, L., Loechel, F., Wewer, U. M., and Engvall, E. (1998)J. Clin. Invest. 102, 844-852). Therefore, high serum levels of creatine kinase for TR4−/− animals compared with TR4+/− and wildtype mice can provide further evidence that myopathy is contributing to the observed size defect. Serum creatine kinase can be measured in 6 mice from each genotype (TR4−/−, TR4+/−, and wildtype). Mice can be anesthetized with 100 mg/kg sodium pentobarbitol and blood collected by cardiac puncture. Samples can spun at 8000 g for 10 min and creatine kinase activity can be assayed with the CK10 kit (Sigma) (Kuang, W., Xu, H., Vachon, P. H., Liu, L., Loechel, F., Wewer, U. M., and Engvall, E. (1998)J. Clin. Invest. 102, 844-852). TR4−/−, TR4+/−, and wildtype mice (6 of each genotype) can be sacrificed at 4 weeks of age and the quadraceps and tibia anterior muscles removed and trimmed of fat and connective tissue. Quadraceps and diaphram muscles can be frozen in liquid nitrogen cooled isopentane and sectioned at 8 μm (Deconinck, A. E., Rafael, J. A., Skinner, J. A., Brown, S.C., Potter, A. C., Metzinger, L., Watt, D. J., Dickson, G., Tinsley, J. M., and Davies, K B. (1997) Cell 901, 717-727, Grady, R. M., Teng, H., Nichol, M. C., Cunningham, J. C., Wilkenson, R S., and Sanes, J. R (1997) Cell 90, 729-738). Tibia anterior muscles can be fixed in 0.2% paraformaldehyde in PBS, incubated overnight in 30% sucrose, and embedded in OCT (Hogan, B., Beddington, R., Constntini, F., and Lacy, E. (1994) Manipulating the mouse embryo, second Ed., Cold Spring Harbor Laboratory Press, Cold Spring Harbor). Quadraceps and tibia anterior muscles can be stained with hematoxylin and eosin (Culling, C. F. A. (1963) Handbook ofhistopathogical techniques, Butterworths, Inc., Washington) and X-gal (Hogan, B., Beddington, R, Constntini, F., and Lacy, E. (1994) Manipulating the mouse embryo, second Ed., Cold Spring Harbor Laboratory Press, Cold Spring Harbor). Acetylcholine receptors can be detected using rhodamine-abungarotoxin plus a flourescien conjugated secondary antibody and visualized by flourescence microscopy as previously described (Grady, R. M., Teng, H., Nichol, M. C., Cunningham, J. C., Wilkenson, R. S., and Sanes, J. R. (1997) Cell 90, 729-738). Ultrastructural anaylsis can be performed on sectioned diaphram muscles from mice of all three genotypes. Muscles can be fixed in 4% glutaraldehyde and 4% paraformaldehyde in PBS and then post-fixed in 1% OsO4. Muscle samples can be embedded in TAAB resin (TAAB Laboratories), sectioned using an ultramicrotome, and stained using lead citrate and uranyl acetate. Ultramicrotome sectioning and visualiztion with an Hitachi 7100 Electron Microscope with digital interface is available.

d) Characterization of Infertility, and the Specific Reproductive Phenotype in TR4−/− Mice

Initial indications are that the TR4−/− mice are infertile while the TR4+/− mice exhibit normal fertility. The number of animals assessed for infertility can be extended to determine the degree of penetrance of this phenotype. Results indicate that TR4 is normally expressed in the testes and prostate. TR4 expression was reduced in the cryptorchid testes of rhesus monkeys, as well as in monkeys rendered azoospermic by high dose testosterone treatment, indicatinging a correlation between TR4 expression and normal spermatogenesis (Mu, X., Liu, Y., Collins, L. L., Kim, E., and Chang, C. (2000) J. BioI. Chern. 275, 23877-23883). Lack of TR4 expression in male reproductive tissues can therefore result in developmental abnormalities or failure of spermatogenesis. Consistent with this hypothesis, TR4−/− males show a 66% reduction in epididymal sperm number at 7 weeks of age and a 33-39% reduction at 12-14 weeks of age. The role of TR4 in female reproductive physiology is less clear. TR4 has been reported by the P.I. and others (Harada, H., Kuboi, Y., Miki, R., Honda, C., Masushige, S., Nakatsuka, M., Koga, Y., and Kato, S. (1998) Endocrinology 139, 204-212) to repress ER mediated transcription. During normal follicular development, the level of ER protein and estradiol synthesis in the granulosa cells increases until the gonadatropin surge. Upon ovulation, ER levels decline and the steroidogenic activity of the forming corpus luteum is shifted towards progesterone secretion (Carr, B. R. (1998) in Williams textbook of Endocrinology (Wilson, J. D., Foster, D. W., Kronenberg, H. M., and Larsen, P. R., eds), pp. 751-817, WB saunders Company, Philadelphia, Horie, K., Takakura, K., Fujiwara, H., Suginami, H., Liao, S., and Mori, T. (1992) Human Reproduction 7,184-190). An interaction between TR4 and ER can facilitate the cyclical regulation of ovarian steroidogenesis, and disruption of this pathway could potentially lead to ovarian dysfunction. However, female infertility in TR4−/− mice can also be due to developmental defects in the female reproductive tract or through failure of the hypothalamic-pituitary axis. TR4 is highly expressed in the hypothalamus (Chang, C., Da Silva, S. L., Ideta, R., Lee, Y. F., Yeh, S., and Burbach, J. P. H. (1994) Proc. Natl. Acad. Sci. USA 1994, 6040-6044) and it is possible that loss of TR4 expression alters GnRH expression. The interaction between TR4 and ER can also influence mammary gland development or lactational function.

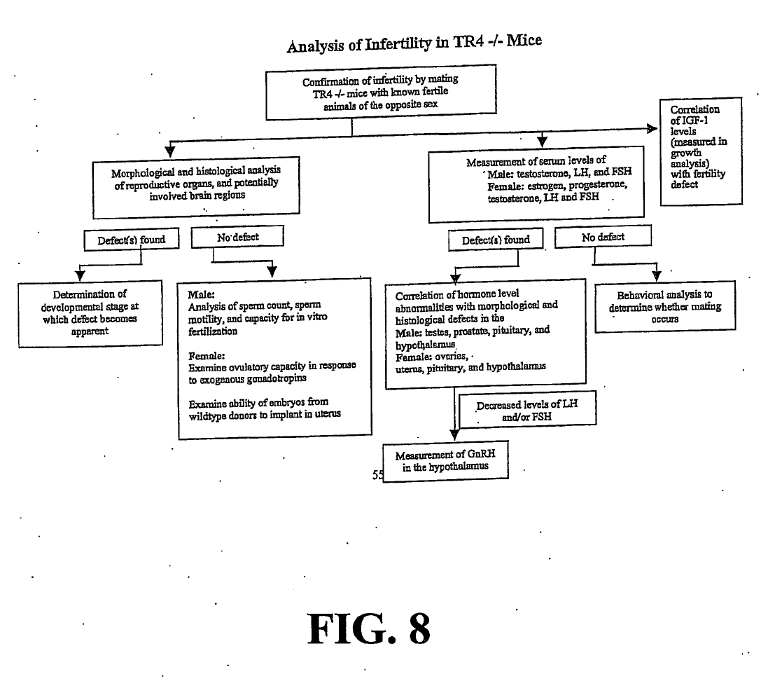

Results indicate that both male and female TR4−/− mice are infertile. To confirm this observation, male and female TR4−/− animals can be paired with known fertile wildtype animals of the opposite sex, and assess whether pregnancy and birth of pups occurs. If no pups are born, the TR4−/− animals can be paired with another known fertile mouse of the opposite gender. If the second pairing still does not result in a litter, the TR4−/− animals can be considered infertile. TR4−/−, TR4+/− and wildtype animals can be sacrificed, necropsies can be performed, and tissues can be prepared for sectioning and histology to determine the anatomical and histological phenotypes of the reproductive organs (FIG. 8). In the female TR4−/− mice, particular attention can be paid to the histology of the mammary gland, ovaries and uterus. Because TR4 is known to be highly expressed in the ventral prostate and is implicated in spermatogenesis (Mu, X., Liu, Y., Collins, L. L., Kim, E., and Chang, C. (2000) J. BioI. Chern. 275, 23877-23883), prostatic histology and histology of the testes and epidiymis, with an emphasis on germ cell phenotype, can be carried out for the TR4−/− males. For all fertility analyses, TR4−/− animals can be compared to TR4+/− and wildtype age and gender matched controls. Results indicate that TR4−/− males have a reduced number of epididymal sperm at all ages examined. A further analysis of sperm motility using an automated IVOS system can be carried out in concert with a morphological analysis of TR4−/− epididymal sperm. Organs in which abnormalities are observed can be examined in pre-pubertal TR4−/− animals, and during embryonic development, to determine the stage at which abnormalities first become apparent.

Much of both male and female sexual function depends on proper signaling by both gonadal and pituitary hormones. Sexually mature TR4−/− male and female mice can be assayed for serum levels of gonadal steroids (estradiol, progesterone, and testosterone) and for anterior pituitary hormones (LH and FSH) for comparison to TR4+/− and wildtype values. Serum IGF-1 levels obtained in the growth analysis aspect of the study, if abnormal in the TR4−/−, can also be considered in the fertility analysis, as IGF-1 null mutants display dramatic reduction in reproductive organ size, as well as infertility (Baker, J., Liu, J. P., Robertson, E. J., and Efstratiadis, A. (1993) Cell 75, 73-82). In addition to the other gonadal hormones, measurement of serum testosterone can be carried out in the female TR4−/−, as female αERKO mice are infertile and have both high serum estradiol and increased levels of serum testosterone (Couse, J. F., and Korach, K. S. (1999) Endocrine Reviews 20,358-417 69. Luo, X., Ikeda, Y., and Parker, K. L. (1994) Cell 74, 481-490). Analysis of hormonal levels in TR4−/− mice can further help to delineate the primary cause of infertility, and can assist us in determining whether the TR4-mediated defect is in the gonadal, steroid producing cells, or related to disruption of the hypothalamic-pituitary axis. If significant differences are observed in the levels of LH and/or FSH in TR4−/− mice compared to levels of the same hormones in TR4+/− and wildtype animals, investigations can look further upstream and determine gonadotropin releasing hormone (GnRH) levels in the hypothalamus (FIG. 8B).

The direction of further study of male and female TR4−/− infertility can depend on the phenotype found in the initial analysis. For example, TR4−/− and wildtype female mice could be treated with exogenous gonadotropins, with subsequent collection of oocytes. The total number of oocytes could then be compared between animals to assess whether intraovarian TR4 is essential for ovulation. This experiment would not be undertaken, however, if TR4−/− females lack ovaries, as it the case with mice carrying a targeted disruption of the orphan receptor SF-1 (Luo, X., Ikeda, Y., and Parker, K. L. (1994) Cell 74, 481-490). For mammary gland analyses, mammary gland histology can initially be compared in adult 8 week old females, comparing wildtype, TR4−/−, and TR4+/− females. If TR4−/− females are found to be fertile, mammary gland development can be compared between the three genotypes at partuition. Alternatively, if TR4−/− females do not become pregnant, virgin female adult mice of each genotype can be ovarectomized and treated for 21 days with E2 and progesterone as previously reported (Smith, C. L., DeVera, D. G., Lamb, D. J., Nawaz, Z., Jiang, Y. H., Beaudet, A. L., and O'Malley, B. W. (2002) Mol. Cell Bioi. 22, 525-535., Mulac-Jericevic, B., Mullinax, R. A., DeMayo, F. J., Lydon, J. P., and Conneely, O. M. (2000) Science 289, 1751-1754.), a treatment regime that mimics early pregnancy induced mammary gland differentiation. In the case of TR4−/− male mice, epididymal sperm counts, sperm motility, and in vitro fertilization capacity can be examined (FIG. 8) compared to TR4+/− and wildtype mice. As detailed below, sperm motility analyses can be performed using an automated image analysis system (IVOS). The studies can be expanded to include mice of 6 weeks of age in addition to the 7 and 12-14 weeks of age previously examined.

To confirm male and female infertility, a total of five male and five female TR4−/− animals can be paired with known fertile wildtype animals of the opposite sex for two weeks. After a total of four weeks from the initial time of pairing, each female showing no sign of pregnancy, and each male can be paired with another known fertile wildtype animal of the opposite sex. Each morning after pairing, female mice can be checked for copulatory plugs. After a plug is observed for a particular animal, that animal can no longer be checked, yet each pair can remain together for the full two weeks. In parallel, matings between wildtype animals can be set up as controls in the event that TR4−/− mice are not infertile, but show reduced fertility.

For histological analysis, tissues can be fixed overnight in 0.2% paraformaldehyde in PBS, incubated in a solution containing 30% sucrose, overnight, and embedded in OCT (Hogan, B., Beddington, R., Constntini, F., and Lacy, E. (1994) Manipulating the mouse embryo, second Ed., Cold Spring Harbor Laboratory Press, Cold Spring Harbor). Sections can be cut via cryostat, mounted on slides, and hematoxylin and eosin (Culling, C. F. A. (1963) Handbook of histopathogical techniques, Butterworths, Inc., Washington), as well as X-gal, staining (Hogan, B., Beddington, R., Constntini, F., and Lacy, E. (1994) Manipulating the mouse embryo, second Ed., Cold Spring Harbor Laboratory Press, Cold Spring Harbor) can be carried out. Organs in which abnormalities are observed can be examined in each of five TR4−/−, TR4+/−, and wildtype prepubertal animals of each gender, as well as in embryos from six TR4+/− females that had been paired with TR4+/− males. Initially, embryos from E14 can be analyzed to begin to narrow down the time of onset of the TR4−/− fertility defect. Further embryonic analyses at additional time points can be carried out, if necessary. To assay serum levels of gonadal hormones in 10 week old TR4−/−, TR4+/− and wildtype mice, the Testosterone radioimmunoassay (RIA), Estradiol RIA, and Progesterone RIA systems can be employed which are commercially available (Diagnostic Systems Laboratories, Inc., Webster, Tex.). The number of mice required to observe a 20% difference between genotypes with an 80 power magnification to achieve p<0.05 varies with different hormones due to a different standard deviation between replicate samples seen for each hormone assay (Xu, J., Liao, L., Ning, G., Yoshida-Komiya, H., Deng, C., and O'Malley, B. W. (2000) Proc. Natl. A cad. Sci. USA 97, 6379-6384, Eddy, E. M., Washburn, T. F., Bunch, D. O., Goulding, E. H., Gladen, B. C., Lubahn, D. B., and Korach, K. S. (1996) Endocrinol. 137, 4796-4805). In the case of testosterone, an estimated 58 animals of each genotype can be required to observe a 20% difference with 80 power to achieve p<0.05. In contrast, seven animals of each genotype are necessary for the same power and difference for serum estradiol. Here, 58 animals of each genotype for testosterone and 14 animals of each genotype can be assayed for estardiol and progesterone. Serum LH and FSH levels assayed via radioimmunoassay (Wu, T. J., Silverman, A. J., and Gibson, M. J. (1996) J. Neurosci. 31, 67-76) in fourteen 10-week old mice of each gender and each genotype. A power analysis for these hormone assays (Schwartz, N. B., Szabo, M., Verina, T., Wei, J., Dlouhy, S. R., Won, L., Heller, A., Hodes, M. E., and Ghetti, B. (1998) Neuroendocrinology 68,374-385) indicates that fourteen mice of each genotype are sufficient to detect a 20% difference with 80 power (p<0.05) for LH levels and two mice of each genotype can be needed for FSH. Hypothalamic levels of GnRH in TR4−/− mice can be compared to those of TR4+/− and wildtype animals. Fourteen animals of each gender and each genotype can be sacrificed, and the hypothalamus of each animal can be dissected from the brain. Total RNA can be isolated from the samples and used in Northern blot analysis (. Sambrook, J., Fritsch, E. F., and Maniatis, T. (1989) Molecular Cloning: A Laboratory Manual, 2 Ed., 3, Cold Spring Harbor Laboratory Press, Cold Spring Harbor, N.Y.), employing a probe specific for GnRH.

To analyze sperm motility and to extend the sperm number data, epididymal sperm can be collected from approximately 10 of each TR4−/−, TR4+/−, and wildtype mice at weekly time points from 7-14 weeks of age and at one year of age. Cauda epididymi can be minced and placed in commercially available M16 medium supplemented with 20 mg/ml BSA. Samples can be incubated at 37° C. to allow sperm dispersal. Aliquots can be diluted 1:5 and 1:10. Sperm counts, percentages of motile sperm, and types of flagellar movement can be determined using an IVOS Sperm Analyzer 10.7 q (Hamilton Thorne Research) computer integrated visual optical system. The IVOS system allows sperm motility to be analyzed in terms of path velocity, progressive velocity, track speed, lateral amplitude, beat frequency, and velocity distribution. The settings of the IVOS Sperm Analyzer have been modified to detect mouse sperm with the assistance of Hamilton Thorne Research. Based on the data, to detect a 20% difference in sperm numbers using the highest variance found in the results with a 80 power to achieve p<0.05, 10 mice per genotype are needed.

For in vitro fertilization studies, 8 week old wildtype female mice can be primed with hCG for two days, and 2 hours before analysis is to begin. Cumulus masses containing eggs can be collected from the oviducts of primed females 13-16 hrs after hCG injection. Cumulus masses can be transferred to medium, and eggs can be inseminated with motile sperm from wildtype, TR4−/−, or TR4+/− males at a concentration of 1-2×10⁶ motile sperm/ml. After 8-10 hrs of incubation at 37° C., eggs can be washed in M2 medium, fixed in 2.5% gluteraldehyde and transferred to slides. After overnight fixation in 3.7% formaldehyde, dehydration in 95% ethanol, and staining with acetolacmoid, eggs can be analyzed for fertilization via microscopy. Eggs can be scored as fertilized if two pronuclei and a sperm tail are present within the vitellus (Eddy, E. M., Washburn, T. F., Bunch, D. O., Goulding, E. H., Gladen, B. C., Lubahn, D. B., and Korach, K. S. (1996) Endocrinol. 137, 4796-4805).

While the data indicates that TR4 plays an important role in the development of fertility in both males and females, the reproductive phenotype can be confirmed through detailed histological analysis of the testes and ovaries. Further, it is possibile that TR4−/− mice may experience a delay in sex organ development. Therefore, the fertility analysis can be continued with TR4−/− mice at various ages, as more homozygous knockout animals become available. Lack of significant differences in reproductive organ structure, either grossly or via histological analysis, between TR4−/− mice and TR4+/− or wildtype animals, as well as no differences in serum hormone levels or hypothalamic GnRH expression can indicate a behavioral abnormality. To determine if TR4−/− animals are actually mating, a video recorder can be trained on a cage, containing a newly introduced pair, for 6 hours each night (from 00:00-06:00) for one week after their pairing, or until mating is observed. At least 5 pairs with TR4−/− animals of each gender, matched with a known fertile wildtype animal of the opposite sex, can be observed for mating behavior in this way. The female of each pair can be checked each morning for a copulatory plug as well. If a behavioral defect is observed, further experiments to characterize the behavioral phenotype can be carried out.

e) Investigation of Potential Neurological and Behavioral Defects in TR4−/− Mice.

TR4 is normally expressed at high levels in the brain, particularly in the hippocampus hypothalamus, and cerebellum (Chang, C., Da Silva, S. L., Ideta, R., Lee, Y. F., Yeh, S., and Burbach, J. P. H. (1994) Proc. Natl. Acad. Sci. USA 1994, 6040-6044). Results show a 20-30% reduction in the density granular neurons of the cerebellum in TR4−/− mice as compared to age matched wildtype mice. Results also show that TR4 positively regulates the iNOS promoter, the predominant form of NOS in glial cells (Guo, L., Sawkar, A., Zasadzki, M., Watterson, D. M., and Van Eldik, L. J. (2001) Neurobiol. Aging 22, 975-981). The results therefore indicate the presence of several neurological defects in the TR4−/− animals. Further histological examination of the brain of TR4−/−, TR4+/−, and wildtype mice can be performed to confirm the data. The point in the embryological development of the cerebellum the differences in granular neuron cell density is manifested in TR4−/− mice compared to TR4+/− and wildtype mice. In addition to brain, histological examination of the spinal cord can also be performed. Observations indicate that TR4−/− animals are generally inactive and do not exhibit the exploratory behavior seen in TR4+/− and wildtype mice. When TR4−/− mice do move, they exhibit an abnormal gait characterized by lack of coordinated reciprocal stepping of hind limbs. Reciprocal stepping and rhythmic stepping movements are predominantly controlled by the lumbar spinal cord. The abnormal gait of the TR4−/− animals can therefore reflect a spinal cord or peripheral nerve defect. A contributing factor to the general immobility of TR4−/− mice can be impairment of the interaction between the cerebellum and cerebral cortex in regulating voluntary movement. The hypothalamus, where TR4 is normally highly expressed, is involved not only in regulation of endocrine functions, but is also involved in the control of feeding reflexes and of determining thirst and satiety (Ruffin, M., and Nicolaidis, S. (1999) Brain. Res. 846, 23-29).

There is considerable evidence that, in rodents, the hippocampus is involved in the learning of spatially guided tasks (Morris, R G. M., Garrud, P., Rawlins, J. N. P., and O'Keefe, J. (1982) Nature 297,681-683). Lack of TR4 in the hippocampus can potentially alter the learning capacity of TR4−/− mice. The hippocampus can be examined for histological abnormalities and test the spatial learning ability of TR4−/− and control mice using a Morris water maze (Morris, R. G. M., Garrud, P., Rawlins, J. N. P., and O'Keefe, J. (1982) Nature 297,681-683).

To characterize the defect which results in reduction in overall cerebellum size, as well as in granule neuron number in TR4−/− mice, brain samples from TR4−/−, heterozygous and wildtype mice undergoing different stages of cerebellar development can be collected and analyzed. In the mouse, cerebellar development begins with appearance of neurons in the germinal matricies, the ventricular zone and the rhombic lip, from which the cerebellum is derived (Goldowitz, D., and Hamre, K. (1998) Trends Neurosci. 21,375-382.). A significant event in late embryonic and early postnatal cerebellar development is the formation of the external granule cell layer (EGL) at approximately 15 days of gestation (E15), followed by migration of the granule cells to their final position in internal granule cell layer (IGL), and the eventual disappearance of the EGL by the end of the third week after birth (Goldowitz, D., and Hamre, K. (1998) Trends Neurosci. 21,375-382.). From the reduction in granule cell number in the brains of the adult TR4−/− mice analyzed, it is suspected that there may be a defect in formation of the EGL, migration of granule cells to the IGL or an inability of the granule cells, once inward migration has occurred, to populate the internal granule layer. To determine the developmental time point at which the cerebellar defect becomes apparent, the brains of TR4KO mice and control animals of the same sex can be collected at the following embryonic and early postnatal time points: E16, day of birth (P0), seven days of age (P7) and P14. Once tissue samples have been collected and processed, they can be analyzed using histological and morphometric techniques. Once the stage at which the cerebellar defect occurs is identified, immunohistochemical staining techniques can be used to stain for markers of cerebellar development. Genes known to be important in the regulation of granule cell production or apoptosis, such as Pax6, RU49, and BDNF (Goldowitz, D., and Hamre, K. (1998) Trends Neurosci. 21, 375-382.) may be found to be controlled, either directly or indirectly, by TR4. Further analysis of other brain regions can be performed to examine degree of mylination the presence of other morphological differences between TR4−/−, TR4+/− and wildtype mice.

Histological examination of the spinal cord can initially be determined in 4 week old TR4−/− mice and compared to 4 week old TR4+/− and wildtype mice. If defects are found in the TR4−/− mice, the spinal cord can be examined to determine the developmental stage at which the defect is first detectable. If spinal cord defects are found at 4 weeks in TR4−/− mice, developmental analysis can be performed from gestational day 8, at the time of neural tube closure (Hogan, B., Beddington, R., Constntini, F., and Lacy, E. (1994) Manipulating the mouse embryo, second Ed., Cold Spring Harbor Laboratory Press, Cold Spring Harbor) to birth. Defects of the brain and/or spinal cord can be correlated with the presence or absence of appropriate markers. Potential decreases in hunger and thirst in TR4−/− animals can be determined by housing TR4−/− and wildtype mice in individual metabolic cages (Ruffin, M., and Nicolaidis, S. (1999) Brain. Res. 846, 23-29). Although TR4−/− mice are inactive relative to TR4+/− and wildtype mice, they can swim if placed in water. The spatial learning ability of TR4−/−, TR4+/−, and wildtype mice can be compared using a Morris water maze (Morris, R. G. M., Garrud, P., Rawlins, J. N. P., and O'Keefe, J. (1982) Nature 297,681-683). For histological analysis, ten 4-week old TR4−/−, TR4+/−, and wildtype animals can be sacrificed, and the brain and spine can be removed. The spine/spinal cord can be fixed in 0.2% paraformaldehyde in PBS (Hogan, B., Beddington, R., Constntini, F., and Lacy, E. (1994) Manipulating the mouse embryo, second Ed., Cold Spring Harbor Laboratory Press, Cold Spring Harbor), decalcified in 0.2 N HCl for several days (until bones become soft) (Mundos, S. (1998) Methods in Molecular Biology 136, 61-70), and embedded in OCT for subsequent sectioning. Brain tissue can be fixed overnight in 0.2% paraformaldehyde in PBS, incubated in a solution containing 30% sucrose overnight and embedded in OCT (Hogan, B., Beddington, R., Constntini, F., and Lacy, E. (1994) Manipulating the mouse embryo, second Ed., Cold Spring Harbor Laboratory Press, Cold Spring Harbor). Brain tissue from separate animals can be fixed in 10% buffered formalin, dehydrated with graded ethanol, and embedded in glycol methacrylate resin (Uno, H., Lohmiller, L., Thieme, C., Kemnitz, J. W., Engle, M. J., Roecker, E. B., and Farrell, P. M. (1990) Dev. Brain Res. 53, 157-167). Paraffin sections can be cut via cryostat, mounted on slides, and hematoxylin and eosin (Culling, C. F. A. (1963) Handbook of histopathogical techniques, Butterworths, Inc., Washington), as well as X-gal staining (Hogan, B., Beddington, R., Constntini, F., and Lacy, E. (1994) Manipulating the mouse embryo, second Ed., Cold Spring Harbor Laboratory Press, Cold Spring Harbor) can be carried out. Glycol methacrylate resin embedded samples can be section with a glass knife using a JB-4 microtome (Soval). To test for potential learning defects in the knockout animals, the Morris water maze behavioral analysis of spatial learning (Morris, R. G. M., Garrud, P., Rawlins, J. N. P., and O'Keefe, J. (1982) Nature 297,681-683) can be performed on 15 each of TR4−/−, TR4+/−, and wildtype mice of 4 weeks of age. Briefly, the water maze apparatus is a tank with a radius of approximately 55 cm containing a platform that is 3.5 cm square. Water is added to the tank such that the platform is submerged 0.5 cm below the surface of the water. The water is rendered opaque by the addition of a non-toxic latex compound. The goal of the trial is to determine how quickly the mouse learns the position of the platform in the tank. The platform can remain in the same position in the tank for each block of trials. Each mouse can have 6 blocks of three 60 second swim trials. If the mouse has not found the platform after 60 seconds, the animal can be guided to the platform and allowed to remain there for 60 seconds. There can be a 10-minute interval between trials, and 2 blocks can be run per day over a three day period. The mouse performance in the trial can be recorded on videotape. The TR4−/− animals can display impairment of spatial learning ability, and that this defect can be apparent in that it can take TR4−/− mice significantly longer than TR4+/−, or wildtype animals to find the platform after several trials in the Morris water maze.

f) Investigation of Potential Defects in Other Organs of TR4−/− Mice.

Because TR4−/− mice do not show an elevated mortality, serious defects in other organ systems apart from the skeletal, reproductive, and nervous systems are not expected. However, morphological and histological analysis of other tissues including liver, lung, intestine, pancreas, kidney, adrenal gland, spleen, heart, and bone marrow can be carried out.

For histological analysis, ten 4-week old TR4−/−, TR4+/−, and wildtype animals can be sacrificed, and the tissues of interest can be removed. The tissue can be fixed overnight in 0.2% paraformaldehyde in PBS, incubated in a solution containing 30% sucrose, overnight, and embedded in OCT (Hogan, B., Beddington, R., Constntini, F., and Lacy, E. (1994) Manipulating the mouse embryo, second Ed., Cold Spring Harbor Laboratory Press, Cold Spring Harbor). Sections can be cut via cryostat, mounted on slides, and hematoxylin and eosin (Culling, C. F. A. (1963) Handbook of histopathogical techniques, Butterworths, Inc., Washington), as well as X-gal, staining (Hogan, B., Beddington, R., Constntini, F., and Lacy, E. (1994) Manipulating the mouse embryo, second Ed., Cold Spring Harbor Laboratory Press, Cold Spring Harbor) can be carried out.

g) Determination of TR2 Expression Levels in the TR4−/− Mice.

TR2 and TR4 share 51% homology in their N-terminal domains, and 82% and 65% homology in their DBDs and LBDs, respectively (Chang, C., Da Silva, S. L., Ideta, R., Lee, Y. F., Yeh, S., and Burbach, J. P. H. (1994) Proc. Natl. Acad. Sci. USA 1994, 6040-6044). Because they show a relatively high degree of homology to each other compared to other members of the nuclear receptor superfamily, and because they have highly overlapping patterns of expression and regulate many of the same target genes, it is possible that, in some physiological systems, TR2 is able to compensate for the lack of TR4. To formally determine this, the TR4−/− phenotype can be compared to the TR2−/− phenotype and TR2/TR4 double knockouts. However, a possible indication as to where TR2 may be compensating for the lack of TR4, is the expression level of TR2 in tissues of the TR4−/− animal. It is possible that TR2 is upregulated in tissues where it is compensating for lack of TR4 expression. However, if TR2 protein is already present at a high level, allowing compensation for loss of TR4 without increase expression, no alteration in protein levels can be observed.

The protein levels of TR2 can be examined by Western blot in tissues from adult TR4−/−, TR4+/−, and wildtype mice. Any differences observed can be traced back to the time of onset through analyses of tissues from pups, as well as from embryos. To determine a more precise indication of differences in spatial or temporal expression of TR2 under TR4 null conditions, cryosections of various tissues can be prepared and immunohistochemical analysis of embryos at different stages, or of tissues from mice at different ages throughout postnatal development, can be performed.

Five adult animals of each genotype can be sacrificed, and organs from each animal can be removed and used to produce total protein extract (Sambrook, J., Fritsch, E. F., and Maniatis, T. (1989) Molecular Cloning: A Laboratory Manual, 2 Ed., 3, Cold Spring Harbor Laboratory Press, Cold Spring Harbor, N.Y.). A monoclonal antibody to TR2 (G204) can be used in Western blot analysis (Sambrook, J., Fritsch, E. F., and Maniatis, T. (1989) Molecular Cloning: A Laboratory Manual, 2 Ed., 3, Cold Spring Harbor Laboratory Press, Cold Spring Harbor, N.Y.) of the isolated protein samples. As the tissues of most interest are the brain, spinal cord, and reproductive organs, these organs can be the initial focus.

h) Investigation of Hematopoiesis in TR4−/− Mice.

Nuclear receptors have been found to be important for aspects of hematopoiesis, in particular erythropoiesis. Both ER and GR are required for efficient self-renewal of erythroid progenitor cells (Wessely, 0., Deiner, E. M., Beug, H., and von Lindern, M. (1997) EMBO J. 16,267-280, Bauer, A., Mikulits, W., Lagger, G., Stengl, G., Brosch, G., and Beug, H. (1998) EMBO J. 17, 4291-4303.). TR and RXR participate in the regulation of erythroid progenitor proliferation and differentiation (Bauer, A., Mikulits, W., Lagger, G., Stengl, G., Brosch, G., and Beug, H. (1998) EMBO J. 17, 4291-4303, Bartunek, P., and Zenke, M. (1998) Mol. Endocrinol. 12, 1269-1279.). TR4 was found to be highly expressed in human and mouse hematopoietic cell lines, and in dendritic cells, erythroblasts, T-cells, and monocytes (Koritschoner, N. P., Madruga, J., Knespel, S., Blendinger, G., Anzinger, B., Otto, A., Zenke, M., and Bartunek, P. (2001) Cell Growth Differ. 12, 563-572.). Retrovirus mediated transfection of TR4 into multi-potential chicken embryo fibroblasts induces the proliferation of promyelocytes, suggesting that TR4 can play a role in myelopoiesis. It is possible that mice lacking TR4 can have defects in hematopoiesis, particularly in the myeloid compartment.

An initial assessment of hematopoiesis in TR4−/−, TR4+/−, and wildtype mice can be determined from examination of peripheral blood smears and bone marrow films from adult mice. Because overexpression of TR4 results in increased proliferation of myeloid precursor cells (Koritschoner, N. P., Madruga, J., Knespel, S., Blendinger, G., Anzinger, B., Otto, A., Zenke, M., and Bartunek, P. (2001) Cell Growth Differ. 12,563-572), it is possible that the absence of TR4 can result in a reduction in neutrophils, basophils, and/or eosinophils. However, a comparison of all blood cell types can be made between mice of all three genotypes. If an altered number of particular blood cell types is observed in the TR4−/− animals, the analysis can be altered to determine the particular precursor or progenitor cell types effected. For example, if eosinophil and neutrophil cell numbers are altered in TR4−/− mice compared to wildtype controls, the number of colony forming units-granulocyte/macrophage (CFU-GM) can be examined from bone marrow and spleen from mice of each genotype. Fluorescence-activated cell analysis with flow cytometry using lineage specific markers can be used to further define the specific cell types affected by loss of TR4. If a deficiency or excess of a specific cell types is identified, future experiments can include the use of three dimensional long term bone culture (Wang, T. Y., Brennan, J. K, and Wu, J. H. (1995) Exp. Hematol. 23, 26-32.) to examine the effect of specific hematopoietic growth factors on hematopoiesis in TR4−/− derived cells.

Peripheral blood samples from the retro-orbital sinus can be collected from five six week old mice of each genotype (TR4−/−, TR4+/−, and wildtype). Samples can be collected into polyproplyene tubes containing EDTA as an anitcoagulant. Complete blood cell counts can be performed using a Coulter counter and differential blood cell counts to determine the number of each type of blood cell present can be performed on blood smears using a Wright-Giemsa. For bone marrow films, femoral bone marrow can be collected from five six week old mice of each genotype by flushing the central cavity with 50 μl of PBS/10 mM EDTA as previously described (. Frenette, P. S., Mayadas, T. N., Rayburn, H., Hynes, R. O., and Wagner, D. D. (1996) Cell 84, 563-574.). Smears of the cell suspension can be made and stained with Wright-Giemsa for determining differential cell counts. To perform colony forming assays, femoral bones can be flushed asceptically with MEMα (with 2% FBS, 100 U/ml penicillin, 100 μg/ml streptomycin) using a 21 gauge needle and a single cell suspension can be made by gentle aspiration. The cell suspension can be mixed 1:10 with Methocult GF (Stemcell Technologies). Spleens can be removed, washed in Hanks buffered saline and cells extracted through a stainless steel grid. Spleen cells can also be mixed 1:10 with Methocult GF. Colony forming units-granulocyte/macrophage or erythroid burst forming units can be counted after 7 days in culture. Flow cytometric analysis can be performed on asecptically collected femoral bone marrow. The choice of lineage antibody markers to be used can be determined by the results of the differential cell counts from peripheral blood an bone marrow. However, the myeloid markers (eg. MC47-83, MC-51-2, and MC-22-3), macrophage markers (eg. K1), erythroid markers (eg.JS4), and T cell markers (eg. CD3, CD4, and CD8) can be used. Cells can be incubated with the primary antibody for 1 hr and then incubated with FITC conjugated antimouse IgG. After washing, cells can be resuspended in PBS supplemented with 1% BSA and 2 μg/ml propidium iodide. Propidium iodide staining can allow for gating to remove nonviable cells from the analysis. Cells can then be analyzed by flow cytometry.

i) Characterization of TR2 Knockout/β-gal Knockin (TR2−/−) Mice.

The TR2 shares a 65% overall homology to TR4, with the DNA homology between the N-terminal, DBD, and LBD being 51%, 82%, and 65%, respectively (Chang, C., Da Silva, S. L., Ideta, R., Lee, Y. F., Yeh, S., and Burbach, J. P. H. (1994) Proc. Natl. A cad. Sci. USA 1994, 6040-6044). While there is considerable overlap in the tissue distribution of both receptors, they exhibit differential, stage-specific onset of expression during embryogenesis, with TR2 expression beginning earlier than TR4. It is unclear whether TR2 and TR4 share any functional redundancy. To determine whether disruption of TR2 during embryogenesis causes a more severe phenotype than does disruption of TR4, and to determine the degree of functional overlap between the two receptors, the generation of TR2 knockout/β-gal knockin (TR2−/−) mice has been initiated. As shown in the results, the TR2 locus in ES cells has been successfully targeted and chimeric mice have been generated. TR2+/− mice have been generated and bred to produce TR2−/− animals. TR2−/− mice can be analyzed as TR4 mice, and aspects of development, physiology, and behavior can be investigated in a similar manner to those proposed for the analysis of the TR4−/− line, and compare the TR2−/− mice to the TR4 null animals. An alternative way can be designed to account for any difference in phenotype observed in the TR2 knockout mice.

Because TR2 is expressed prior to TR4 during development, it is possible that TR2−/− mice can die pre- or postnatally. Pups from heterozygous pairings that die postnatally can be genotyped to determine if they are TR2−/−. If TR2−/− pups show early mortality, necropsies, as well as histological analyses, can be performed to determine the cause of death. If no significant pup mortality is seen in litters from heterozygous pairings, whether TR2−/− animals are present at weaning in Mendelian ratios can be ascertained. If it becomes apparent that TR2−/− mice die embryonically, it can be determined the embryonic stage at which the pups are no longer viable, and the probable cause of mortality. If TR2−/− mice survive to adulthood, they can be assessed for growth rate and fertility as proposed in the analysis of the TR4−/− mice.

Genotyping can be carried out with primers to a region of the inserted IRES βgal MC1-Neo selection cassette, as well as with primers to a region of genomic DNA within the portion replaced by the cassette after homologous recombination occurs. Histological analysis can be performed as described. If prenatal mortality is observed, pregnant TR4+/− animals, that had been mated to TR4+/−males, can be sacrificed at various time points throughout pregnancy, starting at E10.5, to determine the time at which TR4−/− animals are no longer viable. The analysis of embryos can continue at earlier or later times during embryogenesis based on the initial results at E10.5. For growth rate and fertility analyses.

j) Characterization of TR2/TR4 Double Knockout Mice

Because TR2 and TR4 share a high degree of homology and considerable overlap in expression, it is possible that a degree of functional redundancy exists between the two receptors. It is clear from the analysis of the TR4−/− mice that TR2 is unable to compensate for TR4 in reproductive and growth physiology. By generating a TR2/TR4 double knockout line, the systems in which these receptors show functional redundancy can be determined.

Because TR4−/− mice are infertile and it is possible that TR2−/− mice can be infertile or die prior to sexual maturity, TR2/TR4 double knockout mice have been generated from animals that are heterozygous for both the TR2 and TR4 targeted loci. Analysis of the phenotype of the TR2/TR4−/− animals can initially be carried out in the same manner as for TR4, and the phenotype of the double knockouts can be compared to TR2−/−, TR4−/−, and wildtype mice. Additional experimental protocols can be designed for use in analysis of any TR2/TR4−/− phenotype that differs significantly from those observed in either the TR4−/− or TR2−/− animals.

An alternative that would allow the study of TR2 function later in development, would be the production of a conditional knockout of TR2, utilizing Cre/LoxP technology (Le, Y., and Sauer, B. (2000) Methods Mol Bioi 136, 477-485). Based on the promoter driving Cre expression, a TR2 knockout can be focused in particular tissues of interest (Xu, X., Wagner, K.-U., Larson, D., Weaver, Z., Li, C., Ried, T., Henninghausen, L., Wynshaw-Boris, A., and Deng, C.-X. (1999) Nat. Genet. 22, 37-43, Stec, D. E., DAvisson, R. L., Haskell, P E., Davidson, B. L., and Sigmund, C. D. (1999) J Bioi. Chern.) or expressed at particular time points during development with a reasonable degree of specificity.

### 8. EXAMPLE

### Vertebrate Animals

Embryonic through adult mice of both sexes were used. The heterozygote knockout mice that were used were F 1 hybrids of strains 126/SvEv and C57BL/6. Both C57BU6 and 126/SvEv mice can be used to maintain the knockout lines, should genetic background effects be important in analysis of the phenotype. To perform the analysis disclosed 3-10 animals of the appropriate genotypes can be used for each experiment to generate statistically significant data.

Procedures to which the mice can be subjected without anesthesia include ear punching, tail biopsies, and the Morris water maze spatial learning test. Ear punches, for the purpose of animal identification, and tail biopsies, for genotyping, can be performed on each animal at the time of weaning (approximately 3 weeks of age). Biopsies of the distal 7-10 mm of the tail can be taken with a sterile straight edged blade. The tail can be dipped in antibiotic powder after biopsy to aid clotting and prevent infection. For tail biopsies taken after 28 days of age, anesthesia can be used. For the Morris water maze test, animals can be placed in a tank of opaque water containing a submerged platform. Each mouse can be tested in 6 blocks of three 60-second swim trials. Only 2 blocks of trials typically are run per day. If the mouse is unable to find the platform within 60 seconds, it can be guided to the platform. An interval of ten minutes typically will be allowed between trials.

Procedures involving the use of anesthesia include blood collection from mouse tails and euthanasia. In the case of tail bleeds, mice can be sedated with an intraperitoneal injection of ketamine. Upon reaching the surgical plane (monitored via toe and ear pinch reflexes), blood can be collected. After completion of the procedure, toe and ear punch reflexes can be monitored until full recovery of the animal. When euthanasia is required, animals can be sedated with an intraperitoneal injection of pentobarbital, and the level of sedation can again be monitored via toe and ear pinch reflexes. Once the surgical plane of anesthesia has been reached, cervical dislocation will be performed.

G. References
Adams, D. D., W. O. Lucas, B. G. Williams, B. B. Berkeley, K. W. Turner, and J. C. Schofield. 2001. A mouse genetic locus with death clock and life clock features. Mechanisms of Ageing and Development 122: 173-189.
Alvarez-Dolado, et al., Journal of Neuroscience 19, 6979-6993 (1999).
Andersson, K. E. and C. G. Stief. 1997. Neurotransmission and the contraction and relaxation of penile erectile tissues. World Journal of Urology 15: 14-20.
Andrew, M. & Owen, M. J., British Journal of Psychiatry 170, 106-108 (1997).
Argiolas, A. and M. R. Melis. 1998. The neuropharmacology of yawning. European Journal of Pharmacology. 343: 1-16.
Baker, J., J. P. Liu, E. J. Robertson, and A. Efstratiadis. 1993. Role of insulin-like growth factors in embryonic and postnatal growth. Cell 75: 73-82.
Bartanek, P., and Zenke, M. (1998) Mol. Endocrinol. 12, 1269-1279.
Bauer, A., Mikulits, W., Lagger, G., Stengl, G., Brosch, G., and Beug, H. (1998) EMBO J. 17, 4291-4303.
Beamer, W. G., Eicher, E. M., Maltais, L. J., and Southard, J. L. (1981) Science 212, 61-63
Beato, M. (1989) Cell 56,335-344
Beato, M., Herrlich, P., and Schutz, G. (1995) Cell 83, 851-857
Beuzard, Y. 1996. Transgenic mouse models of sickle cell disease. Current Opinion in Hematology 3: 150-155.
Bhat, G. K., V. B. Mahesh, C. A. Lamar, L. Ping, K. Aguan, and D. W. Brann. 1995. Histochemical localization of nitric oxide neurons in the hypothalamus: association with gonadotropin-releasing hormone neurons and co-localization with N-methyl-D-aspartate receptors. Neuroendocrinology 62: 187-197.
Bouchon, R., and Ropartz, P. (1990) Physiology and Behavior 48,501-505
Burbach, J. P., H. S. Van Schaick, C. Da Silva, H. J. Asbreuk, and M. P. Schmidt. 1998. Hypothalamic transcription factors and the regulation of the hypothalamo-neurohypophysial hormone. Advances in Experimental Medicine and Biology 449: 29-37.
Burnett, A. L. 1995. Nitric oxide in the penis: physiology and pathology. Journal of Urology 157: 320-324.
Burnett, A. L., R. J. Nelson, D. C. Calvin, J. X. Liu, G. E. Demas, S. L. Klein, L. J. Kriegsfeld, V. L. Dawson, T. M. Dawson, and S. H. Snyder. 1996. Nitric oxide-dependent penile erection in mice lacking neuronal nitric oxide synthase. Molecular Medicine 2: 288-296.
Carr, B. R. (1998) in Williams textbook of Endocrin6logy (Wilson, J. D., Foster, D. W., Kronenberg, H. M., and Larsen, P. R., eds), pp. 751-817, WB saunders Company, Philadelphia
Ceccatelli, S., A. L. Hutting, X. Zhang, L. Gustafsson, M. Villar, and T. Hokfelt. 1993. Nitric oxide in the rat anterior pituitary gland and the role of nitric oxide in regulation of luteinizing hormone secretion. Proceedings of the National Academy of Sciences USA 90: 11292-11296.
Chang, C., and H. J. Pan. 1998. Thyroid hormone direct repeat 4 response element is a positive regulatory element for the human TR2 orphan receptor, a member of steroid receptor superfamily. Mol Cell Biochem 189: 195-200.
Chang, C., and J. Kokontis. 1988. Identification of a new member of the steroid receptor super-family by cloning and sequence analysis. Biochem Biophys Res Commun 155: 971-7.
Chang, C., and Pan, H. (1998) Mol. Cell. Biochem. 189, 195-200
Chang, C., J. Kokontis, L. Acakpo-Satchivi, S. Liao, H. Takeda, and Y. Chang. 1989. Molecular cloning of new human TR2 receptors: a class of steroid receptor with multiple ligand-binding domains. Biochem Biophys Res Commun 165: 735-41.
Chang, C., Lee, H., and Lee, Y. (1997) Biochem. Biophys. Res. Commun. 235,205-211
Chang, C., S. L. Da Silva, R. Ideta, Y. Lee, S. Yeb, and J. P. Burbach. 1994. Human and rat TR4 orphan receptors specify a subclass of the steroid receptor superfamily. Proc Natl Acad Sci USA 91:6040-4.
Chen, J., et al.,. Development 125, 1943-1949 (1998).
Chen, W. S., K. Manova, D.C. Weinstein, S. A. Duncan, A. S. Plump, V. R. Prezioso, R. F. Bachvarova, and J. E. Darnell, Jr. 1994. Disruption of the HNF-4 gene, expressed in visceral endoderm, leads to cell death in embryonic ectoderm and impaired gastrulation of mouse embryos. Genes Dev 8:2466-77.
Chomez, P., et al., Development 127, 1489-1498 (2000).
Colin, F., R is, L. & Godaux, E. in The Cerebellum and its Disorders (eds. Manto, M. & Pandolfo, M.) 6-29 (Cambridge University Press, Cambridge, UK, 2002).
Collins, L. L., D. Lin, X. Mu, and C. Chang. 2000. Feedback regulation between the TR2 orphan receptor and the human papilloma virus type 16. Journal of Biological Chemistry 276: 27316-27321.
Coolen, L. M. and R. I. Wood. 1999. Testosterone stimulation of the medial preoptic area and medial amygdala in the control of male hamster sexual behavior: redundancy without amplification. Behavioural Brain Research 98: 143-153.
Cooney, A. J., C. T. Lee, S. Lin, S. Y. Tsai, and M. Tsai. 2001. Physiological function of the orphans GCNF and COUP-TF. Trends in Endocrinology and Metabolism 12: 247-251.
Couse, J. F., and Korach, K. S. (1999) Endocrine Reviews 20,358-417
Culling, C. F. A. (1963) Handbook of histopathogical techniques, Butterworths, Inc., Washington
De Vries, G. J. and P. A. Boyle. 1998. Double duty for sex differences in the brain. Behavioural Brain Research 92: 205-213.
DeChiara, T. M., R. Vejsada, W. T. Poueymirou, A. Acheson, C. Suri, J. C. Conover, B. Friedman, J. McClain, L. Pan, N. Stahl, et al. 1995. Mice lacking the CNTF receptor, unlike mice lacking CNTF, exhibit profound motor neuron deficits at birth. Cell 83:313-22.
Deconinck, A. E., Rafael, J. A., Skinner, J. A., Brown, S. C., Potter, A. C., Metzinger, L., Watt, D. J., Dickson, G., Tinsley, J. M., and Davies, K. E. (1997) Cell 901, 717-727
Delany, A. M., Amling, M., Priemel, M., Howe, C., Baron, R., and Canalis, E. (2000) J. Clin. Invest. 105,915-923
Donatucci, C. F. and T. F. Lue. 1993. Physiology of penile tumescence. In The Penis (ed. A.I. Hashmat and S. Das). Lea & Febiger, Malvern, Pa., U.S.A.
Donoso, A. 0., Banzan, A. M., and Borzino, M. I. (1976) J Endocrinol. 68, 171-172
Durand, B., Saunders, M., Gaudon, C., Roy, B., Losson, R., and Chambon, P. (1994) EMBO J. 13, 5370-5382
Eddy, E. M., T. F. Washburn, D. O. Bunch, E. H. Goulding, B. C. Gladen, D. B. Lubahn, and K. S. Korach. 1996a. Targeted disruption of the estrogen receptor gene in male mice causes alteration of spermatogenesis and infertility. Endocrinology 137: 4796-.
Enmark, E., and J. A. Gustafsson. 1996. Orphan nuclear receptors—the first eight years. Mol Endocrinol 10:1293-307.
Evans, R. M. 1988. The steroid and thyroid hormone receptor superfamily. Science 240:889-95.
Farhat, M., Herrlich, P., and Schutz, G. (1995) Biochem. Pharmacal. 51,571-576 10. Chang, C., and Kokontis, J. (1988) Biochem. Biophys. Res. Comm. 155,971-977
Fraichard, A., Chassande, 0., Plateroti, M., Roux, J. P., Trouillas, J., Dehay, C., Legrand, C., Gauthier, K., Kedinger, M., Malayal, L., Rousset, B., and Samarut, J. (1997) EMBO J. 16, 4412-4420
Franco, P. J., M. Farooqui, E. Seto, and L. N. Wei. 2001. The orphan nuclear receptor TR2 interacts directly with both class I and class II histone deacetylases. Mol Endocrinol 15:1318-28.
Freedman, L. P. Endocrine Reviews 13, 129-145 (1992).
Frenette, P. S., Mayadas, T. N., Rayburn, H., Hynes, R. O., and Wagner, D. D. (1996) Cell 84, 563-574.
Garrel, G., Y. Lerrant, C. Siriostis, A. Berault, S. Magre, C. Bouchaud, and R Counis. 1998. Evidence that gonadotropin-releasing hormone stimulates gene expression and levels of active nitric oxide synthase type I in pituitary gonadotrophs, a process altered by desensitization and, indirectly, by gonadal steroids. Endocrinology 139: 2163-2170.
Gimpl, G. and F. Fahrenholz. 2001. The oxytocin receptor system: structure, function, and regulation. Physiological Reviews 81: 629-683.
Godfrey, P., Rahal, J. 0., Beamer, W. G., Copeland, N. G., Jenkins, N. A., and Mayo, K. E. (1993) Nat. Genet. 4,227-232
Goldowitz, D. & Hamre, K., Trends in Neuroscience 21,375-382 (1998)
Gonzalez-Cadavid, N. F., A. L. Bumett, T. R. Magee, C. B. Zeller, D. Vemet, N. Smith, J. Gitter, and J. Rajfer. 2000. Expression of penile neuronal nitric oxide synthase variants in the rat and mouse penile nerves. Biology of Reproduction 63: 704-714.
Gothe, S., Z. Wang, L. Ng, J. M. Kindblom, A. C. Barros, C. Ohisson, B. Vennstrom, and D. Forrest. 1999. Mice devoid of all known thyroid hormone receptors are viable but exhibit disorders of the pituitary-thyroid axis, growth, and bone maturation. Genes and Development 13: 1329-1341.
Grady, R. M., Teng, H., Nichol, M. C., Cunningham, J. C., Wilkenson, R. S., and Sanes, J. R. (1997) Cell 90, 729-738
Gronmeyer, H., and Laudet, V. (1995) Protein Profile 2(11), 1173-1308
Guiochon-Mantel, A., Delabre, K., Lescop, P., and Milgrom, E. (1994) Proc. Natl. Acad. Sci. USA 91, 7179-7183
Guo, L., Sawkar, A., Zasadzki, M., Watterson, D. M., and Van Eldik, L. J. (2001) Neurobiol. Aging 22, 975-981
Hamilton, B. A., et al., Nature 379, 736-739 (1996).
Harada, H., Kuboi, Y., Miki, R., Honda, C., Masushige, S., Nakatsuka, M., Koga, Y., and Kato, S. (1998) Endocrinology 139,204-212
Harding, H. P. & Lazar, M. A., Molecular & Cellular Biology. 15, 4791-4802 (1995).
Harding, H. P., et al., Molecular Endocrinology 11, 1737-1746 (1997).
Hashmat, A.I. and J. Rehman. 1993. Priapism. In The Penis (ed. A.I. Hashmat and S. Das), pp. 219-243. Lea & Febiger, Malvern, Pa.
Hauri, D., M. Spycher, and W. Bruhlmann. 1983. Erection and priapism: A new physiopathological concept. Urol. Int. 88: 138-145.
Hill, S. J. (1990) Pharmacol. Rev. 42, 45-83
Hirose, T., W. Fujimoto, T. Yamaai, H. K. Kim, H. Matsuura, and A. M. Jetten. 1994. TAK1: Molecular cloning and characterization of a new member of the nuclear receptor superfamily. Molecular Endocrinology 8: 1667-1680.
Hogan, B., Beddington, R., Constntini, F., and Lacy, E. (1994) Manipulating the mouse embryo, second Ed., Cold Spring Harbor Laboratory Press, Cold Spring Harbor
Horie, K., Takakura, K., Fujiwara, H., Suginami, H., Liao, S., and Mori, T. (1992) Human Reproduction 7,184-190
Howell, D.C. in Statistical Methods for Psychology 198-205 (Duxbury, Pacific Grove, Calif., 2002).
Hwang, S., P. H. Burbach, and C. Chang. 1998. TR4 orphan receptor crosstalks to chicken ovalbumin upstream protein-transcription factor and thyroid hormone receptor to induce the transcriptional activity of the human immunodeficiency virus type I long-terminal repeat. Endocrine 8: 169-175.
Ignarro, L. J. and A. Jacobs. 2000. Nitric oxide synthase and the production of nitric oxide. In Functional Neuroanatomy of the Nitric Oxide System (ed. H. W. M. Steinbusch, J. De Vente, and S. R. Vincent), pp. 1-17. Elsevier Science B.V., Amsterdam.
Ingleton, P. M., Rodgers, M. F., and Parsons, M. A. (1992) Exp. Mol. Pathol. 56, 119-131
Ingraham, H. A., D. S. Lala, Y. Ikeda, X. Luo, W. H. Shen, M. W. Nachtigal, R. Abbud, J. H. Nilson, and K. L. Parker. 1994. The nuclear receptor steroidogenic factor 1 acts at multiple levels of the reproductive axis. Genes Dev 8:2302-12.
Jarvis, C. I., et al., Molecular and Cellular Endocrinology 186, 1-5 (2002).
Kaneshige, M., K. Kaneshige, X. Zhu, A. Dace, L. Garrett, T. A. Carter, R. Kazlauskaite, D. G. Pankratz, A. Wynshaw-Boris, S. Refetoff, B. Weintraub, M. C. Willingham, C. Barlow, and S. Cheng. 2000. Mice with a targeted mutation in the thyroid hormone b receptor gene exhibit impaired growth and resistance to thyroid hormone. Proceedings of the National Academy of Sciences USA 97: 13209-13214.
Kendall, S. K., L. C. Samuelson, T. L. Saunders, R. I. Wood, and S. A. Camper. 1995. Targeted disruption of the pituitary glycoprotein hormone a-subunit produces hypogonadal and hypothyroid mice. Genes and Development 9: 2007-2019.
Kishimoto, J., T. Tsuchiya, P.C. Emson, and Y. Nakayama. 1996. Immobilization-induced stress activates neuronal nitric oxide synthase (nNOS) mRNA and protein in hypothalamic-pituitary-adrenal axis in rats. Brain Research 720: 159-171.
Klockgether, T. & Evert, B., Trends in Neuroscience 21, 413-418 (1998).
Korach. 1996b. Targeted disruption of the estrogen receptor in male mice causes alteration of spermatogenesis and infertility. Endocrinology 137: 4796-4805.
Koritschoner, N. P., Madruga, J., Knespel, S., Blendinger, G., Anzinger, B., Otto, A., Zenke, M., and Bartunek, P. (2001) Cell Growth Differ. 12,563-572.
Krantz, S. B. (1991) Blood 77,419-434. 29. Lee, H., Lee, Y., and Chang, C. (1999) Mol. Cell. Biochem. 194, 199-207 30.
Kriegsfeld, L. J., G. E. Demas, P. L. Huang, A. L. Burnett, and R. J. Nelson. 1999. Ejaculatory abnormalities in mice lacking the gene for endothelial nitric oxide synthase (eNOS-/). Physiology and Behavior 67: 561-566.
Kuang, W., Xu, H., Vachon, P. H., Liu, L., Loechel, F., Wewer, U. M., and Engvall, E. (1998)J. Clin. Invest. 102, 844-852
Labosky, P. A., G. E. Winnier, T. L. Jetton, L. Hargett, A. K. Ryan, M. G. Rosenfeld, A. F. Parlow, and B. L. M. Hogan. 1997. The winged helix gene, Mf3, is required for normal development of the diencephalon and midbrain, postnatal growth and the milk-ejection reflex. Development 124: 1263-1274.
Laudet, V. (1997) J. Mol. Endocrinol. 19,207-226
Le, Y., and Sauer, B. (2000) Methods Mol Bioi 136, 477-485
Lecanda, F., Warlow, P. M., Sheikh, S., Furlan, F., Steinberg, T. H., and Civitelli, R. (2000) J. Cell. Biol. 151, 931-943
Lee, C. H., L. Chang, and L. N. Wei. 1996. Molecular cloning and characterization of a mouse nuclear orphan receptor expressed in embryos and testes. Mol Reprod Dev 44:305-14.
Lee, C. H., N. G. Copeland, D. J. Gilbert, N. A. Jenkins, and L. N. Wei. 1995. Genomic structure, promoter identification, and chromosomal mapping of a mouse nuclear orphan receptor expressed in embryos and adult testes. Genomics 30:46-52.
Lee, C. H., C. Chinpaisal, and L. N. Wei. 1998a. A novel nuclear receptor heterodimerization pathway mediated by orphan receptors TR2 and TR4. Journal of Biological Chemistry 273: 25209-25215.
Lee, H. J., Young, W. J., Shih, C. C. Y., and Chang, C. (1996) J. Biol. Chem. 271, 10405-10412
Lee, H., and Chang, C. (1995)J. Biol. Chem. 270, 5434-5440
Lee, H., et al., Journal of Biological Chemistry 270, 30129-30133 (1995).
Lee, H., Y. Lee, P. Burbach, and C. Chang. 1995. Suppression of gene expression on the SV40 major late promoter by TR4 orphan receptors: a member of the steroid receptor superfamily. Journal of Biological Chemistry 270: 30129-30133.
Lee, S., C. K. Kim, and C. Rivier. 1999a. Nitric oxide stimulates ACTH secretion and the transcription of the genes encoding for NGFI-B, corticotropin-releasing factor type 1, and vasopressin in the hypothalamus of the intact rat. Journal of Neuroscience 19: 7640-7647.
Lee, V. W. K., D. M. de Kretser, B. Hudson, and C. Wang. 1975. Variations in serum FSH, LH, and testosterone levels in male rats from birth to sexual maturity. Journal of Reproduction and Fertility 42: 121-126.
Lee, Y. F., et al., Journal of Steroid Biochemistry & Molecular Biology 81, 291-308 (2002).
Lee, Y. F., H. J. Pan, J. P. Burbach, E. Morkin, and C. Chang. 1997. Identification of direct repeat 4 as a positive regulatory element for the human TR4 orphan receptor. A modulator for the thyroid hormone target genes. J Biol Chem 272:12215-20.
Lee, Y. F., W. J. Young, J. P. Burbach, and C. Chang. 1998. Negative feedback control of the retinoid-retinoic acid/retinoid X receptor pathway by the human TR4 orphan receptor, a member of the steroid receptor superfamily. J Biol Chem 273:13437-43.
Lee, Y. F., W. J. Young, W. J. Lin, C. R. Shyr, and C. Chang. 1999. Differential regulation of direct repeat 3 vitamin D3 and direct repeat 4 thyroid hormone signaling pathways by the human TR4 orphan receptor. J Biol Chem 274:16198-205.
Lee, Y.-F., C. R Shyr, T. H. Thin, W. J. Lin, and C. Chang. 1999b. Convergence of two repressors through heterodimer formation of androgen receptor and testicular orphan receptor-4: a unique signaling pathway in the steroid receptor superfamily. Proceedings of the National Academy of Sciences USA 96: 14724-14729.
Lee, Y. F., H.J. Lee, and C. Chang. 2002. Recent advances in the TR2 and TR4 orphan receptors of the nuclear receptor superfamily. Journal of Steroid Biochemistry & Molecular Biology 81: 291-308.
Li, S., E. B. I. Crenshaw, E. J. Rawson, D. M. Soimmons, L. W. Swanson, and M. G. Rosenfeld. 1990. Dwarf locus mutants lacking three pituitary cell types result from mutations in the POU-domain gene pit-1. Nature 347: 528-533.
Lin, D. L., S. Q. Wu, and C. Chang. 1998. The genomic structure and chromosomal location of the human TR2 orphan receptor, a member of the steroid receptor superfamily. Endocrine 8:123-34
Lin, S., C. R. Lin, I. Gukovsky, A. J. Lusis, P. E. Sawchenko, and M. G. Rosenfeld. 1993. Molecular basis of the little mouse phenotype and implications for cell type-specific growth. Nature 364: 208-213.
Lin, T. M., W. J. Young, and C. Chang. 1995. Multiple functions of the TR2-11 orphan receptor in modulating activation of two key cis-acting elements involved in the retinoic acid signal transduction system. J Biol Chem 270:30121-8.
Baker, J., Liu, J. P., Robertson, E. J., and Efstratiadis, A. (1993) Cell 75, 73-82
Liu, J. P., J. Baker, A. S. Perkins, E. J. Robertson, and A. Efstratiadis. 1993. Mice carrying null mutations of the genes encoding insulin-like growth factor 1 (IGF-1) and type 1 IGR receptor (Igf1r). Cell 75: 59-72.
Livak, K. J. and T. D. Schmittgen. 2001. Analysis of relative gene expression data using real-time quantitative PCR and the 2-DDCT method. Methods 25: 402-408.
Lohnes, D., Kastner, P., Dierich, A., Mark, M., LeMeur, M., and Chambon, P. (1993) Cell 73, 643-658
Lubahn, D. B., J. S. Moyer, T. S. Golding, J. F. Couse, K. S. Korach, and O. Smithies. 1993. Alteration of reproductive function but not prenatal sexual development after insertional disruption of the mouse estrogen receptor gene. Proceedings of the National Academy of Sciences 90: 11162-11166.
Luo, X., Ikeda, Y., and Parker, K. L. (1994) Cell 74, 481-490
Mangelsdorf, D. J., C. Thummel, M. Beato, P. Herrlich, G. Schutz, K. Umesono, B. Blumberg, P. Kastner, M. Mark, P. Chambon, and et al. 1995. The nuclear receptor superfamily: the second decade. Cell 83:835-9.
Mangelsdorf, D. J., Thummel, C., Beato, M., Herrlich, P., Schutz, G., Umesono, K., Blumberg, B., Kastner, P., Mark, M., Chambon, P., and Evans, R. M. (1995) Cell 83, 835-839 3. Freedman, L. P. (1992) Endocrine Reviews 13, 129-145
Manto, M. in The Cerebellum and its Disorders (eds. Manto, M. & Pandolfo, M.) 1-5 (Cambridge University Press, Cambridge, UK, 2002).
Marin, R., A. Escrig, P. Abreu, and M. Mas. 1999. Androgen-dependent nitric oxide release in rat penis correlates with levels of constitutive nitric oxide synthase isozymes. Biology of Reproduction 61: 1012-1016.
Masu, Y., E. Wolf, B. Holtmann, M. Sendtner, G. Brem, and H. Thoenen. 1993. Disruption of the CNTF gene results in motor neuron degeneration. Nature 365:27-32.
McCarthy, M. M. 1995. Estrogen modulation of oxytocin and its relation to behavior. Advances in Experimental Medicine and Biology 395: 235-245.
McCarthy, M. M., C. H. McDonald, P. J. Brooks, and D. Goldman. 1996. An anxiolytic action of oxytocin is enhanced by estrogen in the mouse. Physiology and Behavior 60: 1209-1215.
McGinnis, M. Y., G. W. Williams, and A. R Lumia. 1996. Inhibition of male sex behavior by androgen receptor blockade in preoptic area or hypothalamus, but not amygdala or septum. Physiology and Behavior 60: 783-789.
McGinnis, M. Y., R. C. Montana, and A. R. Lumia. 2002. Effects of hydroxyflutamide in the medial preoptic area or lateral septum on reproductive behaviors in male rats. Brain Research Bulletin 59: 227-234.
McKenna, K. E. 2000. Some proposals regarding the organization of the central nervous system. Neuroscience and Biobehavioral Reviews 24: 535-540.
Meshul, C. K., et al., Brain Research 648, 181-195 (1994).
Miyagoe, Y., Hanaoka, K., Nonaka, I., Hayasaka, M., Nabeshima, Y., Arahata, K., Nabeshima, Y., and Takeda, S. (1997) FEBS Lett. 415, 33-39
Morris, R. G. M., Garrud, P., Rawlins, J. N. P., and O'Keefe, J. (1982) Nature 297,681-683
Mu, X., Y. Liu, L. L. Collins, E. Kim, and C. Chang. 2000. The p53/retinoblastoma-mediated repression of testicular orphan receptor-2 in the Rhesus monkey with cryptorchidism. Journal of Biological Chemistry 275: 23877-23883.
Mulac-Jericevic, B., Mullinax, R. A., DeMayo, F. J., Lydon, J. P., and Conneely, O. M. (2000) Science 289, 1751-1754.
Mundos, S. (1998) Methods in Molecular Biology 136, 61-70
Murty, V. V. V. S., Houldsworth, J., Balswin, S., REuter, V., Hunziker, W., Besmer, P., Bosl, G., and Chaganti, R. S. K. (1992) FroG. Natl. Acad. Sci. USA 89, 11006-11010
Murty, V. V. V. S., Renault, B., Falk, C. T., Bosl, G., Kucherlapati, R., and Changanti, R. S. K. (1996) Genomics 35, 562-570
Nelson, R. J., L. J. Kriegsfeld, V. L. Dawson, and T. M. Dawson. 1997. Effects of nitric oxide on neuroendocrine function and behavior. Frontiers in Neuroendocrinology 18: 463-491.
Nieschlag E. and H. M. Behre The role of testosterone in spermatogenesis. In Testosterone: action, deficiency, substitution (ed. E. Nieschlag and H. M. Behre), pp. 143-168. Springer-Verlag, Heidelberg. 1998.
Off, E. L., and Quay, W. B. (1975) Endocrinology 96,941-945 33. Sawyer, C. H. (1955) Am. J. Physiol. 180, 37-46
Ogawa, S., D. B. Lubahn, K. S. Korach, and D. W. Pfaff. 1997. Behavioral effects of estrogen receptor gene disruption in male mice. Proceedings of the National Academy of Sciences USA 94: 1476-1481.
Ogawa, S., J. Chan, A. E. Chester, J. Gustafsson, K. K. S., and D. W. Pfaff. 1999. Survival of reproductive behaviors in estrogen receptor b gene-deficient (bERKO) male and female mice. Proceedings of the National Academy of Sciences USA 96: 12887-12892.
Ostrowski, N. L. 1998. Oxytocin receptor mRNA expression in rat brain: implications for behavioral integration and reproductive success. Psychoneuroendocrinology 23: 989-1004.
Panteleo-Gandais, M., R. Chalbaud, O. Chacon, and N. Plaza. 1984. Priapism: evaluation and treatment. Urology 24: 345-346.
Pereira, F. A., Y. Qiu, G. Zhou, M. J. Tsai, and S. Y. Tsai. 1999. The orphan nuclear receptor COUP-TFII is required for angiogenesis and heart development. Genes Dev 13:1037-49.
Phend, K. D., et al., Journal of Histochemistry and Cytochemistry 40, 1011-1020 (1992).
Qiu, Y., F. A. Pereira, F. J. DeMayo, J. P. Lydon, S. Y. Tsai, and M. J. Tsai. 1997. Null mutation of mCOUP-TFI results in defects in morphogenesis of the glossopharyngeal ganglion, axonal projection, and arborization. Genes Dev 11: 1925-37.
Qiu, Y., F. A. Pereira, F. J. DeMayo, J. P. Lydon, S. Y. Tsai, and M. J. Tsai. 1997. Null mutation of mCOUP-TFI results in defects in morphogenesis of the glossopharyngeal ganglion, axonal projection, and arborization. Genes and Development 11: 1925-1937.
Rabinowicz, T., et al., Journal of Child Neurology 14, 98-107 (1999).
Rakic, P. & Sidman, R. L., Proceedings of the National Academy of Sciences USA 70, 240-244 (1973).
Rampin, O. and F. Guiliano. 2001. Brain control of penile erection. World Journal of Urology 19: 1-8.
Richman, C., Baylink, D. J., Lang, K., Dony, C., and Mohan, S. (1999) Endocrinology 140, 4699-4705
Romeo, R. D., E. Cook-Wiens, H. N. Richardson, and C. L. Sisk. 2001. Dihydrotestosterone activates sexual behavior in adult male hamsters but not in juveniles. Physiology and Behavior 73: 579-584.
Roosen-Runge, E. C. 1962. The process of spermatogenesis in mammals. Biological Review 37: 343-377.
Ruffin, M., and Nicolaidis, S. (1999) Brain. Res. 846, 23-29
Sambrook, J., Fritsch, E. F., and Maniatis, T. (1989) Molecular Cloning: A Laboratory Manual, 2 Ed., 3, Cold Spring Harbor Laboratory Press, Cold Spring Harbor, N.Y. Schrader, M., et al., Journal of Biological Chemistry 271, 19732-19736 (1996).
Schwartz, N. B., Szabo, M., Verina, T., Wei, J., Dlouhy, S. R., Won, L., Heller, A., Hodes, M. E., and Ghetti, B. (1998) Neuroendocrinology 68,374-385
Shyr, C., L. L. Collins, X. Mu, K. A. Platt, and C. Chang. 2002a. Spermatogenesis and testis development are normal in mice lacking testicular orphan nuclear receptor 2. Molecular and Cellular Biology 22: 4661-4666.
Shyr, C., Y. Hu, E. Kim, and C. Chang. 2002b. Modulation of estrogen receptor-mediated transactivation by orphan receptor TR4 in MCF-7 cells. Journal of Biological Chemistry 277: 14622-14628.
Sidman, R. L., et al., Science 136, 610-612 (1962).
Simon, E. S. et al., Movement Disorders 12, 221-228 (1997).
Simonsen, U., D. Prieto, J. A. Delgado, M. Hernandez, L. Resel, I. Saenz de Tejada, and A. Garcia-Sacristan. 1997. Nitric oxide is involved in the inhibitory neurotransrnission and endothelium-dependent relaxations of human small penile arteries. Clinical Science 92: 269-275.
Singh, J. and D. J. Handelsman. 1996. Neonatal administration of FSH increases Sertoli cell numbers and spermatogenesis in gonadotropin-deficient (hpg) mice. Journal of Endocrinology 151: 37-48.
Skoglund, T. S., et al., Neuroscience Letters 208, 97-100 (1996).
Slabaugh, M. B., Lieberman, M. E., Rutledge, J. J., and Gorsky, J. (1981) Endocrinology 109, 1040-1046
Smith, C. A., D. G. DeVera, D. J. Lamb, Z. Nawaz, Y. H. Jiang, A. L. Beaudet, and B. W. O'Malley. 2002. Genetic ablation of the steroid receptor coactivator-ubiquitin ligase, E6-AP, results in tissue-selective steroid hormone resistance and defects in reproduction. Molecular & Cellular Biology. 22: 525-535.
Smock, T., D. Albeck, and P. Stark. 1998. A peptidergic basis for sexual behavior in mammals. Progress in Brain Research 119: 467-481
Somsen, M. W., W. Wu, J. S. Dasen, S. E. Flynn, D. J. Norman, S. M. O'Connell, I. Gukovsky, C. Carriere, A. K. Ryan, A. P. Miller, L. Zuo, A. S. Gleiberman, B. Andersen, W. Beamer, and M. G. Rosenfeld. 1996. Pituitary lineage determination by the Prophet of Pit-1 homeodomain factor defective in Ames dwarfism. Nature 384: 327-333.
Stec, D. E., DAvisson, R. L., Haskell, R. E., Davidson, B. L., and Sigmund, C. D. (1999) J Bioi. Chern. 274, 21285-21290
Steers, W. D. 2000. Neural pathways and central sites involved in penile erection: neuroanatomy and clinical implications. Neuroscience and Biobehavioral Reviews 24: 507-516.
Steinlein, P., Wessely, 0., Meyer, S., Deiner, E. M., Hayman, M. J., and Beug, H. (1995) Curro Bioi. 5, 191-204.
Steinmayr, M., E. Andre, F. Conquet, L. Rondi-Reig, N. Delhaye-Bouchaud, N. Auclair, H. Daniel, F. Crepel, J. Mariani, C. Sotelo, and M. Becker-Andre. 1998. Staggerer phenotype in retinoid-related orphan receptor a-deficient mice. Proceedings of the National Academy of Sciences USA 95: 3960-3965.
Strouboulis, J., et al., Genes and Development 6, 1857-1864 (1992).
Tanabe, O., F. Katsuoka, A. D. Campbell, W. Song, M. Yamamoto, K. Tanimoto, and J. D. Engel. 2002. An embryonic/fetal b-type globin gene repressor contains a nuclear receptor TR2/TR4 heterodimer. The EMBO Journal 21: 3434-3442.
Trudel, M., M. E. De Paepe, N. Chretien, N. Saadane, J. Jacmain, M. Sorette, T. Hoang, and Y. Beuzard. 1994. Sickle cell disease of transgenic SAD mice. Blood 84: 3189-3197.
Tusher, V. G., Tibshirani, R., and Chu, G. (2001) Proc. Natl. Acad. Sci. USA 98, 5116-5121
Uno, H., Lohmiller, L., Thieme, C., Kemnitz, J. W., Engle, M. J., Roecker, E. B., and Farrell, P. M. (1990) Dev. Brain Res. 53, 157-167
Uvnas, M. K., S. Ahlenius, V. Hillegaart, and P. Alster. 1994. High doses of oxytocin cause sedation and low doses cause an anxiolytic-like effect in male rats. Pharmacological Biochemistry and Behavior 49: 101-106.
Vidal, 0., Lindberg, M. K., Hollberg, K., Baylink, D. J., Andersson, G., Lubahn, D. B., Mohan, S., Gustafsson, J.-A., and Ohlsson, C. (2000) Proc. Natl. Acad. Sci. USA 97, 5474-5479
Voogd, J. & Glickstein, M., Trends in Neuroscience 21, 370-375 (1998).
Voss, J. W. and M. G. Rosenfeld. 1992. Anterior pituitary development: short tales from dwarf mice. Cell 70: 527-530.
Wang, T. Y., Brennan, J. K., and Wu, J. H. (1995) Exp. Hematol. 23, 26-32.
Wattler, S., M. Kelly, and M. Nehls. 1999. Construction of gene targeting vectors from lambda KOS genomic libraries. BioTechniques 26: 1150-1156, 1158, 1160.
Wei, X., M. Sasaki, H. Huang, V. L. Dawson, and T. M. Dawson. 2002. The orphan nuclear receptor, Steroidogenic Factor 1, regulates neuronal Nitric Oxide Synthase gene expression in pituitary gonadotropes. Molecular Endocrinology 16: 2828-2839.
Weinbauer, G. F. and E. Nieschlag. 1993. Hormonal control of spermatogenesis. In Molecular Biology of the Male Reproductive System (ed. D. de Kretser), pp. 101-142.
Wessely, 0., Deiner, E. M., Beug, H., and von Lindern, M. (1997) EMBO J. 16,267-280.
Windle, R. J., N. Shanks, S. L. Lightman, and C. D. Ingram. 1997. Central oxytocin administration reduces stress-induced corticosterone release and anxiety behavior in rats. Endocrinology 138: 2829-2834.
Winter, C. C. 1978. Priapism. Urological Surgery 28: 163.
Witt, D. M. 1995. Oxytocin and rodent sociosexual responses: from behavior to gene expression. Neuroscience and Biobehavioral Reviews 19: 315-324.
Wu, T. J., Silverman, A. J., and Gibson, M. J. (1996) J. Neurosci. 31, 67-76
Xu, J., L. Liao, G. Ning, H. Yoshida-Komiya, C. Deng, and B. W. O'Malley. 2000. The steroid receptor coactivator SRC-3 (p/CIP/RAC-3/AIB1/ACTR/TRAM-1) is required for normal growth, puberty, female reproductive function, and mammary gland development. Proceedings of the National Academy of Sciences USA 97: 6379-6384.
Xu, X., Wagner, K-U., Larson, D., Weaver, Z., Li, C., Ried, T., Henninghausen, L., Wynshaw-Boris, A., and Deng, C.-X. (1999) Nat. Genet. 22, 37-43
Yamaguchi, M., Nakamoto, M., Honda, H., Nakagawa, T., Fujita, H., Nakamura, T., Hirai, H., Narumiya, S., and Kakizuka, A. (1998) Proc. Natl. Acad. Sci. USA 95, 7491-7496
Yamashita, M., Fuki, H., Sugama, K., Horio, Y., Ito, S., Mizaguchi, H., and Wada, H. (1991) FroG. Natl. A cad. Sci. USA 88, 11515-11519
Yeh, S., M. Tsai, Q. Xu, X. Mu, H. Lardy, K. Huang, H. Lin, S. Yeh, S. Altuwaijri, X. Zhou, L. Xing, B.F. Boyce, M. Hung, S. Zhang, L. Gan, and C. Chang. 2002. Generation and characterization of androgen receptor knockout (ARKO) mice: an in vivo model for the study of androgen functions in selective tissues. Proceedings of the National Academy of Sciences USA 99: 13498-13503.
Yoshizawa, T., Handa, Y., Uematsu, Y., Takeda, S., Sekine, K., Yoshihara, Y., Kawakami, T., Arioka, K., Sato, H., Uchiyama, Y., Masushige, S., Fukamizu, A., Matsumoto, T., and Kato, S. (1997) Nat. Genet. 16, 391-396 I
Young, W. J., S. M. Smith, and C. Chang. 1997. Induction of the intronic enhancer of the human ciliary neurotrophic factor receptor (CNTFRalpha) gene by the TR4 orphan receptor. A member of steroid receptor superfamily. J Biol Chem 272:3109-16.
Young, W. J., Y. F. Lee, S. M. Smith, and C. Chang. 1998. A bidirectional regulation between the TR2/TR4 orphan receptors (TR2/TR4) and the ciliary neurotrophic factor (CNTF) signaling pathway. J Biol Chem 273:20877-85.
Yun, H. Y., V. L. Dawson, and T. M. Dawson. 1996. Neurobiology of nitric oxide. Critical Reviews in Neurobiology 10: 291-316.
Zhang, Y. and M. L. Dufau. 2000. Nuclear orphan receptors regulate transcription of the gene for the human luteinizing hormone receptor. Journal of Biological Chemistry 275: 2763-2770.

H. Sequences
1. SEQ ID NO:1 Human TR2 Protein Genbank accession number P13056.
2. SEQ ID NO₂ Genbank Accession no. A36738. orphan receptor TR2, splice form TR2-9-human.
3. SEQ ID NO:3 Genbank Accession No. B36738 orphan receptor TR2, splice form TR2-11-human.
4. SEQ ID NO:4 Genbank Accession No. A31521. orphan receptor TR2, splice form TR2-5-human.
5. SEQ ID NO:5 Genbank Accession No. 154075. gene mTR2R1 protein-mouse
6.:SEQ ID NO:6 Genbank Accession No. M29959. Human steroid receptor (TR2-9) Protein.
7. SEQ ID NO:7 Genbank Accession No. M29959. Human steroid receptor (TR2-9) mRNA, complete cds. Encodes protein designated M29959 herein.
8. SEQ ID NO:8 Genbank Accession No. M29960. Human steroid receptor (TR2-11) protein
9. SEQ ID NO:9 Genbank Accession No. M29960. Human steroid receptor (TR2-11) mRNA, complete cds encoding protein M299960 disclosed herein
10. SEQ ID NO:10 Genbank Accession No. M21985. Human steroid receptor TR2 protein.
11. SEQ ID NO:11 Genbank Accession No. M21985. Human steroid receptor TR2 mRNA, complete cds encodes protein disclosed herein as M21985
12. SEQ ID NO:12 Genbank Accession No P49116. Human Orphan nuclear receptor TR4 (Orphan nuclear receptor TAK1)
13. SEQ ID NO:13 TR4/TR2 binding site
14. (SEQ ID NO:14) Primer for wild type TR2
15. (SEQ ID NO15) Primer for wild type TR2
16. (SEQ ID NO:16) Primers for mutant TR2
17. (SEQ ID NO:17) Primers for mutant TR2
18. SEQ ID NO:18 Primer for making TR2 knockout
19. SEQ ID NO:19 Primer for making TR2 knockout
20. SEQ ID NO:20 Primer for making TR2 knockout
21. SEQ ID NO:21 Primer for making TR2 knockout
22. SEQ ID NO:22 Primer for Wildtype TR4
23. SEQ ID NO:23 Primer for Wildtype TR4
24. SEQ ID NO:24 Primer for mutant TR4
25. SEQ ID NO:25 Primer for mutant TR4
26. SEQ ID NO:26 TR4-107 TR4 genotype primer (WT, forward)
27. SEQ ID NO:27 TR4-111 TR4 genotype primer (WT, reverse)
28. SEQ ID NO:28 Neo-3a TR4 genotype primer (TR4KO, forward)
29. SEQ ID NO:29 TR4-34 TR4 genotype primer (TR4KO, reverse)
30. SEQ ID NO: 30 mTR4(S)
31. SEQ ID NO:31 mTR4(AS) product size: 137 bp
32. SEQ ID NO:32 mTR2(S)
33. SEQ ID NO:33 mTR2(AS) product size: 100 bp
34. SEQ ID NO:34 β-actin(S)
35. SEQ ID NO:35 β-actin(AS) product size: 448 bp
36. SEQ ID NO: 36 hAR(S)
37. SEQ ID NO:37 hAR(AS) product size: 88 bp
38. SEQ ID NO:38 mERα(S)
39. SEQ ID NO:39 mERα(AS) product size: 119 bp
40. SEQ ID NO:40 mERβ(S)
41. SEQ ID NO:41 mERβ(AS) product size: 118 bp
42. SEQ ID NO:42 mVP (S):
43. SEQ ID NO:43 mVP(AS) product size: 119 bp
44. SEQ ID NO:44 mOT (S)
45. SEQ ID NO:45 mOT(AS): product size: 143 bp
46. SEQ ID NO:46 nNOS-NHR, bp-198 to -211
47. SEQ ID NO:47 Genbank Accession No P49116. Human Orphan nuclear receptor TR4 (Orphan nuclear receptor TAK1) DNA

What is claimed is:

1. A transgenic animal, comprising a disrupted TR2 or TR4 gene.

2. The transgenic animal of claim 1, wherein the animal is a mammal.

3. The mammal of claim 2, wherein the mammal is a murine.

4. The murine of claim 3, wherein the murine is a mouse.

5. The mouse of claim 4, wherein the disrupted TR2 or TR4 gene encodes a non-functional TR2 or TR4 protein.

6. The mouse of claim 5, wherein the TR2 or TR4 gene comprises a deleted exon.

7. The mouse of claim 6, wherein the exon is exon 3, 4, 5, 6, or 7.

8. The mouse of claim 6, wherein the exon is exon 4.

9. The mouse of claim 6, wherein the exon is exon 5.

10. The mouse of claim 5, wherein the TR2 or TR4 gene comprises a point mutation.

11. The mouse of claim 5, wherein the TR2 or TR4 gene comprises a missense mutation.

12. The mouse of claim 5, wherein the the TR2 or TR4 gene further comprises a marker gene.

13. The mouse of claim 12, wherein the marker gene is a lacZ gene.

14. The mouse of claim 5, wherein the TR2 or TR4 gene further comprises a loxP site.

15. The mouse of claim 14, wherein the TR2 or TR4 gene further comprises a second loxP site.

16. The mouse of claim 5, wherein the TR2 or TR4 gene further comprises recombinase sites.

17. The mouse of claim 16, wherein the recombinase sites flank all of a TR2 or TR4 exon.

18. The mouse of claim 5, further comprising a nucleic acid encoding a recombinase and operably linked to a promoter.

19. The mouse of claim 18, wherein the recombinase is a Cre or Flp recombinase.

20. The mouse of claim 18, wherein the recombinase is under the control of an induclble promoter.

21. The mouse of claim 18, wherein the promoter is a tissue specific promoter.

22. The mouse of claim 18, wherein the reporter is a constitutive promoter.

23. A transgenic animal cell, comprising a disrupted TR2 or TR4 or TR4 gene.

24. The transgenic animal cell of claim 23, wherein the animal cell is a mammal cell.

25. The mammal cell of claim 24, wherein the mammal cell is a murine cell.

26. The murine cell of claim 25, wherein the murine cell is a mouse cell.

27. The mouse cell of claim 26, wherein the disrupted TR2 or TR4 gene encodes a non-functional TR2 or TR4 protein.

28. The mouse of claim 27, wherein the TR2 or TR4 gene comprises a deleted exon.

29. The mouse cell of claim 26, wherein the cell comprises an embryonic stem cell or an embryonic germ cell.

30. The mouse cell of claim 26, wherein the cell comprises an immortal cell line.

31. The mouse cell of claim 26, wherein the cell comprises a breast cell, a breast cancer cell, an ovary cell, or an ovary cancer cell.

32. The mouse cell of claim 26, wherein the cell comprises a cell wherein TR2 or TR4 is expressed.

33. The mouse cell of claim 26, wherein the cell comprises a prostate cell, testis cell, bone cell, brain cell, or muscle cell.

34. A vector, the vector comprising a portion of the TR2 or TR4 gene, wherein the portion of the TR2 or TR4 gene produce a disrupted TR2 or TR4 gene, and wherein the vector can homologously recombine with the TR2 or TR4 gene.

35. A vector comprising a region 1 for homologous recombination with a region of the TR2 or TR4 gene, and a region of an exon of the TR2 or TR4 gene, and a region 2 for homologous recombination.

36. A vector comprising a region 1 for homologous recombination with a region of the TR2 or TR4 gene, and a region of an exon of the TR2 or TR4 gene, a region encoding a selectable marker, and a region 2 for homologous recombination.

37. The vector of claim 36, wherein the region 1 can homologously recombine with intron an intron.

38. The vector of claim 37, wherein the intron is intron 1, 2, 3, 4, 5, 6, or 7 or the TR2 or TR4 gene.

39. The vector of claim 37, wherein the intron is inton 3 of the TR2 or TR4 gene.

40. The vector of claim 36, wherein the exon is exon 3, 4, 5, 6, or 7.

41. The vector of claim 36, wherein the exon is exon 4.

42. The vector of claim 36, wherein the exon is exon 5.

43. The vector of claim 36, wherein the region 1 comprises 300 nucleotides.

44. The vector of claim 36, wherein the region 1 comprises 750 nucleotides.

45. The vector of claim 36, wherein the region 1 comprises 1000 nucleotides.

46. The vector of claim 36, wherein the region 1 comprises 1100 nucleotides.

47. The vector of claim 36, wherein the homologous recombination region 1 and region 2 comprise sequence that has at least 70% homology to a region of the TR2 or TR4 gene.

48. The vector of claim 34, further comprising a selectable marker.

49. The vector of claim 48, wherein the marker is a neo marker.

50. The vector of claim 48, wherein the marker is positive selection marker.

51. The vector of claim 48, wherein the marker is a negative selection marker.

52. A cell comprising the vector of claim 36.

53. An animal comprising the cell of claim 52.

54. An animal comprising the vector of claim 36.

55. A nucleic acid molecule produced by the process, the process comprising linking in an operative way a nucleic acid comprising the sequence of a TR2 or TR4 exon and sequence recognized by a recombinase enzyme.

56. A cell produced by the process of transforming the cell with the nucleic acid of claim 55.

57. A method of determining the effect of steroids on a cell comprising, administering a steroid to a cell comprising a disrupted TR2 or TR4 gene.

58. A method of identifying a gene regulated by TR4 comprising performing a microarray gene expression analysis of a TR4 knockout mouse obtaining an expression analysis, comparing the expression analysis to a microarray gene expression analysis of a wildtype mouse, comparing the expression analysis to a nicroarray expression analysis of a TR2 or TR4 mouse, and identifying the genes in the TR4 mouse that are regulated differently than the wildtype mouse and the TR2 or TR4 mouse.

59. A method of making a mouse, comprising breeding the mouse of claim 4 with a second mouse.

60. The method of claim 59, wherein the second mouse is also a mouse of claim 4.

61. A method of drug discovery comprising administering a candidate drug to the mouse of claim 4.

62. A method of producing an animal, the method comprising administering the vector of claim 36 to an ES cell, culturing the cell, selecting a cell comprising the vector, fusing the selected cell with a blastocyst, and allowing the fused blastocyst to produce a live birth, forming a chimera.

63. A method producing an animal, the method comprising fusing the chimera of claim 61, with another chimera, and selecting live animals homozygous for vector DNA.
